(19) United States
(12) Patent Application Publication
(10) Pub. No.: US 2025/0034269 A1
(43) Pub. Date: Jan. 30, 2025

(54) AGONISTIC LTBR ANTIBODIES AND BISPECIFIC ANTIBODIES COMPRISING THEM

(71) Applicant: Hoffmann-La Roche Inc., Little Falls, NJ (US)

(72) Inventors: Roberta Bianchi, Zurich (CH); Felix Bormann, Bichl (DE); Michelle Victoria Brydon, Dietikon (CH); Stefan Dengl, Geretsried (DE); Harald Duerr, Starnberg (DE); Guy Georges, Habach (DE); Lydia Jasmin Hanisch, Birmensdorf (CH); Monika Heidrich, Eglfing (DE); Ralf Hosse, Mettmenstetten (CH); Leo Frederik Kunz, Basel (CH); Stephane Leclair, Gauting (DE); Desirée Leisibach, Baden (CH); Fanny Mende, Oberengstringen (CH); Olaf Mundigl, Polling (DE); Miroslav Nikolov, München (DE); Pablo Umaña, Lachen (CH); Cornelia Wagner, Penzberg (DE)

(73) Assignee: Hoffmann-La Roche Inc., Little Falls, NJ (US)

(21) Appl. No.: 18/747,055

(22) Filed: Jun. 18, 2024

Related U.S. Application Data
(63) Continuation of application No. PCT/EP2022/086540, filed on Dec. 19, 2022.

(30) Foreign Application Priority Data
Dec. 20, 2021 (EP) 21215804.2
Nov. 22, 2022 (EP) 22208828.8

(51) Int. Cl. C07K 16/28; A61P 35/00; C07K 16/40

(52) U.S. Cl. CPC C07K 16/2878; A61P 35/00; C07K 16/40; C07K 2317/31; C07K 2317/75; C07K 2317/92

(57) ABSTRACT

The invention relates to novel antibodies that bind to lymphotoxin beta receptor (LTBR) and to bispecific antigen binding molecules comprising these novel LTBR antibodies and an antigen binding domain that binds a tumor associated antigen, in particular to Fibroblast Activation Protein (FAP), to methods of producing these molecules and to methods of using the same.

### CROSS-REFERENCE TO RELATED APPLICATIONS

This application is a continuation of International Application No. PCT/EP2022/086540 having an international filing date of Dec. 19, 2022, which claims benefit of priority to European Patent Application No. 21215804.2, filed Dec. 20, 2021 and European Patent Application No. 22208828.8, filed Nov. 22, 2022, each of which is incorporated herein by reference in its entirety.

### SEQUENCE LISTING

This application contains a Sequence Listing which has been submitted electronically in XML format and is hereby incorporated by reference in its entirety. Said XML copy, created on Jun. 10, 2024 is named P37281-US_SL.xml and is 519,953 bytes in size.

### FIELD OF THE INVENTION

The invention relates to novel antibodies that bind to lymphotoxin beta receptor (LTBR) and to bispecific antigen binding molecules comprising these novel LTBR antibodies and an antigen binding domain that binds a tumor associated antigen, in particular to Fibroblast Activation Protein (FAP), to methods of producing these molecules and to methods of using the same.

### BACKGROUND

The landscape of cancer treatment profoundly changed in recent years after the development and approval of cancer immunotherapies such as agents blocking the immune checkpoints PD-1/PD-L1 and CTLA-4. These cancer immunotherapies can achieve durable responses in cancer indications such as melanoma, non-small cell lung cancer and bladder cancer, and are therefore emerging as the new standard of care alone or in combination with other therapies. However, only a subset of patients (<30%) experience a durable benefit from these therapies and the majority of patients relapse due to primary or acquired resistance mechanisms. In addition, several common cancer indications (e.g. colorectal cancer and pancreatic cancer) are mostly refractory to these immunomodulators. Consequently, there is an urgent medical need in developing therapies to tackle resistance mechanisms and increase responses to cancer immunotherapies, including checkpoint inhibitors.

Clinical data shows that patients who do not respond or respond poorly to checkpoint inhibitors display a non T-cell inflamed immune phenotype characterized either by the absence of cytotoxic T cells, or by their restricted localization to the tumor stroma. Therefore, novel therapies aimed at increasing immune infiltration, would be of great utility in improving the response rate to checkpoint inhibitors, expanding their clinical benefit.

The TNFR superfamily is composed of 19 ligands and 29 receptors, which share some structural similarity, but are involved in many diverse physiological functions. In general terms, members of this superfamily can either induce cell death (e.g. DR5-TRAIL, Fas-FasL) or they can promote survival and inflammation (e.g. TNF-TNFR). The lymphotoxin beta receptor (LTBR) is a member of the TNFR superfamily that belongs to this second category, and is expressed by a variety of cells, including stromal cells of the tumor microenvironment, myeloid cells and tumor cells of epithelial origin. Ligand-receptor interactions within the TNFR superfamily may be mono- and polyvalent. For example, OX40 and its ligand (OX40L) form a monotypic ligand-receptor pair, whereas LTBR, LTα, LTβ and LIGHT display multivalent interactions that create a complex network of interconnected pathways. Activation of LTBR by lymphotoxin α1β1 (LTα1β1) or LIGHT ligand binding induces receptor oligomerization and signal transduction via the canonical and non-canonical NFκB pathways, leading to the upregulation of inflammatory and developmental genes such as adhesion molecules (ICAM and VCAM), chemoattractants (CXCL9, 10, 11) and lymphoid tissue organizing chemokines (CCL21, CCL19, CXCL13) (Lu and Browning, Front Immunol. 2014, 5:47, doi: 10.3389/fimmu.2014.00047). This pathway is essential for the development and maintenance of secondary lymphoid organs as shown by the phenotype of ltbr knock-out mice, which fail to develop lymph nodes and Peyer's patches (Fütterer et al, Immunity 1998, 9 (1), 59-70, doi: 10.1016/s1074-7613 (00) 80588-9). Moreover, it is also key to the development and maintenance of high endothelial venules (HEVs) (Browning et al, Immunity 2005, 23 (5), 539-550, doi: 10.1016/j.immuni.2005.10.002). Multicellular aggregates resembling secondary lymphoid organs, composed of T cells and activated dendritic cells (DCs), B-cells and HEVs, are detected histologically in many solid tumors. Clinical evidence shows that the presence of such ectopic lymphoid organs, also called tertiary lymphoid structures (TLS) or the presence of HEVs correlate with better prognosis in several tumor indications (Dieu-Nosjean et al, Immunol. Rev. 2016, 271 (1), 260-275, doi: 10.1111/imr.12405). TLS and HEVs are thought to be formed in response to the same molecular cues involved in lymph node development, such as the LTBR pathway activation. Activation of LTBR has therefore the potential to promote TLS formation in the tumor microenvironment and induce anti-tumor immune responses.

Indeed, preclinical evidence supports the hypothesis that LTBR activation can increase immune infiltration, boost responses to checkpoint inhibitors and induce TLS formation. LTBR activation by means of an agonistic antibody or a targeted ligand, increased T cell infiltration in several mouse tumor models (Lukashev et al, Cancer Res. 2006, 66 (19), 9617-9624, doi: 10.1158/0008-5472.CAN-06-0217). Consequently, combination therapy with checkpoint inhibitors was more efficacious when combined with an LTBR agonist (Allen et al, Sci Transl Med. 2017, 9 (385), eaak9679, doi: 10.1126/scitranslmed.aak9679; Johansson-Percival et al, Cell Rep. 2017, 13 (12), 2687-2698, doi: 10.1016/j.celrep.2015.12.004; and Tang et al, Cancer Cell 2016, 29 (3), 285-296, doi: 10.1016/j.ccell.2016.02.004). Moreover, evidence of TLS formation and HEV development in response to such agonists were reported.

In addition to the key role of LTBR in development of TLS and HEVs, activation of LTBR has been found to induce cell death of certain carcinoma cell lines (Browning et al, j Exp Med 1996, 183 (3), 867-878, doi: 10.1084/jem. 183.3.867). A phase I study assessed the safety and tolerability of an LTBR agonistic humanized antibody (hCBE11) in patients with advanced solid tumors (ClinicalTrials.gov, NCT00105170). The study was suspended and subsequently terminated before completing the patient enrollment. This suggests the potential for serious safety issues associated to widespread LTBR agonism in humans.

Given the huge therapeutic potential of LTBR agonists to improve cancer immunotherapies, there is a need to provide agonistic LTBR antibodies with an excellent pharmacological profile or bispecific antibodies with an advantageous safety profile and the ability to activate LTBR only in the tumor microenvironment and not in other LTBR-expressing tissues.

### SUMMARY OF THE INVENTION

This invention relates to new antibodies that specifically bind to human lymphotoxin beta receptor (LTBR), in particular agonistic hu LTBR antibodies. These antibodies are able to bind to human LTBR and to cynomolgus LTBR with less than a 2-fold difference in affinity. Some of the new agonistic hu LTBR antibodies are even able to bind to human LTBR, cynomolgus LTBR and murine LTBR. The invention also relates to multispecific antibodies comprising these new agonistic antibodies.

Thus, provided herein is an agonistic lymphotoxin beta receptor (LTBR) antibody that specifically binds to human LTBR and to cynomolgus LTBR, wherein said antibody comprises
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32; or
(ii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; or
(iii) a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80; or
(iv) a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88; or
(v) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(vi) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(vii) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(viii) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

In one aspect, provided is an agonistic LTBR antibody as described herein before, wherein the agonistic antibody has at least one of the following properties:
(a) binds to human LTBR and to cynomolgus LTBR with less than a 2-fold difference in affinity; or
(b) binds to human LTBR extracellular domain with an EC₅₀ of less than 4 nM as measured by ELISA and binds to the cynomolgus LTBR extracellular domain with an EC₅₀ of less than 5 nM as measured by ELISA; or
(c) requires cross-linking for its agonistic activity to activate human LTBR; or
(d) requires cross-linking for its agonistic activity to induce ICAM upregulation in human umbilical vein endothelial cells or cancer associated fibroblasts; or
(e) inhibits the interaction between human LTBR and its human ligands lymphotoxin α1β2 and LIGHT.

In one aspect, provided is an agonistic LTBR antibody comprising
(i) a heavy chain variable region (V_{H} LTBR) comprising an amino acid sequence that is at least about 95% identical to the amino acid sequence of SEQ ID NO:33 and a light chain variable region (V_{L} LTBR) comprising an amino acid sequence that is at least about 95% identical to the amino acid sequence of SEQ ID NO:34, or
(ii) a heavy chain variable region (V_{H} LTBR) comprising an amino acid sequence that is at least about 95% identical to the amino acid sequence of SEQ ID NO:49 and a light chain variable region (V_{L} LTBR) comprising an amino acid sequence that is at least about 95% identical to the amino acid sequence of SEQ ID NO:50, or
(iii) a heavy chain variable region (V_{H} LTBR) comprising an amino acid sequence that is at least about 95% identical to the amino acid sequence of SEQ ID NO:81 and a light chain variable region (V_{L} LTBR) comprising an amino acid sequence that is at least about 95% identical to the amino acid sequence of SEQ ID NO:82, or
(iv) a heavy chain variable region (V_{H} LTBR) comprising an amino acid sequence that is at least about 95% identical to the amino acid sequence of SEQ ID NO:89 and a light chain variable region (V_{L} LTBR) comprising an amino acid sequence that is at least about 95% identical to the amino acid sequence of SEQ ID NO:90, or
(v) a heavy chain variable region (V_{H} LTBR) comprising an amino acid sequence that is at least about 95% identical to the amino acid sequence of SEQ ID NO:97 and a light chain variable region (V_{L} LTBR) comprising an amino acid sequence that is at least about 95% identical to the amino acid sequence of SEQ ID NO:98,
(vi) a heavy chain variable region (V_{H} LTBR) comprising an amino acid sequence that is at least about 95% identical to the amino acid sequence of SEQ ID NO:41 and a light chain variable region (V_{L} LTBR) comprising an amino acid sequence that is at least about 95% identical to the amino acid sequence of SEQ ID NO:42,
(vii) a heavy chain variable region (V_{H} LTBR) comprising an amino acid sequence that is at least about 95% identical to the amino acid sequence of SEQ ID NO:57 and a light chain variable region (V_{L} LTBR) comprising an amino acid sequence that is at least about 95% identical to the amino acid sequence of SEQ ID NO:58, or
(viii) a heavy chain variable region (V_{H} LTBR) comprising an amino acid sequence that is at least about 95% identical to the amino acid sequence of SEQ ID NO:65 and a light chain variable region (V_{L} LTBR) comprising an amino acid sequence that is at least about 95% identical to the amino acid sequence of SEQ ID NO:66, or
(ix) a heavy chain variable region (V_{H} LTBR) comprising an amino acid sequence that is at least about 95% identical to the amino acid sequence of SEQ ID NO:73 and a light chain variable region (V_{L} LTBR) comprising an amino acid sequence that is at least about 95% identical to the amino acid sequence of SEQ ID NO:74.

In one aspect, the agonistic LTBR antibody as described herein before comprises
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(iii) a heavy chain variable region (V_{H} LTBR) comprising the amino acid sequence of SEQ ID NO:81 and a light chain variable region (V_{L} LTBR) comprising the amino acid sequence of SEQ ID NO:82, or
(iv) a heavy chain variable region (V_{H} LTBR) comprising the amino acid sequence of SEQ ID NO:89 and a light chain variable region (V_{L} LTBR) comprising the amino acid sequence of SEQ ID NO:90, or
(v) a heavy chain variable region (V_{H} LTBR) comprising the amino acid sequence of SEQ ID NO:97 and a light chain variable region (V_{L} LTBR) comprising the amino acid sequence of SEQ ID NO:98, or
(vi) a heavy chain variable region (V_{H} LTBR) comprising the amino acid sequence of SEQ ID NO:41 and a light chain variable region (V_{L} LTBR) comprising the amino acid sequence of SEQ ID NO:42, or
(vii) a heavy chain variable region (V_{H} LTBR) comprising the amino acid sequence of SEQ ID NO:57 and a light chain variable region (V_{L} LTBR) comprising the amino acid sequence of SEQ ID NO:58, or
(viii) a heavy chain variable region (V_{H} LTBR) comprising the amino acid sequence of SEQ ID NO:65 and a light chain variable region (V_{L} LTBR) comprising the amino acid sequence of SEQ ID NO:66,
(ix) a heavy chain variable region (V_{H} LTBR) comprising the amino acid sequence of SEQ ID NO:73 and a light chain variable region (V_{L} LTBR) comprising the amino acid sequence of SEQ ID NO:74.

In one particular aspect, provided is an agonistic LTBR antibody as described herein before, wherein the agonistic LTBR antibody further specifically binds to murine LTBR. In one aspect, the agonistic LTBR antibody binds to the murine LTBR extracellular domain with an EC₅₀ of less than 1 nM as measured by ELISA.

In one aspect, the agonistic LTBR antibody that further specifically binds to murine LTBR comprises
a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48, or
a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80, or
a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

In one aspect, the agonistic LTBR antibody that further specifically binds to murine LTBR comprises
a heavy chain variable region (V_{H} LTBR) comprising the amino acid sequence of SEQ ID NO:33 and a light chain variable region (V_{L} LTBR) comprising the amino acid sequence of SEQ ID NO:34, or
a heavy chain variable region (V_{H} LTBR) comprising the amino acid sequence of SEQ ID NO:49 and a light chain variable region (V_{L} LTBR) comprising the amino acid sequence of SEQ ID NO:50, or
a heavy chain variable region (V_{H} LTBR) comprising the amino acid sequence of SEQ ID NO:81 and a light chain variable region (V_{L} LTBR) comprising the amino acid sequence of SEQ ID NO:82, or
a heavy chain variable region (V_{H} LTBR) comprising the amino acid sequence of SEQ ID NO:89 and a light chain variable region (V_{L} LTBR) comprising the amino acid sequence of SEQ ID NO:90, or
a heavy chain variable region (V_{H} LTBR) comprising the amino acid sequence of SEQ ID NO:97 and a light chain variable region (V_{L} LTBR) comprising the amino acid sequence of SEQ ID NO:98.

In one particular aspect, the agonistic LTBR antibody as described herein binds to the epitope region of SEQ ID NO:351 on human LTBR. In one aspect, such agonistic LTBR antibody comprises a heavy chain variable region (V_{H} LTBR) comprising a CDR-H1 comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32. In one aspect, comprises a heavy chain variable region (V_{H} LTBR) comprising the amino acid sequence of SEQ ID NO:33 and a light chain variable region (V_{L} LTBR) comprising the amino acid sequence of SEQ ID NO:34.

The invention also relates to agonistic LTBR antibodies that are multispecific specific antibodies comprising the agonistic LTBR antibody as described herein before. In one particular aspect, provided are agonistic LTBR antibodies that are bispecific antibodies. In any of the aspects described herein before, the agonistic LTBR antibody preferably comprises a Fc domain of human origin, particularly of human IgG subclass, more particularly of human IgG1 subclass. In one aspect, the agonistic LTBR antibody comprises a Fc domain of human IgG1 subclass comprising one or more amino acid substitution that reduces the binding affinity of the antigen binding molecule to an Fc receptor and/or effector function. In one aspect, the agonistic LTBR antibody comprises a Fc domain of human IgG1 subclass with the amino acid mutations L234A, L235A and P329G (numbering according to Kabat EU index).

In one aspect, the agonistic LTBR antibody is a bispecific antibody that that specifically binds to LTBR and to a tumor associated antigen (TAA).

In one aspect, provided is an agonistic LTBR antibody that is a bispecific antibody capable of specific binding to lymphotoxin beta receptor (LTBR) and Fibroblast activation protein (FAP) and thus combines an antigen binding domain that specifically binds to FAP with at least one antigen binding domain capable of agonistic binding to LTBR, in particular hu LTBR, wherein the activation through LTBR is provided by cross-linking through binding to FAP expressed on tumor stromal cells. In contrast to conventional, non-targeted, agonistic LTBR antibodies, targeting to a tumor associated target (TAA) like FAP enables to restrict LTBR agonism exclusively to the tumor microenvironment (tumor endothelium and cancer associated fibroblasts), thereby reducing potential side effects.

Fibroblast activation protein (FAP) is a serine protease highly expressed on the cell surface of cancer-associated stroma cells, and on fibroblastic reticular cells in secondary lymphoid organs, but has otherwise very limited expression in normal tissues. FAP is highly prevalent in various cancer indications allowing its usage as targeting moiety for drugs that should accumulate within the tumor stroma.

Thus, in one aspect provided is a bispecific agonistic LTBR antibody, comprising
(a) a first antigen binding domain that specifically binds to Fibroblast Activation Protein (FAP),
(b) a second antigen binding domain that specifically binds to lymphotoxin beta receptor (LTBR), and
(c) a Fc domain composed of a first and a second subunit and comprising one or more amino acid substitution that reduces the binding affinity of the antibody to an Fc receptor and/or effector function.

The bispecific antibodies possess a Fc domain composed of a first and a second subunit that comprises one or more amino acid substitution that reduces the binding affinity of the antibody to an Fc receptor and/or effector function. Fc receptor-mediated cross-linking is thereby abrogated and tumor-specific activation is achieved by cross-linking through binding of the antigen binding domain that specifically binds to FAP through binding to its tumor-associated target. Provided is a bispecific antibody that only activates LTBR upon binding to FAP. Thus, a bispecific antibody is provided that activates LTBR in the tumor stroma.

In one aspect, provided is a bispecific agonistic LTBR antibody, comprising
(a) a first Fab fragment that specifically binds to Fibroblast Activation Protein (FAP),
(b) a second Fab fragment that specifically binds to lymphotoxin beta receptor (LTBR), and
(c) a Fc domain composed of a first and a second subunit and comprising one or more amino acid substitution that reduces the binding affinity of the antigen binding molecule to an Fc receptor and/or effector function.

In one aspect, provided is a bispecific agonistic LTBR antibody, wherein the bispecific agonistic LTBR antibody comprises a third antigen binding domain that specifically binds to LTBR, i.e. a bispecific agonistic LTBR antibody comprising (a) a first antigen binding domain that specifically binds to Fibroblast Activation Protein (FAP), (b) a second and a third antigen binding domain that specifically bind to lymphotoxin beta receptor (LTBR), and (c) a Fc domain composed of a first and a second subunit and comprising one or more amino acid substitution that reduces the binding affinity of the antigen binding molecule to an Fc receptor and/or effector function.

In one particular aspect, the third antigen binding domain that specifically binds to LTBR is identical to the second antigen binding domain that specifically binds to LTBR, meaning that the second and a third antigen binding domain that specifically bind to lymphotoxin beta receptor (LTBR) are the same. In one aspect, the second and third antigen binding domains that specifically bind to LTBR are Fab fragments that specifically bind to LTBR. In one aspect, the Fab fragments that specifically bind to LTBR are crossfab fragments. In a further aspect, the first antigen binding domain that specifically binds to FAP is a Fab fragment.

In one aspect, the bispecific agonistic LTBR antibody as disclosed herein comprises a first antigen binding domain that specifically binds to Fibroblast Activation Protein (FAP) comprising
(i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8, or
(ii) a heavy chain variable region (V_{H}FAP) comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21, and a light chain variable region (V_{L}FAP) comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24, or
(iii) a heavy chain variable region (V_{H}FAP) comprising a CDR-H1 comprising the amino acid sequence of SEQ ID NO:11, a CDR-H2 comprising the amino acid sequence of SEQ ID NO:12, and a CDR-H3 comprising the amino acid sequence of SEQ ID NO:13, and a light chain variable region (V_{L}FAP) comprising a CDR-L1 comprising the amino acid sequence of SEQ ID NO:14, a CDR-L2 comprising the amino acid sequence of SEQ ID NO:15, and a CDR-L3 comprising the amino acid sequence of SEQ ID NO:16.

In one aspect, the first antigen binding domain that specifically binds to FAP comprises
(i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8, or (ii) a heavy chain variable region (V_{H}FAP) comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21, and a light chain variable region (V_{L}FAP) comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24. In one particular aspect, the first antigen binding domain that specifically binds to FAP comprises (i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8.

In one aspect, the first antigen binding domain that specifically binds to FAP comprises a heavy chain variable region (V_{H}FAP) comprising an amino acid sequence that is at least about 95% identical to the amino acid sequence of SEQ ID NO:9, and a light chain variable region (V_{L}FAP) comprising an amino acid sequence that is at least about 95% identical to the amino acid sequence of SEQ ID NO:10, or it comprises a heavy chain variable region (V_{H}FAP) comprising an amino acid sequence that is at least about 95% identical to the amino acid sequence of SEQ ID NO:25, and a light chain variable region (V_{L}FAP) comprising an amino acid sequence that is at least about 95% identical to the amino acid sequence of SEQ ID NO:26, or it comprises a heavy chain variable region (V_{H}FAP) comprising an amino acid sequence that is at least about 95% identical to the amino acid sequence of SEQ ID NO:17, and a light chain variable region (V_{L}FAP) comprising an amino acid sequence that is at least about 95% identical to the amino acid sequence of SEQ ID NO:18. In one aspect, the first antigen binding domain that specifically binds to FAP comprises a heavy chain variable region (V_{H}FAP) comprising the amino acid sequence of SEQ ID NO:9 and a light chain variable region (V_{L}FAP) comprising the amino acid sequence of SEQ ID NO:10 or it comprises a heavy chain variable region (V_{H}FAP) comprising the amino acid sequence of SEQ ID NO:25 and a light chain variable region (V_{L}FAP) comprising the amino acid sequence of SEQ ID NO:26 or it comprises a heavy chain variable region (V_{H}FAP) comprising the amino acid sequence of SEQ ID NO:17 and a light chain variable region (V_{L}FAP) comprising the amino acid sequence of SEQ ID NO:18. In one particular aspect, the first antigen binding domain that specifically binds to FAP comprises a heavy chain variable region (V_{H}FAP) comprising the amino acid sequence of SEQ ID NO:9 and a light chain variable region (V_{L}FAP) comprising the amino acid sequence of SEQ ID NO:10. Particularly, the first antigen binding domain that specifically binds to FAP comprises a heavy chain variable region (V_{H}FAP) comprising the amino acid sequence of SEQ ID NO:9 and a light chain variable region (V_{L}FAP) comprising the amino acid sequence of SEQ ID NO:10 and is a Fab fragment.

In another aspect, the bispecific agonistic LTBR antibody as disclosed herein comprises a second antigen binding domain that specifically binds to LTBR comprising
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32; or
(ii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; or
(iii) a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80; or
(iv) a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88; or
(v) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(vi) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(vii) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(viii) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

In one aspect, the second and the third antigen binding domain that specifically bind to LTBR comprise
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32; or
(ii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; or
(iii) a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80; or
(iv) a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88; or
(v) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(vi) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(vii) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(viii) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

In one aspect, provided is a bispecific agonistic LTBR antibody as disclosed herein wherein the second antigen binding domain (and optionally the third antigen binding domain) that specifically binds to LTBR, each comprises
(i) a heavy chain variable region (V_{H} LTBR) comprising an amino acid sequence that is at least about 95% identical to the amino acid sequence of SEQ ID NO:33 and a light chain variable region (V_{L} LTBR) comprising an amino acid sequence that is at least about 95% identical to the amino acid sequence of SEQ ID NO:34, or
(ii) a heavy chain variable region (V_{H} LTBR) comprising an amino acid sequence that is at least about 95% identical to the amino acid sequence of SEQ ID NO:49 and a light chain variable region (V_{L} LTBR) comprising an amino acid sequence that is at least about 95% identical to the amino acid sequence of SEQ ID NO:50, or
(iii) a heavy chain variable region (V_{H} LTBR) comprising an amino acid sequence that is at least about 95% identical to the amino acid sequence of SEQ ID NO:81 and a light chain variable region (V_{L} LTBR) comprising an amino acid sequence that is at least about 95% identical to the amino acid sequence of SEQ ID NO:82, or
(iv) a heavy chain variable region (V_{H} LTBR) comprising an amino acid sequence that is at least about 95% identical to the amino acid sequence of SEQ ID NO:89 and a light chain variable region (V_{L} LTBR) comprising an amino acid sequence that is at least about 95% identical to the amino acid sequence of SEQ ID NO:90, or
(v) a heavy chain variable region (V_{H} LTBR) comprising an amino acid sequence that is at least about 95% identical to the amino acid sequence of SEQ ID NO:97 and a light chain variable region (V_{L} LTBR) comprising an amino acid sequence that is at least about 95% identical to the amino acid sequence of SEQ ID NO:98,
(vi) a heavy chain variable region (V_{H} LTBR) comprising an amino acid sequence that is at least about 95% identical to the amino acid sequence of SEQ ID NO:41 and a light chain variable region (V_{L} LTBR) comprising an amino acid sequence that is at least about 95% identical to the amino acid sequence of SEQ ID NO:42,
(vii) a heavy chain variable region (V_{H} LTBR) comprising an amino acid sequence that is at least about 95% identical to the amino acid sequence of SEQ ID NO:57 and a light chain variable region (V_{L} LTBR) comprising an amino acid sequence that is at least about 95% identical to the amino acid sequence of SEQ ID NO:58, or
(viii) a heavy chain variable region (V_{H} LTBR) comprising an amino acid sequence that is at least about 95% identical to the amino acid sequence of SEQ ID NO:65 and a light chain variable region (V_{L} LTBR) comprising an amino acid sequence that is at least about 95% identical to the amino acid sequence of SEQ ID NO:66, or
(ix) a heavy chain variable region (V_{H} LTBR) comprising an amino acid sequence that is at least about 95% identical to the amino acid sequence of SEQ ID NO:73 and a light chain variable region (V_{L} LTBR) comprising an amino acid sequence that is at least about 95% identical to the amino acid sequence of SEQ ID NO:74.

In one aspect, the bispecific agonistic LTBR antibody as disclosed herein comprises a second antigen binding domain (and optionally a third antigen binding domain) that specifically binds to LTBR comprising
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) (ii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(iii) a heavy chain variable region (V_{H} LTBR) comprising the amino acid sequence of SEQ ID NO:81 and a light chain variable region (V_{L} LTBR) comprising the amino acid sequence of SEQ ID NO:82, or
(iv) a heavy chain variable region (V_{H} LTBR) comprising the amino acid sequence of SEQ ID NO:89 and a light chain variable region (V_{L} LTBR) comprising the amino acid sequence of SEQ ID NO:90, or
(v) a heavy chain variable region (V_{H} LTBR) comprising the amino acid sequence of SEQ ID NO:97 and a light chain variable region (V_{L} LTBR) comprising the amino acid sequence of SEQ ID NO:98, or
(vi) a heavy chain variable region (V_{H} LTBR) comprising the amino acid sequence of SEQ ID NO:41 and a light chain variable region (V_{L} LTBR) comprising the amino acid sequence of SEQ ID NO:42, or
(vii) a heavy chain variable region (V_{H} LTBR) comprising the amino acid sequence of SEQ ID NO:57 and a light chain variable region (V_{L} LTBR) comprising the amino acid sequence of SEQ ID NO:58, or
(viii) a heavy chain variable region (V_{H} LTBR) comprising the amino acid sequence of SEQ ID NO:65 and a light chain variable region (V_{L} LTBR) comprising the amino acid sequence of SEQ ID NO:66,
(ix) a heavy chain variable region (V_{H} LTBR) comprising the amino acid sequence of SEQ ID NO:73 and a light chain variable region (V_{L} LTBR) comprising the amino acid sequence of SEQ ID NO:74.

In another aspect, provided is a bispecific agonistic LTBR antibody as disclosed herein wherein the second antigen binding domain (and optionally the third antigen binding domain) that specifically binds to human LTBR, cynomolgus LTBR and murine LTBR comprises
a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48, or
a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80, or
a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

In one aspect, the bispecific agonistic LTBR antibody comprises a second antigen binding domain (and optionally a third antigen binding domain) that specifically binds to human LTBR, cynomolgus LTBR and murine LTBR comprising
a heavy chain variable region (V_{H} LTBR) comprising the amino acid sequence of SEQ ID NO:33 and a light chain variable region (V_{L} LTBR) comprising the amino acid sequence of SEQ ID NO:34, or
a heavy chain variable region (V_{H} LTBR) comprising the amino acid sequence of SEQ ID NO:49 and a light chain variable region (V_{L} LTBR) comprising the amino acid sequence of SEQ ID NO:50, or
a heavy chain variable region (V_{H} LTBR) comprising the amino acid sequence of SEQ ID NO:81 and a light chain variable region (V_{L} LTBR) comprising the amino acid sequence of SEQ ID NO:82, or
a heavy chain variable region (V_{H} LTBR) comprising the amino acid sequence of SEQ ID NO:89 and a light chain variable region (V_{L} LTBR) comprising the amino acid sequence of SEQ ID NO:90, or
a heavy chain variable region (V_{H} LTBR) comprising the amino acid sequence of SEQ ID NO:97 and a light chain variable region (V_{L} LTBR) comprising the amino acid sequence of SEQ ID NO:98.

In another aspect, the second antigen binding domain (and optionally a third antigen binding domain) that specifically binds to LTBR comprise
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:41 and a light chain variable region (V_{L} LTBR) comprising the amino acid sequence of SEQ ID NO:42, or
(iii) a heavy chain variable region (V_{H} LTBR) comprising the amino acid sequence of SEQ ID NO:49 and a light chain variable region (V_{L} LTBR) comprising the amino acid sequence of SEQ ID NO:50.

In one particular aspect, the second antigen binding domain (and optionally a third antigen binding domain) that specifically binds to LTBR comprise a heavy chain variable region (V_{H} LTBR) comprising the amino acid sequence of SEQ ID NO:33 and a light chain variable region (V_{L} LTBR) comprising the amino acid sequence of SEQ ID NO:34. In another particular aspect, the second antigen binding domain (and optionally a third antigen binding domain) that specifically binds to LTBR comprise a heavy chain variable region (V_{H} LTBR) comprising the amino acid sequence of SEQ ID NO:41 and a light chain variable region (V_{L} LTBR) comprising the amino acid sequence of SEQ ID NO:42. In yet another particular aspect, the second antigen binding domain (and optionally a third antigen binding domain) that specifically binds to LTBR comprise a heavy chain variable region (V_{H} LTBR) comprising the amino acid sequence of SEQ ID NO:49 and a light chain variable region (V_{L} LTBR) comprising the amino acid sequence of SEQ ID NO:50.

Thus, in one particular aspect, the bispecific agonistic LTBR antibody as described herein comprises (a) a first antigen binding domain that specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and (b) a second antigen binding domain (and optionally third antigen binding domain) that specifically binds to LTBR, comprising a heavy chain variable region (V_{H} LTBR) comprising an amino acid sequence of SEQ ID NO:33 and a light chain variable region (V_{L} LTBR) comprising an amino acid sequence of SEQ ID NO:34. In another aspect, the bispecific antigen binding molecule as described herein comprises (a) a first antigen binding domain that specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and (b) a second antigen binding domain (and optionally third antigen binding domain) that specifically binds to LTBR, comprising a heavy chain variable region (V_{H} LTBR) comprising an amino acid sequence of SEQ ID NO:41 and a light chain variable region (V_{L} LTBR) comprising an amino acid sequence of SEQ ID NO:42. In yet another aspect, the bispecific antigen binding molecule as described herein comprises (a) a first antigen binding domain that specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and (b) a second antigen binding domain (and optionally third antigen binding domain) that specifically binds to LTBR, comprising a heavy chain variable region (V_{H} LTBR) comprising an amino acid sequence of SEQ ID NO:49 and a light chain variable region (V_{L} LTBR) comprising an amino acid sequence of SEQ ID NO:50.

In all of these aspects described herein before, the bispecific agonistic LTBR antibody comprises a Fc domain composed of a first and a second subunit and comprising one or more amino acid substitution that reduces the binding affinity of the antigen binding molecule to an Fc receptor and/or effector function. In one aspect, the Fc domain composed of a first and a second subunit is an IgG Fc domain. In one particular aspect, the Fc domain composed of a first and a second subunit is IgG1 Fc domain or an IgG4 Fc domain. In one particular aspect, the Fc domain is of human IgG1 subclass with the amino acid mutations L234A, L235A and P329G (numbering according to Kabat EU index).

In another aspect, provided is a bispecific agonistic LTBR antibody as defined herein before, wherein the first subunit of the Fc region comprises knobs and the second subunit of the Fc region comprises holes according to the knobs into holes method. In particular, provided is a bispecific antigen binding molecule, wherein (i) the first subunit of the Fc region comprises the amino acid substitutions S354C and T366W (numbering according to Kabat EU index) and the second subunit of the Fc region comprises the amino acid substitutions Y349C, T366S and Y407V (numbering according to Kabat EU index), or (ii) the first subunit of the Fc region comprises the amino acid substitutions K392D and K409D (numbering according to Kabat EU index) and the second subunit of the Fc region comprises the amino acid substitutions E356K and D399K (numbering according to Kabat EU index). More particularly, provided is a bispecific agonistic LTBR antibody, wherein the first subunit of the Fc region comprises the amino acid substitutions S354C and T366W (numbering according to Kabat EU index) and the second subunit of the Fc region comprises the amino acid substitutions Y349C, T366S and Y407V (numbering according to Kabat EU index).

In a further aspect, provided is a bispecific agonistic LTBR antibody as defined herein before, comprising
(a) a first Fab fragment that specifically binds to FAP,
(b) a second Fab fragment that specifically binds to LTBR, and
(c) a Fc domain composed of a first and a second subunit and comprising one or more amino acid substitution that reduces the binding affinity of the antibody to an Fc receptor and/or effector function. In one aspect, the second Fab fragment that specifically binds to LTBR is a crossFab fragment. Thus, provided is a bispecific agonistic LTBR antibody that provides monovalent binding towards LTBR and monovalent binding towards FAP.

In another aspect, provided is a bispecific agonistic LTBR antibody, comprising
(a) a first Fab fragment that specifically binds to FAP,
(b) a second and a third Fab fragment that specifically bind to LTBR, and
(c) a Fc domain composed of a first and a second subunit and comprising one or more amino acid substitution that reduces the binding affinity of the antibody to an Fc receptor and/or effector function, wherein the first Fab fragment that specifically binds to FAP is fused at its N-terminus to the C-terminus of one of the Fc domain subunits and the second and a third Fab fragment that specifically bind to LTBR are each fused at its C-terminus to the N-terminus of one of the Fc domain subunits.

Thus, provided is a bispecific agonistic LTBR antibody that provides bivalent binding towards LTBR and monovalent binding towards FAP.

According to another aspect of the invention, there is provided isolated one or more isolated polynucleotide encoding an agonistic LTBR antibody or a bispecific agonistic LTBR antibody as described herein before. The invention further provides a vector, particularly an expression vector, comprising the isolated polynucleotide of the invention and a host cell comprising the isolated nucleic acid or the expression vector of the invention. In some aspects, the host cell is an eukaryotic cell, particularly a mammalian cell. In another aspect, provided is a method of producing an agonistic LTBR antibody or a bispecific agonistic LTBR antibody as described herein before, comprising culturing the host cell as described above under conditions suitable for the expression of the an agonistic LTBR antibody or a bispecific agonistic LTBR antibody, and isolating the an agonistic LTBR antibody or a bispecific agonistic LTBR antibody. The invention also encompasses the an agonistic LTBR antibody or a bispecific agonistic LTBR antibody as produced by the method of the invention.

The invention further provides a pharmaceutical composition comprising an agonistic LTBR antibody or a bispecific agonistic LTBR antibody as described herein before and a pharmaceutically acceptable carrier. In one aspect, the pharmaceutical composition comprises an additional therapeutic agent.

Also encompassed by the invention is the agonistic LTBR antibody or bispecific agonistic LTBR antibody as described herein before, or the pharmaceutical composition comprising the bispecific agonistic LTBR antibody, for use as a medicament.

In one aspect, provided is a bispecific agonistic LTBR antibody as described herein before or the pharmaceutical composition of the invention, for use in (a) inducing ICAM upregulation on endothelial cells or cancer-associated fibroblasts, or (b) enhancing T cell adhesion.

In a specific aspect, provided is the agonistic LTBR antibody or bispecific agonistic LTBR antibody as described herein before or the pharmaceutical composition of the invention, for use in the treatment of cancer. In another specific aspect, the invention provides the agonistic LTBR antibody or bispecific agonistic LTBR antibody as described herein before for use in the treatment of cancer, wherein the agonistic LTBR antibody or bispecific agonistic LTBR antibody is administered in combination with a chemotherapeutic agent, radiation and/or other agents for use in cancer immunotherapy. In one aspect, the agonistic LTBR antibody or bispecific agonistic LTBR antibody as described herein is for use in the treatment of cancer, wherein the agonistic LTBR antibody or bispecific agonistic LTBR antibody is for administration in combination with an agent blocking PD-L1/PD-1 interaction. In another aspect, provided is the agonistic LTBR antibody or bispecific agonistic LTBR antibody as described herein before or the pharmaceutical composition of the invention, for use in up-regulating or prolonging cytotoxic T cell activity.

In a further aspect, the invention provides a method of inhibiting the growth of tumor cells in an individual comprising administering to the individual an effective amount of the agonistic LTBR antibody or bispecific agonistic LTBR antibody as described herein before, or the pharmaceutical composition of the invention, to inhibit the growth of the tumor cells. In another aspect, the invention provides a method of treating or delaying cancer in an individual comprising administering to the individual an effective amount of the agonistic LTBR antibody or bispecific agonistic LTBR antibody as described herein before, or the pharmaceutical composition of the invention.

Also provided is the use of the bispecific antigen binding molecule as described herein before for the manufacture of a medicament for the treatment of a disease in an individual in need thereof, in particular for the manufacture of a medicament for the treatment of cancer, as well as a method of treating a disease in an individual, comprising administering to said individual a therapeutically effective amount of a composition comprising the agonistic LTBR antibody or bispecific agonistic LTBR antibody of the invention in a pharmaceutically acceptable form. In a specific aspect, the disease is cancer. In any of the above aspects the individual is a mammal, particularly a human.

### BRIEF DESCRIPTION OF THE DRAWINGS

FIGS. 1A to 1G show schematic representations of the recombinant soluble proteins (receptors, ligands and tool proteins) used as antigens for phage display and immunization to generate anti-LTBR antibodies. FIG. 1A shows a schematic representation of the so-called biotinylated Fc-depleter (Fc-depleter kh NC avi biotinylated, P1AA0981), which comprises a knob-into-hole (kh) Fc domain that is used as pre-clearing agent to avoid clones binding to the Fc domain. FIG. 1B shows a schematic representation of human lymphotoxin α1β2 single-chain avi his (P1AE1235). FIG. 1C shows a schematic representation of murine lymphotoxin α1β2 single-chain avi his (P1AE1236). FIG. 1D shows a schematic representation of the monomeric huLTBR ectodomain ECD (S28-M227) as Fc-fusion avi biotinylated (P1AE2835). FIG. 1E shows a schematic representation of the monomeric muLTBR ectodomain ECD (S28-L223) Fc-fusion avi biotinylated (P1AE4410). FIG. 1F shows a schematic representation of the monomeric cyno LTBR ectodomain ECD (S28-M227) Fc fusion avi biotinylated (P1AE4411). FIG. 1G shows a schematic representation of the monomeric N-terminal human LTBR (ECD full length) hu IgG1 Fc-fusion kih HRYF avi (P1AE1217).

FIGS. 2A to 2F show schematic representations of additional antigens used for screening and clone characterization of anti-LTBR antibodies and bispecific FAP-LTBR antibodies. FIG. 2A shows a schematic representation of a dimeric hu LTBR-Fc fusion biotinylated (P1AE2401). FIG. 2B shows a schematic representation of human LTBR ECD (S28-M227) N- and C-terminally extended-Fc-fusion wt kih HRYF-Avi-His biotinylated (P1AE7979). FIG. 2C shows a schematic representation of monomeric N-terminal cyno LTbR (ECD full length) hu IgG1 Fc-fusion kih HRYF avi biotinylated (P1AE2656). FIG. 2D shows a schematic representation of dimeric murine LTBR-Fc disulfide-linked homodimer biotinylated (P1AE2655). FIG. 2E shows a schematic representation of human FAP-Avi-His biotinylated (P1AA5347). FIG. 2F shows a schematic representation of murine FAP-Avi-His biotinylated (P1AD9907).

FIG. 3 shows the identification of Fab clone FAPltbr.P218.076 (P1AE5929 after IgG-conversion) as cross-reactive antibody in a Sandwich ELISA wherein the specific binding to human LTBR (P1AE2835) and murine LTBR (P1AE4410) was determined. The antibody originates from Phage Display. A human Fc fragment (P1AA0981) was used to confirm specific binding to LTBR and exclude Fc binding as the antigens used in phage display have been Fc-tagged fusion proteins. The Y-axis represents OD₄₅₀₋₉₀₀.

FIG. 4 shows the inhibition of LTBR-lymphotoxin α1β2 interactions by anti-LTBR IgG antibodies. The selected anti-LTBR IgGs have been titrated in 3-fold dilutions starting at 100 nM (15 μg/ml).

FIG. 5 shows the inhibition of LTBR-LIGHT interactions by anti-LTBR IgG antibodies. The selected anti-LTBR IgGs have been titrated in 3-fold dilutions starting at 100 nM (15 μg/ml).

FIG. 6 shows the results for various anti-LTBR IgGs in the absence or presence of a cross-linking antibody (linker) as determined in the HeLa NFκB luc reporter assay as described in Example 1.7. The concentration of the anti-LTBR IgGs are blotted against the released light units (RLU) measured after incubation and addition of Luciferase detection solution.

FIG. 7 shows the results of the assessment of cell surface LTBR binding (FACS on recombinant human LTBR CHO cells) for anti-LTBR IgGs plotted as median fluorescence intensity against the concentration. Anti-LTBR IgGs have been used in a titration with 3-fold dilution steps starting at 20 μg/ml.

FIG. 8 shows the epitope binning heat map based on the respective binding values in percentage for 8 selected anti-LTBR IgGs.

FIGS. 9A to 9E show schematic representations of anti-LTBR antibodies and bispecific FAP-LTBR antibodies. FIG. 9A shows a schematic representation of a monospecific anti-LTBR human IgG1 PG LALA antibody. FIG. 9B shows a schematic representation of the 1+1 anti-LTBR/anti-FAP bispecific antibody as human IgG1 PG LALA crossMab with crossed V-domains in the anti-LTBR Fab arm and charges in the CH1/Ck domains of the anti-FAP Fab arm. FIG. 9C shows a schematic representation of the 1+1 anti-LTBR/anti-FAP bispecific antibody as human IgG1 PG LALA crossMab with crossed CH1/Ck domains in the anti-FAP Fab arm. FIG. 9D shows a schematic representation of the 2+1 anti-LTBR/anti-FAP bispecific antibody as human IgG1 PG LALA crossMab with crossed CH1/Ck domains in the anti-FAP Fab arm and a second anti-LTBR Fab fragment fused to the C-terminus of the Fc domain.

FIG. 9E shows a schematic representation of the 2+1 anti-LTBR/anti-FAP bispecific antibody as human IgG1 PG LALA crossMab with crossed V-domains in both anti-LTBR Fab arms and charges in the CH1/Ck domains of the anti-FAP Fab fragment that is fused to the C-terminus of the Fc domain.

FIG. 10 shows the inhibition of LTBR-lymphotoxin α1β2 interactions by anti-FAP/anti-LTBR bispecific antibodies. The selected anti-FAP/anti-LTBR bispecific antibodies have been titrated in 3-fold dilutions starting at 100 nM (15 μg/ml).

FIG. 11 shows the inhibition of LTBR-LIGHT interactions by anti-FAP/anti-LTBR bispecific antibodies. The selected anti-FAP/anti-LTBR bispecific antibodies have been titrated in 3-fold dilutions starting at 100 nM (15 μg/ml).

FIGS. 12A and 12B show schematic representations of the murine surrogate bispecific FAP-LTBR antibodies. FIG. 12A shows a schematic representation of the 1+1 anti-LTBR/anti-FAP bispecific antibody as murine IgG1 DA PG crossMab with crossed V-domains in the anti-FAP Fab arm and charges in the CH1/Ck domains of the anti-mu LTBR Fab arm. FIG. 12B shows a schematic representation of the 2+1 anti-LTBR/anti-FAP bispecific antibody as murine IgG1 DA PG crossMab with charges in the CH1/Ck domains of both anti-LTBR Fabs and crossed CH1/Ck domains in the anti-FAP Fab fragment that is fused to the C-terminus of the Fc domain. FIG. 12C shows a schematic representation of a 2+1 anti-LTBR/non-targeting (DP47) bispecific antibody as murine IgG1 DA PG crossmab with charges in the CH1/Ck domains of both anti-LTBR Fabs and crossed CH1/Ck domains in the non-targeting (DP47) Fab fragment that is fused to the C-terminus of the Fc domain.

FIGS. 13A to 13D show that anti-LTBR agonistic antibodies upregulate ICAM on endothelial cells in a crosslinking dependent manner. Shown is the Median fluorescence intensity of ICAM on human umbilical vein endothelial cells (HUVECs) treated with anti-human LTBR agonistic antibodies in the presence (FIG. 13A and FIG. 13C) or absence (FIG. 13B and FIG. 13D) of an Fc crosslinking antibody (anti-Fc crosslinker). Data are normalized to the activity of anti-LTBR antibody CBE11 (P1AE1873).

FIGS. 14A to 14D show that anti-LTBR agonistic antibodies upregulate ICAM on cancer associated fibroblasts (CAFs) in a crosslinking dependent manner. Shown is the Median fluorescence intensity of ICAM on immortalized CAFs treated with anti-human LTBR agonistic antibodies in the presence (FIG. 14A and FIG. 14C) or absence (FIG. 14B and FIG. 14D) of an anti-Fc crosslinker. Data are normalized to the activity of anti-LTBR antibody CBE11 (P1AE1873).

FIGS. 15A to 15F show the binding of FAP-LTBR bispecific antigen binding molecules to human (FIG. 15A and FIG. 15D), cyno (FIG. 15B and FIG. 15E) and murine LTBR (FIG. 15C and FIG. 15F) on CHO-K1 cells engineered to overexpress human, cyno or murine LTBR. Shown is the Median fluorescence intensity of anti-human Fc secondary antibody detecting bound FAP-LTBR bispecific molecules on cells overexpressing human LTBR (FIG. 15A and FIG. 15D), cynomolgus monkey LTBR (FIG. 15B and FIG. 15E) or murine LTBR (FIG. 15C and FIG. 15F). Data are normalized to baseline.

FIGS. 16A to 16D show that FAP-LTBR bispecific antigen binding molecules upregulate ICAM on cancer associated fibroblasts in a FAP-dependent manner. Shown is the Median fluorescence intensity of ICAM on immortalized CAFs endogenously expressing LTBR and FAP (FIG. 16A and FIG. 16C, hTERT CAFs) or only expressing LTBR (FIG. 16B and FIG. 16D, hTERT CAFs_delFAP). Data are normalized to ICAM expression of untreated control.

FIGS. 17A to 17D illustrate that FAP-LTBR bispecific molecules upregulate ICAM on endothelial cells in a FAP-dependent manner. Shown is the Median fluorescence intensity of ICAM on CD31⁺ HUVECs co-cultured with NIH-3T3 cells overexpressing FAP (FIG. 17A and FIG. 17C) or NIH-3 T3 cells devoid of FAP expression (FIG. 17B and FIG. 17D). Data are normalized to ICAM expression of untreated control and the EC₅₀ values are indicated in the legend.

FIGS. 18A to 18F demonstrate that FAP-LTBR bispecific antigen binding molecules induce the secretion of chemo-attractants by endothelial cells in a FAP-dependent manner. The Concentration of different chemokines (CXCL9, CXCL10 and CXCL11) was measured by Bio-plex in supernatants of HUVEC co-cultured with NIH-3T3 cells overexpressing FAP (FIG. 18A, FIG. 18C and FIG. 18E) or NIH-3T3 cells devoid of FAP expression (FIG. 18B, FIG. 18D and FIG. 18F) treated for 48 hours with FAP-LTBR bispecific molecules.

FIG. 19 shows the increase of T cell adhesion on endothelium stimulated with FAP-LTBR bispecific antigen binding molecules. Shown is the Area of labelled T cells adherent to HUVEC cultures that is stimulated with FAP-LTBR bispecific antibodies (2 nM) in the presence (black bars) or absence (white bars) of FAP. TNFα (0.5 ng/mL) is used as a positive control.

FIGS. 20A and 20B demonstrate that FAP-LTBR bispecific antibody surrogate molecules upregulate adhesion molecules on mouse fibroblasts in a FAP dependent manner in vitro. Shown is the Median fluorescence intensity of VCAM on NIH-3T3 cells (FIG. 20A) or NIH-3T3 FAP overexpressing cells (FIG. 20B) treated with FAP-LTBR antibody surrogate molecules or an anti-mouse LTBR agonistic antibody (5G11). Data are normalized to VCAM baseline expression of untreated controls.

FIGS. 21A and 21B show that FAP-LTBR bispecific antibody surrogate molecules upregulate adhesion molecules on endothelial cells in a FAP dependent manner in vitro. Shown is the Median fluorescence intensity of ICAM on CD31⁺ HUVECs co-cultured with NIH-3T3 (FIG. 21A) or NIH-3T3 cells overexpressing FAP (FIG. 21B).

FIG. 22 shows the study design of an in vivo mouse study to evaluate the safety, efficacy and pharmacodynamic profile of FAP-LTBR bispecific antibodies alone or in combination with an anti-PD-L1 antibody in a subcutaneous MC38-hu CEA tumor model. The timeline describes the treatment and sacrifice time points and the table describes the details of treatment groups, the dosing and the scheduling of this study.

In FIG. 23A it is shown that the FAP-LTBR surrogate molecules inhibit tumor growth in a MC38-huCEA subcutaneous tumor model in monotherapy and improve response to aPD-L1 treatment. Tumor growth curves of Vehicle, P1AF4664, P1AF4674, aPD-L1, P1AF4664+aPD-L1 and P1AF4674+aPD-L1 treated mice are plotted in FIG. 23A. Comparison of tumor volumes at 12 days treatment start show statistically significant larger volume in the Vehicle versus P1AF4764, in the Vehicle versus P1AF4664+aPD-L1, in the Vehicle versus P1AF4674+aPD-L1 and in aPD-L1 vs P1AF4674+aPD-L1. The Statistical analysis was done with One-Way Anova, Holm-Sidak's multiple comparison test (*p<0.05, **p<0.01, ***p<0.001). (FIG. 23B). Individual tumor growth curves of Vehicle, P1AF4664, P1AF4674, aPD-L1, P1AF4664+aPD-L1 and P1AF4674+aPD-L1 treated mice are shown in FIGS. 23C to 23H.

FIGS. 24A and 24B demonstrate that the FAP-LTBR surrogate molecules were able to induce endothelial activation in a MC38-huCEA subcutaneous tumor model. Flow cytometry analysis of tumor single cell suspensions was carried out at day 9 and day 16 post treatment start. Endothelial cells were gated as CD45−, huCEA−, CD31⁺ and podoplanin- and median fluorescence intensity for ICAM (FIG. 24A and FIG. 24B) were quantified. The data show that treatment with P1AF4664 and P1AF4674 increases ICAM on tumor endothelial cells throughout treatment. Statistical analysis was done with One-Way Anova, Holm-Sidak's multiple comparison test (*p<0.05, **p<0.01, ***p<0.001).

FIG. 25 shows the study design of an in vivo mouse study to evaluate the safety, efficacy and pharmacodynamic profile of FAP-LTBR bispecific antibodies alone or in combination with an anti-PD-L1 antibody in a subcutaneous KPC4662-huCEA tumor model. The timeline describes the treatment and sacrifice time points and the table describes the details of treatment groups, the dosing and the scheduling of this KPC4662-huCEA study protocol.

FIGS. 26A to 26C demonstrate that FAP-LTBR surrogate molecules induce high endothelial venules in the KPC4662-huCEA subcutaneous tumor model. In the treatment groups a significant increase in Meca79 positive blood vessels, as a fraction of all blood vessels, was recorded in comparison to the Vehicle group. Statistical analysis was done with One-Way Anova, Holm-Sidak's multiple comparison test (*p<0.05, **p<0.01, ***p<0.001) (FIG. 26A). Representative immunofluorescence staining of CD31 (all blood vessels) and Meca79 (HEVs) in a P1AF4664 treated sample from Day 6 post treatment start is shown in FIG. 26B and FIG. 26C, respectively.

FIG. 27 shows the study design of an in vivo mouse study to evaluate the safety, efficacy and pharmacodynamic profile of LTBR bispecific antigen binding molecules alone or in combination with an anti-PD-L1 antibody in an orthotopic EMT6 tumor model. The timeline describes the treatment and sacrifice time points and the table describes the details of treatment groups, the dosing and the scheduling of this EMT6 study protocol. Vehicle and the murine surrogate LTBR bispecific antigen binding molecules P1AG5459 and P1AG5461 (non-targeted control) were injected at all timepoints indicated above (d0, d2, d5, d7 etc.), if not stated otherwise. PDL1 was injected only at timepoints indicated by empty arrows, if not stated otherwise.

In FIGS. 28A and 28B it is shown that the murine surrogate FAP-LTBR bispecific antibodies (P1AG5459) inhibit tumor growth in an EMT6 orthotopic tumor model in monotherapy and improve response to aPD-L1 treatment. Tumor growth curves (median) of Vehicle, P1AG5459, P1AG5461, aPD-L1 and P1AG5459+aPD-L1 treated mice are plotted in FIG. 28A. Comparison of tumor volumes at 22 days post treatment start is plotted in FIG. 28B and show statistically significant larger volume in the Vehicle versus P1AG5459, in the Vehicle versus P1AG5459+aPD-L1, in the P1AG5459 versus P1AG5461 and in P1AG5461 versus P1AG5459+aPD-L1. The Statistical analysis was done with One-Way Anova, Holm-Sidak's multiple comparison test (*p<0.05, **p<0.01, ***p<0.001).

FIGS. 29A to 29C demonstrate that the murine surrogate LTBR bispecific antibody P1AG5459 was able to induce endothelial activation and HEV differentiation in a FAP dependent manner in an EMT6 orthotopic tumor model. Flow cytometry analysis of tumor single cell suspensions was carried out at day 13 post treatment start. Endothelial cells were gated as CD45−, CD31⁺ and podoplanin- and median fluorescence intensity for ICAM (FIG. 29A) and VCAM (FIG. 29B) or the % of Meca79+ endothelial cells (FIG. 29C) were quantified. The data show that treatment with P1AG5459 alone or in combination with aPD-L1, but not with the non-targeted control P1AG5461, increases ICAM and VCAM on tumor endothelial cells and increases the frequency of Meca79+ endothelial cells (FIG. 29C). Effects on endothelial activation and HEV differentiation are more pronounced in responders (Q) than in non-responder mice (e).

FIG. 30A shows treatment with P1AG5459 alone or in combination with aPD-L1, but not P1AG5461 increases the amount of Meca79+ endothelial cells quantified by Immunofluorescent imaging at day 13 post treatment start. An example image shows the localization of the main vasculature (as depicted by CD31 staining) and Meca79+ endothelium.

FIGS. 30B, 30C and 30D show the quantification of the infiltration of CD8 T cells (FIG. 30B), CD4 T cells (FIG. 30C) and B220+ B cells (FIG. 30D) into the EMT6 orthotopic tumor, as measured by Immunofluorescent Imaging at day 13 post treatment start. P1AG5459 alone or in combination with aPD-L1, also aPD-L1 to a certain extend alone, but not P1AG5461 increases the amount of T and B cells in the tumor. In the case of P1AG5459 monotherapy a clear correlation can be observed in responders (Q) compared to non responder mice (e).

FIGS. 31A to 31C demonstrate that the murine surrogate FAP-LTBR bispecific antibody (P1AG5459) induces the upregulation of chemokines in the EMT6 orthotopic tumor model. The concentration of the chemokines CXCL13 (FIG. 31A), CCL5 (FIG. 31B) and CCL10 (FIG. 31C) were measured in tumor lysates collected at day 13 post treatment start. Tumors treated with P1AG5459 tend to have a higher amount of CXCL13, CCL5 and CXCL10. Responder tumors (Q) tend to show higher concentration of chemokines.

In FIGS. 32A to 32C it is shown that the tumor growth inhibition mediated by the murine surrogate FAP-LTBR bispecific antibody (P1AG5459) in an EMT6 orthotopic tumor model is mediated by CD8, CD4 T and B cells. Tumor growth curves (median) are plotted in FIG. 32A. Comparison of tumor volumes at Day 15 is plotted in FIG. 32B and shows that in the absence of CD8 T cells, there is no difference in tumor volume when comparing Vehicle and P1AG5459 treated mice. Comparison of tumor volumes at Day 20 is plotted in FIG. 32C and shows that in the absence of CD4 T cells or CD20⁺ expressing B cells, there is no difference in tumor volume when comparing Vehicle and P1AG5459 treated mice.

FIG. 33 shows representative immunofluorescence images of orthotopic colorectal cancer tumors treated with Vehicle or the murine surrogate FAP-LTBR bispecific antibodies (P1AG5459). The tumor is identified by Ki67 staining for proliferating cells and the tumor border is marked by a dashed line. CD31 marks blood vessels, pNAD/Meca79 marks High Endothelial Venules and CD8 marks CD8 T cells. The representative images show that P1AG5459 induces the differentiation of HEV and the infiltration of CD8 in the tumor, but not in the surrounding normal colon tissue.

FIG. 34 shows the development of a Tertiary Lymphoid Structure (TLS) in an orthotopic CRC bearing mouse upon treatment with P1AG5459. Ki67 staining of the full section guided the identification of the tumor margin, marked by a dashed line. B220 (B cell) staining of the full section highlights a B cell aggregate, which is zoomed in in the subsequent images. The zoom in images characterize in more detail the cellular composition of the TLS, which contains pNAD/Meca79⁺ High Endothelial Venules, CD11c⁺ myeloid cells, B220⁺ Ki67⁺ proliferating cells, indicative of a germinal center, CD8 and CD4 T cells, some of which co express the activation marker PD1 and the stemness marker TCF1.

FIGS. 35A and 35B compare FAP-LTBR bispecific antigen binding molecules containing different LTBR antigen binding domains in their ability to upregulate ICAM on endothelial cells in a FAP-dependent manner. Shown is the Median fluorescence intensity of ICAM on CD31⁺ HUVECs co-cultured with NIH-3T3 cells overexpressing FAP (FIG. 35A) or NIH-3T3 cells devoid of FAP expression (FIG. 35B). Data are normalized to ICAM expression of untreated control.

FIGS. 36A to 36C compare FAP-LTBR bispecific antigen binding molecules containing different LTBR antigen binding domains in their ability to bind human (FIG. 36A), cyno (FIG. 36B) and murine LTBR (FIG. 36C) on CHO-K1 cells engineered to overexpress human, cyno or murine LTBR. Shown is the Median fluorescence intensity of anti-human Fc secondary antibody detecting bound FAP-LTBR bispecific molecules on cells overexpressing human LTBR (FIG. 36A), cynomolgus monkey LTBR (FIG. 36B) or murine LTBR (FIG. 36C). Data are normalized to baseline.

FIGS. 37A to 37E show schematic representations of an anti-LTBR IgG antibody and bispecific FAP-LTBR antibodies. FIG. 37A shows a schematic representation of a monospecific anti-LTBR human IgG1 PG LALA antibody (BHA10, P1AH0119). FIG. 37B shows a schematic representation of a bispecific 2+1 anti-LTBR/anti-FAP (4B9) antibody as human IgG1 PG LALA with 2 N-terminal Fab arms specific for LTBR (CBE11) and C-terminal anti-FAP VL and VH domains of clone 4B9 (P1AE1079). FIG. 37C shows a schematic representation of a 1+1 anti-LTBR (BHA10)/anti-FAP (4B9) bispecific antibody (P1AH5884) as human IgG1 PG LALA crossMab with crossed VL/VH domains in the anti-LTBR Fab arm and charges in the CH1/Ck domains of the anti-FAP Fab arm. FIG. 37D shows a schematic representation of a 2+1 anti-LTBR (BHA10)/anti-FAP (4B9) bispecific antibody (P1AH5885) as human IgG1 PG LALA crossMab with crossed V-domains in both anti-LTBR Fab arms and charges in the CH1/Ck domains of the anti-FAP Fab fragment that is fused to the C-terminus of the Fc domain (Fc knob-chain). FIG. 37E shows a schematic representation of the 2+1 anti-LTBR (P1AE9459)/anti-FAP (212) bispecific antibody (P1AH5886) as human IgG1 PG LALA crossMab with crossed V-domains in both anti-LTBR Fab arms and charges in the CH1/Ck domains of the anti-FAP Fab fragment that is fused to the C-terminus of the Fc domain (Fc knob-chain).

FIGS. 38A to 38D show the deuteration differences maps between the bound human LTBR compared to the non-complexed LTBR. The deuteration difference map shows the primary sequence of human LTBR (P1AH2680), with the deuteration difference between human LTBR plus antibody/ligand compared to LTBR without antibody/ligand for the five different labelling times 15 seconds, 1, 10, 60 and 300 minutes. The bars above the sequence demonstrate the peptides. The boxes indicate the suggested epitopes for the respective antibody/ligand. Below, the proposed epitopes are mapped (black regions) onto the alphafold model AF-P36941-F1-model_v2 S28 to M227. Shown are the proposed epitopes on human LTBR for P1AE9459 (FIG. 38A), P1AE1873 (FIG. 38B); P1AH0119 (FIG. 38C) and P1AE1235 (FIG. 38D, ligand).

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the terms “antigen binding molecule” or “antibody” are used interchangeably and refer in their broadest sense to a molecule that specifically binds an antigenic determinant. Examples of antigen binding molecules are antibodies, bi- or multispecific antibodies, antibody fragments and scaffold antigen binding proteins. The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

As used herein, the term “antigen binding domain capable of specific binding to a target cell antigen” or “moiety capable of specific binding to a target cell antigen” refers to a polypeptide molecule that specifically binds to an antigenic determinant. In one aspect, the antigen binding domain is able to activate signaling through its target cell antigen. In a particular aspect, the antigen binding domain is able to direct the entity to which it is attached (e.g. the LTBR agonistic antibody) to a target site, for example to a specific type of tumor cell or tumor stroma bearing the antigenic determinant. Antigen binding domains capable of specific binding to a target cell antigen include antibodies and fragments thereof as further defined herein. In addition, antigen binding domains capable of specific binding to a target cell antigen include scaffold antigen binding proteins as further defined herein, e.g. binding domains which are based on designed repeat proteins or designed repeat domains (see e.g. WO 2002/020565). In particular, the antigen binding domain capable of specific binding to a target cell antigen is an antigen binding domain capable of specific binding to Fibroblast Activation Protein (FAP). In relation to an antibody or fragment thereof, the term “antigen binding domain capable of specific binding to a target cell antigen” refers to the part of the molecule that comprises the area which specifically binds to and is complementary to part or all of an antigen. An antigen binding domain capable of specific antigen binding may be provided, for example, by one or more antibody variable domains (also called antibody variable regions). Particularly, an antigen binding domain capable of specific antigen binding comprises an antibody light chain variable region (VL) and an antibody heavy chain variable region (VH) of an antibody. In another aspect, the “antigen binding domain capable of specific binding to a target cell antigen” can also be a Fab fragment or a cross-Fab fragment.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells. A bispecific antigen binding molecule as described herein can also form part of a multispecific antibody.

The term “valent” as used within the current application denotes the presence of a specified number of binding sites specific for one distinct antigenic determinant in an antigen binding molecule that are specific for one distinct antigenic determinant. As such, the terms “bivalent”, “tetravalent”, and “hexavalent” denote the presence of two binding sites, four binding sites, and six binding sites specific for a certain antigenic determinant, respectively, in an antigen binding molecule. In particular aspects of the invention, the bispecific antigen binding molecules according to the invention can be monovalent for a certain antigenic determinant, meaning that they have only one binding site for said antigenic determinant or they can be bivalent or tetravalent for a certain antigenic determinant, meaning that they have two binding sites or four binding sites, respectively, for said antigenic determinant.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteines from the antibody hinge region. Fab′-SH are Fab′ fragments wherein the cysteine residue(s) of the constant domains bear a free thiol group. Pepsin treatment yields an F(ab′)₂ fragment that has two antigen-combining sites (two Fab fragments) and a part of the Fc region. According to the present invention, the term “Fab fragment” also includes “cross-Fab fragments” or “crossover Fab fragments” as defined below.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab _{(VLVH)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab _{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1. A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can be engineered to include up to 25 amino acids without affecting the overall fold of the microprotein. For further details of engineered knottin domains, see WO2008098796.

An “antibody that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more. An “antibody that does not bind to the same epitope” as a reference molecule refers to an antigen binding molecule that does not block binding of the reference molecule to its antigen in a competition assay by 50% or more, and conversely, the reference molecule does not block binding of the antigen binding molecule to its antigen in a competition assay by 50% or more.

The term “antigen binding domain” or “antigen-binding site” refers to the part of an antibody that comprises the area which specifically binds to and is complementary to part or all of an antigen. Where an antigen is large, an antibody may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (koff and kon, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

A “target cell antigen” as used herein refers to an antigenic determinant presented on the surface of a target cell, in particular a target cell in a tumor such as a cancer cell or a cell of the tumor stroma. Thus, the target cell antigen is a tumor-associated antigen. In particular, the “tumor-associated antigen” or TAA is Fibroblast Activation Protein (FAP).

The term “Fibroblast activation protein (FAP)”, also known as Prolyl endopeptidase FAP or Seprase (EC 3.4.21), refers to any native FAP from any vertebrate source, including mammals such as primates (e.g. humans) non-human primates (e.g. cynomolgus monkeys) and rodents (e.g. mice and rats), unless otherwise indicated. The term encompasses “full-length,” unprocessed FAP as well as any form of FAP that results from processing in the cell. The term also encompasses naturally occurring variants of FAP, e.g., splice variants or allelic variants. In one embodiment, the antigen binding molecule of the invention is capable of specific binding to human, mouse and/or cynomolgus FAP. The amino acid sequence of human FAP is shown in UniProt (www.uniprot.org) accession no. Q12884 (version 149, SEQ ID NO:2), or NCBI (www.ncbi.nlm.nih.gov/) RefSeq NP_004451.2. The extracellular domain (ECD) of human FAP extends from amino acid position 26 to 760. The amino acid sequence of an Avi-His-tagged human FAP is shown in SEQ ID NO: 264. The amino acid sequence of mouse FAP is shown in UniProt accession no. P97321 (version 126, SEQ ID NO:282), or NCBI RefSeq NP_032012.1. The extracellular domain (ECD) of mouse FAP extends from amino acid position 26 to 761. SEQ ID NO:265 shows the amino acid of a Avi-His-tagged mouse FAP. Preferably, an anti-FAP binding molecule of the invention binds to the extracellular domain of FAP.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG1, IgG2, IgG3, IgG4, IgA1, and IgA2. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

The term “CH1 domain” denotes the part of an antibody heavy chain polypeptide that extends approximately from EU position 118 to EU position 215 (EU numbering system according to Kabat). In one aspect, a CH1 domain has the amino acid sequence of ASTKGPSVFP LAPSSKSTSG GTAALGCLVK DYFPEPVTVS WNSGALTSGV HTFPAVLQSS GLYSLSSVVT VPSSSLGTQT YICNVNHKPS NTKVDKKV (SEQ ID NO: 283). Usually, a segment having the amino acid sequence of EPKSC (SEQ ID NO:284) is following to link the CH1 domain to the hinge region, however in the case of a CH1 domain with a free C-terminal end (such as in a crossfab fragment) this segment may also comprise the amino acid sequence of EPKSCD (SEQ ID NO:285) or EPKSCS (SEQ ID NO:286).

The term “hinge region” denotes the part of an antibody heavy chain polypeptide that joins in a wild-type antibody heavy chain the CH1 domain and the CH2 domain, e. g. from about position 216 to about position 230 according to the EU number system of Kabat, or from about position 226 to about position 230 according to the EU number system of Kabat. The hinge regions of other IgG subclasses can be determined by aligning with the hinge-region cysteine residues of the IgG1 subclass sequence. The hinge region is normally a dimeric molecule consisting of two polypeptides with identical amino acid sequence. The hinge region generally comprises up to 25 amino acid residues and is flexible allowing the associated target binding sites to move independently. The hinge region can be subdivided into three domains: the upper, the middle, and the lower hinge domain (see e.g. Roux, et al., J. Immunol. 161 (1998) 4083). In one aspect, the hinge region has the amino acid sequence DKTHTCPXCP (SEQ ID NO: 287), wherein X is either S or P. In one aspect, the hinge region has the amino acid sequence HTCPXCP (SEQ ID NO: 288), wherein X is either S or P. In one aspect, the hinge region has the amino acid sequence CPXCP (SEQ ID NO: 289), wherein X is either S or P.

The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. (EU numbering system according to Kabat). In one aspect, a CH2 domain has the amino acid sequence of APELLGGPSV FLFPPKPKDT LMISRTPEVT CVWDVSHEDP EVKFNWYVDG VEVHNAKTKP REEQESTYRW SVLTVLHQDW LNGKEYKCKV SNKALPAPIE KTISKAK (SEQ ID NO: 290). The CH2 domain is unique in that it is not closely paired with another domain. Rather, two N-linked branched carbohydrate chains are interposed between the two CH2 domains of an intact native Fc-region. It has been speculated that the carbohydrate may provide a substitute for the domain-domain pairing and help stabilize the CH2 domain. Burton, Mol. Immunol. 22 (1985) 161-206. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 according to EU numbering system according to Kabat of an IgG). In one aspect, the CH3 domain has the amino acid sequence of GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTQKS LSLSPG (SEQ ID NO: 291). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The term “wild-type Fc domain” denotes an amino acid sequence identical to the amino acid sequence of an Fc domain found in nature. Wild-type human Fc domains include a native human IgG1 Fc-region (non-A and A allotypes), native human IgG2 Fc-region, native human IgG3 Fc-region, and native human IgG4 Fc-region as well as naturally occurring variants thereof. Wild-type Fc-regions are denoted in SEQ ID NO: 155 (IgG1, caucasian allotype), SEQ ID NO: 156 (IgG1, afroamerican allotype), SEQ ID NO: 157 (IgG2), SEQ ID NO:158 (IgG3) and SEQ ID NO: 159 (IgG4). The term “variant (human) Fc domain” denotes an amino acid sequence which differs from that of a “wild-type” (human) Fc domain amino acid sequence by virtue of at least one “amino acid mutation”. In one aspect, the variant Fc-region has at least one amino acid mutation compared to a native Fc-region, e.g. from about one to about ten amino acid mutations, and in one aspect from about one to about five amino acid mutations in a native Fc-region. In one aspect, the (variant) Fc-region has at least about 95% homology with a wild-type Fc-region.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector function” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492, Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 10³-fold and eliminated the human monocyte response to antibody-sensitized red blood cells (Armour, K. L., et al., Eur. J. Immunol. 29 (1999) 2613-2624).
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells it seems to function to suppress further immunoglobulin production and isotype switching to, for example, the IgE class. On macrophages, FcγRIIB acts to inhibit phagocytosis as mediated through FcγRIIA. On eosinophils and mast cells the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat).
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

The term “ADCC” or “antibody-dependent cellular cytotoxicity” is a function mediated by Fc receptor binding and refers to lysis of target cells by an antibody as reported herein in the presence of effector cells. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA). In particular, binding to FcγR on NK cells is measured.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “LTBR”, as used herein, refers to any native Lymphotoxin beta receptor (LTBR) from any vertebrate source, including mammals such as primates (e.g. humans) and rodents (e.g., mice and rats), unless otherwise indicated. The term encompasses “full-length,” unprocessed LTBR as well as any form of LTBR that results from processing in the cell. The term also encompasses naturally occurring variants of LTBR, e.g., splice variants or allelic variants. The amino acid sequence of an exemplary human LTBR is shown in SEQ ID NO:1 (Uniprot No. P36941) and the amino acid sequence of an exemplary mouse LTBR is shown in SEQ ID NO: 297 (Uniprot No. B2RRV3). The receptor is expressed on the surface of cells in the parenchyma and stroma of most lymphoid organs but is absent on T- and B-lymphocytes. LTBR can also be referred to as “tumor necrosis factor receptor superfamily member 3 (TNFRSF3). Signaling through LTBR by the LTα/β heterotrimer (LTα1β2) is important during lymphoid development. LTBR is also known to bind the ligand LIGHT (TNFSF14). Whereas LTα1β2 is specific for LTBR, LIGHT also binds to and activates HVEM (TNFRSF14), a receptor expressed on and implicated in the regulation of immune cells.

The term “LTBR agonist” as used herein includes any moiety that agonizes the interaction of LTBR with its ligand. LTBR as used in this context refers preferably to human LTBR, thus the LTBR agonist is preferably an agonist of human LTBR (SEQ ID NO:1). Typically, the moiety will be an agonistic LTBR antibody or antibody fragment.

The terms “anti-LTBR antibody”, “anti-LTBR”, “LTBR antibody and “an antibody that specifically binds to LTBR” refer to an antibody that specifically binds to LTBR with sufficient affinity such that the antibody is useful as a diagnostic and/or therapeutic agent in targeting LTBR. In one aspect, the extent of binding of an anti-LTBR antibody to an unrelated, non-LTBR protein is less than about 10% of the binding of the antibody to LTBR as measured, e.g., by a radioimmunoassay (RIA) or flow cytometry (FACS). In certain embodiments, an antibody that binds to LTBR has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁶ M or less, e.g. from 10⁻⁶⁸ M to 10⁻¹³ M, e.g., from 10⁻⁸ M to 10⁻¹⁰ M).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2, i.e. the peptides selected from the group consisting of GGGGS (SEQ ID NO:298), GGGGSGGGGS (SEQ ID NO:299), SGGGGSGGGG (SEQ ID NO:300) and GGGGSGGGGSGGGG (SEQ ID NO:301), but also include the sequences GSPGSSSSGS (SEQ ID NO:302), (G4S)₃ (SEQ ID NO:303), (G4S)₄ (SEQ ID NO:304), GSGSGSGS (SEQ ID NO:305), GSGSGNGS (SEQ ID NO:306), GGSGSGSG (SEQ ID NO:307), GGSGSG (SEQ ID NO:308), GGSG (SEQ ID NO:309), GGSGNGSG (SEQ ID NO:310), GGNGSGSG (SEQ ID NO:311) and GGNGSG (SEQ ID NO:312). Peptide linkers of particular interest are (G4S) (SEQ ID NO:298), (G₄S)₂ or GGGGSGGGGS (SEQ ID NO:299), (G4S)₃ (SEQ ID NO:303) and (G4S)₄ (SEQ ID NO:304).

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

By “fused” or “connected” is meant that the components (e.g. a heavy chain of an antibody and a Fab fragment) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611. Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997), Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

In certain embodiments, amino acid sequence variants of the bispecific antigen binding molecules provided herein are contemplated. For example, it may be desirable to improve the binding affinity and/or other biological properties of the TNF ligand trimer-containing antigen binding molecules. Amino acid sequence variants of the TNF ligand trimer-containing antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE A

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more CDR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in CDRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include bispecific antigen binding molecules of the invention with an N-terminal methionyl residue. Other insertional variants of the molecule include the fusion to the N- or C-terminus to a polypeptide which increases the serum half-life of the bispecific antigen binding molecules.

In certain aspects, the bispecific antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the TNF ligand trimer-containing antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in TNF family ligand trimer-containing antigen binding molecule may be made in order to create variants with certain improved properties. In one aspect, variants of bispecific antigen binding molecules or antibodies of the invention are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). In another aspect, variants of the bispecific antigen binding molecules or antibodies of the invention are provided with bisected oligosaccharides, e.g., in which a biantennary oligosaccharide attached to the Fc region is bisected by GlcNAc. Such variants may have reduced fucosylation and/or improved ADCC function, see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain aspects, it may be desirable to create cysteine engineered variants of the bispecific antigen binding molecules of the invention, e.g., “thioMAbs,” in which one or more residues of the molecule are substituted with cysteine residues. In particular aspects, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain aspects, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

The term “nucleic acid” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a bispecific antigen binding molecule or antibody” refers to one or more nucleic acid molecules encoding the heavy and light chains (or fragments thereof) of the bispecific antigen binding molecule or antibody, including such nucleic acid molecule(s) in a single vector or separate vectors, and such nucleic acid molecule(s) present at one or more locations in a host cell.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, including refractory versions of any of the above cancers, or a combination of one or more of the above cancers.

The term “chemotherapeutic agent” as used herein refers to a chemical compound useful in the treatment of cancer. In one aspect, the chemotherapeutic agent is an antimetabolite. In one aspect, the antimetabolite is selected from the group consisting of Aminopterin, Methotrexate, Pemetrexed, Raltitrexed, Cladribine, Clofarabine, Fludarabine, Mercaptopurine, Pentostatin, Thioguanine, Capecitabine, Cytarabine, Fluorouracil, Floxuridine, and Gemcitabine. In one particular aspect, the antimetabolite is capecitabine or gemcitabine. In another aspect, the antimetabolite is fluorouracil. In one aspect, the chemotherapeutic agent is an agent that affects microtubule formation. In one aspect, the agent that affects microtubule formation is selected from the group consisting of: paclitaxel, docetaxel, vincristine, vinblastine, vindesine, vinorelbin, taxotere, etoposide, and teniposide.

In another aspect, the chemotherapeutic agent is an alkylating agent such as cyclophosphamide. In one aspect, the chemotherapeutic agent is a cytotoxic antibiotic such as a topoisomerase II inhibitor. In one aspect, the topoisomerase II inhibitor is doxorubicin.

### Exemplary Agonistic LTBR Antibodies

Provided herein are novel antibodies and antibody fragments that specifically bind to lymphotoxin beta receptor (LTBR). Provided are novel antibodies and antibody fragments that specifically bind to an antigen comprising the amino acid sequence of SEQ ID NO:1. Thus, these antibodies specifically bind to human LTBR. They are agonistic hu LTBR antibodies.

These antibodies are able to bind to human LTBR and to cynomolgus LTBR with less than a 2-fold difference in affinity. Some of the new agonistic hu LTBR antibodies are even able to bind to human LTBR, cynomolgus LTBR and murine LTBR.

Disclosed herein are agonistic LTBR antibodies, that bind to the human LTBR extracellular domain (ECD of the amino acid sequence of SEQ ID NO: 359) with an EC₅₀ of less than 4 nM as measured by ELISA (see Example 1.5). In one aspect, the agonistic LTBR antibodies bind to the human LTBR ECD with an EC₅₀ of less than 1 nM as measured by ELISA. In one particular aspect, the agonistic LTBR antibodies bind to the human LTBR ECD with an EC₅₀ of less than 0.15 nM as measured by ELISA.

The agonistic LTBR antibodies as disclosed herein bind to the cynomolgus LTBR extracellular domain (ECD of the amino acid sequence of SEQ ID NO: 361) with an EC₅₀ of less than 5 nM as measured by ELISA (see Example 1.5). In one aspect, the agonistic LTBR antibodies bind to the cynomolgus LTBR ECD with an EC₅₀ of less than 0.5 nM as measured by ELISA. In one particular aspect, the agonistic LTBR antibodies bind to the cynomolgus LTBR ECD with an EC₅₀ of less than 0.1 nM as measured by ELISA.

The agonistic LTBR antibodies as described herein require cross-linking for their agonistic activity to activate human LTBR, meaning that they can only stimulate LTBR via a cross-linking dependent mechanism. A “cross linking dependent mechanism” could be for example a Fc cross linking dependent mechanism wherein the antibody has to bind both LTBR and an Fc receptor in order to stimulate LTBR. As such, the antibody has to be capable of binding both LTBR and an Fc receptor. If the antibody would be crosslinking-independent, it could stimulate LTBR in the absence of binding to an Fc receptor. This could lead to widespread LTBR activation in the human body and serious safety issues associated therewith.

The agonistic LTBR antibodies as described herein also require cross-linking for their agonistic activity to induce ICAM upregulation in human umbilical vein endothelial cells or cancer associated fibroblasts, as is shown herein in Example 5.2.

Agonistic LTBR antibodies as described herein are also able to inhibit the interaction between human LTBR and its human ligands lymphotoxin α1β2 and LIGHT. This has been shown in the ligand competition experiments by ELISA as described in Example 1.6.

It has been shown, that agonistic LTBR antibodies as described herein compete for binding to hu LTBR with human LIGHT, i.e a natural ligand of LTBR comprising the amino acid sequence of SEQ ID NO:358.

In one aspect, provided herein is an agonistic LTBR antibody (or an antigen binding domain that specifically binds to LTBR), wherein said antibody or antigen binding domain comprises
a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48, or
a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72, or
a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80, or
a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

In one aspect, provided is an agonistic LTBR antibody (or an antigen binding domain that specifically binds to LTBR), wherein said antibody comprises
a heavy chain variable region (V_{H} LTBR) comprising the CDR-H1, CDR-H2 and CDR-H3 of the amino acid sequence of SEQ ID NO:33 and a light chain variable region (V_{L} LTBR) comprising the CDR-L1, CDR-L2 and CDR-L3 of the amino acid sequence of SEQ ID NO:34, or
a heavy chain variable region (V_{H} LTBR) comprising the CDR-H1, CDR-H2 and CDR-H3 of the amino acid sequence of SEQ ID NO:41 and a light chain variable region (V_{L} LTBR) comprising the CDR-L1, CDR-L2 and CDR-L3 of the amino acid sequence of SEQ ID NO:42, or
a heavy chain variable region (V_{H} LTBR) comprising the CDR-H1, CDR-H2 and CDR-H3 of the amino acid sequence of SEQ ID NO:49 and a light chain variable region (V_{L} LTBR) comprising the CDR-L1, CDR-L2 and CDR-L3 of the amino acid sequence of SEQ ID NO:50, or
a heavy chain variable region (V_{H} LTBR) comprising the CDR-H1, CDR-H2 and CDR-H3 of the amino acid sequence of SEQ ID NO:57 and a light chain variable region (V_{L} LTBR) comprising the CDR-L1, CDR-L2 and CDR-L3 of the amino acid sequence of SEQ ID NO:58, or
a heavy chain variable region (V_{H} LTBR) comprising the CDR-H1, CDR-H2 and CDR-H3 of the amino acid sequence of SEQ ID NO:65 and a light chain variable region (V_{L} LTBR) comprising the CDR-L1, CDR-L2 and CDR-L3 of the amino acid sequence of SEQ ID NO:66,
a heavy chain variable region (V_{H} LTBR) comprising the CDR-H1, CDR-H2 and CDR-H3 of the amino acid sequence of SEQ ID NO:73 and a light chain variable region (V_{L} LTBR) comprising the CDR-L1, CDR-L2 and CDR-L3 of the amino acid sequence of SEQ ID NO:74,
a heavy chain variable region (V_{H} LTBR) comprising the CDR-H1, CDR-H2 and CDR-H3 of the amino acid sequence of SEQ ID NO:81 and a light chain variable region (V_{L} LTBR) comprising the CDR-L1, CDR-L2 and CDR-L3 of the amino acid sequence of SEQ ID NO:82, or
a heavy chain variable region (V_{H} LTBR) comprising the CDR-H1, CDR-H2 and CDR-H3 of the amino acid sequence of SEQ ID NO:89 and a light chain variable region (V_{L} LTBR) comprising the CDR-L1, CDR-L2 and CDR-L3 of the amino acid sequence of SEQ ID NO:90, or
a heavy chain variable region (V_{H} LTBR) comprising the CDR-H1, CDR-H2 and CDR-H3 of the amino acid sequence of SEQ ID NO:97 and a light chain variable region (V_{L} LTBR) comprising the CDR-L1, CDR-L2 and CDR-L3 of the amino acid sequence of SEQ ID NO:98.

In one aspect, provided is an agonistic LTBR antibody (or an antigen binding domain that specifically binds to LTBR), wherein said antibody or antigen binding domain comprises
a heavy chain variable region (V_{H} LTBR) comprising the amino acid sequence of SEQ ID NO:33 and a light chain variable region (V_{L} LTBR) comprising the amino acid sequence of SEQ ID NO:34, or
a heavy chain variable region (V_{H} LTBR) comprising the amino acid sequence of SEQ ID NO:41 and a light chain variable region (V_{L} LTBR) comprising the amino acid sequence of SEQ ID NO:42, or
a heavy chain variable region (V_{H} LTBR) comprising the amino acid sequence of SEQ ID NO:49 and a light chain variable region (V_{L} LTBR) comprising the amino acid sequence of SEQ ID NO:50, or
a heavy chain variable region (V_{H} LTBR) comprising the amino acid sequence of SEQ ID NO:57 and a light chain variable region (V_{L} LTBR) comprising the amino acid sequence of SEQ ID NO:58, or
a heavy chain variable region (V_{H} LTBR) comprising the amino acid sequence of SEQ ID NO:65 and a light chain variable region (V_{L} LTBR) comprising the amino acid sequence of SEQ ID NO:66,
a heavy chain variable region (V_{H} LTBR) comprising the amino acid sequence of SEQ ID NO:73 and a light chain variable region (V_{L} LTBR) comprising the amino acid sequence of SEQ ID NO:74,
a heavy chain variable region (V_{H} LTBR) comprising the amino acid sequence of SEQ ID NO:81 and a light chain variable region (V_{L} LTBR) comprising the amino acid sequence of SEQ ID NO:82, or
a heavy chain variable region (V_{H} LTBR) comprising the amino acid sequence of SEQ ID NO:89 and a light chain variable region (V_{L} LTBR) comprising the amino acid sequence of SEQ ID NO:90, or
a heavy chain variable region (V_{H} LTBR) comprising the amino acid sequence of SEQ ID NO:97 and a light chain variable region (V_{L} LTBR) comprising the amino acid sequence of SEQ ID NO:98.

In one particular aspect, the agonistic LTBR antibodies as described herein are agonistic LTBR antibodies that specifically bind to human LTBR, cynomolgus LTBR and murine LTBR. Disclosed herein are agonistic LTBR antibodies, that bind to the murine LTBR extracellular domain (ECD of the amino acid sequence of SEQ ID NO: 360) with an EC₅₀ of less than 1 nM as measured by ELISA (see Example 1.5). In one aspect, the agonistic LTBR antibodies bind to the murine LTBR ECD with an EC₅₀ of less than 0.15 nM as measured by ELISA.

In one aspect, provided is an agonistic LTBR antibody (or an antigen binding domain) that specifically binds to human LTBR, cynomolgus LTBR and murine LTBR comprising
a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48, or
a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80, or
a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

In one aspect, the agonistic LTBR antibody (or an antigen binding domain) that specifically binds to human LTBR, cynomolgus LTBR and murine LTBR comprises
a heavy chain variable region (V_{H} LTBR) comprising the amino acid sequence of SEQ ID NO:33 and a light chain variable region (V_{L} LTBR) comprising the amino acid sequence of SEQ ID NO:34, or
a heavy chain variable region (V_{H} LTBR) comprising the amino acid sequence of SEQ ID NO:49 and a light chain variable region (V_{L} LTBR) comprising the amino acid sequence of SEQ ID NO:50, or
a heavy chain variable region (V_{H} LTBR) comprising the amino acid sequence of SEQ ID NO:81 and a light chain variable region (V_{L} LTBR) comprising the amino acid sequence of SEQ ID NO:82, or
a heavy chain variable region (V_{H} LTBR) comprising the amino acid sequence of SEQ ID NO:89 and a light chain variable region (V_{L} LTBR) comprising the amino acid sequence of SEQ ID NO:90, or
a heavy chain variable region (V_{H} LTBR) comprising the amino acid sequence of SEQ ID NO:97 and a light chain variable region (V_{L} LTBR) comprising the amino acid sequence of SEQ ID NO:98.

In one particular aspect, an agonistic LTBR antibody as described herein binds to the epitope region of SEQ ID NO:351 on human LTBR. In one aspect, an agonistic LTBR antibody as described herein binds to an epitope which overlaps with the epitope to which human lymphotoxin α1β2 binds to on human LTBR. In one aspect, an agonistic LTBR antibody as described herein binds to a different epitope region on human LTBR than reference antibodies BHA10 and CBE11 This has been demonstrated by hydrogen/deuterium exchange (HDX) mass spectrometry as described in Example 6.4.

In one aspect, such agonistic LTBR antibody comprises a heavy chain variable region (V_{H} LTBR) comprising a CDR-H1 comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32. In one aspect, comprises a heavy chain variable region (V_{H} LTBR) comprising the amino acid sequence of SEQ ID NO:33 and a light chain variable region (V_{L} LTBR) comprising the amino acid sequence of SEQ ID NO:34 (antibody P1AE9459).

In one aspect, provided is an agonistic LTBR antibody (or an antigen binding domain that specifically binds to LTBR) that originates from immunization in transgenic rabbits. In one aspect, this antibody (or antigen binding domain) comprise
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
(ii) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(iii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48, or
(iv) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(v) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(vi) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

In one aspect, the agonistic LTBR antibody (or antigen binding domain) derived from transgenic rabbit immunization is a cross-reactive antibody or antigen binding domain that specifically binds to human, cynomolgus and mouse LTBR. In one aspect, the antibody or antigen binding domain comprises a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48. In one aspect, the antibody or antigen binding domain comprises a heavy chain variable region (V_{H} LTBR) comprising the amino acid sequence of SEQ ID NO:33 and a light chain variable region (V_{L} LTBR) comprising the amino acid sequence of SEQ ID NO:34, or a heavy chain variable region (V_{H} LTBR) comprising the amino acid sequence of SEQ ID NO:49 and a light chain variable region (V_{L} LTBR) comprising the amino acid sequence of SEQ ID NO:50.

In another aspect, provided is a human LTBR antibody or antigen binding domain that originates from rat immunization. In particular, this antibody or antigen binding domain is a cross-reactive antibody or antigen binding domain that specifically binds to human, cynomolgus and mouse LTBR. In one aspect, the antibody or antigen binding domain comprises a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80. In one aspect, the antibody or antigen binding domain comprises a heavy chain variable region (V_{H} LTBR) comprising the amino acid sequence of SEQ ID NO:81 and a light chain variable region (V_{L} LTBR) comprising the amino acid sequence of SEQ ID NO:82.

In yet another aspect, provided is a human LTBR antibody or antigen binding domain that is generated from a phage display library. In particular, this antibody or antigen binding domain is a cross-reactive antibody or antigen binding domain that specifically binds to human, cynomolgus and mouse LTBR. In one aspect, the antibody or antigen binding domain comprises a heavy chain variable region (V_{H} LTBR) comprising a CDR-H1 comprising the amino acid sequence of SEQ ID NO:83, a CDR-H2 comprising the amino acid sequence of SEQ ID NO:84, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88. In one aspect, the antibody or antigen binding domain comprises a heavy chain variable region (V_{H} LTBR) comprising a CDR-H1 comprising the amino acid sequence of SEQ ID NO:91, a CDR-H2 comprising the amino acid sequence of SEQ ID NO:92, and a CDR-H3 comprising the amino acid sequence of SEQ ID NO:93, and a light chain variable region (V_{L} LTBR) comprising a CDR-L1 comprising the amino acid sequence of SEQ ID NO:94, a CDR-L2 comprising the amino acid sequence of SEQ ID NO:95, and a CDR-L3 comprising the amino acid sequence of SEQ ID NO:96. In one aspect, the antibody or antigen binding domain comprises a heavy chain variable region (V_{H} LTBR) comprising the amino acid sequence of SEQ ID NO: 89 and a light chain variable region (V_{L} LTBR) comprising the amino acid sequence of SEQ ID NO:90.

In another aspect, the antibody or antigen binding domain comprises a heavy chain variable region (V_{H} LTBR) comprising the amino acid sequence of SEQ ID NO:97 and a light chain variable region (V_{L} LTBR) comprising the amino acid sequence of SEQ ID NO:98.

In one aspect, the antibody that specifically binds to LTBR is a full-length antibody, in particular of human IgG1 subclass. In one particular aspect, it comprises the amino acid mutations L234A, L235A and P329G (numbering according to Kabat EU index). In one particular aspect, the antibody comprises
(i) two heavy chains comprising the amino acid sequence of SEQ ID NO:227 and two light chains comprising the amino acid sequence of SEQ ID NO:228, or
(ii) two heavy chains comprising the amino acid sequence of SEQ ID NO:229 and two light chains comprising the amino acid sequence of SEQ ID NO:230, or
(iii) two heavy chains comprising the amino acid sequence of SEQ ID NO:231 and two light chains comprising the amino acid sequence of SEQ ID NO:232, or
(iv) two heavy chains comprising the amino acid sequence of SEQ ID NO:233 and two light chains comprising the amino acid sequence of SEQ ID NO:234, or
(v) two heavy chains comprising the amino acid sequence of SEQ ID NO:235 and two light chains comprising the amino acid sequence of SEQ ID NO:236, or
(vi) two heavy chains comprising the amino acid sequence of SEQ ID NO:237 and two light chains comprising the amino acid sequence of SEQ ID NO:238, or
(vii) two heavy chains comprising the amino acid sequence of SEQ ID NO:239 and two light chains comprising the amino acid sequence of SEQ ID NO:240, or
(viii) two heavy chains comprising the amino acid sequence of SEQ ID NO:241 and two light chains comprising the amino acid sequence of SEQ ID NO:242, or
(ix) two heavy chains comprising the amino acid sequence of SEQ ID NO:243 and two light chains comprising the amino acid sequence of SEQ ID NO:244.

### Bispecific Agonistic LTBR Antibodies

Provided herein are also new bispecific antigen binding molecules capable of specific binding to lymphotoxin beta receptor (LTBR) and to a tumor-associated antigen such as Fibroblast activation protein (FAP) that combine an antigen binding domain that specifically binds to FAP with at least one antigen binding domain capable of agonistic binding to LTBR, wherein the activation through LTBR is provided by cross-linking through binding to FAP expressed on tumor stromal cells. The bispecific agonistic LTBR antibodies disclosed herein possess particularly advantageous properties such as producibility, stability, binding affinity, biological activity, targeting efficiency, reduced internalization, acceptable pharmacokinetic (PK) properties, reduced toxicity, an extended dosage range that can be given to a patient and thereby a possibly enhanced efficacy.

### Exemplary Bispecific Agonistic LTBR Antibodies

In one aspect, provided are agonistic LTBR antibodies that are multispecific antibodies comprising the agonistic LTBR antibody as described herein before. In one aspect, provided are agonistic LTBR antibodies that are bispecific antibodies. The bispecific agonistic LTBR antibody preferably comprises a Fc domain of human origin, particularly of human IgG subclass, more particularly of human IgG1 subclass. In one aspect, the bispecific agonistic LTBR antibody comprises a Fc domain of human IgG1 subclass comprising one or more amino acid substitution that reduces the binding affinity of the antigen binding molecule to an Fc receptor and/or effector function. In one aspect, the bispecific agonistic LTBR antibody comprises a Fc domain of human IgG1 subclass with the amino acid mutations L234A, L235A and P329G (numbering according to Kabat EU index).

In one aspect, the agonistic LTBR antibody is a bispecific antibody that that specifically binds to LTBR and to a tumor associated antigen (TAA). In particular, the bispecific agonistic LTBR antibody is a LTBR agonist that is targeted against FAP. In one aspect, the bispecific agonistic LTBR antibody comprises a Fc region composed of a first and a second subunit which comprises mutations that reduce effector function. The use of an Fc region comprising mutations that reduce or abolish effector function will prevent unspecific agonism by crosslinking via Fc receptors and will prevent ADCC of LTBR expressing cells. The bispecific agonistic LTBR antibodies as described herein possess the advantage over conventional antibodies in that they selectively induce immune response at the target cells, which are typically in the tumor stroma, i.e in proximity to the tumor.

The bispecific agonistic LTBR antibodies are thus characterized by FAP-targeted agonistic binding to LTBR. In the presence of FAP-expressing cells the bispecific antigen binding molecules are able to activate cancer associated fibroblasts (CAFs) (Example 5.2.1), to activate endothelial cells (Example 5.2.2) and to modulate human endothelium to upregulate adhesion molecules and chemoattractants, which are critical to the immune infiltration cascade. The bispecific antigen binding molecules described herein are able to induce T cell adhesion (Example 5.2.3).

In one aspect, provided is a bispecific agonistic LTBR antibody comprising
(a) a first antigen binding domain that specifically binds to Fibroblast Activation Protein (FAP),
(b) a second antigen binding domain that specifically binds to lymphotoxin beta receptor (LTBR), and
(c) a Fc domain composed of a first and a second subunit and comprising one or more amino acid substitution that reduces the binding affinity of the antigen binding molecule to an Fc receptor and/or effector function.

The bispecific agonistic LTBR antibodies possess a Fc domain composed of a first and a second subunit that comprises one or more amino acid substitution that reduces the binding affinity of the antigen binding molecule to an Fc receptor and/or effector function. Fc receptor-mediated cross-linking is thereby abrogated and tumor-specific activation is achieved by cross-linking through binding of the antigen binding domain that specifically binds to FAP through binding to its tumor-associated target.

In one aspect, provided is a bispecific agonistic LTBR antibody, comprising
(a) a first Fab fragment that specifically binds to Fibroblast Activation Protein (FAP),
(b) a second Fab fragment that specifically binds to lymphotoxin beta receptor (LTBR), and (c) a Fc domain composed of a first and a second subunit and comprising one or more amino acid substitution that reduces the binding affinity of the antigen binding molecule to an Fc receptor and/or effector function.

In one aspect, the bispecific agonistic LTBR antibody as disclosed herein comprises a first antigen binding domain that specifically binds to Fibroblast Activation Protein (FAP) comprising
(i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8, or
(ii) a heavy chain variable region (V_{H}FAP) comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21, and a light chain variable region (V_{L}FAP) comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24, or
(iii) a heavy chain variable region (V_{H}FAP) comprising a CDR-H1 comprising the amino acid sequence of SEQ ID NO:11, a CDR-H2 comprising the amino acid sequence of SEQ ID NO:12, and a CDR-H3 comprising the amino acid sequence of SEQ ID NO:13, and a light chain variable region (V_{L}FAP) comprising a CDR-L1 comprising the amino acid sequence of SEQ ID NO:14, a CDR-L2 comprising the amino acid sequence of SEQ ID NO:15, and a CDR-L3 comprising the amino acid sequence of SEQ ID NO:16.

In one aspect, the first antigen binding domain that specifically binds to FAP comprises
(i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8, or
(ii) a heavy chain variable region (V_{H}FAP) comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21, and a light chain variable region (V_{L}FAP) comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24.

In one particular aspect, the first antigen binding domain that specifically binds to FAP comprises (i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8. In one aspect, the first antigen binding domain that specifically binds to FAP comprises a heavy chain variable region (V_{H}FAP) comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21, and a light chain variable region (V_{L}FAP) comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24. In another aspect, the first antigen binding domain that specifically binds to FAP comprises a heavy chain variable region (V_{H}FAP) comprising a CDR-H1 comprising the amino acid sequence of SEQ ID NO:11, a CDR-H2 comprising the amino acid sequence of SEQ ID NO:12, and a CDR-H3 comprising the amino acid sequence of SEQ ID NO:13, and a light chain variable region (V_{L}FAP) comprising a CDR-L1 comprising the amino acid sequence of SEQ ID NO:14, a CDR-L2 comprising the amino acid sequence of SEQ ID NO:15, and a CDR-L3 comprising the amino acid sequence of SEQ ID NO:16.

In one aspect, the first antigen binding domain that specifically binds to FAP comprises a heavy chain variable region (V_{H}FAP) comprising the CDR-H1, CDR-H2 and CDR-H3 of the amino acid sequence of SEQ ID NO:9 and a light chain variable region (V_{L}FAP) comprising the CDR-L1, CDR-L2 and CDR-L3 of the amino acid sequence of SEQ ID NO:10. In another aspect, it comprises a heavy chain variable region (V_{H}FAP) comprising the CDR-H1, CDR-H2 and CDR-H3 of the amino acid sequence of SEQ ID NO:25 and a light chain variable region (V_{L}FAP) comprising the CDR-L1, CDR-L2 and CDR-L3 of the amino acid sequence of SEQ ID NO:26. In yet another aspect, it comprises a heavy chain variable region (V_{H}FAP) comprising the CDR-H1, CDR-H2 and CDR-H3 of the amino acid sequence of SEQ ID NO:17 and a light chain variable region (V_{L}FAP) comprising the CDR-L1, CDR-L2 and CDR-L3 of the amino acid sequence of SEQ ID NO:18.

In one aspect, the first antigen binding domain that specifically binds to FAP comprises a heavy chain variable region (V_{H}FAP) comprising an amino acid sequence that is at least about 95% identical to the amino acid sequence of SEQ ID NO:9, and a light chain variable region (V_{L}FAP) comprising an amino acid sequence that is at least about 95% identical to the amino acid sequence of SEQ ID NO:10, or it comprises a heavy chain variable region (V_{H}FAP) comprising an amino acid sequence that is at least about 95% identical to the amino acid sequence of SEQ ID NO:25, and a light chain variable region (V_{L}FAP) comprising an amino acid sequence that is at least about 95% identical to the amino acid sequence of SEQ ID NO:26, or it comprises a heavy chain variable region (V_{H}FAP) comprising an amino acid sequence that is at least about 95% identical to the amino acid sequence of SEQ ID NO:17, and a light chain variable region (V_{L}FAP) comprising an amino acid sequence that is at least about 95% identical to the amino acid sequence of SEQ ID NO:18.

In one aspect, the first antigen binding domain that specifically binds to FAP comprises a heavy chain variable region (V_{H}FAP) comprising the amino acid sequence of SEQ ID NO:9 and a light chain variable region (V_{L}FAP) comprising the amino acid sequence of SEQ ID NO:10 or it comprises a heavy chain variable region (V_{H}FAP) comprising the amino acid sequence of SEQ ID NO:25 and a light chain variable region (V_{L}FAP) comprising the amino acid sequence of SEQ ID NO:26 or it comprises a heavy chain variable region (V_{H}FAP) comprising the amino acid sequence of SEQ ID NO:17 and a light chain variable region (V_{L}FAP) comprising the amino acid sequence of SEQ ID NO:18. In one particular aspect, the first antigen binding domain that specifically binds to FAP comprises a heavy chain variable region (V_{H}FAP) comprising the amino acid sequence of SEQ ID NO:9 and a light chain variable region (V_{L}FAP) comprising the amino acid sequence of SEQ ID NO:10. In one aspect, it comprises a heavy chain variable region (V_{H}FAP) comprising the amino acid sequence of SEQ ID NO:25 and a light chain variable region (V_{L}FAP) comprising the amino acid sequence of SEQ ID NO:26. IN another aspect, it comprises a heavy chain variable region (V_{H}FAP) comprising the amino acid sequence of SEQ ID NO:17 and a light chain variable region (V_{L}FAP) comprising the amino acid sequence of SEQ ID NO:18. Particularly, the first antigen binding domain that specifically binds to FAP comprises a heavy chain variable region (V_{H}FAP) comprising the amino acid sequence of SEQ ID NO:9 and a light chain variable region (V_{L}FAP) comprising the amino acid sequence of SEQ ID NO:10. Particularly, the first antigen binding domain that specifically binds to FAP is a Fab fragment.

In one aspect, the bispecific agonistic LTBR antibody as disclosed herein comprises a second antigen binding domain that specifically binds to LTBR comprising
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32; or
(ii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; or
(iii) a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80; or
(iv) a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88; or
(v) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(vi) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(vii) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(viii) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

In one particular aspect, the second antigen binding domain that specifically binds to LTBR comprises a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32. In another aspect, the second antigen binding domain that specifically binds to LTBR comprises a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40. In another aspect, the second antigen binding domain that specifically binds to LTBR comprises a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48. In a further aspect, the second antigen binding domain that specifically binds to LTBR comprises a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, In another aspect, the second antigen binding domain that specifically binds to LTBR comprises a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64. In another aspect, the second antigen binding domain that specifically binds to LTBR comprises a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72. In yet another aspect, the second antigen binding domain that specifically binds to LTBR comprises a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80. In a further aspect, the second antigen binding domain that specifically binds to LTBR comprises a heavy chain variable region (V_{H} LTBR) comprising a CDR-H1 comprising the amino acid sequence of SEQ ID NO:83, a CDR-H2 comprising the amino acid sequence of SEQ ID NO:84, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

In another aspect, the second antigen binding domain that specifically binds to LTBR may comprise a heavy chain variable region (V_{H} LTBR) comprising a heavy chain complementary determining region (CDR-H1) comprising the amino acid sequence of SEQ ID NO:101, a CDR-H2 comprising the amino acid sequence of SEQ ID NO:102, and a CDR-H3 comprising the amino acid sequence of SEQ ID NO:103, and a light chain variable region (V_{L} LTBR) comprising a light chain complementarity determining region (iv) CDR-L1 comprising the amino acid sequence of SEQ ID NO:104, a CDR-L2 comprising the amino acid sequence of SEQ ID NO:105, and a CDR-L3 comprising the amino acid sequence of SEQ ID NO:106. In one particular aspect, the second antigen binding domain that specifically binds to LTBR comprise a heavy chain variable region (V_{H} LTBR) comprising the amino acid sequence of SEQ ID NO:99 and a light chain variable region (V_{L} LTBR) comprising the amino acid sequence of SEQ ID NO:100 (CBE 11).

In another aspect, the second antigen binding domain that specifically binds to LTBR may comprise a heavy chain variable region (V_{H} LTBR) comprising a heavy chain complementary determining region (CDR-H1) comprising the amino acid sequence of SEQ ID NO:343, a CDR-H2 comprising the amino acid sequence of SEQ ID NO:344, and a CDR-H3 comprising the amino acid sequence of SEQ ID NO:345, and a light chain variable region (V_{L}LTBR) comprising a light chain complementarity determining region (iv) CDR-L1 comprising the amino acid sequence of SEQ ID NO:346, a CDR-L2 comprising the amino acid sequence of SEQ ID NO:347, and a CDR-L3 comprising the amino acid sequence of SEQ ID NO:348. In one particular aspect, the second antigen binding domain that specifically binds to LTBR comprise a heavy chain variable region (V_{H} LTBR) comprising the amino acid sequence of SEQ ID NO:349 and a light chain variable region (V_{L} LTBR) comprising the amino acid sequence of SEQ ID NO:350 (BHA10).

In one aspect, provided is a bispecific agonistic LTBR antibody comprising a second antigen binding domain that specifically binds to LTBR, comprising
(i) a heavy chain variable region (V_{H} LTBR) comprising an amino acid sequence that is at least about 95% identical to the amino acid sequence of SEQ ID NO:33 and a light chain variable region (V_{L} LTBR) comprising an amino acid sequence that is at least about 95% identical to the amino acid sequence of SEQ ID NO:34, or
(ii) a heavy chain variable region (V_{H} LTBR) comprising an amino acid sequence that is at least about 95% identical to the amino acid sequence of SEQ ID NO:49 and a light chain variable region (V_{L} LTBR) comprising an amino acid sequence that is at least about 95% identical to the amino acid sequence of SEQ ID NO:50, or
(iii) a heavy chain variable region (V_{H} LTBR) comprising an amino acid sequence that is at least about 95% identical to the amino acid sequence of SEQ ID NO:81 and a light chain variable region (V_{L} LTBR) comprising an amino acid sequence that is at least about 95% identical to the amino acid sequence of SEQ ID NO:82, or
(iv) a heavy chain variable region (V_{H} LTBR) comprising an amino acid sequence that is at least about 95% identical to the amino acid sequence of SEQ ID NO:89 and a light chain variable region (V_{L} LTBR) comprising an amino acid sequence that is at least about 95% identical to the amino acid sequence of SEQ ID NO:90, or
(v) a heavy chain variable region (V_{H} LTBR) comprising an amino acid sequence that is at least about 95% identical to the amino acid sequence of SEQ ID NO:97 and a light chain variable region (V_{L} LTBR) comprising an amino acid sequence that is at least about 95% identical to the amino acid sequence of SEQ ID NO:98,
(vi) a heavy chain variable region (V_{H} LTBR) comprising an amino acid sequence that is at least about 95% identical to the amino acid sequence of SEQ ID NO:41 and a light chain variable region (V_{L} LTBR) comprising an amino acid sequence that is at least about 95% identical to the amino acid sequence of SEQ ID NO:42,
(vii) a heavy chain variable region (V_{H} LTBR) comprising an amino acid sequence that is at least about 95% identical to the amino acid sequence of SEQ ID NO:57 and a light chain variable region (V_{L} LTBR) comprising an amino acid sequence that is at least about 95% identical to the amino acid sequence of SEQ ID NO:58, or
(viii) a heavy chain variable region (V_{H} LTBR) comprising an amino acid sequence that is at least about 95% identical to the amino acid sequence of SEQ ID NO:65 and a light chain variable region (V_{L} LTBR) comprising an amino acid sequence that is at least about 95% identical to the amino acid sequence of SEQ ID NO:66, or
(ix) a heavy chain variable region (V_{H} LTBR) comprising an amino acid sequence that is at least about 95% identical to the amino acid sequence of SEQ ID NO:73 and a light chain variable region (V_{L} LTBR) comprising an amino acid sequence that is at least about 95% identical to the amino acid sequence of SEQ ID NO:74.

In one aspect, provided is a bispecific agonistic LTBR antibody comprising a second antigen binding domain that specifically binds to LTBR, comprising
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(iii) a heavy chain variable region (V_{H} LTBR) comprising the amino acid sequence of SEQ ID NO:81 and a light chain variable region (V_{L} LTBR) comprising the amino acid sequence of SEQ ID NO:82, or
(iv) a heavy chain variable region (V_{H} LTBR) comprising the amino acid sequence of SEQ ID NO:89 and a light chain variable region (V_{L} LTBR) comprising the amino acid sequence of SEQ ID NO:90, or
(v) a heavy chain variable region (V_{H} LTBR) comprising the amino acid sequence of SEQ ID NO:97 and a light chain variable region (V_{L} LTBR) comprising the amino acid sequence of SEQ ID NO:98, or
(vi) a heavy chain variable region (V_{H} LTBR) comprising the amino acid sequence of SEQ ID NO:41 and a light chain variable region (V_{L} LTBR) comprising the amino acid sequence of SEQ ID NO:42, or
(vii) a heavy chain variable region (V_{H} LTBR) comprising the amino acid sequence of SEQ ID NO:57 and a light chain variable region (V_{L} LTBR) comprising the amino acid sequence of SEQ ID NO:58, or
(viii) a heavy chain variable region (V_{H} LTBR) comprising the amino acid sequence of SEQ ID NO:65 and a light chain variable region (V_{L} LTBR) comprising the amino acid sequence of SEQ ID NO:66,
(ix) a heavy chain variable region (V_{H} LTBR) comprising the amino acid sequence of SEQ ID NO:73 and a light chain variable region (V_{L} LTBR) comprising the amino acid sequence of SEQ ID NO:74.

In one aspect, the second antigen binding domain that specifically binds to LTBR comprises (i) a heavy chain variable region (V_{H} LTBR) comprising the amino acid sequence of SEQ ID NO:33 and a light chain variable region (V_{L} LTBR) comprising the amino acid sequence of SEQ ID NO:34, or (ii) a heavy chain variable region (V_{H} LTBR) comprising the amino acid sequence of SEQ ID NO:41 and a light chain variable region (V_{L} LTBR) comprising the amino acid sequence of SEQ ID NO:42, or (iii) a heavy chain variable region (V_{H} LTBR) comprising the amino acid sequence of SEQ ID NO:49 and a light chain variable region (V_{L} LTBR) comprising the amino acid sequence of SEQ ID NO:50. In one particular aspect, the second antigen binding domain that specifically binds to LTBR comprise a heavy chain variable region (V_{H} LTBR) comprising the amino acid sequence of SEQ ID NO:33 and a light chain variable region (V_{L} LTBR) comprising the amino acid sequence of SEQ ID NO:34. In another particular aspect, the second antigen binding domain that specifically binds to LTBR comprise a heavy chain variable region (V_{H} LTBR) comprising the amino acid sequence of SEQ ID NO:41 and a light chain variable region (V_{L} LTBR) comprising the amino acid sequence of SEQ ID NO:42. In yet another particular aspect, the second antigen binding domain that specifically binds to LTBR comprise a heavy chain variable region (V_{H} LTBR) comprising the amino acid sequence of SEQ ID NO:49 and a light chain variable region (V_{L} LTBR) comprising the amino acid sequence of SEQ ID NO:50.

In another aspect, the second antigen binding domain that specifically binds to LTBR is able to also specifically bind to murine LTBR and comprises
a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48, or
a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80, or
a heavy chain variable region (V_{H} LTBR) comprising a CDR-H1 comprising the amino acid sequence of SEQ ID NO:83, a CDR-H2 comprising the amino acid sequence of SEQ ID NO:84, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

In one aspect, the second antigen binding domain that specifically binds to LTBR is able to also specifically bind to murine LTBR and comprises
a heavy chain variable region (V_{H} LTBR) comprising the amino acid sequence of SEQ ID NO:33 and a light chain variable region (V_{L} LTBR) comprising the amino acid sequence of SEQ ID NO:34, or
a heavy chain variable region (V_{H} LTBR) comprising the amino acid sequence of SEQ ID NO:49 and a light chain variable region (V_{L} LTBR) comprising the amino acid sequence of SEQ ID NO:50, or
a heavy chain variable region (V_{H} LTBR) comprising the amino acid sequence of SEQ ID NO:81 and a light chain variable region (V_{L} LTBR) comprising the amino acid sequence of SEQ ID NO:82, or
a heavy chain variable region (V_{H} LTBR) comprising the amino acid sequence of SEQ ID NO:89 and a light chain variable region (V_{L} LTBR) comprising the amino acid sequence of SEQ ID NO:90, or
a heavy chain variable region (V_{H} LTBR) comprising the amino acid sequence of SEQ ID NO:97 and a light chain variable region (V_{L} LTBR) comprising the amino acid sequence of SEQ ID NO:98.

In one aspect, the second antigen binding domain that specifically binds to LTBR is able to also specifically bind to murine LTBR and comprises a heavy chain variable region (V_{H}LTBR) comprising the amino acid sequence of SEQ ID NO:33 and a light chain variable region (V_{L} LTBR) comprising the amino acid sequence of SEQ ID NO:34.

In one aspect, provided is a bispecific agonistic LTBR antibody, wherein the bispecific antibody comprises a third antigen binding domain that specifically binds to LTBR, meaning a bispecific antigen binding molecule comprising
(a) a first antigen binding domain that specifically binds to Fibroblast Activation Protein (FAP),
(b) a second and a third antigen binding domain that specifically bind to lymphotoxin beta receptor (LTBR), and
(c) a Fc domain composed of a first and a second subunit and comprising one or more amino acid substitution that reduces the binding affinity of the antigen binding molecule to an Fc receptor and/or effector function.

In one particular aspect, the third antigen binding domain that specifically binds to LTBR is identical to the second antigen binding domain that specifically binds to LTBR, meaning that the second and a third antigen binding domain that specifically bind to lymphotoxin beta receptor (LTBR) are the same. In one aspect, the second and third antigen binding domains that specifically bind to LTBR are Fab fragments that specifically bind to LTBR. In one aspect, the Fab fragments that specifically bind to LTBR are crossfab fragments. In a further aspect, the first antigen binding domain that specifically binds to FAP is a Fab fragment.

In one aspect, the bispecific agonistic LTBR antibody as disclosed herein comprises a second and a third antigen binding domain that specifically bind to LTBR, both comprising
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32; or
(ii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; or
(iii) a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80; or
(iv) a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88; or
(v) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(vi) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(vii) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(viii) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

In one particular aspect, the second and the third antigen binding domain are identical.

In one aspect, the second and the third antigen binding domain that specifically bind to LTBR each comprise a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L}LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32. In another aspect, the second and the third antigen binding domain that specifically bind to LTBR each comprise a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40. In another aspect, the second and the third antigen binding domain that specifically bind to LTBR each comprise a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48. In a further aspect, the second and the third antigen binding domain that specifically bind to LTBR each comprise a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56. In another aspect, the second and the third antigen binding domain that specifically bind to LTBR each comprise a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64. In another aspect, the second and the third antigen binding domain that specifically bind to LTBR each comprise a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72. In yet another aspect, the second and the third antigen binding domain that specifically bind to LTBR each comprise a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80. In a further aspect, the second and the third antigen binding domain that specifically bind to LTBR each comprise a heavy chain variable region (V_{H} LTBR) comprising a CDR-H1 comprising the amino acid sequence of SEQ ID NO:83, a CDR-H2 comprising the amino acid sequence of SEQ ID NO:84, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

In one aspect, provided is a bispecific agonistic LTBR antibody comprising a second and a third antigen binding domain that specifically bind to LTBR, comprising
(i) a heavy chain variable region (V_{H} LTBR) comprising an amino acid sequence that is at least about 95% identical to the amino acid sequence of SEQ ID NO:33 and a light chain variable region (V_{L} LTBR) comprising an amino acid sequence that is at least about 95% identical to the amino acid sequence of SEQ ID NO:34, or
(ii) a heavy chain variable region (V_{H} LTBR) comprising an amino acid sequence that is at least about 95% identical to the amino acid sequence of SEQ ID NO:49 and a light chain variable region (V_{L} LTBR) comprising an amino acid sequence that is at least about 95% identical to the amino acid sequence of SEQ ID NO:50, or
(iii) a heavy chain variable region (V_{H} LTBR) comprising an amino acid sequence that is at least about 95% identical to the amino acid sequence of SEQ ID NO:81 and a light chain variable region (V_{L} LTBR) comprising an amino acid sequence that is at least about 95% identical to the amino acid sequence of SEQ ID NO:82, or
(iv) a heavy chain variable region (V_{H} LTBR) comprising an amino acid sequence that is at least about 95% identical to the amino acid sequence of SEQ ID NO:89 and a light chain variable region (V_{L} LTBR) comprising an amino acid sequence that is at least about 95% identical to the amino acid sequence of SEQ ID NO:90, or
(v) a heavy chain variable region (V_{H} LTBR) comprising an amino acid sequence that is at least about 95% identical to the amino acid sequence of SEQ ID NO:97 and a light chain variable region (V_{L} LTBR) comprising an amino acid sequence that is at least about 95% identical to the amino acid sequence of SEQ ID NO:98,
(vi) a heavy chain variable region (V_{H} LTBR) comprising an amino acid sequence that is at least about 95% identical to the amino acid sequence of SEQ ID NO:41 and a light chain variable region (V_{L} LTBR) comprising an amino acid sequence that is at least about 95% identical to the amino acid sequence of SEQ ID NO:42,
(vii) a heavy chain variable region (V_{H} LTBR) comprising an amino acid sequence that is at least about 95% identical to the amino acid sequence of SEQ ID NO:57 and a light chain variable region (V_{L} LTBR) comprising an amino acid sequence that is at least about 95% identical to the amino acid sequence of SEQ ID NO:58, or
(viii) a heavy chain variable region (V_{H} LTBR) comprising an amino acid sequence that is at least about 95% identical to the amino acid sequence of SEQ ID NO:65 and a light chain variable region (V_{L} LTBR) comprising an amino acid sequence that is at least about 95% identical to the amino acid sequence of SEQ ID NO:66, or
(ix) a heavy chain variable region (V_{H} LTBR) comprising an amino acid sequence that is at least about 95% identical to the amino acid sequence of SEQ ID NO:73 and a light chain variable region (V_{L} LTBR) comprising an amino acid sequence that is at least about 95% identical to the amino acid sequence of SEQ ID NO:74.

In one aspect, provided is a bispecific agonistic LTBR antibody comprising a second and a third antigen binding domain that specifically bind to LTBR, comprising
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(iii) a heavy chain variable region (V_{H} LTBR) comprising the amino acid sequence of SEQ ID NO:81 and a light chain variable region (V_{L} LTBR) comprising the amino acid sequence of SEQ ID NO:82, or
(iv) a heavy chain variable region (V_{H} LTBR) comprising the amino acid sequence of SEQ ID NO:89 and a light chain variable region (V_{L} LTBR) comprising the amino acid sequence of SEQ ID NO:90, or
(v) a heavy chain variable region (V_{H} LTBR) comprising the amino acid sequence of SEQ ID NO:97 and a light chain variable region (V_{L} LTBR) comprising the amino acid sequence of SEQ ID NO:98, or
(vi) a heavy chain variable region (V_{H} LTBR) comprising the amino acid sequence of SEQ ID NO:41 and a light chain variable region (V_{L} LTBR) comprising the amino acid sequence of SEQ ID NO:42, or
(vii) a heavy chain variable region (V_{H} LTBR) comprising the amino acid sequence of SEQ ID NO:57 and a light chain variable region (V_{L} LTBR) comprising the amino acid sequence of SEQ ID NO:58, or
(viii) a heavy chain variable region (V_{H} LTBR) comprising the amino acid sequence of SEQ ID NO:65 and a light chain variable region (V_{L} LTBR) comprising the amino acid sequence of SEQ ID NO:66,
(ix) a heavy chain variable region (V_{H} LTBR) comprising the amino acid sequence of SEQ ID NO:73 and a light chain variable region (V_{L} LTBR) comprising the amino acid sequence of SEQ ID NO:74.

In one aspect, the second and the third antigen binding domain that specifically bind to LTBR each comprise (i) a heavy chain variable region (V_{H} LTBR) comprising the amino acid sequence of SEQ ID NO:33 and a light chain variable region (V_{L} LTBR) comprising the amino acid sequence of SEQ ID NO:34, or (ii) a heavy chain variable region (V_{H} LTBR) comprising the amino acid sequence of SEQ ID NO:41 and a light chain variable region (V_{L} LTBR) comprising the amino acid sequence of SEQ ID NO:42, or (iii) a heavy chain variable region (V_{H} LTBR) comprising the amino acid sequence of SEQ ID NO:49 and a light chain variable region (V_{L} LTBR) comprising the amino acid sequence of SEQ ID NO:50. In one particular aspect, the second and the third antigen binding domain that specifically bind to LTBR each comprise a heavy chain variable region (V_{H} LTBR) comprising the amino acid sequence of SEQ ID NO:33 and a light chain variable region (V_{L} LTBR) comprising the amino acid sequence of SEQ ID NO:34. In another particular aspect, the second and the third antigen binding domain that specifically binds to LTBR each comprise a heavy chain variable region (V_{H} LTBR) comprising the amino acid sequence of SEQ ID NO:41 and a light chain variable region (V_{L} LTBR) comprising the amino acid sequence of SEQ ID NO:42. In yet another particular aspect, the second and the third antigen binding domain that specifically bind to LTBR each comprise a heavy chain variable region (V_{H} LTBR) comprising the amino acid sequence of SEQ ID NO:49 and a light chain variable region (V_{L} LTBR) comprising the amino acid sequence of SEQ ID NO:50.

In another aspect, provided is a bispecific agonistic LTBR antibody comprising a second and a third antigen binding domain that specifically bind to LTBR, each comprising a heavy chain variable region (V_{H} LTBR) comprising a heavy chain complementary determining region (CDR-H1) comprising the amino acid sequence of SEQ ID NO:101, a CDR-H2 comprising the amino acid sequence of SEQ ID NO:102, and a CDR-H3 comprising the amino acid sequence of SEQ ID NO:103, and a light chain variable region (V_{L} LTBR) comprising a light chain complementarity determining region (iv) CDR-L1 comprising the amino acid sequence of SEQ ID NO:104, a CDR-L2 comprising the amino acid sequence of SEQ ID NO:105, and a CDR-L3 comprising the amino acid sequence of SEQ ID NO:106. In one particular aspect, the second and third antigen binding domain that specifically bind to LTBR each comprise a heavy chain variable region (V_{H} LTBR) comprising the amino acid sequence of SEQ ID NO:99 and a light chain variable region (V_{L} LTBR) comprising the amino acid sequence of SEQ ID NO:100 (CBE 11).

In another aspect, provided is a bispecific agonistic LTBR antibody comprising a second and a third antigen binding domain that specifically bind to LTBR, each comprising a heavy chain variable region (V_{H} LTBR) comprising a heavy chain complementary determining region (CDR-H1) comprising the amino acid sequence of SEQ ID NO:343, a CDR-H2 comprising the amino acid sequence of SEQ ID NO:344, and a CDR-H3 comprising the amino acid sequence of SEQ ID NO:345, and a light chain variable region (V_{L} LTBR) comprising a light chain complementarity determining region (iv) CDR-L1 comprising the amino acid sequence of SEQ ID NO:346, a CDR-L2 comprising the amino acid sequence of SEQ ID NO:347, and a CDR-L3 comprising the amino acid sequence of SEQ ID NO:348. In one particular aspect, the second and third antigen binding domain that specifically bind to LTBR each comprise a heavy chain variable region (V_{H} LTBR) comprising the amino acid sequence of SEQ ID NO:349 and a light chain variable region (V_{L} LTBR) comprising the amino acid sequence of SEQ ID NO:350 (BHA10).

In another aspect, provided is a bispecific agonistic LTBR antibody comprising a second and a third antigen binding domain that specifically bind to human LTBR and is able to also specifically bind to murine LTBR, each comprising
a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48, or
a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80, or
a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

In one aspect, the bispecific agonistic LTBR antibody comprises a second and a third antigen binding domain that specifically bind to human LTBR and also specifically bind to murine LTBR, each comprising
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(iii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(vii) a heavy chain variable region (V_{H} LTBR) comprising the amino acid sequence of SEQ ID NO:81 and a light chain variable region (V_{L} LTBR) comprising the amino acid sequence of SEQ ID NO:82, or
a heavy chain variable region (V_{H} LTBR) comprising the amino acid sequence of SEQ ID NO:89 and a light chain variable region (V_{L} LTBR) comprising the amino acid sequence of SEQ ID NO:90, or
ix) a heavy chain variable region (V_{H} LTBR) comprising the amino acid sequence of SEQ ID NO:97 and a light chain variable region (V_{L} LTBR) comprising the amino acid sequence of SEQ ID NO:98.

In one particular aspect, the bispecific agonistic LTBR antibody comprises a second and a third antigen binding domain that specifically bind to human LTBR and also specifically bind to murine LTBR, each comprising a heavy chain variable region (V_{H} LTBR) comprising the amino acid sequence of SEQ ID NO:33 and a light chain variable region (V_{L} LTBR) comprising the amino acid sequence of SEQ ID NO:34.

### Bispecific Agonistic LTBR Antibodies Binding to FAP and LTBR

In one aspect, provided is a bispecific agonistic LTBR antibody, comprising
(a) a first antigen binding domain that specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, or
a heavy chain variable region (V_{H}FAP) comprising the amino acid sequence of SEQ ID NO:25 and a light chain variable region (V_{L}FAP) comprising the amino acid sequence of SEQ ID NO:26, or
a heavy chain variable region (V_{H}FAP) comprising the amino acid sequence of SEQ ID NO:17 and a light chain variable region (V_{L}FAP) comprising the amino acid sequence of SEQ ID NO: 18,
(b) a second antigen binding domain that specifically binds to lymphotoxin beta receptor (LTBR), comprising
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(iii) a heavy chain variable region (V_{H} LTBR) comprising the amino acid sequence of SEQ ID NO:81 and a light chain variable region (V_{L} LTBR) comprising the amino acid sequence of SEQ ID NO:82, or
(iv) a heavy chain variable region (V_{H} LTBR) comprising the amino acid sequence of SEQ ID NO:89 and a light chain variable region (V_{L} LTBR) comprising the amino acid sequence of SEQ ID NO:90, or
(v) a heavy chain variable region (V_{H} LTBR) comprising the amino acid sequence of SEQ ID NO:97 and a light chain variable region (V_{L} LTBR) comprising the amino acid sequence of SEQ ID NO:98,
(vi) a heavy chain variable region (V_{H} LTBR) comprising the amino acid sequence of SEQ ID NO:41 and a light chain variable region (V_{L} LTBR) comprising the amino acid sequence of SEQ ID NO:42,
(vii) a heavy chain variable region (V_{H} LTBR) comprising the amino acid sequence of SEQ ID NO:57 and a light chain variable region (V_{L} LTBR) comprising the amino acid sequence of SEQ ID NO:58, or
(viii) a heavy chain variable region (V_{H} LTBR) comprising the amino acid sequence of SEQ ID NO:65 and a light chain variable region (V_{L} LTBR) comprising the amino acid sequence of SEQ ID NO:66, or
(ix) a heavy chain variable region (V_{H} LTBR) comprising the amino acid sequence of SEQ ID NO:73 and a light chain variable region (V_{L} LTBR) comprising the amino acid sequence of SEQ ID NO:74, and
(c) a Fc domain composed of a first and a second subunit and comprising one or more amino acid substitution that reduces the binding affinity of the antigen binding molecule to an Fc receptor and/or effector function.

In another aspect, provided is a bispecific agonistic LTBR antibody, comprising
(a) a first antigen binding domain that specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, or
a heavy chain variable region (V_{H}FAP) comprising the amino acid sequence of SEQ ID NO:25 and a light chain variable region (V_{L}FAP) comprising the amino acid sequence of SEQ ID NO:26, or
a heavy chain variable region (V_{H}FAP) comprising the amino acid sequence of SEQ ID NO:17 and a light chain variable region (V_{L}FAP) comprising the amino acid sequence of SEQ ID NO:18,
(b) a second and a third antigen binding domain that specifically binds to lymphotoxin beta receptor (LTBR), each comprising
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(iii) a heavy chain variable region (V_{H} LTBR) comprising the amino acid sequence of SEQ ID NO:81 and a light chain variable region (V_{L} LTBR) comprising the amino acid sequence of SEQ ID NO:82, or
(iv) a heavy chain variable region (V_{H} LTBR) comprising the amino acid sequence of SEQ ID NO:89 and a light chain variable region (V_{L} LTBR) comprising the amino acid sequence of SEQ ID NO:90, or
(v) a heavy chain variable region (V_{H} LTBR) comprising the amino acid sequence of SEQ ID NO:97 and a light chain variable region (V_{L} LTBR) comprising the amino acid sequence of SEQ ID NO:98,
(vi) a heavy chain variable region (V_{H} LTBR) comprising the amino acid sequence of SEQ ID NO:41 and a light chain variable region (V_{L} LTBR) comprising the amino acid sequence of SEQ ID NO:42,
(vii) a heavy chain variable region (V_{H} LTBR) comprising the amino acid sequence of SEQ ID NO:57 and a light chain variable region (V_{L} LTBR) comprising the amino acid sequence of SEQ ID NO:58, or
(viii) a heavy chain variable region (V_{H} LTBR) comprising the amino acid sequence of SEQ ID NO:65 and a light chain variable region (V_{L} LTBR) comprising the amino acid sequence of SEQ ID NO:66, or
(ix) a heavy chain variable region (V_{H} LTBR) comprising the amino acid sequence of SEQ ID NO:73 and a light chain variable region (V_{L} LTBR) comprising the amino acid sequence of SEQ ID NO:74, and
(c) a Fc domain composed of a first and a second subunit and comprising one or more amino acid substitution that reduces the binding affinity of the antigen binding molecule to an Fc receptor and/or effector function.

In one particular aspect, provided is a bispecific agonistic LTBR antibody which comprises (i) a first antigen binding domain capable of specific binding to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and (ii) a second and third antigen binding domain capable of specific binding to LTBR, comprising a heavy chain variable region (V_{H} LTBR) comprising an amino acid sequence of SEQ ID NO:33 and a light chain variable region (V_{L} LTBR) comprising an amino acid sequence of SEQ ID NO:34.

In another aspect, provided is a bispecific agonistic LTBR antibody which comprises (i) a first antigen binding domain capable of specific binding to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and (ii) a second and third antigen binding domain capable of specific binding to LTBR, comprising a heavy chain variable region (V_{H} LTBR) comprising an amino acid sequence of SEQ ID NO:99 and a light chain variable region (V_{L} LTBR) comprising an amino acid sequence of SEQ ID NO:100.

### Bispecific, Monovalent Agonistic LTBR Antibodies (1+1 Format)

In one aspect, provided is a bispecific agonistic LTBR antibody, comprising a first antigen binding domain that specifically binds to Fibroblast Activation Protein (FAP), a second antigen binding domain that specifically binds to lymphotoxin beta receptor (LTBR), and a Fc domain composed of a first and a second subunit and comprising one or more amino acid substitution that reduces the binding affinity of the antigen binding molecule to an Fc receptor and/or effector function, wherein the bispecific antigen binding molecule comprises
(i) a first light chain and a first heavy chain of a full length antibody which specifically binds to FAP, and
(ii) a second (modified) light chain and a second (modified) heavy chain of a full length antibody which specifically binds to LTBR, wherein the variable domains VL and VH are replaced by each other, and/or wherein the constant domains CL and CH1 are replaced by each other.

The first antigen binding domain that specifically binds to FAP is a Fab fragment and second antigen binding domain that specifically binds to LTBR is a crossFab fragment.

In another aspect, provided is a bispecific agonistic LTBR antibody, comprising a first antigen binding domain that specifically binds to Fibroblast Activation Protein (FAP), a second antigen binding domain that specifically binds to lymphotoxin beta receptor (LTBR), and a Fc domain composed of a first and a second subunit and comprising one or more amino acid substitution that reduces the binding affinity of the antigen binding molecule to an Fc receptor and/or effector function, wherein the bispecific antigen binding molecule comprises
(i) a first (modified) light chain and a first (modified) heavy chain of a full length antibody which specifically binds to FAP, herein the variable domains VL and VH are replaced by each other, and/or wherein the constant domains CL and CH1 are replaced by each other, and
(ii) a second light chain and a second heavy chain of a full length antibody which specifically binds to LTBR.

The first antigen binding domain that specifically binds to FAP is a crossFab fragment and second antigen binding domain that specifically binds to LTBR is a Fab fragment.

In one particular aspect, provided are
(a) a bispecific agonistic LTBR antibody comprising a first heavy chain comprising the amino acid sequence of SEQ ID NO:107, a second heavy chain comprising the amino acid sequence of SEQ ID NO:109, a first light chain comprising the amino acid sequence of SEQ ID NO:108, and a second light chain comprising the amino acid sequence of SEQ ID NO:110, or
(b) a bispecific agonistic LTBR antibody comprising a first heavy chain comprising the amino acid sequence of SEQ ID NO:111, a second heavy chain comprising the amino acid sequence of SEQ ID NO:113, a first light chain comprising the amino acid sequence of SEQ ID NO:112, and a second light chain comprising the amino acid sequence of SEQ ID NO:114, or
(c) a bispecific agonistic LTBR antibody comprising a first heavy chain comprising the amino acid sequence of SEQ ID NO:115, a second heavy chain comprising the amino acid sequence of SEQ ID NO:117, a first light chain comprising the amino acid sequence of SEQ ID NO:116, and a second light chain comprising the amino acid sequence of SEQ ID NO:118, or
(d) a bispecific agonistic LTBR antibody comprising a first heavy chain comprising the amino acid sequence of SEQ ID NO:119, a second heavy chain comprising the amino acid sequence of SEQ ID NO:121, a first light chain comprising the amino acid sequence of SEQ ID NO:120, and a second light chain comprising the amino acid sequence of SEQ ID NO:122, or
(e) a bispecific agonistic LTBR antibody comprising a first heavy chain comprising the amino acid sequence of SEQ ID NO:123, a second heavy chain comprising the amino acid sequence of SEQ ID NO:125, a first light chain comprising the amino acid sequence of SEQ ID NO:124, and a second light chain comprising the amino acid sequence of SEQ ID NO:126, or
(f) a bispecific agonistic LTBR antibody comprising a first heavy chain comprising the amino acid sequence of SEQ ID NO:127, a second heavy chain comprising the amino acid sequence of SEQ ID NO:129, a first light chain comprising the amino acid sequence of SEQ ID NO:128, and a second light chain comprising the amino acid sequence of SEQ ID NO:130, or
(g) a bispecific agonistic LTBR antibody comprising a first heavy chain comprising the amino acid sequence of SEQ ID NO:131, a second heavy chain comprising the amino acid sequence of SEQ ID NO:133, a first light chain comprising the amino acid sequence of SEQ ID NO:132, and a second light chain comprising the amino acid sequence of SEQ ID NO:134, or
(h) a bispecific agonistic LTBR antibody comprising a first heavy chain comprising the amino acid sequence of SEQ ID NO:135, a second heavy chain comprising the amino acid sequence of SEQ ID NO:137, a first light chain comprising the amino acid sequence of SEQ ID NO:136, and a second light chain comprising the amino acid sequence of SEQ ID NO:138, or
(i) a bispecific agonistic LTBR antibody comprising a first heavy chain comprising the amino acid sequence of SEQ ID NO:139, a second heavy chain comprising the amino acid sequence of SEQ ID NO:141, a first light chain comprising the amino acid sequence of SEQ ID NO:140, and a second light chain comprising the amino acid sequence of SEQ ID NO:142, or
(j) a bispecific agonistic LTBR antibody comprising a first heavy chain comprising the amino acid sequence of SEQ ID NO:143, a second heavy chain comprising the amino acid sequence of SEQ ID NO:145, a first light chain comprising the amino acid sequence of SEQ ID NO:144, and a second light chain comprising the amino acid sequence of SEQ ID NO:146, or
(k) a bispecific agonistic LTBR antibody comprising a first heavy chain comprising the amino acid sequence of SEQ ID NO:147, a second heavy chain comprising the amino acid sequence of SEQ ID NO:149, a first light chain comprising the amino acid sequence of SEQ ID NO:148, and a second light chain comprising the amino acid sequence of SEQ ID NO:150, or
(l) a bispecific agonistic LTBR antibody comprising a first heavy chain comprising the amino acid sequence of SEQ ID NO:151, a second heavy chain comprising the amino acid sequence of SEQ ID NO:153, a first light chain comprising the amino acid sequence of SEQ ID NO:152, and a second light chain comprising the amino acid sequence of SEQ ID NO:154, or
(m) a bispecific agonistic LTBR antibody comprising a first heavy chain comprising the amino acid sequence of SEQ ID NO:155, a second heavy chain comprising the amino acid sequence of SEQ ID NO:157, a first light chain comprising the amino acid sequence of SEQ ID NO:156, and a second light chain comprising the amino acid sequence of SEQ ID NO:158, or
(n) a bispecific agonistic LTBR antibody comprising a first heavy chain comprising the amino acid sequence of SEQ ID NO:159, a second heavy chain comprising the amino acid sequence of SEQ ID NO:161, a first light chain comprising the amino acid sequence of SEQ ID NO:160, and a second light chain comprising the amino acid sequence of SEQ ID NO:162, or
(o) a bispecific agonistic LTBR antibody comprising a first heavy chain comprising the amino acid sequence of SEQ ID NO:163, a second heavy chain comprising the amino acid sequence of SEQ ID NO:165, a first light chain comprising the amino acid sequence of SEQ ID NO:164, and a second light chain comprising the amino acid sequence of SEQ ID NO:166, or
(q) a bispecific agonistic LTBR antibody comprising a first heavy chain comprising the amino acid sequence of SEQ ID NO:215, a second heavy chain comprising the amino acid sequence of SEQ ID NO:217, a first light chain comprising the amino acid sequence of SEQ ID NO:216, and a second light chain comprising the amino acid sequence of SEQ ID NO:218.

In one aspect, a bispecific agonistic LTBR antibody comprising a first heavy chain comprising the amino acid sequence of SEQ ID NO:329, a second heavy chain comprising the amino acid sequence of SEQ ID NO:330, a first light chain comprising the amino acid sequence of SEQ ID NO:331, and a second light chain comprising the amino acid sequence of SEQ ID NO:332 is provided.

### Bispecific Agonistic LTBR Antibodies Bivalent for Binding to LTBR and Monovalent for Binding to FAP (2+1 Format)

In one aspect, provided is a bispecific agonistic LTBR antibody, comprising a first antigen binding domain that specifically binds to Fibroblast Activation Protein (FAP), a second and a third antigen binding domain that specifically bind to lymphotoxin beta receptor (LTBR), and a Fc domain composed of a first and a second subunit and comprising one or more amino acid substitution that reduces the binding affinity of the antigen binding molecule to an Fc receptor and/or effector function, wherein the bispecific antigen binding molecule comprises
(i) two light chains and two heavy chain of a full length antibody which specifically binds to LTBR, and
(ii) a Fab fragment or crossFab fragment that specifically binds to FAP is connected via a peptide linker to one of the C-termini of the two heavy chains.

In another aspect, provided is a bispecific antigen binding molecule, comprising a first antigen binding domain that specifically binds to Fibroblast Activation Protein (FAP), a second antigen binding domain that specifically binds to lymphotoxin beta receptor (LTBR), and a Fc domain composed of a first and a second subunit and comprising one or more amino acid substitution that reduces the binding affinity of the antigen binding molecule to an Fc receptor and/or effector function, wherein the bispecific antigen binding molecule comprises
(i) a first light chain and a first heavy chain of a full length antibody which specifically binds to FAP, and
(ii) a second (modified) light chain and a second (modified) heavy chain of a full length antibody which specifically binds to LTBR, wherein a Fab fragment or CrossFab fragment that specifically binds to LTBR is connected via a peptide linker to the C-terminus of the second heavy chain.

Thus, provided is a bispecific antigen binding molecule, wherein the bispecific antigen binding molecule has bivalent binding to LTBR and monovalent binding to FAP.

In one particular aspect, provided are:
(a) a bispecific agonistic LTBR antibody comprising first heavy chain comprising the amino acid sequence of SEQ ID NO:167, and a second heavy chain comprising the amino acid sequence of SEQ ID NO:169, two light chains, each comprising the amino acid sequence of SEQ ID NO:168, and one light chain comprising the amino acid sequence of SEQ ID NO:170, or
(b) a bispecific agonistic LTBR antibody comprising first heavy chain comprising the amino acid sequence of SEQ ID NO:171, and a second heavy chain comprising the amino acid sequence of SEQ ID NO:173, two light chains, each comprising the amino acid sequence of SEQ ID NO:172, and one light chain comprising the amino acid sequence of SEQ ID NO:174, or
(c) a bispecific agonistic LTBR antibody comprising first heavy chain comprising the amino acid sequence of SEQ ID NO:175, and a second heavy chain comprising the amino acid sequence of SEQ ID NO:177, two light chains, each comprising the amino acid sequence of SEQ ID NO:176, and one light chain comprising the amino acid sequence of SEQ ID NO:178, or
(d) a bispecific agonistic LTBR antibody comprising first heavy chain comprising the amino acid sequence of SEQ ID NO:179, and a second heavy chain comprising the amino acid sequence of SEQ ID NO:181, two light chains, each comprising the amino acid sequence of SEQ ID NO:180, and one light chain comprising the amino acid sequence of SEQ ID NO:182, or
(e) a bispecific agonistic LTBR antibody comprising first heavy chain comprising the amino acid sequence of SEQ ID NO:183, and a second heavy chain comprising the amino acid sequence of SEQ ID NO:185, two light chains, each comprising the amino acid sequence of SEQ ID NO:184, and one light chain comprising the amino acid sequence of SEQ ID NO:186, or
(f) a bispecific agonistic LTBR antibody comprising first heavy chain comprising the amino acid sequence of SEQ ID NO:187, and a second heavy chain comprising the amino acid sequence of SEQ ID NO:189, two light chains, each comprising the amino acid sequence of SEQ ID NO:188, and one light chain comprising the amino acid sequence of SEQ ID NO:190, or
(g) a bispecific agonistic LTBR antibody comprising first heavy chain comprising the amino acid sequence of SEQ ID NO:191, and a second heavy chain comprising the amino acid sequence of SEQ ID NO:193, two light chains, each comprising the amino acid sequence of SEQ ID NO:192, and one light chain comprising the amino acid sequence of SEQ ID NO:194, or
(h) a bispecific agonistic LTBR antibody comprising first heavy chain comprising the amino acid sequence of SEQ ID NO:195, and a second heavy chain comprising the amino acid sequence of SEQ ID NO:197, two light chains, each comprising the amino acid sequence of SEQ ID NO:196, and one light chain comprising the amino acid sequence of SEQ ID NO:198, or
(i) a bispecific agonistic LTBR antibody comprising first heavy chain comprising the amino acid sequence of SEQ ID NO:199, and a second heavy chain comprising the amino acid sequence of SEQ ID NO:201, two light chains, each comprising the amino acid sequence of SEQ ID NO:200, and one light chain comprising the amino acid sequence of SEQ ID NO:202, or
(j) a bispecific agonistic LTBR antibody comprising first heavy chain comprising the amino acid sequence of SEQ ID NO:203, and a second heavy chain comprising the amino acid sequence of SEQ ID NO:205, two light chains, each comprising the amino acid sequence of SEQ ID NO:204, and one light chain comprising the amino acid sequence of SEQ ID NO:206 or
(k) a bispecific agonistic LTBR antibody comprising first heavy chain comprising the amino acid sequence of SEQ ID NO:207, and a second heavy chain comprising the amino acid sequence of SEQ ID NO:209, two light chains, each comprising the amino acid sequence of SEQ ID NO:208, and one light chain comprising the amino acid sequence of SEQ ID NO:210, or
(l) a bispecific agonistic LTBR antibody comprising first heavy chain comprising the amino acid sequence of SEQ ID NO:211, and a second heavy chain comprising the amino acid sequence of SEQ ID NO:213, two light chains, each comprising the amino acid sequence of SEQ ID NO:212, and one light chain comprising the amino acid sequence of SEQ ID NO:214.

Furthermore, provided are
(m) a bispecific agonistic LTBR antibody comprising first heavy chain comprising the amino acid sequence of SEQ ID NO:326, and a second heavy chain comprising the amino acid sequence of SEQ ID NO:328, two light chains, each comprising the amino acid sequence of SEQ ID NO:327, and one light chain comprising the amino acid sequence of SEQ ID NO:329, or
(n) a bispecific agonistic LTBR antibody comprising first heavy chain comprising the amino acid sequence of SEQ ID NO:333, and a second heavy chain comprising the amino acid sequence of SEQ ID NO:334, two light chains, each comprising the amino acid sequence of SEQ ID NO:335, and one light chain comprising the amino acid sequence of SEQ ID NO:336, or
(o) a bispecific agonistic LTBR antibody comprising first heavy chain comprising the amino acid sequence of SEQ ID NO:337, and a second heavy chain comprising the amino acid sequence of SEQ ID NO:338, two light chains, each comprising the amino acid sequence of SEQ ID NO:339, and one light chain comprising the amino acid sequence of SEQ ID NO:340.

In one particular aspect, provided is a bispecific agonistic LTBR antibody comprising a bispecific antigen binding molecule comprising first heavy chain comprising the amino acid sequence of SEQ ID NO:195, and a second heavy chain comprising the amino acid sequence of SEQ ID NO:197, two light chains, each comprising the amino acid sequence of SEQ ID NO:196, one light chain comprising the amino acid sequence of SEQ ID NO:198.

### Murine Surrogate Agonistic LTBR Antibodies

Herein provided are also murine surrogate molecules. In one aspect, provided is a bispecific agonistic LTBR antibody, comprising
(a) a first antigen binding domain that specifically binds to FAP, comprising a heavy chain variable region (V_{H}FAP) comprising the amino acid sequence of SEQ ID NO:17 and a light chain variable region (V_{L}FAP) comprising the amino acid sequence of SEQ ID NO:18,
(b) a second antigen binding domain that specifically binds to lymphotoxin beta receptor (LTBR), comprising a heavy chain variable region (V_{H} LTBR) comprising the amino acid sequence of SEQ ID NO:49 and a light chain variable region (V_{L} LTBR) comprising the amino acid sequence of SEQ ID NO:50, and
(c) a Fc domain composed of a first and a second subunit and comprising one or more amino acid substitution that reduces the binding affinity of the antigen binding molecule to an Fc receptor and/or effector function.

In one particular aspect, provided is a bispecific agonistic LTBR antibody comprising a first heavy chain comprising the amino acid sequence of SEQ ID NO:219, a second heavy chain comprising the amino acid sequence of SEQ ID NO:221, a first light chain comprising the amino acid sequence of SEQ ID NO:220, a second light chain comprising the amino acid sequence of SEQ ID NO:222 (1+1 format).

In one aspect, provided is a bispecific agonistic LTBR antibody, comprising
(a) a first antigen binding domain that specifically binds to FAP, comprising a heavy chain variable region (V_{H}FAP) comprising the amino acid sequence of SEQ ID NO:17 and a light chain variable region (V_{L}FAP) comprising the amino acid sequence of SEQ ID NO:18,
(b) a second and a third antigen binding domain that specifically bind to lymphotoxin beta receptor (LTBR), each comprising a heavy chain variable region (V_{H} LTBR) comprising the amino acid sequence of SEQ ID NO:49 and a light chain variable region (V_{L} LTBR) comprising the amino acid sequence of SEQ ID NO:50, and
(c) a Fc domain composed of a first and a second subunit and comprising one or more amino acid substitution that reduces the binding affinity of the antigen binding molecule to an Fc receptor and/or effector function.

In one aspect, provided is a bispecific agonistic LTBR antibody comprising first heavy chain comprising the amino acid sequence of SEQ ID NO:223, and a second heavy chain comprising the amino acid sequence of SEQ ID NO:225, two light chains, each comprising the amino acid sequence of SEQ ID NO:224, one light chain comprising the amino acid sequence of SEQ ID NO:226.

In one particular aspect, provided is a a bispecific agonistic LTBR antibody comprising first heavy chain comprising the amino acid sequence of SEQ ID NO:318, and a second heavy chain comprising the amino acid sequence of SEQ ID NO:320, two light chains, each comprising the amino acid sequence of SEQ ID NO:319, and one light chain comprising the amino acid sequence of SEQ ID NO:321.

Fc Domain Modifications Reducing Fc Receptor Binding and/or Effector Function

The bispecific agonistic LTBR antibodies described herein further comprise a Fc domain composed of a first and a second subunit and comprising one or more amino acid substitution that reduces the binding affinity of the antigen binding molecule to an Fc receptor and/or effector function. Thus, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

The Fc domain confers favorable pharmacokinetic properties to the bispecific antibodies of the invention, including a long serum half-life which contributes to good accumulation in the target tissue and a favorable tissue-blood distribution ratio. At the same time it may, however, lead to undesirable targeting of the bispecific agonistic LTBR antibodies to cells expressing Fc receptors rather than to the preferred antigen-bearing cells. Accordingly, in particular aspects the Fc domain of the bispecific agonistic LTBR antibodies exhibits reduced binding affinity to an Fc receptor and/or reduced effector function, as compared to a native IgG Fc domain, in particular an IgG1 Fc domain or an IgG4 Fc domain. More particularly, the Fc domain is an IgG1 Fc domain.

In one such aspect, the Fc domain (or the bispecific antigen binding molecule of the invention comprising said Fc domain) exhibits less than 50%, preferably less than 20%, more preferably less than 10% and most preferably less than 5% of the binding affinity to an Fc receptor, as compared to a native IgG1 Fc domain (or the bispecific antigen binding molecule of the invention comprising a native IgG1 Fc domain), and/or less than 50%, preferably less than 20%, more preferably less than 10% and most preferably less than 5% of the effector function, as compared to a native IgG1 Fc domain (or the bispecific antigen binding molecule of the invention comprising a native IgG1 Fc domain). In one aspect, the Fc domain (or the bispecific antigen binding molecule of the invention comprising said Fc domain) does not substantially bind to an Fc receptor and/or induce effector function. In a particular aspect the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. In one aspect, the Fc receptor is an inhibitory Fc receptor. In a specific aspect, the Fc receptor is an inhibitory human Fcγ receptor, more specifically human FcγRIIB. In one aspect the effector function is one or more of CDC, ADCC, ADCP, and cytokine secretion. In a particular aspect, the effector function is ADCC. In one aspect, the Fc domain exhibits substantially similar binding affinity to neonatal Fc receptor (FcRn), as compared to a native IgG1 Fc domain. Substantially similar binding to FcRn is achieved when the Fc domain (or the bispecific antigen binding molecule of the invention comprising said Fc domain) exhibits greater than about 70%, particularly greater than about 80%, more particularly greater than about 90% of the binding affinity of a native IgG1 Fc domain (or the bispecific agonistic LTBR antibody comprising a native IgG1 Fc domain) to FcRn.

In a particular aspect, the Fc domain is engineered to have reduced binding affinity to an Fc receptor and/or reduced effector function, as compared to a non-engineered Fc domain. In a particular aspect, the Fc domain of the bispecific agonistic LTBR antibody comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In another aspect, the amino acid mutation reduces the binding affinity of the Fc domain to an Fc receptor by at least 2-fold, at least 5-fold, or at least 10-fold. In one aspect, the bispecific agonistic LTBR antibody comprising an engineered Fc domain exhibits less than 20%, particularly less than 10%, more particularly less than 5% of the binding affinity to an Fc receptor as compared to bispecific antibodies of the invention comprising a non-engineered Fc domain. In a particular aspect, the Fc receptor is an Fcγ receptor. In other aspects, the Fc receptor is a human Fc receptor. In one aspect, the Fc receptor is an inhibitory Fc receptor. In a specific aspect, the Fc receptor is an inhibitory human Fcγ receptor, more specifically human FcγRIIB. In some aspects the Fc receptor is an activating Fc receptor. In a specific aspect, the Fc receptor is an activating human Fcγ receptor, more specifically human FcγRIIIa, FcγRI or FcγRIIa, most specifically human FcγRIIIa. Preferably, binding to each of these receptors is reduced. In some aspects, binding affinity to a complement component, specifically binding affinity to C1q, is also reduced. In one aspect, binding affinity to neonatal Fc receptor (FcRn) is not reduced. Substantially similar binding to FcRn, i.e. preservation of the binding affinity of the Fc domain to said receptor, is achieved when the Fc domain (or the bispecific agonistic LTBR antibody comprising said Fc domain) exhibits greater than about 70% of the binding affinity of a non-engineered form of the Fc domain (or the bispecific agonistic LTBR antibody comprising said non-engineered form of the Fc domain) to FcRn. The Fc domain, or the bispecific agonistic LTBR antibody comprising said Fc domain, may exhibit greater than about 80% and even greater than about 90% of such affinity. In certain aspects the Fc domain of the bispecific agonistic LTBR antibody is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (e.g. U.S. Pat. No. 6,737,056; WO 2004/056312, and Shields, R. L. et al., J. Biol. Chem. 276 (2001) 6591-6604).

In one aspect, the Fc domain comprises an amino acid substitution at a position of E233, L234, L235, N297, P331 and P329. In some aspects, the Fc domain comprises the amino acid substitutions L234A and L235A (“LALA”). In one such aspect, the Fc domain is an IgG1 Fc domain, particularly a human IgG1 Fc domain. In one aspect, the Fc domain comprises an amino acid substitution at position P329. In a more specific aspect, the amino acid substitution is P329A or P329G, particularly P329G. In one embodiment the Fc domain comprises an amino acid substitution at position P329 and a further amino acid substitution selected from the group consisting of E233P, L234A, L235A, L235E, N297A, N297D or P331S. In more particular aspects the Fc domain comprises the amino acid mutations L234A, L235A and P329G (“P329G LALA”). The “P329G LALA” combination of amino acid substitutions almost completely abolishes Fcγ receptor binding of a human IgG1 Fc domain, as described in PCT Patent Application No. WO 2012/130831 A1. Said document also describes methods of preparing such mutant Fc domains and methods for determining its properties such as Fc receptor binding or effector functions. Such antibody is an IgG1 with mutations L234A and L235A or with mutations L234A, L235A and P329G (numbering according to EU index of Kabat et al, Kabat et al., Sequences of Proteins of Immunological Interest, 5th Ed. Public Health Service, National Institutes of Health, Bethesda, MD, 1991).

In one aspect, the Fc domain is an IgG4 Fc domain. In a more specific aspect, the Fc domain is an IgG4 Fc domain comprising an amino acid substitution at position S228 (Kabat numbering), particularly the amino acid substitution S228P. In a more specific embodiment, the Fc domain is an IgG4 Fc domain comprising amino acid substitutions L235E and S228P and P329G. This amino acid substitution reduces in vivo Fab arm exchange of IgG4 antibodies (see Stubenrauch et al., Drug Metabolism and Disposition 38, 84-91 (2010)).

Antibodies with increased half-lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan, A. R. and Winter, G., Nature 322 (1988) 738-740; U.S. Pat. Nos. 5,648,260; 5,624,821; and WO 94/29351 concerning other examples of Fc region variants.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor. Effector function of an Fc domain, or bispecific agonistic LTBR antibodies comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (Cell Technology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in an animal model such as that disclosed in Clynes et al., Proc Natl Acad Sci USA 95, 652-656 (1998).

The following section describes preferred aspects of the bispecific agonistic LTBR antibody described herein comprising Fc domain modifications reducing Fc receptor binding and/or effector function. In one aspect, provided is a bispecific agonistic LTBR antibody comprising (a) a first antigen binding domain that specifically binds to Fibroblast Activation Protein (FAP), (b) a second (and optionally a third) antigen binding domain that specifically binds to lymphotoxin beta receptor (LTBR), and (c) a Fc domain composed of a first and a second subunit, wherein the Fc domain comprises one or more amino acid substitution that reduces the binding affinity of the antibody to an Fc receptor, in particular towards Fcγ receptor. In another aspect, provided is a bispecific agonistic LTBR antibody comprising (a) a first antigen binding domain that specifically binds to Fibroblast Activation Protein (FAP), (b) a second (and optionally a third) antigen binding domain that specifically binds to lymphotoxin beta receptor (LTBR), and (c) a Fc domain composed of a first and a second subunit capable of stable association, wherein the Fc domain comprises one or more amino acid substitution that reduces effector function. In particular aspect, the Fc domain is of human IgG1 subclass with the amino acid mutations L234A, L235A and P329G (numbering according to Kabat EU index).

### Fc Domain Modifications Promoting Heterodimerization

The bispecific agonistic LTBR antibodies described herein comprise different antigen-binding sites, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific agonistic LTBR antibodies in recombinant production, it will thus be advantageous to introduce in the Fc domain of the bispecific antigen binding molecules of the invention a modification promoting the association of the desired polypeptides.

Accordingly, in particular aspects provided is a bispecific agonistic LTBR antibody comprising (a) a first antigen binding domain that specifically binds to Fibroblast Activation Protein (FAP), (b) a second antigen binding domain that specifically binds to lymphotoxin beta receptor (LTBR), and (c) a Fc domain composed of a first and a second subunit and comprising one or more amino acid substitution that reduces the binding affinity of the antigen binding molecule to an Fc receptor and/or effector function, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect, said modification is a so-called “knob-into-hole” modification, comprising a “knob” modification in one of the two subunits of the Fc domain and a “hole” modification in the other one of the two subunits of the Fc domain. Thus, provided is a bispecific agonistic LTBR antibody comprising (a) at least one antigen binding domain capable of specific binding to LTBR, (b) at least one antigen binding domain capable of specific binding to a target cell antigen, and (c) a Fc domain composed of a first and a second subunit capable of stable association, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

The C-terminus of the heavy chain of the bispecific agonistic LTBR antibody as reported herein can be a complete C-terminus ending with the amino acid residues PGK. The C-terminus of the heavy chain can be a shortened C-terminus in which one or two of the C terminal amino acid residues have been removed. In one particular aspect, the C-terminus of the heavy chain is a shortened C-terminus ending PG. In one particular aspect, the C-terminus of the heavy chain is a shortened C-terminus ending P. In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one aspect of all aspects as reported herein, a bispecific agonistic LTBR antibody comprising a heavy chain including a C-terminal CH3 domain, as specified herein, comprises a C-terminal glycine residue (G446, numbering according to Kabat EU index).

### Modifications in the Fab Domains

In one aspect, provided is a bispecific agonistic LTBR antibody comprising (a) a first Fab fragment capable of specific binding to FAP, (b) a second Fab fragment capable of specific binding to LTBR, and (c) a Fc domain composed of a first and a second subunit, wherein in one of the Fab fragments either the variable domains VH and VL or the constant domains CH1 and CL are exchanged. The bispecific antibodies are prepared according to the Crossmab technology.

Multispecific antibodies with a domain replacement/exchange in one binding arm (CrossMabVH-VL or CrossMabCH-CL) are described in detail in WO2009/080252 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In one aspect, provided is a bispecific agonistic LTBR antibody comprising (a) a first Fab fragment capable of specific binding to FAP, (b) a second Fab fragment capable of specific binding to LTBR, and (c) a Fc domain composed of a first and a second subunit capable of stable association, wherein in one of the Fab fragments the variable domains VL and VH are replaced by each other so that the VH domain is part of the light chain and the VL domain is part of the heavy chain. More particularly, in the second Fab fragment capable of specific binding to LTBR the variable domains VL and VH are replaced by each other so that the VH domain is part of the light chain and the VL domain is part of the heavy chain.

In another aspect, provided is a bispecific agonistic LTBR antibody comprising (a) a first Fab fragment capable of specific binding to FAP, wherein the constant domains CL and CH1 are replaced by each other so that the CH1 domain is part of the light chain and the CL domain is part of the heavy chain, and (b) a second Fab fragment capable of specific binding to LTBR. Such a molecule provides monovalent binding to both LTBR and FAP.

In another aspect, provided is a bispecific agonistic LTBR antibody comprising (a) a first Fab fragment that specifically binds to Fibroblast Activation Protein (FAP), (b) a second and a third Fab fragment that specifically bind to lymphotoxin beta receptor (LTBR), and (c) a Fc domain composed of a first and a second subunit and comprising one or more amino acid substitution that reduces the binding affinity of the antigen binding molecule to an Fc receptor and/or effector function, wherein in the second and third Fab fragment that specifically to LTBR, the variable domains VL and VH are replaced by each other so that the VH domain is part of the light chain and the VL domain is part of the heavy chain.

In another aspect, and to further improve correct pairing, the bispecific a agonistic LTBR antibody comprising (a) a first Fab fragment capable of specific binding to LTBR, (b) a second Fab fragment capable of specific binding to FAP, and (c) a Fc domain composed of a first and a second subunit capable of stable association, can contain different charged amino acid substitutions (so-called “charged residues”). These modifications are introduced in the crossed or non-crossed CH1 and CL domains. In a particular aspect, the invention relates to a bispecific antigen binding molecule, wherein in one of CL domains the amino acid at position 123 (EU numbering) has been replaced by arginine (R) and/or wherein the amino acid at position 124 (EU numbering) has been substituted by lysine (K) and wherein in one of the CH1 domains the amino acids at position 147 (EU numbering) and/or at position 213 (EU numbering) have been substituted by glutamic acid (E).

### Polynucleotides

Further provided are isolated polynucleotides encoding an agonistic LTBR antibody or a bispecific agonistic LTBR antibody as described herein or a fragment thereof.

The isolated polynucleotides encoding an agonistic LTBR antibody or a bispecific agonistic LTBR antibody may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes a polypeptide comprised in the agonistic LTBR antibody or bispecific agonistic LTBR antibody as described herein.

In one aspect, provided is an isolated polynucleotide encoding a bispecific agonistic LTBR antibody, comprising (a) a first antigen binding domain that specifically binds to Fibroblast Activation Protein (FAP), (b) a second antigen binding domain that specifically binds to lymphotoxin beta receptor (LTBR), and (c) a Fc domain composed of a first and a second subunit and comprising one or more amino acid substitution that reduces the binding affinity of the antigen binding molecule to an Fc receptor and/or effector function.

In certain aspects, the polynucleotide or nucleic acid is DNA. In other embodiments, a polynucleotide of the present invention is RNA, for example, in the form of messenger RNA (mRNA). RNA as described herein may be single stranded or double stranded.

### Recombinant Methods

Agonistic LTBR antibodies or bispecific agonistic LTBR antibodies as described herein may be obtained, for example, by recombinant production. For recombinant production one or more polynucleotide encoding the agonistic LTBR antibody or the bispecific agonistic LTBR antibody or polypeptide fragments thereof are provided. The one or more polynucleotide encoding the agonistic LTBR antibody or the bispecific agonistic LTBR antibody are isolated and inserted into one or more vectors for further cloning and/or expression in a host cell. Such polynucleotide may be readily isolated and sequenced using conventional procedures. In one aspect, a vector, preferably an expression vector, comprising one or more of the polynucleotides described herein is provided. Methods which are well known to those skilled in the art can be used to construct expression vectors containing the coding sequence of the agonistic LTBR antibody or bispecific agonistic LTBR antibody (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the agonistic LTBR antibody or bispecific agonistic LTBR antibody or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid as described herein may encode heterologous coding regions, either fused or unfused to a polynucleotide encoding the agonistic LTBR antibody or bispecific agonistic LTBR antibody or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit â-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions as described herein may be associated with additional coding regions which encode secretory or signal peptides, which direct the secretion of a polypeptide encoded by a polynucleotide described herein. For example, if secretion of the agonistic LTBR antibody or bispecific agonistic LTBR antibody or polypeptide fragments thereof is desired, DNA encoding a signal sequence may be placed upstream of the nucleic acid encoding the agonistic LTBR antibody or bispecific agonistic LTBR antibody or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding the agonistic LTBR antibody or bispecific agonistic LTBR antibody or polypeptide fragments thereof.

In a further aspect, a host cell comprising one or more polynucleotides described herein is provided. In certain aspects, a host cell comprising one or more vectors described herein is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) an agonistic LTBR antibody or a bispecific agonistic LTBR antibody as disclosed herein. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, NJ), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing an agonistic LTBR antibody or a bispecific agonistic LTBR antibody or polypeptide fragments thereof is provided, wherein the method comprises culturing a host cell comprising polynucleotides encoding the agonistic LTBR antibody or bispecific agonistic LTBR antibody or polypeptide fragments thereof, as provided herein, under conditions suitable for expression of the agonistic LTBR antibody or bispecific agonistic LTBR antibody or polypeptide fragments thereof, and recovering the agonistic LTBR antibody or bispecific agonistic LTBR antibody or polypeptide fragments thereof from the host cell (or host cell culture medium).

Agonistic LTBR antibodies or bispecific agonistic LTBR antibodies prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the agonistic LTBR antibody or bispecific agonistic LTBR antibody binds. For example, for affinity chromatography purification of antibodies, a matrix with protein A or protein G may be used. Sequential Protein A or G affinity chromatography and size exclusion chromatography can be used to isolate an antigen binding molecule essentially as described in the examples. The purity of the agonistic LTBR antibody or bispecific agonistic LTBR antibody or fragments thereof can be determined by any of a variety of well-known analytical methods including gel electrophoresis, high pressure liquid chromatography, and the like. For example, the agonistic LTBR antibodies or bispecific agonistic LTBR antibodies expressed as described in the Examples were shown to be intact and properly assembled as demonstrated by reducing and non-reducing SDS-PAGE.

### Assays

The agonistic LTBR antibodies or bispecific agonistic LTBR antibodies provided herein may be characterized for their binding properties and/or biological activity by various assays known in the art. In particular, they are characterized by the assays described in more detail in the examples.

### 1. Binding Assay

Binding of the agonistic LTBR antibodies or bispecific agonistic LTBR antibodies provided herein to the corresponding target expressing cells may be evaluated for example by using a murine fibroblast cell line expressing human Fibroblast Activation Protein (FAP) and flow cytometry (FACS) analysis. Binding of the bispecific antigen binding molecules provided herein to LTBR may be determined by using CHO-K1 cells engineered to overexpress human, cyno or murine LTBR as described in Example 5.1.

### 2. Activity Assays

Agonistic LTBR antibodies or bispecific agonistic LTBR antibodies as described herein are tested for biological activity. Biological activity may include efficacy and specificity of the bispecific antigen binding molecules. Efficacy and specificity are demonstrated by assays measuring their ability to upregulate ICAM in human endothelial cells and cancer associated fibroblasts (CAFs) in vitro (Example 4). The activation of LTBR can be further measured by upregulation of inflammatory and developmental genes such as adhesion molecules (ICAM and VCAM) and chemoattractants (CXCL9, CXCL10 and CXCL11) by stromal cells (Example 5.2).

### Pharmaceutical Compositions, Formulations and Routes of Administration

In a further aspect, provided are pharmaceutical compositions comprising any of the agonistic LTBR antibodies or bispecific agonistic LTBR antibodies provided herein, e.g., for use in any of the below therapeutic methods. In one aspect, a pharmaceutical composition comprises any of the agonistic LTBR antibodies or bispecific agonistic LTBR antibodies provided herein and at least one pharmaceutically acceptable excipient. In another embodiment, a pharmaceutical composition comprises any of the agonistic LTBR antibodies or bispecific agonistic LTBR antibodies provided herein and at least one additional therapeutic agent, e.g., as described below.

Pharmaceutical compositions as disclosed herein comprise a therapeutically effective amount of one or more agonistic LTBR antibodies or bispecific agonistic LTBR antibodies dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one agonistic LTBR antibody or bispecific agonistic LTBR antibody and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intra-lesional, intravenous, intra-arterial, intramuscular, intrathecal or intraperitoneal injection. For injection, the agonistic LTBR antibodies or bispecific agonistic LTBR antibodies may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the agonistic LTBR antibodies or bispecific agonistic LTBR antibodies may be in powder form for constitution with a suitable vehicle, e.g., sterile pyrogen-free water, before use. Sterile injectable solutions are prepared by incorporating the agonistic LTBR antibodies or bispecific agonistic LTBR antibodies in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less than 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the antigen binding molecules may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as emulsion in an acceptable oil) or ion exchange resins, or as sparingly soluble derivatives, for example, as a sparingly soluble salt.

Pharmaceutical compositions comprising the agonistic LTBR antibodies or bispecific agonistic LTBR antibodies disclosed herein may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The agonistic LTBR antibodies or bispecific agonistic LTBR antibodies may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The compositions herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

### Therapeutic Methods and Compositions

Any of the agonistic LTBR antibodies or bispecific agonistic LTBR antibodies provided herein, in particular the bispecific agonistic LTBR antibodies, may be used in therapeutic methods. For use in therapeutic methods, bispecific agonistic LTBR antibodies can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, agonistic LTBR antibodies or bispecific agonistic LTBR antibodies for use as a medicament are provided.

In further aspects, bispecific agonistic LTBR antibodies as described herein for use in inducing immune stimulation by activation of cancer associated fibroblasts (CAFs) or endothelial cells are provided. Furthermore, bispecific antigen binding molecules as described herein are provided for the (i) the treatment of cancer, (ii) delaying progression of cancer, and (iii) prolonging the survival of a patient suffering from cancer, in particular in the presence of FAP-expressing cells. In a particular aspect, bispecific agonistic LTBR antibodies for use in treating a disease, in particular for use in the treatment of cancer, are provided.

In certain aspects, agonistic LTBR antibodies or bispecific agonistic LTBR antibodies as described herein for use in a method of treatment are provided. In one aspect, provided is a bispecific agonistic LTBR antibody as described herein for use in the treatment of a disease in an individual in need thereof. In certain aspects, provided is a bispecific agonistic LTBR antibody for use in a method of treating an individual having a disease comprising administering to the individual a therapeutically effective amount of the bispecific agonistic LTBR antibody. In certain aspects the disease to be treated is cancer. The subject, patient, or “individual” in need of treatment is typically a mammal, more specifically a human.

In one aspect, provided is a method for i) inducing immune stimulation by CD40+ antigen-presenting cells (APCs), (ii) stimulating tumor-specific T cell response, (iii) causing apoptosis of tumor cells, (iv) treating of cancer, (v) delaying progression of cancer, (vi) prolonging the survival of a patient suffering from cancer, or (vii) treating of infections, wherein the method comprises administering a therapeutically effective amount of the bispecific agonistic LTBR antibody disclosed herein to an individual in need thereof.

In a further aspect, provided is the use of the bispecific agonistic LTBR antibody described herein in the manufacture or preparation of a medicament for the treatment of a disease in an individual in need thereof. In one aspect, the medicament is for use in a method of treating a disease comprising administering to an individual having the disease a therapeutically effective amount of the medicament. In certain aspects, the disease to be treated is a proliferative disorder, particularly cancer. Examples of cancers include, but are not limited to, bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other examples of cancer include carcinoma, lymphoma (e.g., Hodgkin's and non-Hodgkin's lymphoma), blastoma, sarcoma, and leukemia. Other cell proliferation disorders that can be treated using the bispecific antigen binding molecule or antibody of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the bispecific agonistic LTBR antibody may not provide a cure but may provide a benefit. In some aspects, a physiological change having some benefit is also considered therapeutically beneficial. Thus, in some aspects, an amount of the bispecific agonistic LTBR antibody or agonistic LTBR antibody that provides a physiological change is considered an “effective amount” or a “therapeutically effective amount”.

For the prevention or treatment of disease, the appropriate dosage of a bispecific agonistic LTBR antibody described herein (when used alone or in combination with one or more other additional therapeutic agents) will depend on the type of disease to be treated, the route of administration, the body weight of the patient, the specific molecule, the severity and course of the disease, whether the bispecific antigen binding molecule of the invention is administered for preventive or therapeutic purposes, previous or concurrent therapeutic interventions, the patient's clinical history and response to the bispecific antigen binding molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The bispecific agonistic LTBR antibody is suitably administered to the patient at one time or over a series of treatments. Depending on the type and severity of the disease, about 1 μg/kg to 15 mg/kg (e.g. 0.1 mg/kg-10 mg/kg) of the bispecific antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the bispecific antigen binding molecule of the invention would be in the range from about 0.005 mg/kg to about 10 mg/kg. In other examples, a dose may also comprise from about 1 μg/kg body weight, about 5 μg/kg body weight, about 10 g/kg body weight, about 50 μg/kg body weight, about 100 μg/kg body weight, about 200 g/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 0.1 mg/kg body weight to about 20 mg/kg body weight, about 5 μg/kg body weight to about 1 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). In a particular aspect, the bispecific agonistic LTBR antibody will be administered every three weeks. In one aspect, the bispecific agonistic LTBR antibody will be administered every two weeks. An initial higher loading dose, followed by one or more lower doses may be administered. However, other dosage regimens may be useful. The progress of this therapy is easily monitored by conventional techniques and assays.

The bispecific agonistic LTBR antibody will generally be used in an amount effective to achieve the intended purpose. For use to treat or prevent a disease condition, the bispecific agonistic LTBR antibody, or pharmaceutical compositions thereof, are administered or applied in a therapeutically effective amount. Determination of a therapeutically effective amount is well within the capabilities of those skilled in the art, especially in light of the detailed disclosure provided herein. For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans. Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the bispecific antigen binding molecule of the invention which are sufficient to maintain therapeutic effect. Usual patient dosages for administration by injection range from about 0.1 to 50 mg/kg/day, typically from about 0.1 to 1 mg/kg/day. Therapeutically effective plasma levels may be achieved by administering multiple doses each day. Levels in plasma may be measured, for example, by HPLC. In cases of local administration or selective uptake, the effective local concentration of the bispecific agonistic LTBR antibody may not be related to plasma concentration. One skilled in the art will be able to optimize therapeutically effective local dosages without undue experimentation.

A therapeutically effective dose of the bispecific agonistic LTBR antibody described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Bispecific agonistic LTBR antibodies that exhibit large therapeutic indices are preferred. In one aspect, the bispecific agonistic LTBR antibody exhibits a high therapeutic index. The data obtained from cell culture assays and animal studies can be used in formulating a range of dosages suitable for use in humans. The dosage lies preferably within a range of circulating concentrations that include the ED50 with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with agonistic LTBR antibodies would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### Other Agents and Treatments

The bispecific agonistic LTBR antibody may be administered in combination with one or more other agents in therapy. For instance, the agonistic LTBR antibody or bispecific agonistic LTBR antibody may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain aspects, an additional therapeutic agent is another anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent. In certain aspects, an additional therapeutic agent is an immunomodulatory agent, a cytostatic agent, an inhibitor of cell adhesion, a cytotoxic or cytostatic agent, an activator of cell apoptosis, or an agent that increases the sensitivity of cells to apoptotic inducers.

Thus, provided are bispecific agonistic LTBR antibodies or pharmaceutical compositions comprising them for use in the treatment of cancer, wherein the bispecific antigen binding molecule is administered in combination with a chemotherapeutic agent, radiation and/or other agents for use in cancer immunotherapy.

Such other agents are suitably present in combination in amounts that are effective for the purpose intended. The effective amount of such other agents depends on the amount of bispecific agonistic LTBR antibody used, the type of disorder or treatment, and other factors discussed above. The bispecific agonistic LTBR antibodies are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the bispecific antigen binding molecule or antibody of the invention can occur prior to, simultaneously, and/or following, administration of the additional therapeutic agent and/or adjuvant.

In a further aspect, provided is the bispecific agonistic LTBR antibody as described herein before for use in the treatment of cancer, wherein the bispecific antigen binding molecule is administered in combination with another immunomodulator.

The term “immunomodulator” refers to any substance including a monoclonal antibody that effects the immune system. The molecules disclosed herein can be considered immunomodulators. Immunomodulators can be used as anti-neoplastic agents for the treatment of cancer. In one aspect, immunomodulators include, but are not limited to anti-CTLA4 antibodies (e.g. ipilimumab), anti-PD1 antibodies (e.g. nivolumab or pembrolizumab), PD-L1 antibodies (e.g. atezolizumab, avelumab or durvalumab), OX-40 antibodies, 4-1BB antibodies and GITR antibodies. In a further aspect, provided is the bispecific antigen binding molecule as described herein before for use in the treatment of cancer, wherein the bispecific antigen binding molecule is administered in combination with an agent blocking PD-L1/PD-1 interaction. In one aspect, the agent blocking PD-L1/PD-1 interaction is an anti-PD-L1 antibody or an anti-PD1 antibody. More particularly, the agent blocking PD-L1/PD-1 interaction is an anti-PD-L1 antibody, in particular an anti-PD-L1 antibody selected from the group consisting of atezolizumab, durvalumab, pembrolizumab and nivolumab. In one specific aspect, the agent blocking PD-L1/PD-1 interaction is atezolizumab (MPDL3280A, RG7446). In another aspect, the agent blocking PD-L1/PD-1 interaction is an anti-PD-L1 antibody comprising a heavy chain variable domain VH (PDL-1) of SEQ ID NO:313 and a light chain variable domain VL (PDL-1) of SEQ ID NO:314. In another aspect, the agent blocking PD-L1/PD-1 interaction is an anti-PD-L1 antibody comprising a heavy chain variable domain VH (PD-L1) of SEQ ID NO:315 and a light chain variable domain VL (PD-L1) of SEQ ID NO:316. In another aspect, the agent blocking PD-L1/PD-1 interaction is an anti-PD1 antibody, in particular an anti-PD1 antibody selected from pembrolizumab or nivolumab. Such other agents are suitably present in combination in amounts that are effective for the purpose intended. The effective amount of such other agents depends on the amount of bispecific agonistic LTBR antibody used, the type of disorder or treatment, and other factors discussed above. The bispecific agonistic LTBR antibodies as described herein before are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the bispecific agonistic LTBR antibody can occur prior to, simultaneously, and/or following, administration of the additional therapeutic agent and/or adjuvant.

### Articles of Manufacture

In another aspect, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a bispecific agonistic LTBR antibody disclosed herein.

The label or package insert indicates that the composition is used for treating the condition of choice. Moreover, the article of manufacture may comprise (a) a first container with a composition contained therein, wherein the composition comprises a bispecific agonistic LTBR antibody; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID NO: | Name | Sequence |
| 1 | hu LTBR | UniProt no. P36941 |
| | | MLLPWATSAP GLAWGPLVLG LFGLLAASQP |
| | | QAVPPYASEN QTCRDQEKEY YEPQHRICCS |
| | | RCPPGTYVSA KCSRIRDTVC ATCAENSYNE |
| | | HWNYLTICQL CRPCDPVMGL EEIAPCTSKR |
| | | KTQCRCQPGM FCAAWALECT HCELLSDCPP |
| | | GTEAELKDEV GKGNNHCVPC KAGHFQNTSS |
| | | PSARCQPHTR CENQGLVEAA PGTAQSDTTC |
| | | KNPLEPLPPE MSGTMLMLAV LLPLAFFLLL |
| | | ATVESCIWKS HPSLCRKLGS LLKRRPQGEG |
| | | PNPVAGSWEP PKAHPYFPDL VQPLLPISGD |
| | | VSPVSTGLPA APVLEAGVPQ QQSPLDLTRE |
| | | PQLEPGEQSQ VAHGINGIHV TGGSMTITGN |
| | | IYIYNGPVLG GPPGPGDLPA TPEPPYPIPE |
| | | EGDPGPPGLS TPHQEDGKAW HLAETEHCGA |
| | | TPSNRGPRNQ FITH D |
| 2 | hu FAP | UniProt no. Q12884, version 168 |
| | | MKTWVKIVFG VATSAVLALL VMCIVLRPSR |
| | | VHNSEENTMR ALTLKDILNG TFSYKTFFPN |
| | | WISGQEYLHQ SADNNIVLYN IETGQSYTIL |
| | | SNRTMKSVNA SNYGLSPDRQ FVYLESDYSK |
| | | LWRYSYTATY YIYDLSNGEF VRGNELPRPI |
| | | QYLCWSPVGS KLAYVYQNNI YLKQRPGDPP |
| | | FQITENGREN KIFNGIPDWV YEEEMLATKY |
| | | ALWWSPNGKF LAYAEENDTD IPVIAYSYYG |
| | | DEQYPRTINI PYPKAGAKNP VVRIFIIDTT |
| | | YPAYVGPQEV PVPAMIASSD YYFSWLTWVT |
| | | DERVCLQWLK RVQNVSVLSI CDFREDWQTW |
| | | DCPKTQEHIE ESRTGWAGGF FVSTPVESYD |
| | | AISYYKIFSD KDGYKHIHYI KDTVENAIQI |
| | | TSGKWEAINI FRVTQDSLFY SSNEFEEYPG |
| | | RRNIYRISIG SYPPSKKCVT CHLRKERCQY |
| | | YTASFSDYAK YYALVCYGPG IPISTLHDGR |
| | | TDQEIKILEE NKELENALKN IQLPKEEIKK |
| | | LEVDEITLWY KMILPPQFDR SKKYPLLIQV |
| | | YGGPCSQSVR SVFAVNWISY LASKEGMVIA |
| | | LVDGRGTAFQ GDKLLYAVYR KLGVYEVEDQ |
| | | ITAVRKFIEM GFIDEKRIAI WGWSYGGYVS |
| | | SLALASGTGL FKCGIAVAPV SSWEYYASVY |
| | | TERFMGLPTK DDNLEHYKNS TVMARAEYFR |
| | | NVDYLLIHGT ADDNVHFQNS AQIAKALVNA |
| | | QVDFQAMWYS DQNHGLSGLS TNHLYTHMTH |
| | | FLKQCFSLSD |
| 3 | FAP (4B9) CDR-H1 | SYAMS |
| 4 | FAP (4B9) CDR-H2 | AIIGSGASTYYADSVKG |
| 5 | FAP (4B9) CDR-H3 | GWFGGFNY |
| 6 | FAP (4B9) CDR-L1 | RASQSVTSSYLA |
| 7 | FAP (4B9) CDR-L2 | VGSRRAT |
| 8 | FAP (4B9) CDR-L3 | QQGIMLPPT |
| 9 | FAP (4B9) VH | EVQLLESGGGLVQPGGSLRLSCAASGFTFSSYAMS |
| | | WVRQAPGKGLEWVSAIIGSGASTYYADSVKGRFTI |
| | | SRDNSKNTLYLQMNSLRAEDTAVYYCAKGWEGGEN |
| | | YWGQGTLVTVSS |
| 10 | FAP (4B9) VL | EIVLTQSPGTLSLSPGERATLSCRASQSVTSSYLA |
| | | WYQQKPGQAPRLLINVGSRRATGIPDRFSGSGSGT |
| | | DFTLTISRLEPEDFAVYYCQQGIMLPPTFGQGTKV |
| | | EIK |
| 11 | FAP (28H1) CDR-H1 | SHAMS |
| 12 | FAP (28H1) CDR-H2 | AIWASGEQYYADSVKG |
| 13 | FAP (28H1) CDR-H3 | GWLGNEDY |
| 14 | FAP (28H1) CDR-L1 | RASQSVSRSYLA |
| 15 | FAP (28H1) CDR-L2 | GASTRAT |
| 16 | FAP (28H1) CDR-L3 | QQGQVIPPT |
| 17 | FAP (28H1) VH | EVQLLESGGGLVQPGGSLRLSCAASGFTFSSHAMS |
| | | WVRQAPGKGLEWVSAIWASGEQYYADSVKGRFTIS |
| | | RDNSKNTLYLQMNSLRAEDTAVYYCAKGWLGNEDY |
| | | WGQGTLVTVSS |
| 18 | FAP (28H1) VL | EIVLTQSPGTLSLSPGERATLSCRASQSVSRSYLA |
| | | WYQQKPGQAPRLLIIGASTRATGIPDRFSGSGSGT |
| | | DFTLTISRLEPEDFAVYYCQQGQVIPPTFGQGTKV |
| | | EIK |
| 19 | FAP (1G1a) CDR-H1 | DYNMD |
| 20 | FAP (1G1a) CDR-H2 | DIYPNTGGTIYNQKFKG |
| 21 | FAP (1G1a) CDR-H3 | ERGIHYAMDY |
| 22 | FAP (1G1a) CDR-L1 | RASESVDNYGLSFIN |
| 23 | FAP (1G1a) CDR-L2 | GTSNRGS |
| 24 | FAP (1G1a) CDR-L3 | QQSNEVPYT |
| 25 | FAP (1G1a) VH | QVQLVQSGAEVKKPGASVKVSCKASGYTLTDYNMD |
| | | WVRQAPGQGLEWIGDIYPNTGGTIYNQKFKGRVTM |
| | | TIDTSTSTVYMELSSLRSEDTAVYYCTRERGIHYA |
| | | MDYWGQGTTVTVSS |
| 26 | FAP (1G1a) VL | EIVLTQSPATLSLSPGERATLSCRASESVDNYGLS |
| | | FINWFQQKPGQAPRLLIYGTSNRGSGIPARFSGSG |
| | | SGTDFTLTISSLEPEDFAVYFCQQSNEVPYTFGGG |
| | | TKVEIK |
| 27 | LTBR (9459) CDR-H1 | SYTMN |
| 28 | LTBR (9459) CDR-H2 | GVAPSGDRTYYTDSVKG |
| 29 | LTBR (9459) CDR-H3 | GFAFTN |
| 30 | LTBR (9459) CDR-L1 | RASQGISSALA |
| 31 | LTBR (9459) CDR-L2 | DASSLDS |
| 32 | LTBR (9459) CDR-L3 | QQYNNYPLT |
| 33 | LTBR (9459) VH | EVQLVESGGGLVQPGGSLRLSCAASGFTFSSYTMN |
| | | WVRQAPGKGLEWVSGVAPSGDRTYYTDSVKGRFTI |
| | | SRDNAKNTLDLQMTSLRAEDAAIYICAQGFAFTNW |
| | | GQGTMVTVSS |
| 34 | LTBR (9459) VL | DIQMTQSPSSLSASVGDRVTITCRASQGISSALAW |
| | | FQQKPGKAPKRLIFDASSLDSGVPSRFSGSGSGTD |
| | | FTLTISSLQPEDFATYYCQQYNNYPLTFGGGTKVE |
| | | MK |
| 35 | LTBR (9452) CDR-H1 | SYPMT |
| 36 | LTBR (9452) CDR-H2 | LISPSGATTYYADSVKG |
| 37 | LTBR (9452) CDR-H3 | NHYPDDVIIKNYYFYGVDV |
| 38 | LTBR (9452) CDR-L1 | RASQDISNYLN |
| 39 | LTBR (9452) CDR-L2 | AASSLQS |
| 40 | LTBR (9452) CDR-L3 | QQTHTIPYS |
| 41 | LTBR (9452) VH | EVQLLESGGGLVQPGGSLRLSCAAAGFTFSSYPMT |
| | | WVRQAPGKGLEWVSLISPSGATTYYADSVKGRFTI |
| | | SRDTSKNTLYLQMNSLRAEDTAVYYCAKNHYPDDV |
| | | IIKNYYFYGVDVWGQGTTVTVSS |
| 42 | LTBR (9452) VL | DIQMTQSPSSLSASVGDRVTITCRASQDISNYLNW |
| | | YQQKPGKAPKSLIYAASSLQSGVPSRFSGSGSGTD |
| | | FTLTISSLQPEDFANYYCQQTHTIPYSFGQGTNLE |
| | | IK |
| 43 | LTBR (0080) CDR-H1 | YYAMT |
| 44 | LTBR (0080) CDR-H2 | VINSGGGTYYTDSVKG |
| 45 | LTBR (0080) CDR-H3 | ASTADV |
| 46 | LTBR (0080) CDR-L1 | QASQGISNYLS |
| 47 | LTBR (0080) CDR-L2 | AASSLES |
| 48 | LTBR (0080) CDR-L3 | QQYNSYSLT |
| 49 | LTBR (0080) VH | EVQLLESGGGLVQPGGSLRLSCAASGFTFSYYAMT |
| | | WVRQAPGKGLEWIGVINSGGGTYYTDSVKGRFTMS |
| | | RDNSKNTLYLQVNSLRAEDTAVYYCAKASTADVWG |
| | | QGTTVTVSS |
| 50 | LTBR (0080) VL | DIQMTQSPSSLSASVGDRVTITCQASQGISNYLSW |
| | | YQQKPGKAPKRLIFAASSLESGVPSRFSGSGSGTD |
| | | FTLTISSLQPEDFATYYCQQYNSYSLTFGGGTKLE |
| | | IK |
| 51 | LTBR (9450) CDR-H1 | SYVMK |
| 52 | LTBR (9450) CDR-H2 | SISGSGGRPFYADSVKG |
| 53 | LTBR (9450) CDR-H3 | DMDV |
| 54 | LTBR (9450) CDR-L1 | RASQDISSELK |
| 55 | LTBR (9450) CDR-L2 | AASALQS |
| 56 | LTBR (9450) CDR-L3 | QQYNTYPLT |
| 57 | LTBR (9450) VH | EVQLLESGGGLVQPGGSLRLSCAASGFTVSSYVMK |
| | | WFRQAPGKGLEWVSSISGSGGRPFYADSVKGRFTI |
| | | SRDNSKNALYLQMNSLRAEDTAVYYCANDMDVWGQ |
| | | GTTVTVSS |
| 58 | LTBR (9450) VL | DIQMTQSPSSLSASVGDRVTITCRASQDISSELKW |
| | | YQQKPGKAPTLLIYAASALQSGVPSRFSGSGSGTD |
| | | FTLTISSLQPEDFATYYCQQYNTYPLTFGGGTKVD |
| | | IK |
| 59 | LTBR (0079) CDR-H1 | SYAMS |
| 60 | LTBR (0079) CDR-H2 | VITGSSSSIDYADSVKG |
| 61 | LTBR (0079) CDR-H3 | GGSGVWSISTDEDY |
| 62 | LTBR (0079) CDR-L1 | QASQDISNYLN |
| 63 | LTBR (0079) CDR-L2 | AASSLHS |
| 64 | LTBR (0079) CDR-L3 | QQSHSTPPT |
| 65 | LTBR (0079) VH | EVQLLESGGALVQPGGSLRLSCAASGFTFSSYAMS |
| | | WVRQAPGKGLEWVSVITGSSSSIDYADSVKGRFTI |
| | | SRDNSKNTLYLQMNSLRAEDTAVYYCAKGGSGVWS |
| | | ISTDFDYWGQGTLVTVSS |
| 66 | LTBR (0079) VL | DIQMTQSPSFLSASVGDRVTITCQASQDISNYLNW |
| | | YQQKPGKAPKHLIYAASSLHSGVPSRFSGSGSGAY |
| | | FTLTITSLQPEDFATYYCQQSHSTPPTFGPGTKVD |
| | | IK |
| 67 | LTBR (9457) CDR-H1 | NYAMT |
| 68 | LTBR (9457) CDR-H2 | EISGSGGSTYYADSVKG |
| 69 | LTBR (9457) CDR-H3 | DDDGAYGTMDV |
| 70 | LTBR (9457) CDR-L1 | RASQDISNYLN |
| 71 | LTBR (9457) CDR-L2 | AASALQS |
| 72 | LTBR (9457) CDR-L3 | QHYKSYPYT |
| 73 | LTBR (9457) VH | EVQLLESGGGLVQPGGSLRLSCAASGFTFSNYAMT |
| | | WVRQAPGKGLEWVSEISGSGGSTYYADSVKGRFTI |
| | | SRDNSKNTLYLQMNSLRAEDTAVYYCAKDDDGAYG |
| | | DTMDVWGQGTTVTVSS |
| 74 | LTBR (9457) VL | DIQMTQSPSSLSASIGDRVTITCRASQDISNYLNW |
| | | YQQKPGKAPKLLIYAASALQSGVPSRFSGSGSGTD |
| | | FTLTISSLQPEDFATYYCQHYKSYPYTFGQGTKVE |
| | | IR |
| 75 | LTBR (0064) CDR-H1 | NTYIH |
| 76 | LTBR (0064) CDR-H2 | RIDPTNGNTICTEKFKN |
| 77 | LTBR (0064) CDR-H3 | TTLTTPDSFAY |
| 78 | LTBR (0064) CDR-L1 | KASQNIYKYLN |
| 79 | LTBR (0064) CDR-L2 | STNILQT |
| 80 | LTBR (0064) CDR-L3 | QKYYSGFT |
| 81 | LTBR (0064) VH | EVQLQQYGAELGKPGTSVKLSCKVSGYNIGNTYIH |
| | | WVIQRPGKGLEWIGRIDPTNGNTICTEKFKNKATL |
| | | TADLSSNTAYMQLSQLKSDDTAIYFCAPTTLTTPD |
| | | SFAYWGQGTLVTVSS |
| 82 | LTBR (0064) VL | DIQMTQSPSQLSASVGDRVTLSCKASQNIYKYLNW |
| | | YQQKLGEAPRLLIYSTNILQTGIPSRFSGSGSGTD |
| | | YTLTISSLQPEDAATYFCQKYYSGFTFGSGTKLEM |
| | | K |
| 83 | LTBR (5929) CDR-H1 | SYAIS |
| 84 | LTBR (5929) CDR-H2 | GIIPIFGTANYAQKFQG |
| 85 | LTBR (5929) CDR-H3 | PSYYLDGWLDY |
| 86 | LTBR (5929) CDR-L1 | KSSQSVLYSSNNKNYLA |
| 87 | LTBR (5929) CDR-L2 | WASTRES |
| 88 | LTBR (5929) CDR-L3 | SQYYTTHYT |
| 89 | LTBR (5929) VH | QVQLVQSGAEVKKPGSSVKVSCKASGGTFSSYAIS |
| | | WVRQAPGQGLEWMGGIIPIFGTANYAQKFQGRVTI |
| | | TADKSTSTAYMELSSLRSEDTAVYYCARPSYYLDG |
| | | WLDYWGQGTTVTVSS |
| 90 | LTBR (5929) VL | DIVMTQSPDSLAVSLGERATINCKSSQSVLYSSNN |
| | | KNYLAWYQQKPGQPPKLLIYWASTRESGVPDRFSG |
| | | SGSGTDFTLTISSLQAEDVAVYYCSQYYTTHYTFG |
| | | QGTKVEIK |
| 91 | LTBR (7213) CDR-H1 | SYAIS |
| 92 | LTBR (7213) CDR-H2 | GIIPIHGTANYAQKFQG |
| 93 | LTBR (7213) CDR-H3 | PSYYLDGWLDY |
| 94 | LTBR (7213) CDR-L1 | KSSQSVLYSSNNKNYLA |
| 95 | LTBR (7213) CDR-L2 | WASTRES |
| 96 | LTBR (7213) CDR-L3 | SQYYTTHYT |
| 97 | LTBR (7213) VH | QVQLVQSGAEVKKPGSSVKVSCKASGGTFSSYAIS |
| | | WVRQAPGQGLEWMGGIIPIHGTANYAQKFQGRVTI |
| | | TADKSTSTAYMELSSLRSEDTAVYYCARPSYYLDG |
| | | WLDYWGQGTTVTVSS |
| 98 | LTBR (7213) VL | DIVMTQSPDSLAVSLGERATINCKSSQSVLYSSNN |
| | | KNYLAWYQQKPGQPPKLLIYWASTRESGVPDRFSG |
| | | SGSGTDFTLTISSLQAEDVAVYYCSQYYTTHYTFG |
| | | QGTKVEIK |
| 99 | LTBR (CBE11) VH | EVQLVESGGGLVKPGGSLRLSCAASGFTFSDYYMY |
| | | WFRQAPGKGLEWVATISDGGSYTYYPDSVKGRFTI |
| | | SRDNAKNSLYLQMSSLRAEDTAVYYCAREENGNFY |
| | | YFDYWGQGTTVTVSS |
| 100 | LTBR (CBE11) VL | DIQMTQSPSSLSASVGDRVTITCKAGQDIKSYLSW |
| | | YQQKPGKAPKLLIYYATRLADGVPSRFSGSGSGTD |
| | | YTLTISSLQPEDFATYYCLQHGESPWTFGGGTKLE |
| | | IK |
| 101 | LTBR (CBE11) CDR-H1 | DYYMY |
| 102 | LTBR (CBE11) CDR-H2 | TISDGGSYTYYPDSVKG |
| 103 | LTBR (CBE11) CDR-H3 | EENGNFYYFDY |
| 104 | LTBR (CBE11) CDR-L1 | KAGQDIKSYLS |
| 105 | LTBR (CBE11) CDR-L2 | YATRLAD |
| 106 | LTBR (CBE11) CDR-L3 | LQHGESPWT |
| 107 | P1AF0532 HC knob LTBR | DIVMTQSPDSLAVSLGERATINCKSSQSVLYSSNN |
| | crossed | KNYLAWYQQKPGQPPKLLIYWASTRESGVPDRFSG |
| | | SGSGTDFTLTISSLQAEDVAVYYCSQYYTTHYTFG |
| | | QGTKVEIKSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVD |
| | | KKVEPKSCDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALGAPIEKTISKAKGQPREPQVYTLP |
| | | PCRDELTKNQVSLWCLVKGFYPSDIAVEWESNGQP |
| | | ENNYKTTPPVLDSDGSFFLYSKLTVDKSRWQQGNV |
| | | FSCSVMHEALHNHYTQKSLSLSP |
| 108 | P1AF0532 LC LTBR | QVQLVQSGAEVKKPGSSVKVSCKASGGTESSYAIS |
| | crossed | WVRQAPGQGLEWMGGIIPIFGTANYAQKFQGRVTI |
| | | TADKSTSTAYMELSSLRSEDTAVYYCARPSYYLDG |
| | | WLDYWGQGTTVTVSSASVAAPSVFIFPPSDEQLKS |
| | | GTASVVCLLNNFYPREAKVQWKVDNALQSGNSQES |
| | | VTEQDSKDSTYSLSSTLTLSKADYEKHKVYACEVT |
| | | HQGLSSPVTKSFNRGEC |
| 109 | P1AF0532 HC hole FAP | EVQLLESGGGLVQPGGSLRLSCAASGFTFSSYAMS |
| | with charges | WVRQAPGKGLEWVSAIIGSGASTYYADSVKGRFTI |
| | | SRDNSKNTLYLQMNSLRAEDTAVYYCAKGWEGGEN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSP |
| 110 | P1AF0532 LC FAP with | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 111 | P1AF9728 HC knob LTBR | QVQLVQSGAEVKKPGSSVKVSCKASGGTFSSYAIS |
| | | WVRQAPGQGLEWMGGIIPIHGTANYAQKFQGRVTI |
| | | TADKSTSTAYMELSSLRSEDTAVYYCARPSYYLDG |
| | | WLDYWGQGTTVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPEAAGGPSVEL |
| | | FPPKPKDTLMISRTPEVTCVVVDVSHEDPEVKENW |
| | | YVDGVEVHNAKTKPREEQYNSTYRVVSVLTVLHQD |
| | | WLNGKEYKCKVSNKALGAPIEKTISKAKGQPREPQ |
| | | VYTLPPCRDELTKNQVSLWCLVKGFYPSDIAVEWE |
| | | SNGQPENNYKTTPPVLDSDGSFFLYSKLTVDKSRW |
| | | QQGNVFSCSVMHEALHNHYTQKSLSLSP |
| 112 | P1AF9728 LC LTBR | DIVMTQSPDSLAVSLGERATINCKSSQSVLYSSNN |
| | | KNYLAWYQQKPGQPPKLLIYWASTRESGVPDRFSG |
| | | SGSGTDFTLTISSLQAEDVAVYYCSQYYTTHYTFG |
| | | QGTKVEIKRTVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC |
| 113 | P1AF9728 HC hole FAP | EVQLLESGGGLVQPGGSLRLSCAASGFTFSSYAMS |
| | crossed | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASVAAPSVFIFPPSDEQLKSGTA |
| | | SVVCLLNNFYPREAKVQWKVDNALQSGNSQESVTE |
| | | QDSKDSTYSLSSTLTLSKADYEKHKVYACEVTHQG |
| | | LSSPVTKSFNRGECDKTHTCPPCPAPEAAGGPSVE |
| | | LFPPKPKDTLMISRTPEVTCVVVDVSHEDPEVKFN |
| | | WYVDGVEVHNAKTKPREEQYNSTYRVVSVLTVLHQ |
| | | DWLNGKEYKCKVSNKALGAPIEKTISKAKGQPREP |
| | | QVCTLPPSRDELTKNQVSLSCAVKGFYPSDIAVEW |
| | | ESNGQPENNYKTTPPVLDSDGSFFLVSKLTVDKSR |
| | | WQQGNVFSCSVMHEALHNHYTQKSLSLSP |
| 114 | P1AF9728 LC FAP | EIVLTQSPGTLSLSPGERATLSCRASQSVTSSYLA |
| | crossed | WYQQKPGQAPRLLINVGSRRATGIPDRFSGSGSGT |
| | | DFTLTISRLEPEDFAVYYCQQGIMLPPTFGQGTKV |
| | | EIKSSASTKGPSVFPLAPSSKSTSGGTAALGCLVK |
| | | DYFPEPVTVSWNSGALTSGVHTFPAVLQSSGLYSL |
| | | SSVVTVPSSSLGTQTYICNVNHKPSNTKVDKKVEP |
| | | KSC |
| 115 | P1AG7504 HC knob LTBR | QVQLVQSGAEVKKPGSSVKVSCKASGGTFSSYAIS |
| | | WVRQAPGQGLEWMGGIIPIHGTANYAQKFQGRVTI |
| | | TADKSTSTAYMELSSLRSEDTAVYYCARPSYYLDG |
| | | WLDYWGQGTTVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPEAAGGPSVFL |
| | | FPPKPKDTLMISRTPEVTCVVVDVSHEDPEVKENW |
| | | YVDGVEVHNAKTKPREEQYNSTYRVVSVLTVLHQD |
| | | WLNGKEYKCKVSNKALGAPIEKTISKAKGQPREPQ |
| | | VYTLPPCRDELTKNQVSLWCLVKGFYPSDIAVEWE |
| | | SNGQPENNYKTTPPVLDSDGSFFLYSKLTVDKSRW |
| | | QQGNVFSCSVMHEALHNHYTQKSLSLSPG |
| 116 | P1AG7504 LC LTBR | DIVMTQSPDSLAVSLGERATINCKSSQSVLYSSNN |
| | | KNYLAWYQQKPGQPPKLLIYWASTRESGVPDRFSG |
| | | SGSGTDFTLTISSLQAEDVAVYYCSQYYTTHYTFG |
| | | QGTKVEIKRTVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC |
| 117 | P1AG7504 HC hole FAP | EVQLLESGGGLVQPGGSLRLSCAASGFTFSSYAMS |
| | crossed | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASVAAPSVFIFPPSDEQLKSGTA |
| | | SVVCLLNNFYPREAKVQWKVDNALQSGNSQESVTE |
| | | QDSKDSTYSLSSTLTLSKADYEKHKVYACEVTHQG |
| | | LSSPVTKSFNRGECDKTHTCPPCPAPEAAGGPSVE |
| | | LFPPKPKDTLMISRTPEVTCVVVDVSHEDPEVKEN |
| | | WYVDGVEVHNAKTKPREEQYNSTYRVVSVLTVLHQ |
| | | DWLNGKEYKCKVSNKALGAPIEKTISKAKGQPREP |
| | | QVCTLPPSRDELTKNQVSLSCAVKGFYPSDIAVEW |
| | | ESNGQPENNYKTTPPVLDSDGSFFLVSKLTVDKSR |
| | | WQQGNVFSCSVMHEALHNHYTQKSLSLSPG |
| 118 | P1AG7504 LC FAP | EIVLTQSPGTLSLSPGERATLSCRASQSVTSSYLA |
| | crossed | WYQQKPGQAPRLLINVGSRRATGIPDRFSGSGSGT |
| | | DFTLTISRLEPEDFAVYYCQQGIMLPPTFGQGTKV |
| | | EIKSSASTKGPSVFPLAPSSKSTSGGTAALGCLVK |
| | | DYFPEPVTVSWNSGALTSGVHTFPAVLQSSGLYSL |
| | | SSVVTVPSSSLGTQTYICNVNHKPSNTKVDKKVEP |
| | | KSCD |
| 119 | P1AF0535 HC knob LTBR | DIQMTQSPSSLSASVGDRVTITCRASQDISSELKW |
| | crossed | YQQKPGKAPTLLIYAASALQSGVPSRESGSGSGTD |
| | | FTLTISSLQPEDFATYYCQQYNTYPLTFGGGTKVD |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSP |
| 120 | P1AF0535 LC LTBR | EVQLLESGGGLVQPGGSLRLSCAASGFTVSSYVMK |
| | crossed | WFRQAPGKGLEWVSSISGSGGRPFYADSVKGRFTI |
| | | SRDNSKNALYLQMNSLRAEDTAVYYCANDMDVWGQ |
| | | GTTVTVSSASVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC |
| 121 | P1AF0535 HC hole FAP | EVQLLESGGGLVQPGGSLRLSCAASGFTFSSYAMS |
| | with charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSP |
| 122 | P1AF0535 LC FAP with | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 123 | P1AG7506 HC knob LTBR | DIQMTQSPSSLSASVGDRVTITCRASQDISSELKW |
| | crossed | YQQKPGKAPTLLIYAASALQSGVPSRFSGSGSGTD |
| | | FTLTISSLQPEDFATYYCQQYNTYPLTFGGGTKVD |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPG |
| 124 | P1AG7506 LC LTBR | EVQLLESGGGLVQPGGSLRLSCAASGFTVSSYVMK |
| | crossed | WFRQAPGKGLEWVSSISGSGGRPFYADSVKGRFTI |
| | | SRDNSKNALYLQMNSLRAEDTAVYYCANDMDVWGQ |
| | | GTTVTVSSASVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC |
| 125 | P1AG7506 HC hole FAP | EVQLLESGGGLVQPGGSLRLSCAASGFTFSSYAMS |
| | charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSPG |
| 126 | P1AG7506 LC FAP | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 127 | P1AF0534 HC knob LTBR | DIQMTQSPSSLSASVGDRVTITCRASQDISNYLNW |
| | crossed | YQQKPGKAPKSLIYAASSLQSGVPSRESGSGSGTD |
| | | FTLTISSLQPEDFANYYCQQTHTIPYSFGQGTNLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSP |
| 128 | P1AF0534 LC LTBR | EVQLLESGGGLVQPGGSLRLSCAAAGFTFSSYPMT |
| | crossed | WVRQAPGKGLEWVSLISPSGATTYYADSVKGRFTI |
| | | SRDTSKNTLYLQMNSLRAEDTAVYYCAKNHYPDDV |
| | | IIKNYYFYGVDVWGQGTTVTVSSASVAAPSVFIFP |
| | | PSDEQLKSGTASVVCLLNNFYPREAKVQWKVDNAL |
| | | QSGNSQESVTEQDSKDSTYSLSSTLTLSKADYEKH |
| | | KVYACEVTHQGLSSPVTKSFNRGEC |
| 129 | P1AF0534 HC hole FAP | EVQLLESGGGLVQPGGSLRLSCAASGFTFSSYAMS |
| | with charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSP |
| 130 | P1AF0534 LC FAP with | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 131 | P1AG7505 HC knob LTBR | DIQMTQSPSSLSASVGDRVTITCRASQDISNYLNW |
| | crossed | YQQKPGKAPKSLIYAASSLQSGVPSRFSGSGSGTD |
| | | FTLTISSLQPEDFANYYCQQTHTIPYSFGQGTNLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPG |
| 132 | P1AG7505 LC LTBR | EVQLLESGGGLVQPGGSLRLSCAAAGFTFSSYPMT |
| | crossed | WVRQAPGKGLEWVSLISPSGATTYYADSVKGRFTI |
| | | SRDTSKNTLYLQMNSLRAEDTAVYYCAKNHYPDDV |
| | | IIKNYYFYGVDVWGQGTTVTVSSASVAAPSVFIFP |
| | | PSDEQLKSGTASVVCLLNNFYPREAKVQWKVDNAL |
| | | QSGNSQESVTEQDSKDSTYSLSSTLTLSKADYEKH |
| | | KVYACEVTHQGLSSPVTKSFNRGEC |
| 133 | P1AG7505 HC hole FAP | EVQLLESGGGLVQPGGSLRLSCAASGFTFSSYAMS |
| | charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSPG |
| 134 | P1AG7505 LC FAP | EIVLTQSPGTLSLSPGERATLSCRASQSVTSSYLA |
| | charges | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 135 | P1AF5257 HC knob LTBR | DIQMTQSPSSLSASIGDRVTITCRASQDISNYLNW |
| | crossed | YQQKPGKAPKLLIYAASALQSGVPSRFSGSGSGTD |
| | | FTLTISSLQPEDFATYYCQHYKSYPYTFGQGTKVE |
| | | IRSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSP |
| 136 | P1AF5257 LC LTBR | EVQLLESGGGLVQPGGSLRLSCAASGFTFSNYAMT |
| | crossed | WVRQAPGKGLEWVSEISGSGGSTYYADSVKGRFTI |
| | | SRDNSKNTLYLQMNSLRAEDTAVYYCAKDDDGAYG |
| | | DTMDVWGQGTTVTVSSASVAAPSVFIFPPSDEQLK |
| | | SGTASVVCLLNNFYPREAKVQWKVDNALQSGNSQE |
| | | SVTEQDSKDSTYSLSSTLTLSKADYEKHKVYACEV |
| | | THQGLSSPVTKSFNRGEC |
| 137 | P1AF5257 HC hole FAP | EVQLLESGGGLVQPGGSLRLSCAASGFTFSSYAMS |
| | charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSP |
| 138 | P1AF5257 LC FAP | EIVLTQSPGTLSLSPGERATLSCRASQSVTSSYLA |
| | charges | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 139 | P1AF0537 HC knob LTBR | DIQMTQSPSSLSASVGDRVTITCRASQGISSALAW |
| | crossed | FQQKPGKAPKRLIFDASSLDSGVPSRFSGSGSGTD |
| | | FTLTISSLQPEDFATYYCQQYNNYPLTFGGGTKVE |
| | | MKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSP |
| 140 | P1AF0537 LC LTBR | EVQLVESGGGLVQPGGSLRLSCAASGFTFSSYTMN |
| | crossed | WVRQAPGKGLEWVSGVAPSGDRTYYTDSVKGRFTI |
| | | SRDNAKNTLDLQMTSLRAEDAAIYICAQGFAFTNW |
| | | GQGTMVTVSSASVAAPSVFIFPPSDEQLKSGTASV |
| | | VCLLNNFYPREAKVQWKVDNALQSGNSQESVTEQD |
| | | SKDSTYSLSSTLTLSKADYEKHKVYACEVTHQGLS |
| | | SPVTKSFNRGEC |
| 141 | P1AF0537 HC hole FAP | EVQLLESGGGLVQPGGSLRLSCAASGFTFSSYAMS |
| | with charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSP |
| 142 | P1AF0537 LC FAP with | EIVLTQSPGTLSLSPGERATLSCRASQSVTSSYLA |
| | charges | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 143 | P1AG7507 HC knob LTBR | DIQMTQSPSSLSASVGDRVTITCRASQGISSALAW |
| | crossed | FQQKPGKAPKRLIFDASSLDSGVPSRFSGSGSGTD |
| | | FTLTISSLQPEDFATYYCQQYNNYPLTFGGGTKVE |
| | | MKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPG |
| 144 | P1AG7507 LC LTBR | EVQLVESGGGLVQPGGSLRLSCAASGFTFSSYTMN |
| | crossed | WVRQAPGKGLEWVSGVAPSGDRTYYTDSVKGRFTI |
| | | SRDNAKNTLDLQMTSLRAEDAAIYICAQGFAFTNW |
| | | GQGTMVTVSSASVAAPSVFIFPPSDEQLKSGTASV |
| | | VCLLNNFYPREAKVQWKVDNALQSGNSQESVTEQD |
| | | SKDSTYSLSSTLTLSKADYEKHKVYACEVTHQGLS |
| | | SPVTKSFNRGEC |
| 145 | P1AG7507 HC hole FAP | EVQLLESGGGLVQPGGSLRLSCAASGFTFSSYAMS |
| | charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVELEPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSPG |
| 146 | P1AG7507 LC FAP | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 147 | P1AG0694 HC knob LTBR | QVQLVQSGAEVKKPGASVKVSCKASGYNIGNTYIH |
| | | WVRQAPGKGLEWMGRIDPTNGNTIYAQKFQGRVTS |
| | | TRDTSISTAYMELSRLRSDDTVVYYCAPTTLTTPD |
| | | SFAYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPEAAGGPSVFL |
| | | FPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALGAPIEKTISKAKGQPREPQ |
| | | VYTLPPCRDELTKNQVSLWCLVKGFYPSDIAVEWE |
| | | SNGQPENNYKTTPPVLDSDGSFFLYSKLTVDKSRW |
| | | QQGNVFSCSVMHEALHNHYTQKSLSLSP |
| 148 | P1AG0694 LC LTBR | DIQMTQSPSSLSASVGDRVTITCRASQNIYKYLNW |
| | | YQQKPGKAPKLLIYSTNILQSGVPSRFSGSGSGTD |
| | | FTLTISSLQPEDFATYFCQKYYSGFTFGQGTKLEI |
| | | KRTVAAPSVFIFPPSDEQLKSGTASVVCLLNNFYP |
| | | REAKVQWKVDNALQSGNSQESVTEQDSKDSTYSLS |
| | | STLTLSKADYEKHKVYACEVTHQGLSSPVTKSFNR |
| | | GEC |
| 149 | P1AG0694 HC hole FAP | EVQLLESGGGLVQPGGSLRLSCAASGFTFSSYAMS |
| | crossed | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASVAAPSVFIFPPSDEQLKSGTA |
| | | SVVCLLNNFYPREAKVQWKVDNALQSGNSQESVTE |
| | | QDSKDSTYSLSSTLTLSKADYEKHKVYACEVTHQG |
| | | LSSPVTKSFNRGECDKTHTCPPCPAPEAAGGPSVF |
| | | LFPPKPKDTLMISRTPEVTCVVVDVSHEDPEVKFN |
| | | WYVDGVEVHNAKTKPREEQYNSTYRVVSVLTVLHQ |
| | | DWLNGKEYKCKVSNKALGAPIEKTISKAKGQPREP |
| | | QVCTLPPSRDELTKNQVSLSCAVKGFYPSDIAVEW |
| | | ESNGQPENNYKTTPPVLDSDGSFFLVSKLTVDKSR |
| | | WQQGNVFSCSVMHEALHNHYTQKSLSLSP |
| 150 | P1AG0694 LC FAP | EIVLTQSPGTLSLSPGERATLSCRASQSVTSSYLA |
| | crossed | WYQQKPGQAPRLLINVGSRRATGIPDRFSGSGSGT |
| | | DFTLTISRLEPEDFAVYYCQQGIMLPPTFGQGTKV |
| | | EIKSSASTKGPSVFPLAPSSKSTSGGTAALGCLVK |
| | | DYFPEPVTVSWNSGALTSGVHTFPAVLQSSGLYSL |
| | | SSVVTVPSSSLGTQTYICNVNHKPSNTKVDKKVEP |
| | | KSC |
| 151 | P1AF9727 HC knob LTBR | EVQLQQYGAELGKPGTSVKLSCKVSGYNIGNTYIH |
| | | WVIQRPGKGLEWIGRIDPTNGNTISTEKFKNKATL |
| | | TADLSSNTAYMQLSQLKSDDTAIYFCAPTTLTTPD |
| | | SFAYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPEAAGGPSVFL |
| | | FPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALGAPIEKTISKAKGQPREPQ |
| | | VYTLPPCRDELTKNQVSLWCLVKGFYPSDIAVEWE |
| | | SNGQPENNYKTTPPVLDSDGSFFLYSKLTVDKSRW |
| | | QQGNVFSCSVMHEALHNHYTQKSLSLSP |
| 152 | P1AF9727 LC LTBR | DIQMTQSPSQLSASVGDRVTLSCKASQNIYKYLNW |
| | | YQQKLGEAPRLLIYSTNILQTGIPSRFSGSGSGTD |
| | | YTLTISSLQPEDAATYFCQKYYSGFTFGSGTKLEM |
| | | KRTVAAPSVFIFPPSDEQLKSGTASVVCLLNNFYP |
| | | REAKVQWKVDNALQSGNSQESVTEQDSKDSTYSLS |
| | | STLTLSKADYEKHKVYACEVTHQGLSSPVTKSFNR |
| | | GEC |
| 153 | P1AF9727 HC hole FAP | EVQLLESGGGLVQPGGSLRLSCAASGFTFSSYAMS |
| | crossed | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASVAAPSVFIFPPSDEQLKSGTA |
| | | SVVCLLNNFYPREAKVQWKVDNALQSGNSQESVTE |
| | | QDSKDSTYSLSSTLTLSKADYEKHKVYACEVTHQG |
| | | LSSPVTKSFNRGECDKTHTCPPCPAPEAAGGPSVE |
| | | LFPPKPKDTLMISRTPEVTCVVVDVSHEDPEVKFN |
| | | WYVDGVEVHNAKTKPREEQYNSTYRVVSVLTVLHQ |
| | | DWLNGKEYKCKVSNKALGAPIEKTISKAKGQPREP |
| | | QVCTLPPSRDELTKNQVSLSCAVKGFYPSDIAVEW |
| | | ESNGQPENNYKTTPPVLDSDGSFFLVSKLTVDKSR |
| | | WQQGNVFSCSVMHEALHNHYTQKSLSLSP |
| 154 | P1AF9727 LC FAP | EIVLTQSPGTLSLSPGERATLSCRASQSVTSSYLA |
| | crossed | WYQQKPGQAPRLLINVGSRRATGIPDRFSGSGSGT |
| | | DFTLTISRLEPEDFAVYYCQQGIMLPPTFGQGTKV |
| | | EIKSSASTKGPSVFPLAPSSKSTSGGTAALGCLVK |
| | | DYFPEPVTVSWNSGALTSGVHTFPAVLQSSGLYSL |
| | | SSVVTVPSSSLGTQTYICNVNHKPSNTKVDKKVEP |
| | | KSC |
| 155 | P1AF5260 HC knob LTBR | DIQMTQSPSFLSASVGDRVTITCQASQDISNYLNW |
| | crossed | YQQKPGKAPKHLIYAASSLHSGVPSRESGSGSGAY |
| | | FTLTITSLQPEDFATYYCQQSHSTPPTFGPGTKVD |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSP |
| 156 | P1AF5260 LC LTBR | EVQLLESGGALVQPGGSLRLSCAASGFTFSSYAMS |
| | crossed | WVRQAPGKGLEWVSVITGSSSSIDYADSVKGRFTI |
| | | SRDNSKNTLYLQMNSLRAEDTAVYYCAKGGSGVWS |
| | | ISTDFDYWGQGTLVTVSSASVAAPSVFIFPPSDEQ |
| | | LKSGTASVVCLLNNFYPREAKVQWKVDNALQSGNS |
| | | QESVTEQDSKDSTYSLSSTLTLSKADYEKHKVYAC |
| | | EVTHQGLSSPVTKSFNRGEC |
| 157 | P1AF5260 HC hole FAP | EVQLLESGGGLVQPGGSLRLSCAASGFTFSSYAMS |
| | charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSP |
| 158 | P1AF5260 LC FAP | EIVLTQSPGTLSLSPGERATLSCRASQSVTSSYLA |
| | charges | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 159 | P1AF5261 HC knob LTBR | DIQMTQSPSSLSASVGDRVTITCQASQGISNYLSW |
| | crossed | YQQKPGKAPKRLIFAASSLESGVPSRFSGSGSGTD |
| | | FTLTISSLQPEDFATYYCQQYNSYSLTFGGGTKLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSP |
| 160 | P1AF5261 LC LTBR | EVQLLESGGGLVQPGGSLRLSCAASGFTFSYYAMT |
| | crossed | WVRQAPGKGLEWIGVINSGGGTYYTDSVKGRFTMS |
| | | RDNSKNTLYLQVNSLRAEDTAVYYCAKASTADVWG |
| | | QGTTVTVSSASVAAPSVFIFPPSDEQLKSGTASVV |
| | | CLLNNFYPREAKVQWKVDNALQSGNSQESVTEQDS |
| | | KDSTYSLSSTLTLSKADYEKHKVYACEVTHQGLSS |
| | | PVTKSFNRGEC |
| 161 | P1AF5261 HC hole FAP | EVQLLESGGGLVQPGGSLRLSCAASGFTFSSYAMS |
| | charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSP |
| 162 | P1AF5261 LC FAP | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 163 | P1AG7508 HC knob LTBR | DIQMTQSPSSLSASVGDRVTITCQASQGISNYLSW |
| | crossed | YQQKPGKAPKRLIFAASSLESGVPSRFSGSGSGTD |
| | | FTLTISSLQPEDFATYYCQQYNSYSLTFGGGTKLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPG |
| 164 | P1AG7508 LC LTBR | EVQLLESGGGLVQPGGSLRLSCAASGFTFSYYAMT |
| | crossed | WVRQAPGKGLEWIGVINSGGGTYYTDSVKGRFTMS |
| | | RDNSKNTLYLQVNSLRAEDTAVYYCAKASTADVWG |
| | | QGTTVTVSSASVAAPSVFIFPPSDEQLKSGTASVV |
| | | CLLNNFYPREAKVQWKVDNALQSGNSQESVTEQDS |
| | | KDSTYSLSSTLTLSKADYEKHKVYACEVTHQGLSS |
| | | PVTKSFNRGEC |
| 165 | P1AG7508 HC hole FAP | EVQLLESGGGLVQPGGSLRLSCAASGFTFSSYAMS |
| | charges | WVRQAPGKGLEWVSAIIGSGASTYYADSVKGRFTI |
| | | SRDNSKNTLYLQMNSLRAEDTAVYYCAKGWFGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSPG |
| 166 | P1AG7508 LC FAP | EIVLTQSPGTLSLSPGERATLSCRASQSVTSSYLA |
| | charges | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 167 | P1AG5684(2 + 1) HC knob | DIVMTQSPDSLAVSLGERATINCKSSQSVLYSSNN |
| | LTBR-FAP | KNYLAWYQQKPGQPPKLLIYWASTRESGVPDRFSG |
| | | SGSGTDFTLTISSLQAEDVAVYYCSQYYTTHYTFG |
| | | QGTKVEIKSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVD |
| | | KKVEPKSCDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALGAPIEKTISKAKGQPREPQVYTLP |
| | | PCRDELTKNQVSLWCLVKGFYPSDIAVEWESNGQP |
| | | ENNYKTTPPVLDSDGSFFLYSKLTVDKSRWQQGNV |
| | | FSCSVMHEALHNHYTQKSLSLSPGGGGGSGGGGSG |
| | | GGGSGGGGGEVQLLESGGGLVQPGGSLRLSCAASG |
| | | FTFSSYAMSWVRQAPGKGLEWVSAIIGSGASTYYA |
| | | DSVKGRFTISRDNSKNTLYLQMNSLRAEDTAVYYC |
| | | AKGWFGGFNYWGQGTLVTVSSASTKGPSVFPLAPS |
| | | SKSTSGGTAALGCLVEDYFPEPVTVSWNSGALTSG |
| | | VHTFPAVLQSSGLYSLSSVVTVPSSSLGTQTYICN |
| | | VNHKPSNTKVDEKVEPKSCD |
| 168 | P1AG5684(2 + 1) LC LTBR | QVQLVQSGAEVKKPGSSVKVSCKASGGTFSSYAIS |
| | (2x) crossed | WVRQAPGQGLEWMGGIIPIHGTANYAQKFQGRVTI |
| | | TADKSTSTAYMELSSLRSEDTAVYYCARPSYYLDG |
| | | WLDYWGQGTTVTVSSASVAAPSVFIFPPSDEQLKS |
| | | GTASVVCLLNNFYPREAKVQWKVDNALQSGNSQES |
| | | VTEQDSKDSTYSLSSTLTLSKADYEKHKVYACEVT |
| | | HQGLSSPVTKSFNRGEC |
| 169 | P1AG5684(2 + 1) HC hole | DIVMTQSPDSLAVSLGERATINCKSSQSVLYSSNN |
| | LTBR crossed | KNYLAWYQQKPGQPPKLLIYWASTRESGVPDRFSG |
| | | SGSGTDFTLTISSLQAEDVAVYYCSQYYTTHYTFG |
| | | QGTKVEIKSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVD |
| | | KKVEPKSCDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALGAPIEKTISKAKGQPREPQVCTLP |
| | | PSRDELTKNQVSLSCAVKGFYPSDIAVEWESNGQP |
| | | ENNYKTTPPVLDSDGSFFLVSKLTVDKSRWQQGNV |
| | | FSCSVMHEALHNHYTQKSLSLSPG |
| 170 | P1AG5684(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 171 | P1AG7576(2 + 1) HC knob | DIVMTQSPDSLAVSLGERATINCKSSQSVLYSSNN |
| | LTBR-FAP | KNYLAWYQQKPGQPPKLLIYWASTRESGVPDRFSG |
| | | SGSGTDFTLTISSLQAEDVAVYYCSQYYTTHYTFG |
| | | QGTKVEIKSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVD |
| | | KKVEPKSCDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALGAPIEKTISKAKGQPREPQVYTLP |
| | | PCRDELTKNQVSLWCLVKGFYPSDIAVEWESNGQP |
| | | ENNYKTTPPVLDSDGSFFLYSKLTVDKSRWQQGNV |
| | | FSCSVMHEALHNHYTQKSLSLSPGGGGGSGGGGSG |
| | | GGGSGGGGSEVQLLESGGGLVQPGGSLRLSCAASG |
| | | FTFSSYAMSWVRQAPGKGLEWVSAIIGSGASTYYA |
| | | DSVKGRFTISRDNSKNTLYLQMNSLRAEDTAVYYC |
| | | AKGWFGGFNYWGQGTLVTVSSASTKGPSVFPLAPS |
| | | SKSTSGGTAALGCLVEDYFPEPVTVSWNSGALTSG |
| | | VHTFPAVLQSSGLYSLSSVVTVPSSSLGTQTYICN |
| | | VNHKPSNTKVDEKVEPKSCD |
| 172 | P1AG7576(2 + 1) LC LTBR | QVQLVQSGAEVKKPGSSVKVSCKASGGTFSSYAIS |
| | (2x) crossed | WVRQAPGQGLEWMGGIIPIHGTANYAQKFQGRVTI |
| | | TADKSTSTAYMELSSLRSEDTAVYYCARPSYYLDG |
| | | WLDYWGQGTTVTVSSASVAAPSVFIFPPSDEQLKS |
| | | GTASVVCLLNNFYPREAKVQWKVDNALQSGNSQES |
| | | VTEQDSKDSTYSLSSTLTLSKADYEKHKVYACEVT |
| | | HQGLSSPVTKSFNRGEC |
| 173 | P1AG7576(2 + 1) HC hole | DIVMTQSPDSLAVSLGERATINCKSSQSVLYSSNN |
| | LTBR crossed | KNYLAWYQQKPGQPPKLLIYWASTRESGVPDRFSG |
| | | SGSGTDFTLTISSLQAEDVAVYYCSQYYTTHYTFG |
| | | QGTKVEIKSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVD |
| | | KKVEPKSCDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALGAPIEKTISKAKGQPREPQVCTLP |
| | | PSRDELTKNQVSLSCAVKGFYPSDIAVEWESNGQP |
| | | ENNYKTTPPVLDSDGSFFLVSKLTVDKSRWQQGNV |
| | | FSCSVMHEALHNHYTQKSLSLSPG |
| 174 | P1AG7576(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 175 | P1AG7509(2 + 1) HC knob | DIVMTQSPDSLAVSLGERATINCKSSQSVLYSSNN |
| | LTBR-FAP | KNYLAWYQQKPGQPPKLLIYWASTRESGVPDRFSG |
| | | SGSGTDFTLTISSLQAEDVAVYYCSQYYTTHYTFG |
| | | QGTKVEIKSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVD |
| | | KKVEPKSCDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALGAPIEKTISKAKGQPREPQVYTLP |
| | | PCRDELTKNQVSLWCLVKGFYPSDIAVEWESNGQP |
| | | ENNYKTTPPVLDSDGSFFLYSKLTVDKSRWQQGNV |
| | | FSCSVMHEALHNHYTQKSLSLSPGGGGGSGGGGSG |
| | | GGGSGGGGEVQLLESGGGLVQPGGSLRLSCAASGE |
| | | TFSSYAMSWVRQAPGKGLEWVSAIIGSGASTYYAD |
| | | SVKGRFTISRDNSKNTLYLQMNSLRAEDTAVYYCA |
| | | KGWFGGFNYWGQGTLVTVSSASTKGPSVFPLAPSS |
| | | KSTSGGTAALGCLVEDYFPEPVTVSWNSGALTSGV |
| | | HTFPAVLQSSGLYSLSSVVTVPSSSLGTQTYICNV |
| | | NHKPSNTKVDEKVEPKSCD |
| 176 | P1AG7509(2 + 1) LC LTBR | QVQLVQSGAEVKKPGSSVKVSCKASGGTFSSYAIS |
| | (2x) crossed | WVRQAPGQGLEWMGGIIPIHGTANYAQKFQGRVTI |
| | | TADKSTSTAYMELSSLRSEDTAVYYCARPSYYLDG |
| | | WLDYWGQGTTVTVSSASVAAPSVFIFPPSDEQLKS |
| | | GTASVVCLLNNFYPREAKVQWKVDNALQSGNSQES |
| | | VTEQDSKDSTYSLSSTLTLSKADYEKHKVYACEVT |
| | | HQGLSSPVTKSFNRGEC |
| 177 | P1AG7509(2 + 1) HC hole | DIVMTQSPDSLAVSLGERATINCKSSQSVLYSSNN |
| | LTBR crossed | KNYLAWYQQKPGQPPKLLIYWASTRESGVPDRFSG |
| | | SGSGTDFTLTISSLQAEDVAVYYCSQYYTTHYTFG |
| | | QGTKVEIKSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVD |
| | | KKVEPKSCDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALGAPIEKTISKAKGQPREPQVCTLP |
| | | PSRDELTKNQVSLSCAVKGFYPSDIAVEWESNGQP |
| | | ENNYKTTPPVLDSDGSFFLVSKLTVDKSRWQQGNV |
| | | FSCSVMHEALHNHYTQKSLSLSPG |
| 178 | P1AG7509(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 179 | P1AF0544(2+1) HC knob | DIQMTQSPSSLSASVGDRVTITCRASQDISSFLKW |
| | LTBR-FAP | YQQKPGKAPTLLIYAASALQSGVPSRESGSGSGTD |
| | | FTLTISSLQPEDFATYYCQQYNTYPLTFGGGTKVD |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPGGGGGSGGGGSGGGGSGG |
| | | GGSEVQLLESGGGLVQPGGSLRLSCAASGFTESSY |
| | | AMSWVRQAPGKGLEWVSAIIGSGASTYYADSVKGR |
| | | FTISRDNSKNTLYLQMNSLRAEDTAVYYCAKGWFG |
| | | GFNYWGQGTLVTVSSASTKGPSVFPLAPSSKSTSG |
| | | GTAALGCLVEDYFPEPVTVSWNSGALTSGVHTFPA |
| | | VLQSSGLYSLSSVVTVPSSSLGTQTYICNVNHKPS |
| | | NTKVDEKVEPKSCD |
| 180 | P1AF0544(2 + 1) LC LTBR | EVQLLESGGGLVQPGGSLRLSCAASGFTVSSYVMK |
| | (2x) crossed | WFRQAPGKGLEWVSSISGSGGRPFYADSVKGRFTI |
| | | SRDNSKNALYLQMNSLRAEDTAVYYCANDMDVWGQ |
| | | GTTVTVSSASVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC |
| 181 | P1AF0544(2 + 1) HC hole | DIQMTQSPSSLSASVGDRVTITCRASQDISSFLKW |
| | LTBR crossed | YQQKPGKAPTLLIYAASALQSGVPSRFSGSGSGTD |
| | | FTLTISSLQPEDFATYYCQQYNTYPLTFGGGTKVD |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVCTLPPSRDEL |
| | | TKNQVSLSCAVKGFYPSDIAVEWESNGQPENNYKT |
| | | TPPVLDSDGSFFLVSKLTVDKSRWQQGNVFSCSVM |
| | | HEALHNHYTQKSLSLSPGK |
| 182 | P1AF0544(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 183 | P1AG7511(2 + 1) HC knob | DIQMTQSPSSLSASVGDRVTITCRASQDISSFLKW |
| | LTBR-FAP | YQQKPGKAPTLLIYAASALQSGVPSRESGSGSGTD |
| | | FTLTISSLQPEDFATYYCQQYNTYPLTFGGGTKVD |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPGGGGGSGGGGSGGGGSGG |
| | | GGEVQLLESGGGLVQPGGSLRLSCAASGFTESSYA |
| | | MSWVRQAPGKGLEWVSAIIGSGASTYYADSVKGRE |
| | | TISRDNSKNTLYLQMNSLRAEDTAVYYCAKGWEGG |
| | | FNYWGQGTLVTVSSASTKGPSVFPLAPSSKSTSGG |
| | | TAALGCLVEDYFPEPVTVSWNSGALTSGVHTEPAV |
| | | LQSSGLYSLSSVVTVPSSSLGTQTYICNVNHKPSN |
| | | TKVDEKVEPKSCD |
| 184 | P1AG7511(2 + 1) LC LTBR | EVQLLESGGGLVQPGGSLRLSCAASGFTVSSYVMK |
| | (2x) crossed | WFRQAPGKGLEWVSSISGSGGRPFYADSVKGRFTI |
| | | SRDNSKNALYLQMNSLRAEDTAVYYCANDMDVWGQ |
| | | GTTVTVSSASVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC |
| 185 | P1AG7511(2 + 1) HC hole | DIQMTQSPSSLSASVGDRVTITCRASQDISSFLKW |
| | LTBR crossed | YQQKPGKAPTLLIYAASALQSGVPSRESGSGSGTD |
| | | FTLTISSLQPEDFATYYCQQYNTYPLTFGGGTKVD |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVCTLPPSRDEL |
| | | TKNQVSLSCAVKGFYPSDIAVEWESNGQPENNYKT |
| | | TPPVLDSDGSFFLVSKLTVDKSRWQQGNVFSCSVM |
| | | HEALHNHYTQKSLSLSPG |
| 186 | P1AG7511(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 187 | P1AF9729(2 + 1) HC knob | EVQLLESGGGLVQPGGSLRLSCAAAGFTFSSYPMT |
| | LTBR (2x) | WVRQAPGKGLEWVSLISPSGATTYYADSVKGRFTI |
| | | SRDTSKNTLYLQMNSLRAEDTAVYYCAKNHYPDDV |
| | | IIKNYYFYGVDVWGQGTTVTVSSASTKGPSVFPLA |
| | | PSSKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCDKTHTCPPCPAPEA |
| | | AGGPSVFLFPPKPKDTLMISRTPEVTCVVVDVSHE |
| | | DPEVKFNWYVDGVEVHNAKTKPREEQYNSTYRVVS |
| | | VLTVLHQDWLNGKEYKCKVSNKALGAPIEKTISKA |
| | | KGQPREPQVYTLPPCRDELTKNQVSLWCLVKGFYP |
| | | SDIAVEWESNGQPENNYKTTPPVLDSDGSFFLYSK |
| | | LTVDKSRWQQGNVFSCSVMHEALHNHYTQKSLSLS |
| | | PGKGGGGSGGGGSEVQLLESGGGLVQPGGSLRLSC |
| | | AAAGFTESSYPMTWVRQAPGKGLEWVSLISPSGAT |
| | | TYYADSVKGRFTISRDTSKNTLYLQMNSLRAEDTA |
| | | VYYCAKNHYPDDVIIKNYYFYGVDVWGQGTTVTVS |
| | | SASTKGPSVFPLAPSSKSTSGGTAALGCLVKDYFP |
| | | EPVTVSWNSGALTSGVHTFPAVLQSSGLYSLSSVV |
| | | TVPSSSLGTQTYICNVNHKPSNTKVDKKVEPKSC |
| 188 | P1AF9729(2 + 1) LC LTBR | DIQMTQSPSSLSASVGDRVTITCRASQDISNYLNW |
| | (2x) | YQQKPGKAPKSLIYAASSLQSGVPSRFSGSGSGTD |
| | | FTLTISSLQPEDFANYYCQQTHTIPYSFGQGTNLE |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| 189 | P1AF9729(2 + 1) HC hole | EVQLLESGGGLVQPGGSLRLSCAASGFTFSSYAMS |
| | FAP crossed | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASVAAPSVFIFPPSDEQLKSGTA |
| | | SVVCLLNNFYPREAKVQWKVDNALQSGNSQESVTE |
| | | QDSKDSTYSLSSTLTLSKADYEKHKVYACEVTHQG |
| | | LSSPVTKSFNRGECDKTHTCPPCPAPEAAGGPSVF |
| | | LFPPKPKDTLMISRTPEVTCVVVDVSHEDPEVKFN |
| | | WYVDGVEVHNAKTKPREEQYNSTYRVVSVLTVLHQ |
| | | DWLNGKEYKCKVSNKALGAPIEKTISKAKGQPREP |
| | | QVCTLPPSRDELTKNQVSLSCAVKGFYPSDIAVEW |
| | | ESNGQPENNYKTTPPVLDSDGSFFLVSKLTVDKSR |
| | | WQQGNVFSCSVMHEALHNHYTQKSLSLSP |
| 190 | P1AF9729(2 + 1) LC FAP | EIVLTQSPGTLSLSPGERATLSCRASQSVTSSYLA |
| | crossed | WYQQKPGQAPRLLINVGSRRATGIPDRFSGSGSGT |
| | | DETLTISRLEPEDFAVYYCQQGIMLPPTFGQGTKV |
| | | EIKSSASTKGPSVFPLAPSSKSTSGGTAALGCLVK |
| | | DYFPEPVTVSWNSGALTSGVHTFPAVLQSSGLYSL |
| | | SSVVTVPSSSLGTQTYICNVNHKPSNTKVDKKVEP |
| | | KSC |
| 191 | P1AG7510(2 + 1) HC knob | EVQLLESGGGLVQPGGSLRLSCAAAGFTFSSYPMT |
| | LTBR (2x) | WVRQAPGKGLEWVSLISPSGATTYYADSVKGRFTI |
| | | SRDTSKNTLYLQMNSLRAEDTAVYYCAKNHYPDDV |
| | | IIKNYYFYGVDVWGQGTTVTVSSASTKGPSVEPLA |
| | | PSSKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCDKTHTCPPCPAPEA |
| | | AGGPSVFLFPPKPKDTLMISRTPEVTCVVVDVSHE |
| | | DPEVKFNWYVDGVEVHNAKTKPREEQYNSTYRVVS |
| | | VLTVLHQDWLNGKEYKCKVSNKALGAPIEKTISKA |
| | | KGQPREPQVYTLPPCRDELTKNQVSLWCLVKGFYP |
| | | SDIAVEWESNGQPENNYKTTPPVLDSDGSFFLYSK |
| | | LTVDKSRWQQGNVFSCSVMHEALHNHYTQKSLSLS |
| | | PGGGGGSGGGGSGGGGSGGGGEVQLLESGGGLVQP |
| | | GGSLRLSCAAAGFTESSYPMTWVRQAPGKGLEWVS |
| | | LISPSGATTYYADSVKGRFTISRDTSKNTLYLQMN |
| | | SLRAEDTAVYYCAKNHYPDDVIIKNYYFYGVDVWG |
| | | QGTTVTVSSASTKGPSVFPLAPSSKSTSGGTAALG |
| | | CLVKDYFPEPVTVSWNSGALTSGVHTFPAVLQSSG |
| | | LYSLSSVVTVPSSSLGTQTYICNVNHKPSNTKVDK |
| | | KVEPKSCD |
| 192 | P1AG7510(2 + 1) LC LTBR | DIQMTQSPSSLSASVGDRVTITCRASQDISNYLNW |
| | (2x) | YQQKPGKAPKSLIYAASSLQSGVPSRFSGSGSGTD |
| | | FTLTISSLQPEDFANYYCQQTHTIPYSFGQGTNLE |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| 193 | P1AG7510(2 + 1) HC hole | EVQLLESGGGLVQPGGSLRLSCAASGFTFSSYAMS |
| | FAP crossed | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASVAAPSVFIFPPSDEQLKSGTA |
| | | SVVCLLNNFYPREAKVQWKVDNALQSGNSQESVTE |
| | | QDSKDSTYSLSSTLTLSKADYEKHKVYACEVTHQG |
| | | LSSPVTKSFNRGECDKTHTCPPCPAPEAAGGPSVF |
| | | LFPPKPKDTLMISRTPEVTCVVVDVSHEDPEVKFN |
| | | WYVDGVEVHNAKTKPREEQYNSTYRVVSVLTVLHQ |
| | | DWLNGKEYKCKVSNKALGAPIEKTISKAKGQPREP |
| | | QVCTLPPSRDELTKNQVSLSCAVKGFYPSDIAVEW |
| | | ESNGQPENNYKTTPPVLDSDGSFFLVSKLTVDKSR |
| | | WQQGNVFSCSVMHEALHNHYTQKSLSLSPG |
| 194 | P1AG7510(2 + 1) LC FAP | EIVLTQSPGTLSLSPGERATLSCRASQSVTSSYLA |
| | crossed | WYQQKPGQAPRLLINVGSRRATGIPDRFSGSGSGT |
| | | DFTLTISRLEPEDFAVYYCQQGIMLPPTFGQGTKV |
| | | EIKSSASTKGPSVFPLAPSSKSTSGGTAALGCLVK |
| | | DYFPEPVTVSWNSGALTSGVHTFPAVLQSSGLYSL |
| | | SSVVTVPSSSLGTQTYICNVNHKPSNTKVDKKVEP |
| | | KSCD |
| 195 | P1AF0546(2 + 1) HC knob | DIQMTQSPSSLSASVGDRVTITCRASQGISSALAW |
| | LTBR-FAP | FQQKPGKAPKRLIFDASSLDSGVPSRESGSGSGTD |
| | | FTLTISSLQPEDFATYYCQQYNNYPLTFGGGTKVE |
| | | MKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPGGGGGSGGGGSGGGGSGG |
| | | GGSEVQLLESGGGLVQPGGSLRLSCAASGFTESSY |
| | | AMSWVRQAPGKGLEWVSAIIGSGASTYYADSVKGR |
| | | FTISRDNSKNTLYLQMNSLRAEDTAVYYCAKGWFG |
| | | GENYWGQGTLVTVSSASTKGPSVFPLAPSSKSTSG |
| | | GTAALGCLVEDYFPEPVTVSWNSGALTSGVHTFPA |
| | | VLQSSGLYSLSSVVTVPSSSLGTQTYICNVNHKPS |
| | | NTKVDEKVEPKSCD |
| 196 | P1AF0546(2 + 1) LC LTBR | EVQLVESGGGLVQPGGSLRLSCAASGFTFSSYTMN |
| | (2x) crossed | WVRQAPGKGLEWVSGVAPSGDRTYYTDSVKGRFTI |
| | | SRDNAKNTLDLQMTSLRAEDAAIYICAQGFAFTNW |
| | | GQGTMVTVSSASVAAPSVFIFPPSDEQLKSGTASV |
| | | VCLLNNFYPREAKVQWKVDNALQSGNSQESVTEQD |
| | | SKDSTYSLSSTLTLSKADYEKHKVYACEVTHQGLS |
| | | SPVTKSFNRGEC |
| 197 | P1AF0546(2 + 1) HC hole | DIQMTQSPSSLSASVGDRVTITCRASQGISSALAW |
| | LTBR crossed | FQQKPGKAPKRLIFDASSLDSGVPSRFSGSGSGTD |
| | | FTLTISSLQPEDFATYYCQQYNNYPLTFGGGTKVE |
| | | MKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVCTLPPSRDEL |
| | | TKNQVSLSCAVKGFYPSDIAVEWESNGQPENNYKT |
| | | TPPVLDSDGSFFLVSKLTVDKSRWQQGNVFSCSVM |
| | | HEALHNHYTQKSLSLSP |
| 198 | P1AF0546(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 199 | P1AG7512(2 + 1) HC knob | DIQMTQSPSSLSASVGDRVTITCRASQGISSALAW |
| | LTBR-FAP | FQQKPGKAPKRLIFDASSLDSGVPSRFSGSGSGTD |
| | | FTLTISSLQPEDFATYYCQQYNNYPLTFGGGTKVE |
| | | MKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPGGGGGSGGGGSGGGGSGG |
| | | GGEVQLLESGGGLVQPGGSLRLSCAASGFTESSYA |
| | | MSWVRQAPGKGLEWVSAIIGSGASTYYADSVKGRF |
| | | TISRDNSKNTLYLQMNSLRAEDTAVYYCAKGWEGG |
| | | FNYWGQGTLVTVSSASTKGPSVFPLAPSSKSTSGG |
| | | TAALGCLVEDYFPEPVTVSWNSGALTSGVHTFPAV |
| | | LQSSGLYSLSSVVTVPSSSLGTQTYICNVNHKPSN |
| | | TKVDEKVEPKSCD |
| 200 | P1AG7512(2 + 1) LC LTBR | EVQLVESGGGLVQPGGSLRLSCAASGFTFSSYTMN |
| | (2x) crossed | WVRQAPGKGLEWVSGVAPSGDRTYYTDSVKGRFTI |
| | | SRDNAKNTLDLQMTSLRAEDAAIYICAQGFAFTNW |
| | | GQGTMVTVSSASVAAPSVFIFPPSDEQLKSGTASV |
| | | VCLLNNFYPREAKVQWKVDNALQSGNSQESVTEQD |
| | | SKDSTYSLSSTLTLSKADYEKHKVYACEVTHQGLS |
| | | SPVTKSFNRGEC |
| 201 | P1AG7512(2 + 1) HC hole | DIQMTQSPSSLSASVGDRVTITCRASQGISSALAW |
| | LTBR crossed | FQQKPGKAPKRLIFDASSLDSGVPSRFSGSGSGTD |
| | | FTLTISSLQPEDFATYYCQQYNNYPLTFGGGTKVE |
| | | MKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVCTLPPSRDEL |
| | | TKNQVSLSCAVKGFYPSDIAVEWESNGQPENNYKT |
| | | TPPVLDSDGSFFLVSKLTVDKSRWQQGNVFSCSVM |
| | | HEALHNHYTQKSLSLSPG |
| 202 | P1AG7512(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 203 | P1AG7513(2 + 1) HC knob | DIQMTQSPSSLSASVGDRVTITCQASQGISNYLSW |
| | LTBR-FAP | YQQKPGKAPKRLIFAASSLESGVPSRESGSGSGTD |
| | | FTLTISSLQPEDFATYYCQQYNSYSLTFGGGTKLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPGGGGGSGGGGSGGGGSGG |
| | | GGEVQLLESGGGLVQPGGSLRLSCAASGFTESSYA |
| | | MSWVRQAPGKGLEWVSAIIGSGASTYYADSVKGRF |
| | | TISRDNSKNTLYLQMNSLRAEDTAVYYCAKGWFGG |
| | | FNYWGQGTLVTVSSASTKGPSVFPLAPSSKSTSGG |
| | | TAALGCLVEDYFPEPVTVSWNSGALTSGVHTFPAV |
| | | LQSSGLYSLSSVVTVPSSSLGTQTYICNVNHKPSN |
| | | TKVDEKVEPKSCD |
| 204 | P1AG7513(2 + 1) LC LTBR | EVQLLESGGGLVQPGGSLRLSCAASGFTFSYYAMT |
| | (2x) crossed | WVRQAPGKGLEWIGVINSGGGTYYTDSVKGRFTMS |
| | | RDNSKNTLYLQVNSLRAEDTAVYYCAKASTADVWG |
| | | QGTTVTVSSASVAAPSVFIFPPSDEQLKSGTASVV |
| | | CLLNNFYPREAKVQWKVDNALQSGNSQESVTEQDS |
| | | KDSTYSLSSTLTLSKADYEKHKVYACEVTHQGLSS |
| | | PVTKSFNRGEC |
| 205 | P1AG7513(2 + 1) HC hole | DIQMTQSPSSLSASVGDRVTITCQASQGISNYLSW |
| | LTBR crossed | YQQKPGKAPKRLIFAASSLESGVPSRFSGSGSGTD |
| | | FTLTISSLQPEDFATYYCQQYNSYSLTFGGGTKLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVCTLPPSRDEL |
| | | TKNQVSLSCAVKGFYPSDIAVEWESNGQPENNYKT |
| | | TPPVLDSDGSFFLVSKLTVDKSRWQQGNVFSCSVM |
| | | HEALHNHYTQKSLSLSPG |
| 206 | P1AG7513(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 207 | P1AG5683(2 + 1) HC knob | DIQMTQSPSSLSASVGDRVTITCQASQGISNYLSW |
| | LTBR-FAP | YQQKPGKAPKRLIFAASSLESGVPSRFSGSGSGTD |
| | | FTLTISSLQPEDFATYYCQQYNSYSLTFGGGTKLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPGGGGGSGGGGSGGGGSGG |
| | | GGGEVQLLESGGGLVQPGGSLRLSCAASGFTESSY |
| | | AMSWVRQAPGKGLEWVSAIIGSGASTYYADSVKGR |
| | | FTISRDNSKNTLYLQMNSLRAEDTAVYYCAKGWEG |
| | | GFNYWGQGTLVTVSSASTKGPSVFPLAPSSKSTSG |
| | | GTAALGCLVEDYFPEPVTVSWNSGALTSGVHTFPA |
| | | VLQSSGLYSLSSVVTVPSSSLGTQTYICNVNHKPS |
| | | NTKVDEKVEPKSCD |
| 208 | P1AG5683(2 + 1) LC LTBR | EVQLLESGGGLVQPGGSLRLSCAASGFTFSYYAMT |
| | (2x) crossed | WVRQAPGKGLEWIGVINSGGGTYYTDSVKGRFTMS |
| | | RDNSKNTLYLQVNSLRAEDTAVYYCAKASTADVWG |
| | | QGTTVTVSSASVAAPSVFIFPPSDEQLKSGTASVV |
| | | CLLNNFYPREAKVQWKVDNALQSGNSQESVTEQDS |
| | | KDSTYSLSSTLTLSKADYEKHKVYACEVTHQGLSS |
| | | PVTKSFNRGEC |
| 209 | P1AG5683(2 + 1) HC hole | DIQMTQSPSSLSASVGDRVTITCQASQGISNYLSW |
| | LTBR crossed | YQQKPGKAPKRLIFAASSLESGVPSRFSGSGSGTD |
| | | FTLTISSLQPEDFATYYCQQYNSYSLTFGGGTKLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVCTLPPSRDEL |
| | | TKNQVSLSCAVKGFYPSDIAVEWESNGQPENNYKT |
| | | TPPVLDSDGSFFLVSKLTVDKSRWQQGNVFSCSVM |
| | | HEALHNHYTQKSLSLSPG |
| 210 | P1AG5683(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 211 | P1AG7575(2 + 1) HC knob | DIQMTQSPSSLSASVGDRVTITCQASQGISNYLSW |
| | LTBR-FAP | YQQKPGKAPKRLIFAASSLESGVPSRFSGSGSGTD |
| | | FTLTISSLQPEDFATYYCQQYNSYSLTFGGGTKLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPGGGGGSGGGGSGGGGSGG |
| | | GGSEVQLLESGGGLVQPGGSLRLSCAASGFTESSY |
| | | AMSWVRQAPGKGLEWVSAIIGSGASTYYADSVKGR |
| | | FTISRDNSKNTLYLQMNSLRAEDTAVYYCAKGWFG |
| | | GENYWGQGTLVTVSSASTKGPSVFPLAPSSKSTSG |
| | | GTAALGCLVEDYFPEPVTVSWNSGALTSGVHTFPA |
| | | VLQSSGLYSLSSVVTVPSSSLGTQTYICNVNHKPS |
| | | NTKVDEKVEPKSCD |
| 212 | P1AG7575(2 + 1) LC LTBR | EVQLLESGGGLVQPGGSLRLSCAASGFTFSYYAMT |
| | (2x) crossed | WVRQAPGKGLEWIGVINSGGGTYYTDSVKGRFTMS |
| | | RDNSKNTLYLQVNSLRAEDTAVYYCAKASTADVWG |
| | | QGTTVTVSSASVAAPSVFIFPPSDEQLKSGTASVV |
| | | CLLNNFYPREAKVQWKVDNALQSGNSQESVTEQDS |
| | | KDSTYSLSSTLTLSKADYEKHKVYACEVTHQGLSS |
| | | PVTKSFNRGEC |
| 213 | P1AG7575(2+1) HC hole | DIQMTQSPSSLSASVGDRVTITCQASQGISNYLSW |
| | LTBR crossed | YQQKPGKAPKRLIFAASSLESGVPSRFSGSGSGTD |
| | | FTLTISSLQPEDFATYYCQQYNSYSLTFGGGTKLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVCTLPPSRDEL |
| | | TKNQVSLSCAVKGFYPSDIAVEWESNGQPENNYKT |
| | | TPPVLDSDGSFFLVSKLTVDKSRWQQGNVFSCSVM |
| | | HEALHNHYTQKSLSLSPG |
| 214 | P1AG7575(2 + 1) LC FAP | EIVLTQSPGTLSLSPGERATLSCRASQSVTSSYLA |
| | charges | WYQQKPGQAPRLLINVGSRRATGIPDRFSGSGSGT |
| | | DFTLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 215 | P1AG1326 HC knob LTBR | DIQMTQSPSSLSASVGDRVTITCKAGQDIKSYLSW |
| | crossed | YQQKPGKAPKLLIYYATRLADGVPSRFSGSGSGTD |
| | | YTLTISSLQPEDFATYYCLQHGESPWTFGGGTKLE |
| | | IKSSASTKGPSVFPLAPSSKSTSGGTAALGCLVKD |
| | | YFPEPVTVSWNSGALTSGVHTFPAVLQSSGLYSLS |
| | | SVVTVPSSSLGTQTYICNVNHKPSNTKVDKKVEPK |
| | | SCDKTHTCPPCPAPEAAGGPSVFLFPPKPKDTLMI |
| | | SRTPEVTCVVVDVSHEDPEVKFNWYVDGVEVHNAK |
| | | TKPREEQYNSTYRVVSVLTVLHQDWLNGKEYKCKV |
| | | SNKALGAPIEKTISKAKGQPREPQVYTLPPCRDEL |
| | | TKNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSP |
| 216 | P1AG1326 LC LTBR | EVQLVESGGGLVKPGGSLRLSCAASGFTFSDYYMY |
| | crossed | WFRQAPGKGLEWVATISDGGSYTYYPDSVKGRFTI |
| | | SRDNAKNSLYLQMSSLRAEDTAVYYCAREENGNFY |
| | | YFDYWGQGTTVTVSSASVAAPSVFIFPPSDEQLKS |
| | | GTASVVCLLNNFYPREAKVQWKVDNALQSGNSQES |
| | | VTEQDSKDSTYSLSSTLTLSKADYEKHKVYACEVT |
| | | HQGLSSPVTKSFNRGEC |
| 217 | P1AG1326 HC hole FAP | EVQLLESGGGLVQPGGSLRLSCAASGFTFSSYAMS |
| | charges | WVRQAPGKGLEWVSAIIGSGASTYYADSVKGRFTI |
| | | SRDNSKNTLYLQMNSLRAEDTAVYYCAKGWEGGFN |
| | | YWGQGTLVTVSSASTKGPSVFPLAPSSKSTSGGTA |
| | | ALGCLVEDYFPEPVTVSWNSGALTSGVHTFPAVLQ |
| | | SSGLYSLSSVVTVPSSSLGTQTYICNVNHKPSNTK |
| | | VDEKVEPKSCDKTHTCPPCPAPEAAGGPSVFLFPP |
| | | KPKDTLMISRTPEVTCVVVDVSHEDPEVKFNWYVD |
| | | GVEVHNAKTKPREEQYNSTYRVVSVLTVLHQDWLN |
| | | GKEYKCKVSNKALGAPIEKTISKAKGQPREPQVCT |
| | | LPPSRDELTKNQVSLSCAVKGFYPSDIAVEWESNG |
| | | QPENNYKTTPPVLDSDGSFFLVSKLTVDKSRWQQG |
| | | NVFSCSVMHEALHNHYTQKSLSLSP |
| 218 | P1AG1326 LC FAP charges | EIVLTQSPGTLSLSPGERATLSCRASQSVTSSYLA |
| | | WYQQKPGQAPRLLINVGSRRATGIPDRFSGSGSGT |
| | | DETLTISRLEPEDFAVYYCQQGIMLPPTFGQGTKV |
| | | EIKRTVAAPSVFIFPPSDRKLKSGTASVVCLLNNF |
| | | YPREAKVQWKVDNALQSGNSQESVTEQDSKDSTYS |
| | | LSSTLTLSKADYEKHKVYACEVTHQGLSSPVTKSF |
| | | NRGEC |
| 219 | P1AF4664 HC KK+ LTBR | EVQLLESGGGLVQPGGSLRLSCAASGFTFSYYAMT |
| | charges (murine 1 + 1 | WVRQAPGKGLEWIGVINSGGGTYYTDSVKGRFTMS |
| | surrogate) | RDNSKNTLYLQVNSLRAEDTAVYYCAKASTADVWG |
| | | QGTTVTVSSAKTTPPSVYPLAPGSAAQTNSMVTLG |
| | | CLVEGYFPEPVTVTWNSGSLSSGVHTFPAVLQSDL |
| | | YTLSSSVTVPSSTWPSQTVTCNVAHPASSTKVDEK |
| | | IVPRDCGCKPCICTVPEVSSVFIFPPKPKDVLTIT |
| | | LTPKVTCVVVAISKDDPEVQFSWFVDDVEVHTAQT |
| | | KPREEQINSTFRSVSELPIMHQDWLNGKEFKCRVN |
| | | SAAFGAPIEKTISKTKGRPKAPQVYTIPPPKKQMA |
| | | KDKVSLTCMITNFFPEDITVEWQWNGQPAENYKNT |
| | | QPIMKTDGSYFVYSKLNVQKSNWEAGNTFTCSVLH |
| | | EGLHNHHTEKSLSHSPG |
| 220 | P1AF4664 LC LTBR | DIQMTQSPSSLSASVGDRVTITCQASQGISNYLSW |
| | charges (murine 1 + 1 | YQQKPGKAPKRLIFAASSLESGVPSRESGSGSGTD |
| | surrogate) | FTLTISSLQPEDFATYYCQQYNSYSLTFGGGTKLE |
| | | IKRADAAPTVSIFPPSSRKLTSGGASVVCELNNFY |
| | | PKDINVKWKIDGSERQNGVLNSWTDQDSKDSTYSM |
| | | SSTLTLTKDEYERHNSYTCEATHKTSTSPIVKSEN |
| | | RNEC |
| 221 | P1AF4664 HC DD− FAP | EIVLTQSPGTLSLSPGERATLSCRASQSVSRSYLA |
| | crossed (murine 1 + 1 | WYQQKPGQAPRLLIIGASTRATGIPDRFSGSGSGT |
| | surrogate) | DFTLTISRLEPEDFAVYYCQQGQVIPPTFGQGTKV |
| | | EIKSSAKTTPPSVYPLAPGSAAQTNSMVTLGCLVK |
| | | GYFPEPVTVTWNSGSLSSGVHTFPAVLQSDLYTLS |
| | | SSVTVPSSTWPSQTVTCNVAHPASSTKVDKKIVPR |
| | | DCGCKPCICTVPEVSSVFIFPPKPKDVLTITLTPK |
| | | VTCVVVAISKDDPEVQFSWFVDDVEVHTAQTKPRE |
| | | EQINSTFRSVSELPIMHQDWLNGKEFKCRVNSAAF |
| | | GAPIEKTISKTKGRPKAPQVYTIPPPKEQMAKDKV |
| | | SLTCMITNFFPEDITVEWQWNGQPAENYDNTQPIM |
| | | DTDGSYFVYSDLNVQKSNWEAGNTFTCSVLHEGLH |
| | | NHHTEKSLSHSPG |
| 222 | P1AF4664 LC FAP crossed | EVQLLESGGGLVQPGGSLRLSCAASGFTFSSHAMS |
| | (murine 1 + 1 surrogate) | WVRQAPGKGLEWVSAIWASGEQYYADSVKGRFTIS |
| | | RDNSKNTLYLQMNSLRAEDTAVYYCAKGWLGNEDY |
| | | WGQGTLVTVSSRADAAPTVSIFPPSSEQLTSGGAS |
| | | VVCFLNNFYPKDINVKWKIDGSERQNGVLNSWTDQ |
| | | DSKDSTYSMSSTLTLTKDEYERHNSYTCEATHKTS |
| | | TSPIVKSFNRNEC |
| 223 | P1AF4674 HC KK+ LTBR | EVQLLESGGGLVQPGGSLRLSCAASGFTFSYYAMT |
| | charges-FAP crossed | WVRQAPGKGLEWIGVINSGGGTYYTDSVKGRFTMS |
| | (murine 2 + 1 surrogate) | RDNSKNTLYLQVNSLRAEDTAVYYCAKASTADVWG |
| | | QGTTVTVSSAKTTPPSVYPLAPGSAAQTNSMVTLG |
| | | CLVEGYFPEPVTVTWNSGSLSSGVHTFPAVLQSDL |
| | | YTLSSSVTVPSSTWPSQTVTCNVAHPASSTKVDEK |
| | | IVPRDCGCKPCICTVPEVSSVFIFPPKPKDVLTIT |
| | | LTPKVTCVVVAISKDDPEVQFSWFVDDVEVHTAQT |
| | | KPREEQINSTFRSVSELPIMHQDWLNGKEFKCRVN |
| | | SAAFGAPIEKTISKTKGRPKAPQVYTIPPPKKQMA |
| | | KDKVSLTCMITNFFPEDITVEWQWNGQPAENYKNT |
| | | QPIMKTDGSYFVYSKLNVQKSNWEAGNTFTCSVLH |
| | | EGLHNHHTEKSLSHSPGGGGGSGGGGSGGGGSGGG |
| | | GSEVQLLESGGGLVQPGGSLRLSCAASGFTESSHA |
| | | MSWVRQAPGKGLEWVSAIWASGEQYYADSVKGRFT |
| | | ISRDNSKNTLYLQMNSLRAEDTAVYYCAKGWLGNE |
| | | DYWGQGTLVTVSSRADAAPTVSIFPPSSEQLTSGG |
| | | ASVVCFLNNFYPKDINVKWKIDGSERQNGVLNSWT |
| | | DQDSKDSTYSMSSTLTLTKDEYERHNSYTCEATHK |
| | | TSTSPIVKSFNRNEC |
| 224 | P1AF4674 LC LTBR | DIQMTQSPSSLSASVGDRVTITCQASQGISNYLSW |
| | charges (murine 2 + 1 | YQQKPGKAPKRLIFAASSLESGVPSRFSGSGSGTD |
| | surrogate) | FTLTISSLQPEDFATYYCQQYNSYSLTFGGGTKLE |
| | | IKRADAAPTVSIFPPSSRKLTSGGASVVCFLNNFY |
| | | PKDINVKWKIDGSERQNGVLNSWTDQDSKDSTYSM |
| | | SSTLTLTKDEYERHNSYTCEATHKTSTSPIVKSFN |
| | | RNEC |
| 225 | P1AF4674 HC DD− LTBR | EVQLLESGGGLVQPGGSLRLSCAASGFTFSYYAMT |
| | charges (murine 2 + 1 | WVRQAPGKGLEWIGVINSGGGTYYTDSVKGRFTMS |
| | surrogate) | RDNSKNTLYLQVNSLRAEDTAVYYCAKASTADVWG |
| | | QGTTVTVSSAKTTPPSVYPLAPGSAAQTNSMVTLG |
| | | CLVEGYFPEPVTVTWNSGSLSSGVHTFPAVLQSDL |
| | | YTLSSSVTVPSSTWPSQTVTCNVAHPASSTKVDEK |
| | | IVPRDCGCKPCICTVPEVSSVFIFPPKPKDVLTIT |
| | | LTPKVTCVVVAISKDDPEVQFSWFVDDVEVHTAQT |
| | | KPREEQINSTFRSVSELPIMHQDWLNGKEFKCRVN |
| | | SAAFGAPIEKTISKTKGRPKAPQVYTIPPPKEQMA |
| | | KDKVSLTCMITNFFPEDITVEWQWNGQPAENYDNT |
| | | QPIMDTDGSYFVYSDLNVQKSNWEAGNTFTCSVLH |
| | | EGLHNHHTEKSLSHSPG |
| 226 | P1AF4674 LC FAP crossed | EIVLTQSPGTLSLSPGERATLSCRASQSVSRSYLA |
| | (murine 2 + 1 surrogate) | WYQQKPGQAPRLLIIGASTRATGIPDRFSGSGSGT |
| | | DFTLTISRLEPEDFAVYYCQQGQVIPPTFGQGTKV |
| | | EIKSSAKTTPPSVYPLAPGSAAQTNSMVTLGCLVK |
| | | GYFPEPVTVTWNSGSLSSGVHTFPAVLQSDLYTLS |
| | | SSVTVPSSTWPSQTVTCNVAHPASSTKVDKKIVPR |
| | | DC |
| 227 | P1AE5929 HC (hu IgG1 PG | QVQLVQSGAEVKKPGSSVKVSCKASGGTFSSYAIS |
| | LALA) | WVRQAPGQGLEWMGGIIPIFGTANYAQKFQGRVTI |
| | | TADKSTSTAYMELSSLRSEDTAVYYCARPSYYLDG |
| | | WLDYWGQGTTVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPEAAGGPSVFL |
| | | FPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALGAPIEKTISKAKGQPREPQ |
| | | VYTLPPSRDELTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSP |
| 228 | P1AE5929 LC (hu IgG1 PG | DIVMTQSPDSLAVSLGERATINCKSSQSVLYSSNN |
| | LALA) | KNYLAWYQQKPGQPPKLLIYWASTRESGVPDRFSG |
| | | SGSGTDFTLTISSLQAEDVAVYYCSQYYTTHYTFG |
| | | QGTKVEIKRTVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC |
| 229 | P1AF7213 HC (hu IgG1 PG | QVQLVQSGAEVKKPGSSVKVSCKASGGTFSSYAIS |
| | LALA) | WVRQAPGQGLEWMGGIIPIHGTANYAQKFQGRVTI |
| | | TADKSTSTAYMELSSLRSEDTAVYYCARPSYYLDG |
| | | WLDYWGQGTTVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPEAAGGPSVFL |
| | | FPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALGAPIEKTISKAKGQPREPQ |
| | | VYTLPPSRDELTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSP |
| 230 | P1AF7213 LC (hu IgG1 PG | DIVMTQSPDSLAVSLGERATINCKSSQSVLYSSNN |
| | LALA) | KNYLAWYQQKPGQPPKLLIYWASTRESGVPDRFSG |
| | | SGSGTDFTLTISSLQAEDVAVYYCSQYYTTHYTFG |
| | | QGTKVEIKRTVAAPSVFIFPPSDEQLKSGTASVVC |
| | | LLNNFYPREAKVQWKVDNALQSGNSQESVTEQDSK |
| | | DSTYSLSSTLTLSKADYEKHKVYACEVTHQGLSSP |
| | | VTKSFNRGEC |
| 231 | P1AE9452 HC (hu IgG1 PG | EVQLLESGGGLVQPGGSLRLSCAAAGFTFSSYPMT |
| | LALA) | WVRQAPGKGLEWVSLISPSGATTYYADSVKGRFTI |
| | | SRDTSKNTLYLQMNSLRAEDTAVYYCAKNHYPDDV |
| | | IIKNYYFYGVDVWGQGTTVTVSSASTKGPSVEPLA |
| | | PSSKSTSGGTAALGCLVKDYFPEPVTVSWNSGALT |
| | | SGVHTFPAVLQSSGLYSLSSVVTVPSSSLGTQTYI |
| | | CNVNHKPSNTKVDKKVEPKSCDKTHTCPPCPAPEA |
| | | AGGPSVFLFPPKPKDTLMISRTPEVTCVVVDVSHE |
| | | DPEVKFNWYVDGVEVHNAKTKPREEQYNSTYRVVS |
| | | VLTVLHQDWLNGKEYKCKVSNKALGAPIEKTISKA |
| | | KGQPREPQVYTLPPSRDELTKNQVSLTCLVKGFYP |
| | | SDIAVEWESNGQPENNYKTTPPVLDSDGSFFLYSK |
| | | LTVDKSRWQQGNVFSCSVMHEALHNHYTQKSLSLS |
| | | P |
| 232 | P1AE9452 LC (hu IgG1 PG | DIQMTQSPSSLSASVGDRVTITCRASQDISNYLNW |
| | LALA) | YQQKPGKAPKSLIYAASSLQSGVPSRFSGSGSGTD |
| | | FTLTISSLQPEDFANYYCQQTHTIPYSFGQGTNLE |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSEN |
| | | RGEC |
| 233 | P1AE9450 HC (hu IgG1 PG | EVQLLESGGGLVQPGGSLRLSCAASGFTVSSYVMK |
| | LALA) | WFRQAPGKGLEWVSSISGSGGRPFYADSVKGRFTI |
| | | SRDNSKNALYLQMNSLRAEDTAVYYCANDMDVWGQ |
| | | GTTVTVSSASTKGPSVFPLAPSSKSTSGGTAALGC |
| | | LVKDYFPEPVTVSWNSGALTSGVHTFPAVLQSSGL |
| | | YSLSSVVTVPSSSLGTQTYICNVNHKPSNTKVDKK |
| | | VEPKSCDKTHTCPPCPAPEAAGGPSVFLFPPKPKD |
| | | TLMISRTPEVTCVVVDVSHEDPEVKFNWYVDGVEV |
| | | HNAKTKPREEQYNSTYRVVSVLTVLHQDWLNGKEY |
| | | KCKVSNKALGAPIEKTISKAKGQPREPQVYTLPPS |
| | | RDELTKNQVSLTCLVKGFYPSDIAVEWESNGQPEN |
| | | NYKTTPPVLDSDGSFFLYSKLTVDKSRWQQGNVES |
| | | CSVMHEALHNHYTQKSLSLSP |
| 234 | P1AE9450 LC (hu IgG1 PG | DIQMTQSPSSLSASVGDRVTITCRASQDISSELKW |
| | LALA) | YQQKPGKAPTLLIYAASALQSGVPSRFSGSGSGTD |
| | | FTLTISSLQPEDFATYYCQQYNTYPLTFGGGTKVD |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| 235 | P1AE9459 HC (hu IgG1 PG | EVQLVESGGGLVQPGGSLRLSCAASGFTFSSYTMN |
| | LALA) | WVRQAPGKGLEWVSGVAPSGDRTYYTDSVKGRFTI |
| | | SRDNAKNTLDLQMTSLRAEDAAIYICAQGFAFTNW |
| | | GQGTMVTVSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVD |
| | | KKVEPKSCDKTHTCPPCPAPEAAGGPSVFLFPPKP |
| | | KDTLMISRTPEVTCVVVDVSHEDPEVKFNWYVDGV |
| | | EVHNAKTKPREEQYNSTYRVVSVLTVLHQDWLNGK |
| | | EYKCKVSNKALGAPIEKTISKAKGQPREPQVYTLP |
| | | PSRDELTKNQVSLTCLVKGFYPSDIAVEWESNGQP |
| | | ENNYKTTPPVLDSDGSFFLYSKLTVDKSRWQQGNV |
| | | FSCSVMHEALHNHYTQKSLSLSP |
| 236 | P1AE9459 LC (hu IgG1 PG | DIQMTQSPSSLSASVGDRVTITCRASQGISSALAW |
| | LALA) | FQQKPGKAPKRLIFDASSLDSGVPSRFSGSGSGTD |
| | | FTLTISSLQPEDFATYYCQQYNNYPLTFGGGTKVE |
| | | MKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| 237 | P1AF0080 HC (hu IgG1 PG | EVQLLESGGGLVQPGGSLRLSCAASGFTFSYYAMT |
| | LALA) | WVRQAPGKGLEWIGVINSGGGTYYTDSVKGRFTMS |
| | | RDNSKNTLYLQVNSLRAEDTAVYYCAKASTADVWG |
| | | QGTTVTVSSASTKGPSVFPLAPSSKSTSGGTAALG |
| | | CLVKDYFPEPVTVSWNSGALTSGVHTFPAVLQSSG |
| | | LYSLSSVVTVPSSSLGTQTYICNVNHKPSNTKVDK |
| | | KVEPKSCDKTHTCPPCPAPEAAGGPSVFLFPPKPK |
| | | DTLMISRTPEVTCVVVDVSHEDPEVKFNWYVDGVE |
| | | VHNAKTKPREEQYNSTYRVVSVLTVLHQDWLNGKE |
| | | YKCKVSNKALGAPIEKTISKAKGQPREPQVYTLPP |
| | | SRDELTKNQVSLTCLVKGFYPSDIAVEWESNGQPE |
| | | NNYKTTPPVLDSDGSFFLYSKLTVDKSRWQQGNVE |
| | | SCSVMHEALHNHYTQKSLSLSP |
| 238 | P1AF0080 LC (hu IgG1 PG | DIQMTQSPSSLSASVGDRVTITCQASQGISNYLSW |
| | LALA) | YQQKPGKAPKRLIFAASSLESGVPSRFSGSGSGTD |
| | | FTLTISSLQPEDFATYYCQQYNSYSLTFGGGTKLE |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| 239 | P1AF0064 HC (hu IgG1 PG | EVQLQQYGAELGKPGTSVKLSCKVSGYNIGNTYIH |
| | LALA) | WVIQRPGKGLEWIGRIDPTNGNTICTEKFKNKATL |
| | | TADLSSNTAYMQLSQLKSDDTAIYFCAPTTLTTPD |
| | | SFAYWGQGTLVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPEAAGGPSVFL |
| | | FPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALGAPIEKTISKAKGQPREPQ |
| | | VYTLPPSRDELTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSP |
| 240 | P1AF0064 LC (hu IgG1 PG | DIQMTQSPSQLSASVGDRVTLSCKASQNIYKYLNW |
| | LALA) | YQQKLGEAPRLLIYSTNILQTGIPSRFSGSGSGTD |
| | | YTLTISSLQPEDAATYFCQKYYSGFTFGSGTKLEM |
| | | KRTVAAPSVFIFPPSDEQLKSGTASVVCLLNNFYP |
| | | REAKVQWKVDNALQSGNSQESVTEQDSKDSTYSLS |
| | | STLTLSKADYEKHKVYACEVTHQGLSSPVTKSFNR |
| | | GEC |
| 241 | P1AF0079 HC (hu IgG1 PG | EVQLLESGGALVQPGGSLRLSCAASGFTFSSYAMS |
| | LALA) | WVRQAPGKGLEWVSVITGSSSSIDYADSVKGRFTI |
| | | SRDNSKNTLYLQMNSLRAEDTAVYYCAKGGSGVWS |
| | | ISTDEDYWGQGTLVTVSSASTKGPSVFPLAPSSKS |
| | | TSGGTAALGCLVKDYFPEPVTVSWNSGALTSGVHT |
| | | FPAVLQSSGLYSLSSVVTVPSSSLGTQTYICNVNH |
| | | KPSNTKVDKKVEPKSCDKTHTCPPCPAPEAAGGPS |
| | | VFLFPPKPKDTLMISRTPEVTCVVVDVSHEDPEVK |
| | | FNWYVDGVEVHNAKTKPREEQYNSTYRVVSVLTVL |
| | | HQDWLNGKEYKCKVSNKALGAPIEKTISKAKGQPR |
| | | EPQVYTLPPSRDELTKNQVSLTCLVKGFYPSDIAV |
| | | EWESNGQPENNYKTTPPVLDSDGSFFLYSKLTVDK |
| | | SRWQQGNVFSCSVMHEALHNHYTQKSLSLSP |
| 242 | P1AF0079 LC (hu IgG1 PG | DIQMTQSPSFLSASVGDRVTITCQASQDISNYLNW |
| | LALA) | YQQKPGKAPKHLIYAASSLHSGVPSRFSGSGSGAY |
| | | FTLTITSLQPEDFATYYCQQSHSTPPTFGPGTKVD |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| 243 | P1AE9457 HC (hu IgG1 PG | EVQLLESGGGLVQPGGSLRLSCAASGFTESNYAMT |
| | LALA) | WVRQAPGKGLEWVSEISGSGGSTYYADSVKGRFTI |
| | | SRDNSKNTLYLQMNSLRAEDTAVYYCAKDDDGAYG |
| | | DTMDVWGQGTTVTVSSASTKGPSVFPLAPSSKSTS |
| | | GGTAALGCLVKDYFPEPVTVSWNSGALTSGVHTEP |
| | | AVLQSSGLYSLSSVVTVPSSSLGTQTYICNVNHKP |
| | | SNTKVDKKVEPKSCDKTHTCPPCPAPEAAGGPSVF |
| | | LFPPKPKDTLMISRTPEVTCVVVDVSHEDPEVKFN |
| | | WYVDGVEVHNAKTKPREEQYNSTYRVVSVLTVLHQ |
| | | DWLNGKEYKCKVSNKALGAPIEKTISKAKGQPREP |
| | | QVYTLPPSRDELTKNQVSLTCLVKGFYPSDIAVEW |
| | | ESNGQPENNYKTTPPVLDSDGSFFLYSKLTVDKSR |
| | | WQQGNVFSCSVMHEALHNHYTQKSLSLSP |
| 244 | P1AE9457 LC (hu IgG1 PG | DIQMTQSPSSLSASIGDRVTITCRASQDISNYLNW |
| | LALA) | YQQKPGKAPKLLIYAASALQSGVPSRFSGSGSGTD |
| | | FTLTISSLQPEDFATYYCQHYKSYPYTFGQGTKVE |
| | | IRRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| 245 | P1AE1873 (CBE11) HC (hu | EVQLVESGGGLVKPGGSLRLSCAASGFTFSDYYMY |
| | IgG1 PG LALA) | WFRQAPGKGLEWVATISDGGSYTYYPDSVKGRFTI |
| | | SRDNAKNSLYLQMSSLRAEDTAVYYCAREENGNFY |
| | | YFDYWGQGTTVTVSSASTKGPSVFPLAPSSKSTSG |
| | | GTAALGCLVKDYFPEPVTVSWNSGALTSGVHTFPA |
| | | VLQSSGLYSLSSVVTVPSSSLGTQTYICNVNHKPS |
| | | NTKVDKKVEPKSCDKTHTCPPCPAPEAAGGPSVFL |
| | | FPPKPKDTLMISRTPEVTCVVVDVSHEDPEVKFNW |
| | | YVDGVEVHNAKTKPREEQYNSTYRVVSVLTVLHQD |
| | | WLNGKEYKCKVSNKALGAPIEKTISKAKGQPREPQ |
| | | VYTLPPSRDELTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSP |
| 246 | P1AE1873 (CBE11) LC (hu | DIQMTQSPSSLSASVGDRVTITCKAGQDIKSYLSW |
| | IgG1 PG LALA) | YQQKPGKAPKLLIYYATRLADGVPSRESGSGSGTD |
| | | YTLTISSLQPEDFATYYCLQHGESPWTFGGGTKLE |
| | | IKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFN |
| | | RGEC |
| 247 | Fc-depleter kh NC avi B | GLNDIFEAQKIEWHEDKTHTCPPCPAPELLGGPSV |
| | (P1AA0981) (knob chain) | FLFPPKPKDTLMISRTPEVTCVVVDVSHEDPEVKE |
| | | NWYVDGVEVHNAKTKPREEQYNSTYRVVSVLTVLH |
| | | QDWLNGKEYKCKVSNKALPAPIEKTISKAKGQPRE |
| | | PQVYTLPPCRDELTKNQVSLWCLVKGFYPSDIAVE |
| | | WESNGQPENNYKTTPPVLDSDGSFFLYSKLTVDKS |
| | | RWQQGNVFSCSVMHEALHNHYTQKSLSLSPGK |
| 248 | Fc-depleter kh NC avi B | DKTHTCPPCPAPELLGGPSVFLFPPKPKDTLMISR |
| | P1AA0981) (hole chain) | TPEVTCVVVDVSHEDPEVKFNWYVDGVEVHNAKTK |
| | | PREEQYNSTYRVVSVLTVLHQDWLNGKEYKCKVSN |
| | | KALPAPIEKTISKAKGQPREPQVCTLPPSRDELTK |
| | | NQVSLSCAVKGFYPSDIAVEWESNGQPENNYKTTP |
| | | PVLDSDGSFFLVSKLTVDKSRWQQGNVFSCSVMHE |
| | | ALHNHYTQKSLSLSPGKSGGLNDIFEAQKIEWHE |
| 249 | human lymphotoxin α1β2 | KPAAHLIGDPSKQNSLLWRANTDRAFLQDGFSLSN |
| | single-chain avi his | NSLLVPTSGIYFVYSQVVFSGKAYSPKATSSPLYL |
| | (P1AE1235) | AHEVQLESSQYPFHVPLLSSQKMVYPGLQEPWLHS |
| | | MYHGAAFQLTQGDQLSTHTDGIPHLVLSPSTVFFG |
| | | AFALGGSGLPAAHLIGAPLKGQGLGWETTKEQAFL |
| | | TSGTQFSDAEGLALPQDGLYYLYCLVGYRGRAPPG |
| | | GGDPQGRSVTLRSSLYRAGGAYGPGTPELLLEGAE |
| | | TVTPVLDPARRQGYGPLWYTSVGFGGLVQLRRGER |
| | | VYVNISHPDMVDFARGKTFFGAVMVGGSGLPAAHL |
| | | IGAPLKGQGLGWETTKEQAFLTSGTQFSDAEGLAL |
| | | PQDGLYYLYCLVGYRGRAPPGGGDPQGRSVTLRSS |
| | | LYRAGGAYGPGTPELLLEGAETVTPVLDPARRQGY |
| | | GPLWYTSVGFGGLVQLRRGERVYVNISHPDMVDFA |
| | | RGKTFFGAVMVGGSGLNDIFEAQKIEWHEGGHHHH |
| | | HH |
| 250 | murine lymphotoxin α1β2 | KPAAHLVGYPSKQNSLLWRASTDRAFLRHGFSLSN |
| | single-chain avi his | NSLLIPTSGLYFVYSQVVESGESCSPRAIPTPIYL |
| | (P1AE1236) | AHEVQLESSQYPFHVPLLSAQKSVYPGLQGPWVRS |
| | | MYQGAVFLLSKGDQLSTHTDGISHLHESPSSVFFG |
| | | AFALGGSGLPAAHLIGAWMSGQGLSWEASQEEAFL |
| | | RSGAQFSPTHGLALPQDGVYYLYCHVGYRGRTPPA |
| | | GRSRARSLTLRSALYRAGGAYGRGSPELLLEGAET |
| | | VTPVVDPIGYGSLWYTSVGFGGLAQLRSGERVYVN |
| | | ISHPDMVDYRRGKTFFGAVMVGGSGLPAAHLIGAW |
| | | MSGQGLSWEASQEEAFLRSGAQFSPTHGLALPQDG |
| | | VYYLYCHVGYRGRTPPAGRSRARSLTLRSALYRAG |
| | | GAYGRGSPELLLEGAETVTPVVDPIGYGSLWYTSV |
| | | GFGGLAQLRSGERVYVNISHPDMVDYRRGKTFFGA |
| | | VMVGGSGLNDIFEAQKIEWHEGGHHHHHH |
| 251 | monomeric huLTBR ECD | SQPQAVPPYASENQTCRDQEKEYYEPQHRICCSRC |
| | (S28-M227) Fc-fusion avi | PPGTYVSAKCSRIRDTVCATCAENSYNEHWNYLTI |
| | biotinylated (P1AE2835) | CQLCRPCDPVMGLEEIAPCTSKRKTQCRCQPGMFC |
| | (knob chain) | AAWALECTHCELLSDCPPGTEAELKDEVGKGNNHC |
| | | VPCKAGHFQNTSSPSARCQPHTRCENQGLVEAAPG |
| | | TAQSDTTCKNPLEPLPPEMSGTMLMASGSDKTHTC |
| | | PPCPAPELLGGPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALPAP |
| | | IEKTISKAKGQPREPQVYTLPPCRDELTKNQVSLW |
| | | CLVKGFYPSDIAVEWESNGQPENNYKTTPPVLDSD |
| | | GSFFLYSKLTVDKSRWQQGNVFSCSVMHEALHNHY |
| | | TQKSLSLSPGKSGGLNDIFEAQKIEWHE |
| 252 | monomeric huLTBR ECD | DKTHTCPPCPAPELLGGPSVFLFPPKPKDTLMISR |
| | (S28-M227) Fc-fusion avi | TPEVTCVVVDVSHEDPEVKFNWYVDGVEVHNAKTK |
| | biotinylated (P1AE2835) | PREEQYNSTYRVVSVLTVLHQDWLNGKEYKCKVSN |
| | (hole chain) | KALPAPIEKTISKAKGQPREPQVCTLPPSRDELTK |
| | | NQVSLSCAVKGFYPSDIAVEWESNGQPENNYKTTP |
| | | PVLDSDGSFFLVSKLTVDKSRWQQGNVESCSVMHE |
| | | ALHNRFTQKSLSLSPGK |
| 253 | monomeric muLTBR ECD | SQPQLVPPYRIENQTCWDQDKEYYEPMHDVCCSRC |
| | (S28-L223) Fc-fusion avi | PPGEFVFAVCSRSQDTVCKTCPHNSYNEHWNHLST |
| | biotinylated (P1AE4410) | CQLCRPCDIVLGFEEVAPCTSDRKAECRCQPGMSC |
| | (knob chain) | VYLDNECVHCEEERLVLCQPGTEAEVTDEIMDTDV |
| | | NCVPCKPGHFQNTSSPRARCQPHTRCEIQGLVEAA |
| | | PGTSYSDTICKNPPEPGAMLLASGSDKTHTCPPCP |
| | | APELLGGPSVFLFPPKPKDTLMISRTPEVTCVVVD |
| | | VSHEDPEVKFNWYVDGVEVHNAKTKPREEQYNSTY |
| | | RVVSVLTVLHQDWLNGKEYK |
| | | CKVSNKALPAPIEKTISKAKGQPREPQVYTLPPCR |
| | | DELTKNQVSLWCLVKGFYPSDIAVEWESNGQPENN |
| | | YKTTPPVLDSDGSFFLYSKLTVDKSRWQQGNVFSC |
| | | SVMHEALHNHYTQKSLSLSPGKSGGLNDIFEAQKI |
| | | EWHE |
| 254 | monomeric muLTBR ECD | DKTHTCPPCPAPELLGGPSVFLFPPKPKDTLMISR |
| | (S28-L223) Fc-fusion avi | TPEVTCVVVDVSHEDPEVKFNWYVDGVEVHNAKTK |
| | biotinylated | PREEQYNSTYRVVSVLTVLHQDWLNGKEYKCKVSN |
| | (P1AE4410)(hole chain) | KALPAPIEKTISKAKGQPREPQVCTLPPSRDELTK |
| | | NQVSLSCAVKGFYPSDIAVEWESNGQPENNYKTTP |
| | | PVLDSDGSFFLVSKLTVDKSRWQQGNVFSCSVMHE |
| | | ALHNRFTQKSLSLSPGK |
| 255 | monomeric cynoLTBR | SQPQVVPPYGSENQTCRDQEKEYYEPRHRICCSRC |
| | ectodomain (S28-M227) Fc | PPGTYVSAKCSRSRDTVCATCAENSYNEHWNYLTI |
| | fusion avi biotinylated | CQLCRPCDPVMGLEEIAPCTSKRKTQCRCQPGMFC |
| | (P1AE4411) (knob chain) | AAWALECTHCELLSDCPPGTEAELKDEVGKGNNHC |
| | | VPCKAGHFQNTSSPSARCQPHTRCEDQGLVEAAPG |
| | | TAQSDTTCRNPSESLPPEMSGTMLMASGSDKTHTC |
| | | PPCPAPELLGGPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALPAP |
| | | IEKTISKAKGQPREPQVYTLPPCRDELTKNQVSLW |
| | | CLVKGFYPSDIAVEWESNGQPENNYKTTPPVLDSD |
| | | GSFFLYSKLTVDKSRWQQGNVFSCSVMHEALHNHY |
| | | TQKSLSLSPGKSGGLNDIFEAQKIEWHE |
| 256 | monomeric cynoLTBR | DKTHTCPPCPAPELLGGPSVFLFPPKPKDTLMISR |
| | ectodomain (S28-M227) Fc | TPEVTCVVVDVSHEDPEVKFNWYVDGVEVHNAKTK |
| | fusion avi biotinylated | PREEQYNSTYRVVSVLTVLHQDWLNGKEYKCKVSN |
| | (P1AE4411) (hole chain) | KALPAPIEKTISKAKGQPREPQVCTLPPSRDELTK |
| | | NQVSLSCAVKGFYPSDIAVEWESNGQPENNYKTTP |
| | | PVLDSDGSFFLVSKLTVDKSRWQQGNVFSCSVMHE |
| | | ALHNRFTQKSLSLSPGK |
| 257 | monomeric N-terminal | QTCRDQEKEYYEPQHRICCSRCPPGTYVSAKCSRI |
| | human LTbR (ECD full | RDTVCATCAENSYNEHWNYLTICQLCRPCDPVMGL |
| | length) hu IgG1 Fc-fusion | EEIAPCTSKRKTQCRCQPGMFCAAWALECTHCELL |
| | kih HRYF avi | SDCPPGTEAELKDEVGKGNNHCVPCKAGHFQNTSS |
| | (P1AE1217)(knob chain) | PSARCQPHTRCENQGLVEAAPGTAQSDTTCKASGS |
| | | DKTHTCPPCPAPEAAGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKTTP |
| | | PVLDSDGSFFLYSKLTVDKSRWQQGNVFSCSVMHE |
| | | ALHNHYTQKSLSLSPGKSGGLNDIFEAQKIEWHE |
| 258 | monomeric N-terminal | DKTHTCPPCPAPELLGGPSVFLFPPKPKDTLMISR |
| | human LTbR (ECD full | TPEVTCVVVDVSHEDPEVKFNWYVDGVEVHNAKTK |
| | length) hu IgG1 Fc-fusion | PREEQYNSTYRVVSVLTVLHQDWLNGKEYKCKVSN |
| | kih HRYF avi | KALPAPIEKTISKAKGQPREPQVCTLPPSRDELTK |
| | (P1AE1217)(hole chain) | NQVSLSCAVKGFYPSDIAVEWESNGQPENNYKTTP |
| | | PVLDSDGSFFLVSKLTVDKSRWQQGNVFSCSVMHE |
| | | ALHNRFTQKSLSLSPGK |
| 259 | Avi tag | GLNDIFEAQKIEWHE |
| 260 | human LTBR ECD (S28- | SQPQAVPPYASENQTCRDQEKEYYEPQHRICCSRC |
| | M227) N- and C-terminally | PPGTYVSAKCSRIRDTVCATCAENSYNEHWNYLTI |
| | extended-Fc-fusion wt kih | CQLCRPCDPVMGLEEIAPCTSKRKTQCRCQPGMFC |
| | HRYF-Avi His | AAWALECTHCELLSDCPPGTEAELKDEVGKGNNHC |
| | biotinylated | VPCKAGHFQNTSSPSARCQPHTRCENQGLVEAAPG |
| | 261(P1AE7979)(knob chain) | TAQSDTTCKNPLEPLPPEMSGTMLMASGSDKTHTC |
| | | PPCPAPELLGGPSVFLFPPKPKDTLMISRTPEVTC |
| | | VVVDVSHEDPEVKFNWYVDGVEVHNAKTKPREEQY |
| | | NSTYRVVSVLTVLHQDWLNGKEYKCKVSNKALPAP |
| | | IEKTISKAKGQPREPQVYTLPPCRDELTKNQVSLW |
| | | CLVKGFYPSDIAVEWESNGQPENNYKTTPPVLDSD |
| | | GSFFLYSKLTVDKSRWQQGNVFSCSVMHEALHNHY |
| | | TQKSLSLSPGKSGGLNDIFEAQKIEWHEGGHHHHH |
| | | H |
| 261 | human LTBR ECD (S28- | DKTHTCPPCPAPELLGGPSVFLFPPKPKDTLMISR |
| | M227) N- and C-terminally | TPEVTCVVVDVSHEDPEVKFNWYVDGVEVHNAKTK |
| | extended-Fc-fusion wt kih | PREEQYNSTYRVVSVLTVLHQDWLNGKEYKCKVSN |
| | HRYF Avi-His | KALPAPIEKTISKAKGQPREPQVCTLPPSRDELTK |
| | biotinylated | NQVSLSCAVKGFYPSDIAVEWESNGQPENNYKTTP |
| | (P1AE7979)(hole chain) | PVLDSDGSFFLVSKLTVDKSRWQQGNVFSCSVMHE |
| | | ALHNRFTQKSLSLSPGK |
| 262 | monomeric N-terminal cyno | QTCRDQEKEYYEPRHRICCSRCPPGTYVSAKCSRS |
| | LTbR (ECD full length) hu | RDTVCATCAENSYNEHWNYLTICQLCRPCDPVMGL |
| | IgG1 Fc-fusion kih HRYF | EEIAPCTSKRKTQCRCQPGMFCAAWALECTHCELL |
| | avi biotinylated | SDCPPGTEAELKDEVGKGNNHCVPCKAGHFQNTSS |
| | (P1AE2656)(knob chain) | PSARCQPHTRCEDQGLVEAAPGTAQSDTTCRASGS |
| | | DKTHTCPPCPAPEAAGGPSVFLFPPKPKDTLMISR |
| | | TPEVTCVVVDVSHEDPEVKFNWYVDGVEVHNAKTK |
| | | PREEQYNSTYRVVSVLTVLHQDWLNGKEYKCKVSN |
| | | KALGAPIEKTISKAKGQPREPQVYTLPPCRDELTK |
| | | NQVSLWCLVKGFYPSDIAVEWESNGQPENNYKTTP |
| | | PVLDSDGSFFLYSKLTVDKSRWQQGNVFSCSVMHE |
| | | ALHNHYTQKSLSLSPGKSGGLNDIFEAQKIEWHE |
| 263 | monomeric N-terminal cyno | DKTHTCPPCPAPELLGGPSVFLFPPKPKDTLMISR |
| | LTbR (ECD full length) hu | TPEVTCVVVDVSHEDPEVKFNWYVDGVEVHNAKTK |
| | IgG1 Fc-fusion kih HRYF | PREEQYNSTYRVVSVLTVLHQDWLNGKEYKCKVSN |
| | avi biotinylated | KALPAPIEKTISKAKGQPREPQVCTLPPSRDELTK |
| | (P1AE2656)(hole chain) | NQVSLSCAVKGFYPSDIAVEWESNGQPENNYKTTP |
| | | PVLDSDGSFFLVSKLTVDKSRWQQGNVFSCSVMHE |
| | | ALHNRFTQKSLSLSPGK |
| 264 | human FAP-Avi-His | RPSRVHNSEENTMRALTLKDILNGTFSYKTFFPNW |
| | biotinylated (P1AA5347) | ISGQEYLHQSADNNIVLYNIETGQSYTILSNRTMK |
| | | SVNASNYGLSPDRQFVYLESDYSKLWRYSYTATYY |
| | | IYDLSNGEFVRGNELPRPIQYLCWSPVGSKLAYVY |
| | | QNNIYLKQRPGDPPFQITENGRENKIENGIPDWVY |
| | | EEEMLATKYALWWSPNGKFLAYAEFNDTDIPVIAY |
| | | SYYGDEQYPRTINIPYPKAGAKNPVVRIFIIDTTY |
| | | PAYVGPQEVPVPAMIASSDYYFSWLTWVTDERVCL |
| | | QWLKRVQNVSVLSICDFREDWQTWDCPKTQEHIEE |
| | | SRTGWAGGFFVSTPVESYDAISYYKIFSDKDGYKH |
| | | IHYIKDTVENAIQITSGKWEAINIFRVTQDSLFYS |
| | | SNEFEEYPGRRNIYRISIGSYPPSKKCVTCHLRKE |
| | | RCQYYTASFSDYAKYYALVCYGPGIPISTLHDGRT |
| | | DQEIKILEENKELENALKNIQLPKEEIKKLEVDEI |
| | | TLWYKMILPPQFDRSKKYPLLIQVYGGPCSQSVRS |
| | | VFAVNWISYLASKEGMVIALVDGRGTAFQGDKLLY |
| | | AVYRKLGVYEVEDQITAVRKFIEMGFIDEKRIAIW |
| | | GWSYGGYVSSLALASGTGLFKCGIAVAPVSSWEYY |
| | | ASVYTEREMGLPTKDDNLEHYKNSTVMARAEYERN |
| | | VDYLLIHGTADDNVHFQNSAQIAKALVNAQVDFQA |
| | | MWYSDQNHGLSGLSTNHLYTHMTHELKQCFSLSVD |
| | | EQLYFQGGSGLNDIFEAQKIEWHEARAHHHHHH |
| 265 | murine FAP-Avi-His | RPSRVYKPEGNTKRALTLKDILNGTFSYKTYFPNW |
| | biotinylated (P1AD9907) | ISEQEYLHQSEDDNIVFYNIETRESYIILSNSTMK |
| | | SVNATDYGLSPDRQFVYLESDYSKLWRYSYTATYY |
| | | IYDLQNGEFVRGYELPRPIQYLCWSPVGSKLAYVY |
| | | QNNIYLKQRPGDPPFQITYTGRENRIENGIPDWVY |
| | | EEEMLATKYALWWSPDGKFLAYVEENDSDIPIIAY |
| | | SYYGDGQYPRTINIPYPKAGAKNPVVRVFIVDTTY |
| | | PHHVGPMEVPVPEMIASSDYYFSWLTWVSSERVCL |
| | | QWLKRVQNVSVLSICDFREDWHAWECPKNQEHVEE |
| | | SRTGWAGGFFVSTPAFSQDATSYYKIFSDKDGYKH |
| | | IHYIKDTVENAIQITSGKWEAIYIFRVTQDSLFYS |
| | | SNEFEGYPGRRNIYRISIGNSPPSKKCVTCHLRKE |
| | | RCQYYTASFSYKAKYYALVCYGPGLPISTLHDGRT |
| | | DQEIQVLEENKELENSLRNIQLPKVEIKKLKDGGL |
| | | TFWYKMILPPQFDRSKKYPLLIQVYGGPCSQSVKS |
| | | VFAVNWITYLASKEGIVIALVDGRGTAFQGDKELH |
| | | AVYRKLGVYEVEDQLTAVRKFIEMGFIDEERIAIW |
| | | GWSYGGYVSSLALASGTGLFKCGIAVAPVSSWEYY |
| | | ASIYSERFMGLPTKDDNLEHYKNSTVMARAEYERN |
| | | VDYLLIHGTADDNVHFQNSAQIAKALVNAQVDEQA |
| | | MWYSDQNHGILSGRSQNHLYTHMTHFLKQCESLSD |
| | | VDEQLYFQGGSGLNDIFEAQKIEWHEARAHHHHHH |
| 266 | LTBR (5929) CDR-H2 | IPIFG |
| | patch | |
| 267 | HUJH5.HFc-DO3 | CTT GGG CCC TTG GTA CTT GCT GAG GAG |
| | | ACG GTG ACC AGG G |
| 268 | HUJH6.HFc-Do3 | CTT GGG CCC TTG GTA CTT GCT GAG GAG |
| | | ACG GTG ACC GTG G |
| 269 | BcPCR FHLC leader.fw | ACA GAG GAA GCT TGC CGC CAC CAT GGA |
| | (TG) | CAT GAG GGT CCC CGC |
| 270 | HuCK.Do.2AA | TGA AGA CAG ATG GTG CAG CCA CAG |
| 271 | WTRat.VH.FW11 | SAR GTV MAG CTG VWG SAG TCW GGR SS |
| 272 | WTRat.VH.FW12 | SAG GTG MAG CTS SWG SAR WCW GGR SS |
| 273 | WTRat.VH.FW13 | SAR GTK MAR CTG SWG SAG TCW GGR SS |
| 274 | WTRat.VH.FW14 | SMG GTG MAG CTK SWK SAG TCW GGW SS |
| 275 | WTRat.VH.FW15 | AGG TKM MKC TGA WRS ART CWG GMS ST |
| 276 | WTRat.VH.FW16 | SAG GTR MAG CTG VWR SAG TCW GGR SV |
| 277 | WTRat. VH.FW17 | AGG TGC AGC TDV WRS AGW CWG GRS SW |
| 278 | 682.rev do | GGGAAGATGAAGACAGATG |
| 279 | WTRat.VK.FW1 | GRYRTYSWGWTGACMCARDCTCCDB |
| 280 | WTRat.VK.FW2 | GMTRTTGTKMTSAMNCARDCWCCAB |
| 281 | WTRat.CKappa.Do2 | gacgggtgaggatgatgtcttatgaacaa |
| 282 | mouse FAP | UniProt no. P97321 |
| 283 | CH1 domain | ASTKGPSVFPLAPSSKSTSGGTAALGCLVKDYFPE |
| | | PVTVSWNSGALTSGVHTFPAVLQSSGLYSLSSVVT |
| | | VPSSSLGTQTYICNVNHKPSNTKVDKKV |
| 284 | CH1 connector C-terminal | EPKSC |
| | end | |
| 285 | CH1 connector C-terminal | EPKSCD |
| | end D-variant | |
| 286 | CH1 connector C-terminal | EPKSCS |
| | end S-variant | |
| 287 | Hinge full | DKTHTCPXCP with X being S or P |
| 288 | Hinge middle | HTCPXCP with X being S or P |
| 289 | Hinge short | CPXCP with X being S or P |
| 290 | CH2 domain | APELLGGPSVFLFPPKPKDTLMISRTPEVTCVWDV |
| | | SHEDPEVKFNWYVDGVEVHNAKTKPREEQESTYRW |
| | | SVLTVLHQDWLNGKEYKCKVSNKALPAPIEKTISK |
| | | AK |
| 291 | CH3 domain | GQPREPQVYTLPPSRDELTKNQVSLTCLVKGFYPS |
| | | DIAVEWESNGQPENNYKTTPPVLDSDGSFFLYSKL |
| | | TVDKSRWQQGNVFSCSVMHEALHNHYTQKSLSLSP |
| 292 | Fc IgG1, caucasian allotype | ASTKGPSVFPLAPSSKSTSGGTAALGCLVKDYFPE |
| | | PVTVSWNSGALTSGVHTFPAVLQSSGLYSLSSVVT |
| | | VPSSSLGTQTYICNVNHKPSNTKVDKKVEPKSCDK |
| | | THTCPPCPAPELLGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LPAPIEKTISKAKGQPREPQVYTLPPSRDELTKNQ |
| | | VSLTCLVKGFYPSDIAVEWESNGQPENNYKTTPPV |
| | | LDSDGSFFLYSKLTVDKSRWQQGNVFSCSVMHEAL |
| | | HNHYTQKSLSLSPGK |
| 293 | Fc IgG1, afroamerican | ASTKGPSVFPLAPSSKSTSGGTAALGCLVKDYFPE |
| | allotype | PVTVSWNSGALTSGVHTFPAVLQSSGLYSLSSVVT |
| | | VPSSSLGTQTYICNVNHKPSNTKVDKKVEPKSCDK |
| | | THTCPPCPAPELLGGPSVFLFPPKPKDTLMISRTP |
| | | EVTCVVVDVSHEDPEVKFNWYVDGVEVHNAKTKPR |
| | | EEQYNSTYRVVSVLTVLHQDWLNGKEYKCKVSNKA |
| | | LPAPIEKTISKAKGQPREPQVYTLPPSREEMTKNQ |
| | | VSLTCLVKGFYPSDIAVEWESNGQPENNYKTTPPV |
| | | LDSDGSFFLYSKLTVDKSRWQQGNVESCSVMHEAL |
| | | HNHYTQKSLSLSPGK |
| 294 | Fc IgG2 | ASTKGPSVFPLAPCSRSTSESTAALGCLVKDYFPE |
| | | PVTVSWNSGALTSGVHTFPAVLQSSGLYSLSSVVT |
| | | VPSSNFGTQTYTCNVDHKPSNTKVDKTVERKCCVE |
| | | CPPCPAPPVAGPSVFLFPPKPKDTLMISRTPEVTC |
| | | VVVDVSHEDPEVQENWYVDGVEVHNAKTKPREEQF |
| | | NSTFRVVSVLTVVHQDWLNGKEYKCKVSNKGLPAP |
| | | IEKTISKTKGQPREPQVYTLPPSREEMTKNQVSLT |
| | | CLVKGFYPSDISVEWESNGQPENNYKTTPPMLDSD |
| | | GSFFLYSKLTVDKSRWQQGNVFSCSVMHEALHNHY |
| | | TQKSLSLSPGK |
| 295 | Fc IgG3 | ASTKGPSVFPLAPCSRSTSGGTAALGCLVKDYFPE |
| | | PVTVSWNSGALTSGVHTFPAVLQSSGLYSLSSVVT |
| | | VPSSSLGTQTYTCNVNHKPSNTKVDKRVELKTPLG |
| | | DTTHTCPRCPEPKSCDTPPPCPRCPEPKSCDTPPP |
| | | CPRCPEPKSCDTPPPCPRCPAPELLGGPSVFLFPP |
| | | KPKDTLMISRTPEVTCVVVDVSHEDPEVQFKWYVD |
| | | GVEVHNAKTKPREEQYNSTFRVVSVLTVLHQDWLN |
| | | GKEYKCKVSNKALPAPIEKTISKTKGQPREPQVYT |
| | | LPPSREEMTKNQVSLTCLVKGFYPSDIAVEWESSG |
| | | QPENNYNTTPPMLDSDGSFFLYSKLTVDKSRWQQG |
| | | NIFSCSVMHEALHNRFTQKSLSLSPGK |
| 296 | Fc IgG4 | ASTKGPSVFPLAPCSRSTSESTAALGCLVKDYFPE |
| | | PVTVSWNSGALTSGVHTFPAVLQSSGLYSLSSVVT |
| | | VPSSSLGTKTYTCNVDHKPSNTKVDKRVESKYGPP |
| | | CPSCPAPEFLGGPSVFLFPPKPKDTLMISRTPEVT |
| | | CVVVDVSQEDPEVQFNWYVDGVEVHNAKTKPREEQ |
| | | FNSTYRVVSVLTVLHQDWLNGKEYKCKVSNKGLPS |
| | | SIEKTISKAKGQPREPQVYTLPPSQEEMTKNQVSL |
| | | TCLVKGFYPSDIAVEWESNGQPENNYKTTPPVLDS |
| | | DGSFFLYSRLTVDKSRWQEGNVESCSVMHEALHNH |
| | | YTQKSLSLSLGK |
| 297 | Mouse LTBR | Uniprot Acc. NO. B2RRV3 |
| | Mus musculus | MRLPRASSPC GLAWGPLLLG LSGLLVASQP |
| | | QLVPPYRIEN QTCWDODKEY YEPMHDVCCS |
| | | RCPPGEFVFA VCSRSQDTVC KTCPHNSYNE |
| | | HWNHLSTCQL CRPCDIVLGF EEVAPCTSDR |
| | | KAECRCQPGM SCVYLDNECV HCEEERLVLC |
| | | QPGTEAEVTD EIMDTDVNCV PCKPGHFQNT |
| | | SSPRARCOPH TRCEIQGLVE AAPGTSYSDT |
| | | ICKNPPEPGA MLLLAILLSL VLFLLFTTVL |
| | | ACAWMRHPSL CRKLGTLLKR HPEGEESPPC |
| | | PAPRADPHFP DLAEPLLPMS GDLSPSPAGP |
| | | PTAPSLEEVV LQQQSPLVQA RELEAEPGEH |
| | | GQVAHGANGI HVTGGSVTVT GNIYIYNGPV |
| | | LGGTRGPGDP PAPPEPPYPT PEEGAPGPSE |
| | | LSTPYQEDGK AWHLAETETL GCQDL |
| 298 | Peptide linker (G4S) | GGGGS |
| 299 | Peptide linker (G4S)₂ | GGGGSGGGGS |
| 300 | Peptide linker (SG4)₂ | SGGGGSGGGG |
| 301 | Peptide linker G4(SG4)₂ | GGGGSGGGGSGGGG |
| 302 | peptide linker | GSPGSSSSGS |
| 303 | (G4S)₃ peptide linker | GGGGSGGGGSGGGGS |
| 304 | (G4S)₄ peptide linker | GGGGGGGGSGGGGSGGGGS |
| 305 | peptide linker | GSGSGSGS |
| 306 | peptide linker | GSGSGNGS |
| 307 | peptide linker | GGSGSGSG |
| 308 | peptide linker | GGSGSG |
| 309 | peptide linker | GGSG |
| 310 | peptide linker | GGSGNGSG |
| 311 | peptide linker | GGNGSGSG |
| 312 | peptide linker | GGNGSG |
| 313 | VH (PD-L1) | EVQLVESGGGLVQPGGSLRLSCAASGFTFSDSWIHWVRQ |
| | | APGKGLEWVAWISPYGGSTYYADSVKGRFTISADTSKNT |
| | | AYLQMNSLRAEDTAVYYCARRHWPGGFDYWGQGTLVTVS |
| | | S |
| 314 | VL (PD-L1) | DIQMTQSPSSLSASVGDRVTITCRASQDVSTAVAWYQQK |
| | | PGKAPKLLIYSASFLYSGVPSRFSGSGSGTDFTLTISSL |
| | | QPEDFATYYCQQYLYHPATFGQGTKVEIK |
| 315 | VH (PD-L1) | EVQLVESGGGLVQPGGSLRLSCAASGFTFSRYWMSWVRQ |
| | | APGKGLEWVANIKQDGSEKYYVDSVKGRFTISRDNAKNS |
| | | LYLQMNSLRAEDTAVYYCAREGGWFGELAFDYWGQGTLV |
| | | TVSS |
| 316 | VL (PD-L1) | EIVLTQSPGTLSLSPGERATLSCRASQRVSSSYLAWYQQ |
| | | KPGQAPRLLIYDASSRATGIPDRFSGSGSGTDFTLTISR |
| | | LEPEDFAVYYCQQYGSLPWTFGQGTKVEIK |
| 317 | rbHC.up | AAG CTT GCC ACC ATG GAG ACT GGG CTG |
| | | CGC TGG CTT C |
| 318 | P1AG5459 HC KK+ LTBR | EVQLVESGGGLVQPGGSLRLSCAASGFTFSSYTMNWVRQAPGK |
| | with charges-FAP crossed | GLEWVSGVAPSGDRTYYTDSVKGRFTISRDNAKNTLDLQMTSL |
| | (murine 2 + 1 surrogate) | RAEDAAIYICAQGFAFTNWGQGTMVTVSSAKTTPPSVYPLAPG |
| | | SAAQTNSMVTLGCLVEGYFPEPVTVTWNSGSLSSGVHTFPAVL |
| | | QSDLYTLSSSVTVPSSTWPSQTVTCNVAHPASSTKVDEKIVPR |
| | | DCGCKPCICTVPEVSSVFIFPPKPKDVLTITLTPKVTCVVVAI |
| | | SKDDPEVQFSWFVDDVEVHTAQTKPREEQINSTERSVSELPIM |
| | | HQDWLNGKEFKCRVNSAAFGAPIEKTISKTKGRPKAPQVYTIP |
| | | PPKKQMAKDKVSLTCMITNFFPEDITVEWQWNGQPAENYKNTQ |
| | | PIMKTDGSYFVYSKLNVQKSNWEAGNTFTCSVLHEGLHNHHTE |
| | | KSLSHSPGGGGGSGGGGSGGGGSGGGGSEVQLLESGGGLVQPG |
| | | GSLRLSCAASGFTFSSHAMSWVRQAPGKGLEWVSAIWASGEQY |
| | | YADSVKGRFTISRDNSKNTLYLQMNSLRAEDTAVYYCAKGWLG |
| | | NFDYWGQGTLVTVSSRADAAPTVSIFPPSSEQLTSGGASVVCF |
| | | LNNFYPKDINVKWKIDGSERQNGVLNSWTDQDSKDSTYSMSST |
| | | LTLTKDEYERHNSYTCEATHKTSTSPIVKSFNRNEC |
| 319 | P1AG5459 LC LTBR with | DIQMTQSPSSLSASVGDRVTITCRASQGISSALAWFQQKPGKA |
| | charges (murine 2 + 1 | PKRLIFDASSLDSGVPSRFSGSGSGTDFTLTISSLQPEDFATY |
| | surrogate) | YCQQYNNYPLTFGGGTKVEMKRADAAPTVSIFPPSSRKLTSGG |
| | | ASVVCFLNNFYPKDINVKWKIDGSERQNGVLNSWTDQDSKDST |
| | | YSMSSTLTLTKDEYERHNSYTCEATHKTSTSPIVKSFNRNEC |
| 320 | P1AG5459 HC DD− LTBR | EVQLVESGGGLVQPGGSLRLSCAASGFTFSSYTMNWVRQAPGK |
| | with charges (murine 2 + 1 | GLEWVSGVAPSGDRTYYTDSVKGRFTISRDNAKNTLDLQMTSL |
| | surrogate) | RAEDAAIYICAQGFAFTNWGQGTMVTVSSAKTTPPSVYPLAPG |
| | | SAAQTNSMVTLGCLVEGYFPEPVTVTWNSGSLSSGVHTFPAVL |
| | | QSDLYTLSSSVTVPSSTWPSQTVTCNVAHPASSTKVDEKIVPR |
| | | DCGCKPCICTVPEVSSVFIFPPKPKDVLTITLTPKVTCVVVAI |
| | | SKDDPEVQFSWFVDDVEVHTAQTKPREEQINSTFRSVSELPIM |
| | | HQDWLNGKEFKCRVNSAAFGAPIEKTISKTKGRPKAPQVYTIP |
| | | PPKEQMAKDKVSLTCMITNFFPEDITVEWQWNGQPAENYDNTQ |
| | | PIMDTDGSYFVYSDLNVQKSNWEAGNTFTCSVLHEGLHNHHTE |
| | | KSLSHSPG |
| 321 | P1AG5459 LC FAP crossed | EIVLTQSPGTLSLSPGERATLSCRASQSVSRSYLAWYQQKPGQ |
| | (murine 2 + 1 surrogate) | APRLLIIGASTRATGIPDRFSGSGSGTDFTLTISRLEPEDFAV |
| | | YYCQQGQVIPPTFGQGTKVEIKSSAKTTPPSVYPLAPGSAAQT |
| | | NSMVTLGCLVKGYFPEPVTVTWNSGSLSSGVHTFPAVLQSDLY |
| | | TLSSSVTVPSSTWPSQTVTCNVAHPASSTKVDKKIVPRDC |
| 322 | P1AG5461 HC KK+ LTBR | EVQLVESGGGLVQPGGSLRLSCAASGFTFSSYTMNWVRQAPGK |
| | with charges-DP47 crossed | GLEWVSGVAPSGDRTYYTDSVKGRFTISRDNAKNTLDLQMTSL |
| | (non-targeted murine 2 + 1 | RAEDAAIYICAQGFAFTNWGQGTMVTVSSAKTTPPSVYPLAPG |
| | surrogate) | SAAQTNSMVTLGCLVEGYFPEPVTVTWNSGSLSSGVHTFPAVL |
| | | QSDLYTLSSSVTVPSSTWPSQTVTCNVAHPASSTKVDEKIVPR |
| | | DCGCKPCICTVPEVSSVFIFPPKPKDVLTITLTPKVTCVVVAI |
| | | SKDDPEVQFSWFVDDVEVHTAQTKPREEQINSTFRSVSELPIM |
| | | HQDWLNGKEFKCRVNSAAFGAPIEKTISKTKGRPKAPQVYTIP |
| | | PPKKQMAKDKVSLTCMITNFFPEDITVEWQWNGQPAENYKNTQ |
| | | PIMKTDGSYFVYSKLNVQKSNWEAGNTFTCSVLHEGLHNHHTE |
| | | KSLSHSPGGGGGSGGGGSGGGGSGGGGSEVQLLESGGGLVQPG |
| | | GSLRLSCAASGFTFSSYAMSWVRQAPGKGLEWVSAISGSGGST |
| | | YYADSVKGRFTISRDNSKNTLYLQMNSLRAEDTAVYYCAKGSG |
| | | FDYWGQGTLVTVSSRADAAPTVSIFPPSSEQLTSGGASVVCFL |
| | | NNFYPKDINVKWKIDGSERQNGVLNSWTDQDSKDSTYSMSSTL |
| | | TLTKDEYERHNSYTCEATHKTSTSPIVKSFNRNEC |
| 323 | P1AG5461 LC LTBR with | DIQMTQSPSSLSASVGDRVTITCRASQGISSALAWFQQKPGKA |
| | charges (non-targeted | PKRLIFDASSLDSGVPSRFSGSGSGTDFTLTISSLQPEDFATY |
| | murine 2 + 1 surrogate) | YCQQYNNYPLTFGGGTKVEMKRADAAPTVSIFPPSSRKLTSGG |
| | | ASVVCFLNNFYPKDINVKWKIDGSERQNGVLNSWTDQDSKDST |
| | | YSMSSTLTLTKDEYERHNSYTCEATHKTSTSPIVKSFNRNEC |
| 324 | P1AG5461 HC DD− LTBR | EVQLVESGGGLVQPGGSLRLSCAASGFTFSSYTMNWVRQAPGK |
| | with charges (non-targeted | GLEWVSGVAPSGDRTYYTDSVKGRFTISRDNAKNTLDLQMTSL |
| | murine 2 + 1 surrogate) | RAEDAAIYICAQGFAFTNWGQGTMVTVSSAKTTPPSVYPLAPG |
| | | SAAQTNSMVTLGCLVEGYFPEPVTVTWNSGSLSSGVHTFPAVL |
| | | QSDLYTLSSSVTVPSSTWPSQTVTCNVAHPASSTKVDEKIVPR |
| | | DCGCKPCICTVPEVSSVFIFPPKPKDVLTITLTPKVTCVVVAI |
| | | SKDDPEVQFSWFVDDVEVHTAQTKPREEQINSTFRSVSELPIM |
| | | HQDWLNGKEFKCRVNSAAFGAPIEKTISKTKGRPKAPQVYTIP |
| | | PPKEQMAKDKVSLTCMITNFFPEDITVEWQWNGQPAENYDNTQ |
| | | PIMDTDGSYFVYSDLNVQKSNWEAGNTFTCSVLHEGLHNHHTE |
| | | KSLSHSPG |
| 325 | P1AG5461 LC DP47 | EIVLTQSPGTLSLSPGERATLSCRASQSVSSSYLAWYQQKPGQ |
| | crossed (non-targeted | APRLLIYGASSRATGIPDRFSGSGSGTDFTLTISRLEPEDFAV |
| | murine 2 + 1 surrogate) | YYCQQYGSSPLTFGQGTKVEIKSSAKTTPPSVYPLAPGSAAQT |
| | | NSMVTLGCLVKGYFPEPVTVTWNSGSLSSGVHTFPAVLQSDLY |
| | | TLSSSVTVPSSTWPSQTVTCNVAHPASSTKVDKKIVPRDC |
| 326 | P1AE1079(2 + 1) HC knob | EVQLVESGGGLVKPGGSLRLSCAASGFTFSDYYMYWFRQAPGK |
| | LTBR (CBE11)-FAP VH | GLEWVATISDGGSYTYYPDSVKGRFTISRDNAKNSLYLQMSSL |
| | | RAEDTAVYYCAREENGNFYYFDYWGQGTTVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVD |
| | | KKVEPKSCDKTHTCPPCPAPEAAGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALGAPIEKTISKAKG |
| | | QPREPQVYTLPPCRDELTKNQVSLWCLVKGFYPSDIAVEWESN |
| | | GQPENNYKTTPPVLDSDGSFFLYSKLTVDKSRWQQGNVFSCSV |
| | | MHEALHNHYTQKSLSLSPGGGGGSGGGGSGGGGSGGGGSEVQL |
| | | LESGGGLVQPGGSLRLSCAASGFTFSSYAMSWVRQAPGKGLEW |
| | | VSAIIGSGASTYYADSVKGRFTISRDNSKNTLYLQMNSLRAED |
| | | TAVYYCAKGWFGGFNYWGQGTLVTVSS |
| 327 | P1AG1079(2 + 1) LC LTBR | DIQMTQSPSSLSASVGDRVTITCKAGQDIKSYLSWYQQKPGKA |
| | (CBE11) 2x | PKLLIYYATRLADGVPSRFSGSGSGTDYTLTISSLQPEDFATY |
| | | YCLQHGESPWTFGGGTKLEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSENRGEC |
| 328 | P1AG1079(2 + 1) HC hole | EVQLVESGGGLVKPGGSLRLSCAASGFTFSDYYMYWFRQAPGK |
| | LTBR (CBE11)-FAP VL | GLEWVATISDGGSYTYYPDSVKGRFTISRDNAKNSLYLQMSSL |
| | | RAEDTAVYYCAREENGNFYYFDYWGQGTTVTVSSASTKGPSVF |
| | | PLAPSSKSTSGGTAALGCLVKDYFPEPVTVSWNSGALTSGVHT |
| | | FPAVLQSSGLYSLSSVVTVPSSSLGTQTYICNVNHKPSNTKVD |
| | | KKVEPKSCDKTHTCPPCPAPEAAGGPSVFLFPPKPKDTLMISR |
| | | TPEVTCVVVDVSHEDPEVKFNWYVDGVEVHNAKTKPREEQYNS |
| | | TYRVVSVLTVLHQDWLNGKEYKCKVSNKALGAPIEKTISKAKG |
| | | QPREPQVCTLPPSRDELTKNQVSLSCAVKGFYPSDIAVEWESN |
| | | GQPENNYKTTPPVLDSDGSFFLVSKLTVDKSRWQQGNVFSCSV |
| | | MHEALHNHYTQKSLSLSPGGGGGSGGGGSGGGGSGGGGSEIVL |
| | | TQSPGTLSLSPGERATLSCRASQSVTSSYLAWYQQKPGQAPRL |
| | | LINVGSRRATGIPDRFSGSGSGTDFTLTISRLEPEDFAVYYCQ |
| | | QGIMLPPTFGQGTKVEIK |
| 329 | P1AH5884 (1 + 1) HC knob | DIQMTQSPSSLSASVGDRVTITCKASQNVGINVAWYQQKPGKA |
| | LTBR (BHA10) crossed | PKSLISSASYRYSGVPSRFSGSGSGTDFTLTISSLQPEDFATY |
| | | FCQQYDTYPFTFGQGTKVEIKSSASTKGPSVFPLAPSSKSTSG |
| | | GTAALGCLVKDYFPEPVTVSWNSGALTSGVHTFPAVLQSSGLY |
| | | SLSSVVTVPSSSLGTQTYICNVNHKPSNTKVDKKVEPKSCDKT |
| | | HTCPPCPAPEAAGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALGAPIEKTISKAKGQPREPQVYTLP |
| | | PCRDELTKNQVSLWCLVKGFYPSDIAVEWESNGQPENNYKTTP |
| | | PVLDSDGSFFLYSKLTVDKSRWQQGNVFSCSVMHEALHNHYTQ |
| | | KSLSLSPG |
| 330 | P1AH5884 (1 + 1) HC hole | EVQLLESGGGLVQPGGSLRLSCAASGFTFSSYAMSWVRQAPGK |
| | FAP with charges | GLEWVSAIIGSGASTYYADSVKGRFTISRDNSKNTLYLQMNSL |
| | | RAEDTAVYYCAKGWFGGFNYWGQGTLVTVSSASTKGPSVFPLA |
| | | PSSKSTSGGTAALGCLVEDYFPEPVTVSWNSGALTSGVHTFPA |
| | | VLQSSGLYSLSSVVTVPSSSLGTQTYICNVNHKPSNTKVDEKV |
| | | EPKSCDKTHTCPPCPAPEAAGGPSVFLFPPKPKDTLMISRTPE |
| | | VTCVVVDVSHEDPEVKFNWYVDGVEVHNAKTKPREEQYNSTYR |
| | | VVSVLTVLHQDWLNGKEYKCKVSNKALGAPIEKTISKAKGQPR |
| | | EPQVCTLPPSRDELTKNQVSLSCAVKGFYPSDIAVEWESNGQP |
| | | ENNYKTTPPVLDSDGSFFLVSKLTVDKSRWQQGNVFSCSVMHE |
| | | ALHNHYTQKSLSLSPG |
| 331 | P1AH5884 (1 + 1) LC LTBR | QVQLVQSGAEVKKPGSSVKVSCKASGYTFTTYYLHWVRQAPGQ |
| | (BHA10) crossed | GLEWMGWIYPGNVHAQYNEKFKGRVTITADKSTSTAYMELSSL |
| | | RSEDTAVYYCARSWEGFPYWGQGTTVTVSSASVAAPSVFIFPP |
| | | SDEQLKSGTASVVCLLNNFYPREAKVQWKVDNALQSGNSQESV |
| | | TEQDSKDSTYSLSSTLTLSKADYEKHKVYACEVTHQGLSSPVT |
| | | KSFNRGEC |
| 332 | P1AH5884 (1 + 1) LC FAP | EIVLTQSPGTLSLSPGERATLSCRASQSVTSSYLAWYQQKPGQ |
| | with charges | APRLLINVGSRRATGIPDRFSGSGSGTDFTLTISRLEPEDFAV |
| | | YYCQQGIMLPPTFGQGTKVEIKRTVAAPSVFIFPPSDRKLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 333 | P1AH5885 (2 + 1) HC knob | DIQMTQSPSSLSASVGDRVTITCKASQNVGINVAWYQQKPGKA |
| | LTBR (BHA10) crossed, | PKSLISSASYRYSGVPSRFSGSGSGTDFTLTISSLQPEDFATY |
| | FAP with charges | FCQQYDTYPFTFGQGTKVEIKSSASTKGPSVFPLAPSSKSTSG |
| | | GTAALGCLVKDYFPEPVTVSWNSGALTSGVHTFPAVLQSSGLY |
| | | SLSSVVTVPSSSLGTQTYICNVNHKPSNTKVDKKVEPKSCDKT |
| | | HTCPPCPAPEAAGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALGAPIEKTISKAKGQPREPQVYTLP |
| | | PCRDELTKNQVSLWCLVKGFYPSDIAVEWESNGQPENNYKTTP |
| | | PVLDSDGSFFLYSKLTVDKSRWQQGNVFSCSVMHEALHNHYTQ |
| | | KSLSLSPGGGGGSGGGGSGGGGSGGGGEVQLLESGGGLVQPGG |
| | | SLRLSCAASGFTFSSYAMSWVRQAPGKGLEWVSAIIGSGASTY |
| | | YADSVKGRFTISRDNSKNTLYLQMNSLRAEDTAVYYCAKGWFG |
| | | GFNYWGQGTLVTVSSASTKGPSVFPLAPSSKSTSGGTAALGCL |
| | | VEDYFPEPVTVSWNSGALTSGVHTFPAVLQSSGLYSLSSVVTV |
| | | PSSSLGTQTYICNVNHKPSNTKVDEKVEPKSCD |
| 334 | P1AH5885 (2 + 1) HC hole | DIQMTQSPSSLSASVGDRVTITCKASQNVGINVAWYQQKPGKA |
| | LTBR (BHA10) crossed | PKSLISSASYRYSGVPSRFSGSGSGTDFTLTISSLQPEDFATY |
| | | FCQQYDTYPFTFGQGTKVEIKSSASTKGPSVFPLAPSSKSTSG |
| | | GTAALGCLVKDYFPEPVTVSWNSGALTSGVHTFPAVLQSSGLY |
| | | SLSSVVTVPSSSLGTQTYICNVNHKPSNTKVDKKVEPKSCDKT |
| | | HTCPPCPAPEAAGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALGAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVMHEALHNHYTQ |
| | | KSLSLSPG |
| 335 | P1AH5885 (2 + 1) LC LTBR | QVQLVQSGAEVKKPGSSVKVSCKASGYTFTTYYLHWVRQAPGQ |
| | (BHA10)(2x) crossed | GLEWMGWIYPGNVHAQYNEKFKGRVTITADKSTSTAYMELSSL |
| | | RSEDTAVYYCARSWEGFPYWGQGTTVTVSSASVAAPSVFIFPP |
| | | SDEQLKSGTASVVCLLNNFYPREAKVQWKVDNALQSGNSQESV |
| | | TEQDSKDSTYSLSSTLTLSKADYEKHKVYACEVTHQGLSSPVT |
| | | KSFNRGEC |
| 336 | P1AH5885 (2 + 1) LC FAP | EIVLTQSPGTLSLSPGERATLSCRASQSVTSSYLAWYQQKPGQ |
| | with charges | APRLLINVGSRRATGIPDRFSGSGSGTDFTLTISRLEPEDFAV |
| | | YYCQQGIMLPPTFGQGTKVEIKRTVAAPSVFIFPPSDRKLKSG |
| | | TASVVCLLNNFYPREAKVQWKVDNALQSGNSQESVTEQDSKDS |
| | | TYSLSSTLTLSKADYEKHKVYACEVTHQGLSSPVTKSFNRGEC |
| 337 | P1AH5886 (2 + 1) HC knob | DIQMTQSPSSLSASVGDRVTITCRASQGISSALAWFQQKPGKA |
| | LTBR (9459) crossed, FAP | PKRLIFDASSLDSGVPSRFSGSGSGTDFTLTISSLQPEDFATY |
| | (212) with charges | YCQQYNNYPLTFGGGTKVEMKSSASTKGPSVFPLAPSSKSTSG |
| | | GTAALGCLVKDYFPEPVTVSWNSGALTSGVHTFPAVLQSSGLY |
| | | SLSSVVTVPSSSLGTQTYICNVNHKPSNTKVDKKVEPKSCDKT |
| | | HTCPPCPAPEAAGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALGAPIEKTISKAKGQPREPQVYTLP |
| | | PCRDELTKNQVSLWCLVKGFYPSDIAVEWESNGQPENNYKTTP |
| | | PVLDSDGSFFLYSKLTVDKSRWQQGNVFSCSVMHEALHNHYTQ |
| | | KSLSLSPGGGGGSGGGGSGGGGSGGGGQVQLVQSGAEVKKPGA |
| | | SVKVSCKASGYTLTDYNMDWVRQAPGQGLEWIGDIYPNTGGTI |
| | | YNQKFKGRVTMTIDTSTSTVYMELSSLRSEDTAVYYCTRFRGI |
| | | HYAMDYWGQGTTVTVSSASTKGPSVFPLAPSSKSTSGGTAALG |
| | | CLVEDYFPEPVTVSWNSGALTSGVHTFPAVLQSSGLYSLSSVV |
| | | TVPSSSLGTQTYICNVNHKPSNTKVDEKVEPKSCD |
| 338 | P1AH5886 (2 + 1) HC hole | DIQMTQSPSSLSASVGDRVTITCRASQGISSALAWFQQKPGKA |
| | LTBR (9459) crossed | PKRLIFDASSLDSGVPSRFSGSGSGTDFTLTISSLQPEDFATY |
| | | YCQQYNNYPLTFGGGTKVEMKSSASTKGPSVFPLAPSSKSTSG |
| | | GTAALGCLVKDYFPEPVTVSWNSGALTSGVHTFPAVLQSSGLY |
| | | SLSSVVTVPSSSLGTQTYICNVNHKPSNTKVDKKVEPKSCDKT |
| | | HTCPPCPAPEAAGGPSVFLFPPKPKDTLMISRTPEVTCVVVDV |
| | | SHEDPEVKFNWYVDGVEVHNAKTKPREEQYNSTYRVVSVLTVL |
| | | HQDWLNGKEYKCKVSNKALGAPIEKTISKAKGQPREPQVCTLP |
| | | PSRDELTKNQVSLSCAVKGFYPSDIAVEWESNGQPENNYKTTP |
| | | PVLDSDGSFFLVSKLTVDKSRWQQGNVFSCSVMHEALHNHYTQ |
| | | KSLSLSPG |
| 339 | P1AH5886 (2 + 1) LC LTBR | EVQLVESGGGLVQPGGSLRLSCAASGFTFSSYTMNWVRQAPGK |
| | (9459)(2x) crossed | GLEWVSGVAPSGDRTYYTDSVKGRFTISRDNAKNTLDLQMTSL |
| | | RAEDAAIYICAQGFAFTNWGQGTMVTVSSASVAAPSVFIFPPS |
| | | DEQLKSGTASVVCLLNNFYPREAKVQWKVDNALQSGNSQESVT |
| | | EQDSKDSTYSLSSTLTLSKADYEKHKVYACEVTHQGLSSPVTK |
| | | SFNRGEC |
| 340 | P1AH5886 (2 + 1) LC FAP | EIVLTQSPATLSLSPGERATLSCRASESVDNYGLSFINWFQQK |
| | (212) with charges | PGQAPRLLIYGTSNRGSGIPARFSGSGSGTDFTLTISSLEPED |
| | | FAVYFCQQSNEVPYTFGGGTKVEIKRTVAAPSVFIFPPSDRKL |
| | | KSGTASVVCLLNNFYPREAKVQWKVDNALQSGNSQESVTEQDS |
| | | KDSTYSLSSTLTLSKADYEKHKVYACEVTHQGLSSPVTKSFNR |
| | | GEC |
| 341 | P1AH0119 HC (LTBR | QVQLVQSGAEVKKPGSSVKVSCKASGYTFTTYYLHWVRQAPGQ |
| | BHA10 hu IgG1 PG LALA) | GLEWMGWIYPGNVHAQYNEKFKGRVTITADKSTSTAYMELSSL |
| | | RSEDTAVYYCARSWEGFPYWGQGTTVTVSSASTKGPSVFPLAP |
| | | SSKSTSGGTAALGCLVKDYFPEPVTVSWNSGALTSGVHTFPAV |
| | | LQSSGLYSLSSVVTVPSSSLGTQTYICNVNHKPSNTKVDKKVE |
| | | PKSCDKTHTCPPCPAPEAAGGPSVFLFPPKPKDTLMISRTPEV |
| | | TCVVVDVSHEDPEVKFNWYVDGVEVHNAKTKPREEQYNSTYRV |
| | | VSVLTVLHQDWLNGKEYKCKVSNKALGAPIEKTISKAKGQPRE |
| | | PQVYTLPPSRDELTKNQVSLTCLVKGFYPSDIAVEWESNGQPE |
| | | NNYKTTPPVLDSDGSFFLYSKLTVDKSRWQQGNVFSCSVMHEA |
| | | LHNHYTQKSLSLSPG |
| 342 | P1AH0119 LC (LTBR | DIQMTQSPSSLSASVGDRVTITCKASQNVGINVAWYQQKPGKA |
| | BHA10 hu IgG1 PG LALA) | PKSLISSASYRYSGVPSRFSGSGSGTDFTLTISSLQPEDFATY |
| | | FCQQYDTYPFTFGQGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| 343 | LTBR (BHA10) CDR-H1 | TYYLH |
| 344 | LTBR (BHA10) CDR-H2 | WIYPGNVHAQYNEKFKG |
| 345 | LTBR (BHA10) CDR-H3 | SWEGFPY |
| 346 | LTBR (BHA10) CDR-L1 | KASQNVGINVA |
| 347 | LTBR (BHA10) CDR-L2 | SASYRYS |
| 348 | LTBR (BHA10) CDR-L3 | QQYDTYPFT |
| 349 | LTBR (BHA10) VH | QVQLVQSGAEVKKPGSSVKVSCKASGYTFTTYYLHWVRQAPGQ |
| | | GLEWMGWIYPGNVHAQYNEKFKGRVTITADKSTSTAYMELSSL |
| | | RSEDTAVYYCARSWEGFPYWGQGTTVTVSS |
| 350 | LTBR (BHA10) VL | DIQMTQSPSSLSASVGDRVTITCKASQNVGINVAWYQQKPGKA |
| | | PKSLISSASYRYSGVPSRFSGSGSGTDFTLTISSLQPEDFATY |
| | | FCQQYDTYPFTFGQGTKVEIK |
| 351 | Epitope region (P1AE9459) | QCRCQPGMFCAAWALEC |
| 352 | Epitope region (P1AH0119) | AWALECTHCELL |
| 353 | Epitope Region 1 | QTCRDQEKEYYEPQHR |
| | (P1AE1873) | |
| 354 | Epitope Region 2 | SRIRDTVC |
| | (P1AE1873) | |
| 355 | Epitope Region 1 | SYNEHWNYLTICQL |
| | (P1AE1235) | |
| 356 | Epitope Region 2 | WALECTHCELLSDCPPGT |
| | (P1AE1235) | |
| 357 | Human Light (UniProt Acc | MEESVVRPSVFVVDGQTDIPFTRLGRSHRRQSCSVARVG |
| | No. 043557) | LGLLLLLMGAGLAVQGWELLQLHWRLGEMVTRLPDGPAG |
| | | SWEQLIQERRSHEVNPAAHLTGANSSLTGSGGPLLWETQ |
| | | LGLAFLRGLSYHDGALVVTKAGYYYIYSKVQLGGVGCPL |
| | | GLASTITHGLYKRTPRYPEELELLVSQQSPCGRATSSSR |
| | | VWWDSSFLGGVVHLEAGEKVVVRVLDERLVRLRDGTRSY |
| | | FGAFMV |
| 358 | Human Light (74-240) | DGPAGSWEQLIQERRSHEVNPAAHLTGANSSLTGSGGPL |
| | | LWETQLGLAFLRGLSYHDGALVVTKAGYYYIYSKVQLGG |
| | | VGCPLGLASTITHGLYKRTPRYPEELELLVSQQSPCGRA |
| | | TSSSRVWWDSSFLGGVVHLEAGEKVVVRVLDERLVRLRD |
| | | GTRSYFGAFMV |
| 359 | Human LTBR ECD (S28- | SQPQAVPPYASENQTCRDQEKEYYEPQHRICCSRCPPGT |
| | M227) | YVSAKCSRIRDTVCATCAENSYNEHWNYLTICQLCRPCD |
| | | PVMGLEEIAPCTSKRKTQCRCQPGMFCAAWALECTHCEL |
| | | LSDCPPGTEAELKDEVGKGNNHCVPCKAGHFQNTSSPSA |
| | | RCQPHTRCENQGLVEAAPGTAQSDTTCKNPLEPLPPEMS |
| | | GTMLM |
| 360 | Murine LTBR ECD (S28- | SQPQLVPPYRIENQTCWDQDKEYYEPMHDVCCSRCPPGE |
| | L223) | FVFAVCSRSQDTVCKTCPHNSYNEHWNHLSTCQLCRPCD |
| | | IVLGFEEVAPCTSDRKAECRCQPGMSCVYLDNECVHCEE |
| | | ERLVLCQPGTEAEVTDEIMDTDVNCVPCKPGHFQNTSSP |
| | | RARCQPHTRCEIQGLVEAAPGTSYSDTICKNPPEPGAML |
| | | L |
| 361 | Cynomolgus LTBR ECD | SQPQVVPPYGSENQTCRDQEKEYYEPRHRICCSRCPPGT |
| | (S28-M227) | YVSAKCSRSRDTVCATCAENSYNEHWNYLTICQLCRPCD |
| | | PVMGLEEIAPCTSKRKTQCRCQPGMFCAAWALECTHCEL |
| | | LSDCPPGTEAELKDEVGKGNNHCVPCKAGHFQNTSSPSA |
| | | RCQPHTRCEDQGLVEAAPGTAQSDTTCRNPSESLPPEMS |
| | | GTMLM |

The following numbered paragraphs (paras) are aspects of the invention:

1. A bispecific antigen binding molecule, comprising
(a) a first antigen binding domain that specifically binds to Fibroblast Activation Protein (FAP),
(b) a second antigen binding domain that specifically binds to lymphotoxin beta receptor (LTBR), and
(c) a Fc domain composed of a first and a second subunit and comprising one or more amino acid substitution that reduces the binding affinity of the antigen binding molecule to an Fc receptor and/or effector function.

2. The bispecific antigen binding molecule of para 1, wherein the bispecific antigen binding molecule activates LTBR upon binding to FAP.

3. The bispecific antigen binding molecule of paras 1 or 2, wherein the bispecific antigen binding molecule comprises a third antigen binding domain that specifically binds to LTBR.

4. The bispecific antigen binding molecule of para 3, wherein the third antigen binding domain that specifically binds to LTBR is identical to the second antigen binding domain that specifically binds to LTBR.

5. The bispecific antigen binding molecule of any one of paras 1 to 4, wherein the first antigen binding domain that specifically binds to FAP is a Fab fragment.

6. The bispecific antigen binding molecule of any one of paras 1 to 5, wherein the first antigen binding domain that specifically binds to FAP comprises
(i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8, or
(ii) a heavy chain variable region (V_{H}FAP) comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21, and a light chain variable region (V_{L}FAP) comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24.

7. The bispecific antigen binding molecule of any one of paras 1 to 6, wherein the first antigen binding domain that specifically binds to FAP comprises a heavy chain variable region (V_{H}FAP) comprising an amino acid sequence that is at least about 95% identical to the amino acid sequence of SEQ ID NO:9, and a light chain variable region (V_{L}FAP) comprising an amino acid sequence that is at least about 95% identical to the amino acid sequence of SEQ ID NO:10, or it comprises a heavy chain variable region (V_{H}FAP) comprising an amino acid sequence that is at least about 95% identical to the amino acid sequence of SEQ ID NO:25, and a light chain variable region (V_{L}FAP) comprising an amino acid sequence that is at least about 95% identical to the amino acid sequence of SEQ ID NO:26.

8. The bispecific antigen binding molecule of any one of paras 1 to 7, wherein the first antigen binding domain that specifically binds to FAP comprises a heavy chain variable region (V_{H}FAP) comprising the amino acid sequence of SEQ ID NO:9 and a light chain variable region (V_{L}FAP) comprising the amino acid sequence of SEQ ID NO:10 or it comprises a heavy chain variable region (V_{H}FAP) comprising the amino acid sequence of SEQ ID NO:25 and a light chain variable region (V_{L}FAP) comprising the amino acid sequence of SEQ ID NO:26.

9. The bispecific antigen binding molecule of any one of paras 1 to 8, wherein the second antigen binding domain that specifically binds to LTBR comprises
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
(ii) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(iii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48, or
(iv) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(v) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(vi) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72, or
(vii) a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80, or
(viii) a heavy chain variable region (V_{H} LTBR) comprising a CDR-H1 comprising the amino acid sequence of SEQ ID NO:83, a CDR-H2 comprising the amino acid sequence of SEQ ID NO:84, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

10. The bispecific antigen binding molecule of any one of paras 1 to 9, wherein the second antigen binding domain that specifically binds to LTBR comprises
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:41 and a light chain variable region (V_{L} LTBR) comprising the amino acid sequence of SEQ ID NO:42, or
(iii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(iv) a heavy chain variable region (V_{H} LTBR) comprising the amino acid sequence of SEQ ID NO:57 and a light chain variable region (V_{L} LTBR) comprising the amino acid sequence of SEQ ID NO:58, or
(v) a heavy chain variable region (V_{H} LTBR) comprising the amino acid sequence of SEQ ID NO:65 and a light chain variable region (V_{L} LTBR) comprising the amino acid sequence of SEQ ID NO:66, (vi) a heavy chain variable region (V_{H} LTBR) comprising the amino acid sequence of SEQ ID NO:73 and a light chain variable region (V_{L} LTBR) comprising the amino acid sequence of SEQ ID NO:74, (vii) a heavy chain variable region (V_{H} LTBR) comprising the amino acid sequence of SEQ ID NO:81 and a light chain variable region (V_{L} LTBR) comprising the amino acid sequence of SEQ ID NO:82, or
(viii) a heavy chain variable region (V_{H} LTBR) comprising the amino acid sequence of SEQ ID NO:89 and a light chain variable region (V_{L} LTBR) comprising the amino acid sequence of SEQ ID NO:90, or
(ix) a heavy chain variable region (V_{H} LTBR) comprising the amino acid sequence of SEQ ID NO:97 and a light chain variable region (V_{L} LTBR) comprising the amino acid sequence of SEQ ID NO:98.

11. The bispecific antigen binding molecule of any one of paras 1 to 10, wherein the second antigen binding domain that specifically binds to LTBR comprises
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:41 and a light chain variable region (V_{L} LTBR) comprising the amino acid sequence of SEQ ID NO:42, or
(iii) a heavy chain variable region (V_{H} LTBR) comprising the amino acid sequence of SEQ ID NO:49 and a light chain variable region (V_{L} LTBR) comprising the amino acid sequence of SEQ ID NO:50.

12. The bispecific antigen binding molecule of any one of paras 1 to 11, comprising
(a) a first antigen binding domain that specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and
(b) a second antigen binding domain that specifically binds to LTBR, comprising a heavy chain variable region (V_{H} LTBR) comprising an amino acid sequence of SEQ ID NO:33 and a light chain variable region (V_{L} LTBR) comprising an amino acid sequence of SEQ ID NO:34.

13. The bispecific antigen binding molecule of any one of paras 1 to 12, wherein the Fc domain composed of a first and a second subunit is an IgG Fc domain, particularly an IgG1 Fc domain or an IgG4 Fc domain and wherein the Fc domain comprises one or more amino acid substitution that reduces the binding affinity of the antibody to an Fc receptor and/or effector function.

14. The bispecific antigen binding molecule of any one of paras 1 to 13, wherein the Fc domain is of human IgG1 subclass with the amino acid mutations L234A, L235A and P329G (numbering according to Kabat EU index).

15. The bispecific antigen binding molecule of any one of paras 1 to 14, comprising
(a) a first Fab fragment that specifically binds to FAP,
(b) a second Fab fragment that specifically binds to LTBR, and
(c) a Fc domain composed of a first and a second subunit and comprising one or more amino acid substitution that reduces the binding affinity of the antigen binding molecule to an Fc receptor and/or effector function.

16. The bispecific antigen binding molecule of para 15, wherein the second Fab fragment that specifically binds to LTBR is a crossFab fragment.

17. The bispecific antigen binding molecule of any one of paras 1 to 14, comprising
(a) a first Fab fragment that specifically binds to FAP,
(b) a second and a third Fab fragment that specifically bind to LTBR, and
(c) a Fc domain composed of a first and a second subunit and comprising one or more amino acid substitution that reduces the binding affinity of the antigen binding molecule to an Fc receptor and/or effector function,
wherein the first Fab fragment that specifically binds to FAP is fused at its N-terminus to the C-terminus of one of the Fc domain subunits and the second and a third Fab fragment that specifically bind to LTBR are each fused at its C-terminus to the N-terminus of one of the Fc domain subunits.

18. An antibody that specifically binds to LTBR, wherein said antibody comprises
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32, or
(ii) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(iii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48, or
(iv) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(v) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(vi) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72, or
(vii) a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80, or
(viii) a heavy chain variable region (V_{H} LTBR) comprising a CDR-H1 comprising the amino acid sequence of SEQ ID NO:83, a CDR-H2 comprising the amino acid sequence of SEQ ID NO:84, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88.

19. The antibody of para 18, wherein said antibody comprises
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:41 and a light chain variable region (V_{L} LTBR) comprising the amino acid sequence of SEQ ID NO:42, or
(iii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(iv) a heavy chain variable region (V_{H} LTBR) comprising the amino acid sequence of SEQ ID NO:57 and a light chain variable region (V_{L} LTBR) comprising the amino acid sequence of SEQ ID NO:58, or
(v) a heavy chain variable region (V_{H} LTBR) comprising the amino acid sequence of SEQ ID NO:65 and a light chain variable region (V_{L} LTBR) comprising the amino acid sequence of SEQ ID NO:66,
(vi) a heavy chain variable region (V_{H} LTBR) comprising the amino acid sequence of SEQ ID NO:73 and a light chain variable region (V_{L} LTBR) comprising the amino acid sequence of SEQ ID NO:74,
(vii) a heavy chain variable region (V_{H} LTBR) comprising the amino acid sequence of SEQ ID NO:81 and a light chain variable region (V_{L} LTBR) comprising the amino acid sequence of SEQ ID NO:82, or
(viii) a heavy chain variable region (V_{H} LTBR) comprising the amino acid sequence of SEQ ID NO:89 and a light chain variable region (V_{L} LTBR) comprising the amino acid sequence of SEQ ID NO:90, or
(ix) a heavy chain variable region (V_{H} LTBR) comprising the amino acid sequence of SEQ ID NO:97 and a light chain variable region (V_{L} LTBR) comprising the amino acid sequence of SEQ ID NO:98.

20. One or more isolated polynucleotide encoding the bispecific antigen binding molecule of any one of paras 1 to 17 or the antibody of paras 18 or 19.

21. An expression vector comprising the one or more isolated polynucleotide of para 20.

22. A prokaryotic or eukaryotic host cell comprising the one or more isolated polynucleotide of para 20 or the expression vector of para 21.

23. A method of producing a bispecific antigen binding molecule, comprising the steps of a) culturing the prokaryotic or eukaryotic host cell of para 22 under conditions suitable for the expression of the bispecific antigen binding molecule and b) optionally recovering the bispecific antigen binding molecule.

24. A pharmaceutical composition comprising the bispecific antigen binding molecule of any one of paras 1 to 17 or the antibody of paras 18 or 19 and a pharmaceutically acceptable excipient.

25. The pharmaceutical composition of para 24, further comprising an additional therapeutic agent.

26. The bispecific antigen binding molecule of any one of paras 1 to 17, or the pharmaceutical composition of para 24, for use as a medicament.

27. The bispecific antigen binding molecule of any one of paras 1 to 17, or the pharmaceutical composition of para 24, for use in (a) inducing ICAM upregulation on endothelial cells or cancer-associated fibroblasts, or (b) enhancing T cell adhesion.

28. The bispecific antigen binding molecule of any one of paras 1 to 17, or the pharmaceutical composition of claim 24, for use in the treatment of cancer.

29. The bispecific antigen binding molecule according to any one of paras 1 to 17 or the pharmaceutical composition according to para 24 for use in the treatment of cancer, wherein the bispecific antigen binding molecule or pharmaceutical composition is for administration in combination with a chemotherapeutic agent, radiation and/or other agents for use in cancer immunotherapy.

30. The bispecific antigen binding molecule according to any one of paras 1 to 17 or the pharmaceutical composition according to para 24 for use in the treatment of cancer, wherein the bispecific antigen binding molecule is for administration in combination with an agent blocking PD-L1/PD-1 interaction.

31. Use of the bispecific antigen binding molecule of any one of paras 1 to 17, or the pharmaceutical composition of para 24, in the manufacture of a medicament for the treatment of cancer.

32. A method of treating an individual having cancer comprising administering to the individual an effective amount of the bispecific antigen binding molecule of any one of paras 1 to 17, or the pharmaceutical composition of para 24.

### EXAMPLES

The following are examples of methods and compositions of the invention. It is understood that various other embodiments may be practiced, given the general description provided above.

### Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

### DNA Sequencing

DNA sequences were determined by double strand sequencing.

### Gene Synthesis

Desired gene segments were either generated by PCR using appropriate templates or were synthesized from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Protein Purification

Proteins were purified from filtered cell culture supernatants referring to standard protocols. In brief, antibodies were applied to a Protein A Sepharose column (MabSelect™ SuRe™, Cytiva) and washed with PBS. Elution of antibodies was achieved at pH 2.8 followed by immediate neutralization of the sample. Aggregated protein was separated from monomeric antibodies by size exclusion chromatography (SEC) (HiLoad® 16/600 Superdex® S200, Cytiva) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, CE-SDS, analytical size exclusion chromatography (SEC) or mass spectrometry.

### SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

### CE-SDS

Purity, antibody integrity and molecular weight of bispecific and control antibodies were analyzed by CE-SDS using microfluidic LabChip® technology (Caliper Life Science, USA). 5 μl of protein solution was prepared for CE-SDS analysis using the HT Protein Express Reagent Kit according manufacturer's instructions and analyzed on a LabChip® GXII Touch™ protein characterization system using a HT Protein Express Chip. Data were analyzed using LabChip® GX Software version 3.0.618.0.

### Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh (e.g. TSKgel® G3000 SWX_{L}) column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

### Example 1

### Generation of Anti-LTBR Antibodies

### 1.1 Proteins Used for Phage Display and Immunization to Generate Anti-LTBR Antibodies

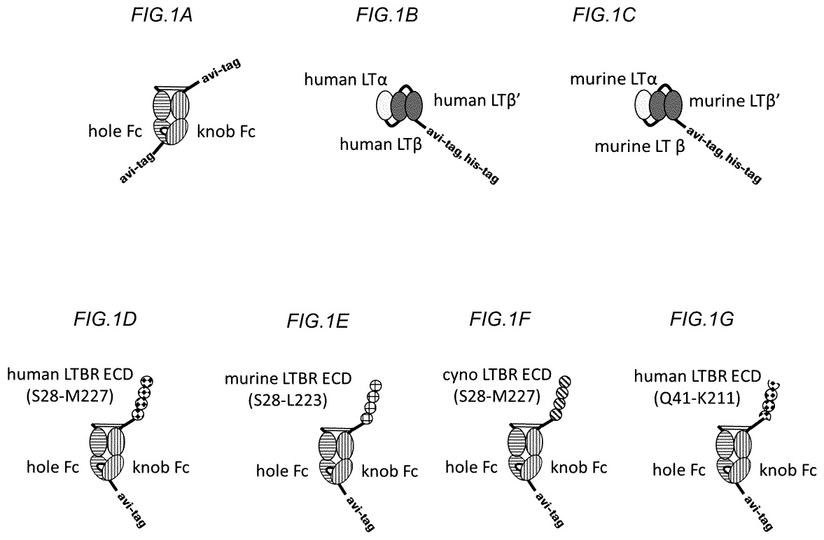

For the generation of anti-LTBR antibodies by phage display and immunization, lymphotoxin R receptors and lymphotoxin α1β2 of different species as well as tool proteins were generated as soluble recombinant proteins. These were biotinylated and Fc-tagged lymphotoxin R receptors used as antigens in phage display and immunogen for immunizations, a biotinylated Fc-fragment (Fc-depleter) used as pre-clearing agent to avoid Fc-binders in phage display, and biotinylated and his-tagged single-chain lymphotoxin α1β2 ligands used for competition during phage display and further characterization. Table 1 provides an overview on these proteins and their respective identifiers, their structures are schematically shown in FIGS. 1A to 1G.

TABLE 1
Recombinant soluble receptors, ligands and tool proteins
used for phage display and immunization

| Identifier | SEQ ID NO. | Recombinant soluble protein |
|---|---|---|
| P1AA0981 | 247, 248 | Fc-depleter kh NC avi |
| | (knob and hole | biotinylated |
| | chain) | |
| P1AE1235 | 249 | human lymphotoxin α1β2 single- |
| | | chain avi his |
| P1AE1236 | 250 | murine lymphotoxin α1β2 single- |
| | | chain avi his |
| P1AE2835 | 251, 252 (knob | monomeric huLTBR ECD (S28- |
| | and hole chain) | M227) Fc-fusion avi biotinylated |
| P1AE4410 | 253, 254 (knob | monomeric murine LTBR ECD |
| | and hole chain) | (S28-L223) |
| | | Fc fusion avi biotinylated |
| P1AE4411 | 255, 256 (knob | monomeric cynomolgus LTBR |
| | and hole chain) | ECD (S28-M227) |
| | | Fc fusion avi biotinylated |
| P1AE1217 | 257, 258 (knob | monomeric N-terminal human |
| | and hole chain) | LTBR (ECD full length) hu IgG1 |
| | | Fc-fusion kih HRYF avi |

All recombinant soluble proteins listed in Table 1 were prepared by evitria using their proprietary vector system with conventional (non-PCR based) cloning techniques and using suspension-adapted CHO K1 cells (originally received from ATCC and adapted to serum-free growth in suspension culture at evitria). For the production, evitria used their proprietary, animal-component free and serum-free media (eviGrow and eviMake2) and their proprietary transfection reagent (eviFect). For in vivo biotinylation, an avi tag (GLNDIFEAQKIEWiE, SEQ ID NO:259) was fused to the C-termini of these constructs (for P1AA0981 also to the N-terminus of the knob heavy chain) that allowed specific biotinylation upon co-expression with BirA biotin ligase. Supernatants were harvested by centrifugation and subsequent filtration (0.2 μm filter) and proteins were purified from the harvested supernatant by standard methods.

Fc-tagged proteins were purified by protein A affinity chromatography (Protein A MabSelect™ SuRe™, Cytiva) and preparative size-exclusion-chromatography (SEC) (HiLoad® 16/600 Superdex® S200, Cytiva) whereas his-tagged proteins were purified by cOmplete His-Tag affinity chromatography followed by preparative size-exclusion-chromatography (SEC) (HiLoad® 26/60 S200), all according to standard procedures and the manufacturers' instructions. Purity of the purified proteins was determined by analytical size-exclusion-chromatography (e.g. TSKgel® G3000 SWX_{L}) and CE-SDS (e.g. Caliper LabChip® GXII).

### 1.2 Additional Proteins Used for Screening and Characterization of Anti-LTBR IgG Antibodies and Anti-FAP/Anti-LTBR Bispecific Antibodies

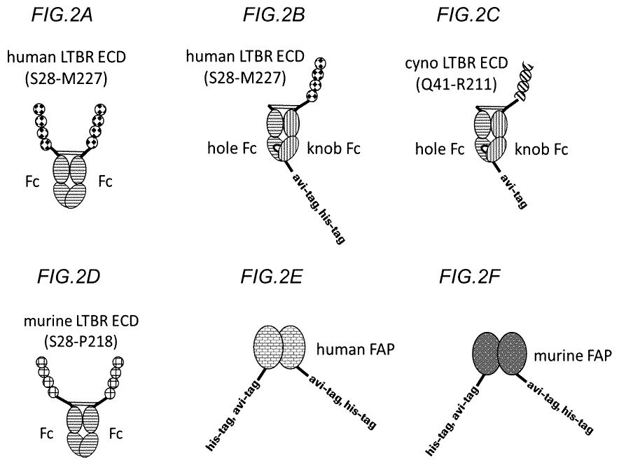

For screening and characterization of the antibodies generated by phage display and immunization, additional recombinant soluble lymphotoxin beta receptors, ligands (human LIGHT) and tumor stroma targets (human and murine FAP) have been expressed and purified or purchased commercially. Furthermore, commercial lymphotoxin β receptors have been purchased and biotinylated in vitro. Table 2 and FIGS. 2A to 2F provide an overview on these proteins and their respective identifiers.

TABLE 2
Additional recombinant soluble receptors,
ligands and tumor stroma targets

| Identifier | SEQ ID NO: | Recombinant soluble protein |
|---|---|---|
| P1AE2401 | — | dimeric human LTBR-Fc |
| | | biotinylated |
| | | (purchased from R&D Systems |
| | | (7538-LR) and subsequently |
| | | biotinylated) |
| P1AE7979 | 260, 261 | human LTBR ECD (S28-M227) |
| | | N- and C-terminally extended— |
| | | Fc-fusion wt kih HRYF—Avi— |
| | | His biotinylated |
| P1AE2656 | 262, 263 | monomeric N-terminal |
| | | cynomolgus LTbR |
| | | (ECD full length) |
| | | hu IgG1 Fc-fusion kih HRYF avi |
| | | biotinylated |
| P1AE2655 | — | dimeric murine LTBR-Fc |
| | | disulfide-linked homodimer |
| | | biotinylated |
| | | (purchased from R&D Systems |
| | | (1008-LR) and subsequently |
| | | biotinylated) |
| — | — | human LIGHT |
| | | (purchased from R&D Systems |
| | | (664-Li) |
| P1AA5347 | 264 | human FAP—Avi—His |
| | | biotinylated |
| P1AD9907 | 265 | murine FAP—Avi—His |
| | | biotinylated |

### 1.3 Generation of Anti-LTBR Fabs (Fragment Antigen-Binding) by Phage Display

### 1.3.1 Selection and Screening of Anti-LTBR Fabs

Anti-LTBR Fabs were selected by phage display from synthetic Fab libraries based on entirely human frameworks with sequence diversity in the CDR3s of the VL (3 different lengths) and VH domains (6 different lengths).

Selection rounds (biopanning) were performed in solution according to the following protocol: 1. pre-clearing of ˜10¹² phagemid particles per library pool on neutravidin-coated 96-well plates coated with 500 nM of an unrelated biotinylated human Fc fragment, 2. incubation of the non human Fc fragment binding phagemid particles in the supernatant with the lymphotoxin ligand and biotinylated LTBR receptor (see Table 3) for 0.5 h in a total volume of 0.8 ml, 3. capture of biotinylated LTBR receptor and specifically binding phage by adding 80 μl of streptavidin-coated magnetic particles for 20 min, 4. washing of respective magnetic particles 5-10× with 0.8 ml PBS/Tween 20 and 5-10× with 0.8 ml PBS using a magnetic particle separator, 5. elution of phage particles by addition of 0.8 ml of 10 mM glycine pH 2 for 5 min followed by 0.8 ml 100 mM triethylamine (TEA) for 5 min and subsequent neutralization by addition of an ½ volume of 1 M Tris/HCl pH 7.4, 6. re-infection of log-phase E. coli TG1 cells with the eluted phage particles, infection with helper phage VCSM13, incubation on a shaker at 30° C. overnight and subsequent PEG/NaCl precipitation of phagemid particles to be used in the next selection round.

Selections were carried out over 4 rounds using decreasing antigen concentrations. Human and murine lymphotoxin ligands and receptors were alternated between the selection rounds to obtain human and mouse LTBR cross-reactive antibodies that do not compete with the natural ligands. Due to the high sequence homology between the ectodomains of human and cynomolgus LTBR, it was assumed to also generate human and cynomolgus LTBR cross-reactive antibodies. Table 3 summarizes which proteins have been used for pre-clearing, ligand competition and selection during the phage display selection campaign for LTBR to obtain non-ligand competing human and murine LTBR-specific antibodies.

TABLE 3
Phage display selection campaign for selection of non-ligand competing
human and murine LTBR-specific antibodies

| selection | | lymphotoxin ligand | LTBR receptor for |
|---|---|---|---|
| round | pre-clearing protein | for competition | selection |
| round 1 | 500 nM biotinylated | 300 nM human | 100 nM monomeric |
| | human Fc-depleter | lymphotoxin α1β2 | human LTBR ECD |
| | kh NC avi B | single-chain avi his | (S28-M227) Fc- |
| | (P1AA0981) | (P1AE1235) | fusion avi |
| | | | biotinylated |
| | | | (P1AE2835) |
| round 2 | 500 nM biotinylated | 300 nM murine | 50 nM monomeric |
| | human Fc-depleter | lymphotoxin α1β2 | murine LTBR ECD |
| | kh NC avi B | single-chain avi his | (S28-L223) Fc- |
| | (P1AA0981) | (P1AE1236) | fusion avi |
| | | | biotinylated |
| | | | (P1AE4410) |
| round 3 | 500 nM biotinylated | 300 nM human | 25 nM monomeric |
| | human Fc-depleter | lymphotoxin α1β2 | human LTBR ECD |
| | kh NC avi B | single-chain avi his | (S28-M227) Fc- |
| | (P1AA0981) | (P1AE1235) | fusion avi |
| | | | biotinylated |
| | | | (P1AE2835) |
| round 4 | 500 nM biotinylated | 300 nM murine | 10 nM monomeric |
| | human Fc-depleter | lymphotoxin α1β2 | murine LTBR ECD |
| | kh NC avi B | single-chain avi his | (S28-L223) Fc- |
| | (P1AA0981) | (P1AE1236) | fusion avi |
| | | | biotinylated |
| | | | (P1AE4410) |

### 1.3.2 Sandwich ELISA to Identify Human and Mouse LTBR Cross-Reactive Fabs Obtained by Phage Display

Individual clones were bacterially expressed as 1 ml cultures in 96-well format and supernatants were subjected to screening by ELISA. Specific binders were defined as having signals higher than 5-times the background signal for monomeric huLTBR LCD (S28-M227) Fc-fusion avi biotinylated (P1AE2835) as well as muLTBR ECD (S28-L223) Fc-fusion avi biotinylated (P1AE4410) and no significant signals for Fc-depleter kh NC avi B (P1AA0981).

More precisely, neutravidin 96 well strip plates (Thermo Fisher) were coated with 100 nM of biotinylated proteins at 37° C. for 30 min, followed by blocking of the plate with 2% MPBS (milk powder phosphate buffered saline) (200 μl/well) for 1 h at room temperature. The plate was washed 3 times with PBS, then Fab containing bacterial supernatants were added and the plate was incubated at room temperature for 1 h. After another 3 washing steps with PBS, anti-FLAG-HRP secondary antibody (1:4000) (Sigma) was added and the plate was incubated on a shaker for 1 h at room temperature. The plate was washed 3 times with PBS and developed by adding 100 μl/well BM Blue POD (Roche). The enzymatic reaction was stopped by adding 50 μl/well 1 M H₂SO₄. The OD was read at 450 nm (reference at 900 nm) for a final read-out of OD₄₅₀₋₉₀₀. ELISA-specific binders were subjected to conversion to IgG format for further characterization by SPR as described below.

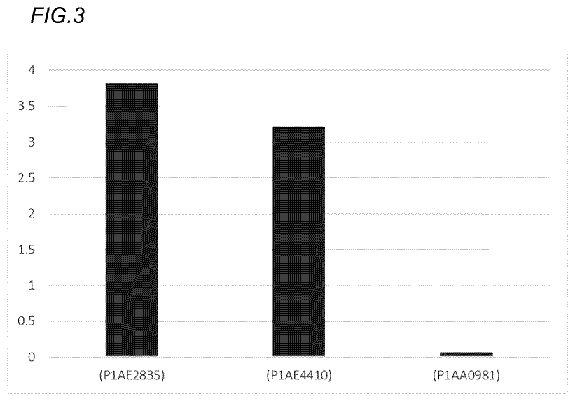

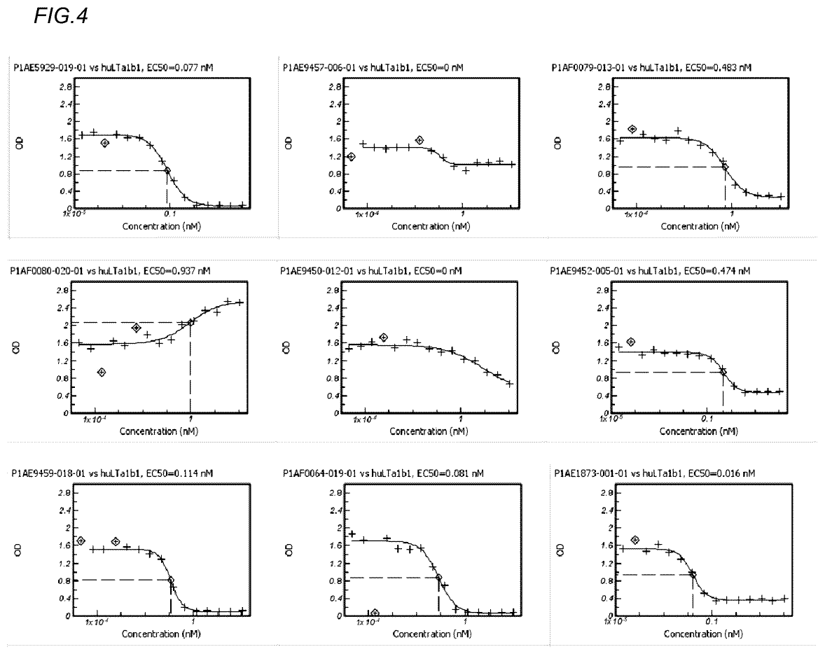

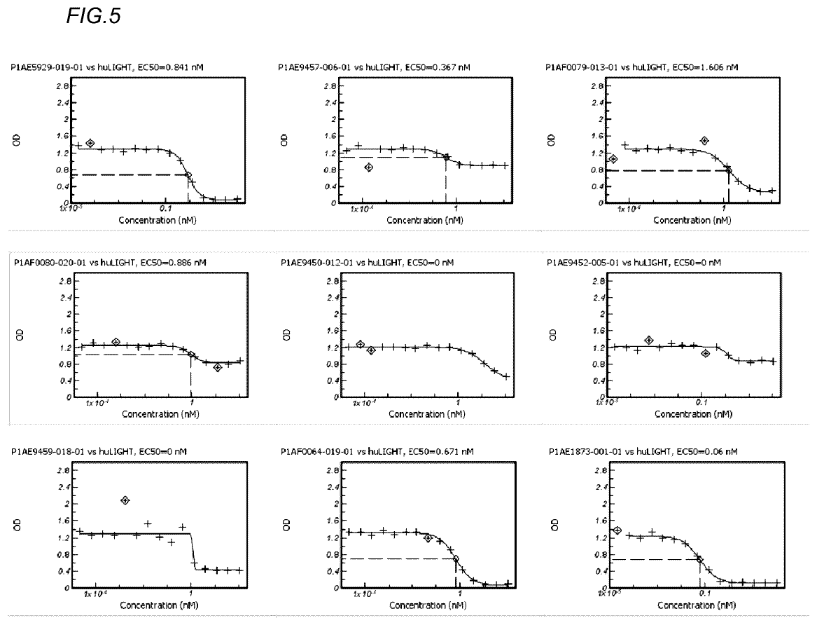

The selected Fab clone FAPltbr.P218.076 fulfilled the criteria of specific binding to human and murine LTBR (FIG. 3). It was subsequently converted into a human IgG1 format for further characterization (P1AE5929 after IgG-conversion). Despite the selection strategy, it does compete with the natural ligands lymphotoxin α1β2 and LIGHT (see Table 5 and FIGS. 4 and 5, respectively). Due to its high affinity of 2 nM it may be possible that this antibody displaced the ligand during the selection.

1.4 Antibody Generation by Rabbit and Rat Immunization with Protein

For immunization, CD rats obtained from Charles River Laboratories International, Inc. as well as Roche proprietary transgenic rabbits comprising a human immunoglobulin locus as reported in WO 2000/46251, WO 2002/12437, WO 2005/007696, WO 2006/047367, US 2007/0033661, and WO 2008/027986 were used. The animals were housed according to the Appendix A “Guidelines for accommodation and care of animals” in an AAALAC-accredited animal facility. All animal immunization protocols and experiments were approved by the Government of Upper Bavaria (permit number 55.2-1-54-2531-66-16 and 55.2-1-54-2532-90-14) and performed according to the German Animal Welfare Act and the Directive 2010/63 of the European Parliament and Council.

Recombinant monomeric N-terminal human LTBR (ECD full length) hu IgG1 Fc-fusion kih HRYF avi (P1AE1217) was used for the immunization of 3 transgenic rabbits. Each rabbit was initially immunized intradermally with 400 μg of the protein, followed by alternating intramuscular and subcutaneous injections of 200 μg of the protein at days 7, 14, 42, 70 and 98. A mixture of TLR agonists was used as adjuvant for each immunization. Blood samples were taken after the third, fourth, fifth and sixth immunization (at 5-7 days post immunization) and used as a source of antigen-specific B-cells. Antigen-specific titers were monitored during the immunization period by ELISA analysis of serum samples.

CD rats (n=4) were immunized with the same immunogen, i.e. recombinant monomeric N-terminal human LTBR (ECD full length) hu IgG1 Fc-fusion kih HRYF avi (P1AE1217). For the initial immunization, 40 μg of the immunogen was emulsified with Incomplete Freund's adjuvant (IFA) and a TLR agonist and one half of the mixture was injected subcutaneously (distributed over several injection sites), while the other half was administered intraperitoneally. After six weeks, a booster immunization was given in the same manner except for the use of IFA, which was substituted by PBS. Four days after the second immunization, blood was drawn and used as a source of antigen-specific B-cells.

### 1.4.1 Isolation of Peripheral Blood Mononuclear Cells (PBMCs)

EDTA containing whole blood was diluted two-fold with 1×PBS (Pan Biotech, Aidenbach, Germany) before density centrifugation using lympholyte mammal (Cedarlane Laboratories, Burlington, Ontario, Canada) according to the specifications of the manufacturer. The PBMCs were washed twice with 1×PBS.

### 1.4.2 Rabbit B-Cell Cloning Procedure

Depletion of cells: Sterile 6-well plates (cell culture grade) covered with a confluent monolayer of CHO cells or uncovered, were used to deplete non-specifically binding lymphocytes as well as macrophages/monocytes through unspecific adhesion to the plastic. Each well was filled at maximum with 4 mL medium and up to 6×10⁶ PBMCs and allowed to bind for 1 h at 37° C. in the incubator. The cells in the supernatant (peripheral blood lymphocytes (PBLs)) were used for the antigen panning step.

Enrichment of B-cells on human and cynomolgus LTBR: 6-well tissue culture plates covered with a monolayer of human or cynomolgus LTBR-positive CHO cells or coated with recombinant human LTBR-Fc fusion protein were seeded with up to 6×10⁶ PBLs per 4 mL medium and allowed to bind for 1 h at 37° C. in the incubator. Non-adherent cells were removed by carefully washing the wells 1-2 times with 1×PBS. The remaining sticky cells were detached by trypsine for 10 min. at 37° C. in the incubator. Trypsination was stopped with EL-4 B5 medium. The cells were kept on ice until the immune fluorescence staining.

Immune fluorescence staining and flow cytometry: The anti-IgG FITC (Abcam, Cambridge, UK) was used for single cell sorting. For surface staining, cells from the depletion and enrichment step were incubated with the anti-IgG FITC antibody in PBS and incubated for 45 min. in the dark at 4° C. After staining, the PBMCs were washed two-fold with ice-cold PBS. Finally, the PBMCs were resuspended in ice-cold PBS and immediately subjected to the FACS analyses. Propidium iodide in a concentration of 5 μg/mL (BD Pharmingen, San Diego, CA, USA) was added prior to the FACS analyses to discriminate between dead and live cells. A Becton Dickinson FACSAria equipped with a computer and the FACSDiva software (BD Biosciences, USA) was used for single cell sort.

B-cell cultivation: The cultivation of the B-cells was prepared by a method similar to that described by Lightwood et al. (J Immunol Methods, 2006, 316: 133-143). Briefly, single sorted rabbit B-cells were incubated in 96-well plates with 200 μL/well EL-4 B5 medium containing Pansorbin Cells (1:100000) (Calbiochem (Merck), Darmstadt, Deutschland), a cytokine mix (Miltenyi, Bergisch Gladbach, Germany) combined with PMA (Sigma, Darmstadt, Germany) according to WO/2018/122147 and gamma-irradiated murine EL-4-B5 thymoma cells (5×10⁴/well) for 7 days at 37° C. in an atmosphere of 5% CO₂ in the incubator. The supernatants of the B-cell cultivation were removed for screening and the remaining cells were harvested immediately and were frozen at −80° C. in 100 μL RLT buffer (Qiagen, Hilden, Germany).

EL-4 B5 medium consists of RPMI 1640 (Pan Biotech, Aidenbach, Germany) supplemented with 10% FCS, 10 mM HEPES (PAN Biotech, Aidenbach, Germany), 2 mM glutamine, 1% penicillin/streptomycin solution (PAA, Pasching, Austria), 2 mM sodium pyruvate and 0.05 mM β-mercaptoethanol (Gibco, Paisley, Scotland).

PCR amplification of rabbit V-domains: Total RNA was prepared from B-cells lysate (resuspended in RLT buffer—Qiagen—Cat. N° 79216) using the NucleoSpin 8/96 RNA kit (Macherey&Nagel; 740709.4, 740698) according to manufacturer's protocol. RNA was eluted with 60 μL RNAse free water. 6 μL of RNA was used to generate cDNA by reverse transcriptase reaction using the Superscript III First-Strand Synthesis SuperMix (Invitrogen 18080-400) and an oligo-dT-primer according to the manufacturer's instructions. All steps were performed on a Hamilton ML Star System. 4 μL of cDNA were used to amplify the immunoglobulin heavy and light chain variable regions (VH and VL) with the AccuPrime Supermix (Invitrogen 12344-040) in a final volume of 50 μL using the primers rbHC.up (SEQ ID NO:317), HUJH5.HFc-DO3 (SEQ ID NO:267) and HUJH6.HFc-DO3 (SEQ ID NO:268) for the heavy chain and BcPCR_FiLC_leader.fw (TG) (SEQ ID NO:269) and HuCK.Do.2AA (SEQ ID NO: for the light chain of transgenic rabbit B-cells. All forward primers were specific for the signal peptide (of VH and VL, respectively) whereas the reverse primers were specific for the framework or constant regions (of VH and VL, respectively). The PCR conditions for the VH and VL were as follows: Hot start at 94° C. for 5 min.; 35 cycles of 20 sec. at 94° C., 20 sec. at 70° C., 45 sec. at 68° C., and a final extension at 68° C. for 7 min. 8 μL of 50 μL PCR solution were loaded on a 48 E-Gel 2% (Invitrogen G8008-02). Positive PCR reactions were cleaned using the NucleoSpin Extract II kit (Macherey&Nagel; 740609250) according to manufacturer's protocol and eluted with 75 μL elution buffer. All cleaning steps were performed on a Hamilton ML Starlet System.

### 1.4.3 Rat B-Cell Cloning Procedure

Depletion of cells: In a first step, sterile 6-well plates (cell culture grade) were used to deplete non-specifically binding lymphocytes as well as macrophages/monocytes through unspecific adhesion to the plastic. Each well was filled at maximum with 4 mL medium and up to 6×10⁶ PBMCs and allowed to bind for 1 h at 37° C. in the incubator. The cells in the supernatant (peripheral blood lymphocytes (PBLs)) were used for a second depletion step. For this, 6-well plates covered with anti-rat CD4 (BD Biosciences, San Jose, US) and anti-rat IgM (Biorad, Hercules, US) were used to deplete adhering cells as described above.

Immune fluorescence staining and flow cytometry: For surface staining, the cells from the depletion steps were incubated with the anti-rat IgG1, anti-rat IgG2a, anti-rat IgG2b (all FITC), anti-kappa light chain (PE), and anti-rat CD8a (Alexa Fluor 647) antibodies (all BD Bioscience, San Jose, US) in PBS and incubated for 45 min. in the dark at 4° C. Subsequently, the PBMCs were washed twice with ice-cold PBS. Finally the PBMCs were resuspended in ice-cold PBS and immediately subjected to the FACS analyses. Propidium iodide in a concentration of 5 μg/mL (BD Pharmingen, San Diego, CA, USA) was added prior to the FACS analyses to discriminate between dead and live cells. After negative gating of CD8a⁺ cells, double positive cells for IgG and kappa light chain were single cell sorted. A Becton Dickinson FACSAria equipped with a computer and the FACSDiva software (BD Biosciences, USA) was used for sorting.

B-cell cultivation: The cultivation of the B-cells was prepared by a method similar to that described by Lightwood et al. (J Immunol Methods, 2006, 316: 133-143). Briefly, single sorted rat B-cells were incubated in 96-well plates with 200 μL/well EL-4 B5 medium containing Pansorbin Cells (1:100000) (Calbiochem (Merck), Darmstadt, Deutschland), a cytokine mix combined with PMA according to WO 2018/122147 in adapted concentrations and gamma-irradiated murine EL-4-B5 thymoma cells (5×10⁴/well) for 12 days at 37° C. in an atmosphere of 5% CO₂ in the incubator. The supernatants of the B-cell cultivation were removed for screening and the remaining cells were harvested immediately and were frozen at −80° C. in 100 μL RLT buffer (Qiagen, Hilden, Germany).

PCR amplification of rat V-domains: Total RNA was prepared from B-cells lysate (resuspended in RLT buffer—Qiagen—Cat. N° 79216) using the NucleoSpin 8/96 RNA kit (Macherey&Nagel; 740709.4, 740698) according to manufacturer's protocol. RNA was eluted with 60 μL RNAse free water. 6 μL of RNA was used to generate cDNA by reverse transcriptase reaction using the Superscript III First-Strand Synthesis SuperMix (Invitrogen 18080-400) and an oligo-dT-primer according to the manufacturer's instructions. All steps were performed on a Hamilton ML Star System. 4 μL of cDNA were used to amplify the immunoglobulin heavy and light chain variable regions (VH and VL) with the AccuPrime Supermix (Invitrogen 12344-040) in a final volume of 50 μL using a mix of forward primers consisting of WTRat.VH.FW11 to WTRat.VH.FW17 (SEQ ID Nos:271 to 277) with 682.rev do (SEQ ID NO:278) as a reverse primer to amplify the VH-domains and WTRat.VK.FW1 (SEQ ID NO:279) and WTRat.VK.FW2 (SEQ ID NO:280) with WTRat.CKappa.Do2 (SEQ ID NO:281) as a reverse primer to amplify the VL-domains. The PCR conditions for the amplification of the VH-domains were as follows: Hot start at 94° C. for 4 min.; 35 cycles of 20 sec. at 94° C., 20 sec. at 57° C., 45 sec. at 68° C., and a final extension at 68° C. for 5 min. The PCR conditions for the amplification of the VL-domains were as follows: Hot start at 94° C. for 4 min.; 35 cycles of 20 sec. at 94° C., 20 sec. at 56° C., 45 sec. at 68° C., and a final extension at 68° C. for 5 min. Positive PCR reactions were cleaned using the NucleoSpin Extract II kit (Macherey&Nagel; 740609250) according to manufacturer's protocol and eluted with 75 μL elution buffer.

### 1.4.4 Expression of Monoclonal LTBR Antibodies

Generation of recombinant vectors for the expression of monoclonal antibodies: For recombinant expression of rabbit monoclonal bivalent antibodies, PCR-products coding for VH or VL were cloned as cDNA into expression vectors by the overhang cloning method (RS Haun et al., Biotechniques (1992) 13, 515-518; M Z Li et al., Nature Methods (2007) 4, 251-256). The expression vectors contained an expression cassette consisting of a 5′ CMV promoter including Intron A, and a 3′ BGH polyadenylation sequence. In addition to the expression cassette, the plasmids contained a pUC18-derived origin of replication and a beta-lactamase gene conferring ampicillin resistance for plasmid amplification in E. coli. Three variants of the basic plasmid were used: One plasmid containing the rabbit IgG constant region designed to accept the VH region from DNA immunized rabbits, one plasmid containing the human IgG constant region with a PG LALA mutation designed to accept the VH regions from protein immunized rabbits and one plasmid containing human kappa LC constant region to accept the VL regions.

Linearized expression plasmids coding for the kappa or gamma constant region and VL/VH inserts were amplified by PCR using overlapping primers. Purified PCR products were incubated with T4 DNA-polymerase which generated single-strand overhangs. The reaction was stopped by dCTP addition. In a next step, plasmid and insert were combined and incubated with recA which induced site specific recombination. The recombined plasmids were transformed into E. coli. The next day, the grown colonies were picked and tested for correct recombined plasmid by plasmid preparation and DNA-sequencing. In contrast to the plasmids encoding the rabbit antibody sequences, the genes for the rat antibody sequences have been synthesized at TWIST Bioscience.

For antibody expression, the isolated HC and LC plasmids were transiently co-transfected into HEK293 cells and the supernatants were harvested after 1 week.

Transient expression of IgGs: The antibodies were produced in transiently transfected Expi293F cells (human embryonic kidney cell line 293-derived) cultivated in Expi293 Medium (Invitrogen Corp.). For transfection, lipid-based ExpiFectamine 293 transfection Reagent (Invitrogen Corp) was used. Antibodies were expressed from individual expression plasmids for the IgG light and heavy chains. Transfections were performed as specified in the manufacturer's instructions. Recombinant protein-containing cell culture supernatants were harvested six days after transfection. Supernatants were stored at reduced temperature (e.g. −80° C.) until purification. General information regarding the recombinant expression of human immunoglobulins in e.g. HEK293 cells is given in: Meissner, P. et al., Biotechnol. Bioeng. 75 (2001) 197-203. The IgGs resulting from rabbit immunization that were selected for further characterization were P1AE9452, P1AE9450, P1AE9459, P1AF0080, P1AF0079, and P1AE9457, as well as P1AE5929 from Phage Display. The IgG resulting from rat immunization that was selected for further characterization was P1AF0064.

### 1.5 Binding of IgG Antibodies to Human, Cynomolgus, and Murine LTBR as Measured by ELISA

Nunc streptavidin coated plates (MicroCoat #11974998001) were coated with 25 μl/well biotinylated human, murine, or cynomolgous LTBR extracellular domain (P1AE2401, P1AE2655, and P1AE2656, respectively) fused to a human IgG1 Fc-Fragment at a concentration of 125 ng/ml and incubated overnight at 4° C. After washing 3×90 μl/well with PBST-buffer (10×PBS, Roche #11666789001+0.1% Tween 20), 25 μl anti-LTBR antibodies were added in 1:3 dilutions starting at a concentration of 15 μg/ml and incubated 1 h at RT. After washing (3×90 μl/well with PBST-buffer), 25 μl/well anti hu kappa POD (Millipore, AP502P, 1:2000) was added and incubated at RT for 1 h. After washing (6×90 μl/well with PBST-buffer), 25 μl/well TMB substrate (Roche, 11835033001) were added. Measurement was performed at 370/492 nm.

Bivalent binding of anti-LTBR IgGs to the extracellular domain of human, cynomolgus and murine LTBR was tested by direct ELISA. All IgGs show strong binding to human LTBR with an EC₅₀ in the range of 3 pM-3 nM. Binding to cynomolgus LTBR is comparable for most of the IgGs. Exceptions are P1AE9452 (˜2-fold stronger binding to cynomolgus LTBR), P1AF0079 (˜1.7-fold stronger binding to cynomolgus LTBR) and no binding to cynomolgus LTBR was observed for P1AE1873, i.e. CBE11. Binding to mouse LTBR is detected for P1AE9459, P1AF0080, P1AF0064 and P1AE5929, all with comparable binding to human LTBR.

Table 4 summarizes the ELISA binding of anti-LTBR IgGs to human, cynomolgus and mouse LTBR. In contrast to P1AE1873 (CBE11), all IgGs are cross-reactive to cynomolgus LTBR and a subset also cross-reacts with murine LTBR.

TABLE 4
Binding of anti-LTBR IgGs the human,
cynomolgus and mouse LTBR.

| | human LTBR | | cynomolgus LTBR | | murine LTBR | |
|---|---|---|---|---|---|---|
| | ECD | | ECD | | ECD | |
| | rel. | max. | rel. | max. | rel. | max. |
| | EC₅₀ | binding | EC₅₀ | binding | EC₅₀ | binding |
| sample ID | (nM) | (OD) | (nM) | (OD) | (nM) | (OD) |
| P1AE9452¹ | 0.238 | 3.2998 | 0.1299 | 3.4837 | | 0.3956 |
| P1AE9450² | 3.8008 | 2.7733 | 4.0175 | 3.1757 | | 0.2347 |
| P1AE9459³ | 0.0600 | 3.0003 | 0.0367 | 3.5118 | 0.0497 | 2.9969 |
| P1AF0080⁴ | 0.0885 | 2.8776 | 0.0937 | 3.2141 | 0.0715 | 3.0746 |
| P1AF0064 | 0.0620 | 3.0795 | 0.0339 | 3.3973 | 0.0649 | 3.0542 |
| P1AF0079 | 0.1396 | 2.9868 | 0.3084 | 2.9696 | | 0.0971 |
| P1AE9457 | 0.1345 | 2.7515 | 0.1397 | 3.234 | | 0.167 |
| P1AE5929 | 0.0864 | 3.1347 | 0.0624 | 3.7559 | 0.1005 | 2.8561 |
| P1AE1873 | 0.0028 | 3.2104 | | 0.1258 | | 0.1118 |

¹⁻⁴indicate LTBR antibodies that are part of bispecific antibodies in Table 12.

1.6 ELISA Measuring Ligand Competition for Anti-LTBR IgGs with Human Lymphotoxin α1β2 and Human LIGHT

Nunc streptavidin coated plates (MicroCoat #11974998001) were coated with 25 μl/well biotinylated human LTBR extracellular domain (P1AE2401) fused to a human IgG1 Fc-fragment at a concentration of 125 ng/ml (500 ng/ml for LIGHT interaction) overnight at 4° C. After washing 3×90 μl/well with PBST-buffer (10×PBS, Roche #11666789001+0,1% Tween 20), 25 μl anti-LTBR antibodies were added in 1:3 dilutions starting at a concentration of 15 μg/ml and incubated 1 h at RT. After washing 3×90 μl/well with PBST-buffer (10×PBS, Roche #11666789001+0.1% Tween 20), 25 μl ligand (either human lymphotoxin α1β2 (P1AE1235) or His6-tagged human LIGHT (R&D systems, 664-Li) were added at a concentration of 250 ng/ml and 1000 ng/ml for LIGHT and incubated 1 h at RT. After washing (3×90 μl/well with PBST-buffer), 25 μl/well anti His6-POD (Bethyl #A190-114P, 1:10000) was added and incubated at RT for 1 h. After washing (6×90 μl/well with PBST-buffer) 25 μl/well TMB substrate (Roche, 11835033001) was added. Measurement was performed at 370/492 nm.

Inhibition of the protein-protein interaction between human LTBR and the human ligands lymphotoxin α1β2 and LIGHT was tested in a multistep ELISA assay. The following anti-LTBR IgGs show a strong inhibition of the LTBR-lymphotoxin α1β2 interaction with IC₅₀ values between ˜20 pM and ˜0.9 nM: P1AE9452, P1AE9459, P1AF0064, P1AF0079, P1AE5929 and P1AE1873. P1AE9450 and P1AE9457 show detectable but weaker inhibition of the interaction (no IC₅₀ calculated, see FIG. 4). P1AF0080 shows increase of the assay signal with increasing concentrations, indicating a stabilization of the LTBR-lymphotoxin α1β2 interaction. Table 5 lists the relative IC₅₀ values (nM) as well as top and bottom ODs.

TABLE 5
Inhibition of LTBR-lymphotoxin α1β2 and
LTBR-LIGHT interactions by anti-LTBR IgGs

| | human lymphotoxin α1β2 interaction | | | human LIGHT interaction | | |
|---|---|---|---|---|---|---|
| | rel. | rel. fit | rel. fit | rel. | rel. fit | rel. fit |
| | IC₅₀ | top | bottom | IC₅₀ | top | bottom |
| sample ID | (nM) | (OD) | (OD) | (nM) | (OD) | (OD) |
| P1AE9452¹ | 0.4741 | 1.4 | 0.5 | | 1.2 | 0.9 |
| P1AE9450² | | | 0.5 | | | 0.4 |
| P1AE9459³ | 0.1145 | 1.5 | 0.1 | | 1.3 | 0.4 |
| P1AF0080⁴ | 0.9371 | 2.56 | 1.6 | 0.8857 | 1.2 | 0.8 |
| P1AF0064 | 0.0815 | 1.7 | 0.1 | 0.6711 | 1.3 | 0.1 |
| P1AF0079 | 0.4828 | 1.6 | 0.3 | 1.6063 | 1.3 | 0.3 |
| P1AE9457 | | 1.4 | 1.0 | 0.3672 | 1.30 | 0.9 |
| P1AE5929 | 0.0770 | 1.7 | 0.1 | 0.8415 | 1.3 | 0.1 |
| P1AE1873 | 0.0160 | 1.5 | 0.4 | 0.0597 | 1.2 | 0.1 |

¹⁻⁴indicate LTBR antibodies that are part of bispecific antibodies in Table 13.

The LTBR-LIGHT interaction was tested in a similar multistep ELISA assay. All tested anti-LTbR IgGs show detectable inhibition of the interaction, in general with higher IC₅₀ values compared to the inhibition of the LTBR-lymphotoxin α1β2 with the same compound (see FIG. 5). P1AF0080 shows detectable inhibition of the LTBR-LIGHT interaction in contrast to the LTBR-lymphotoxin α1β2 interaction, which the compound seems to stabilize.

### 1.7 HeLa NFκB Luc Reporter Assay

In order to identify agonistic anti-LTBR antibodies, HeLa NFκB reporter cells were used to detect NFκB activation, which is downstream of many receptors, including TNFR and LTBR, both expressed endogenously by HeLa cells. The HeLa NFκB Luciferase reporter cells were grown in DMEM Gibco 42430+1% Glutamax+10% FBS+1% P/S+100 ug/mL hygromycin B. They were incubated with anti-LTBR IgG antibodies in a titration series starting at 15 μg/ml for 6 h and the luciferase detection kit (ONE-glow, Promega, E6110) was used according to the manufacturer's instructions. Luminescence was detected as a measure of NFκB activity using a Spectra Max reader (Molecular Devices). As positive control, the CBE11 IgG antibody (P1AE1873) was used. Antibodies were tested in absence or presence of an anti-human Fc cross-linking antibody (Fab goat anti-human Fc (Jackson immunoresearch 109-006-008)) used at 88 μg/ml fixed concentration.

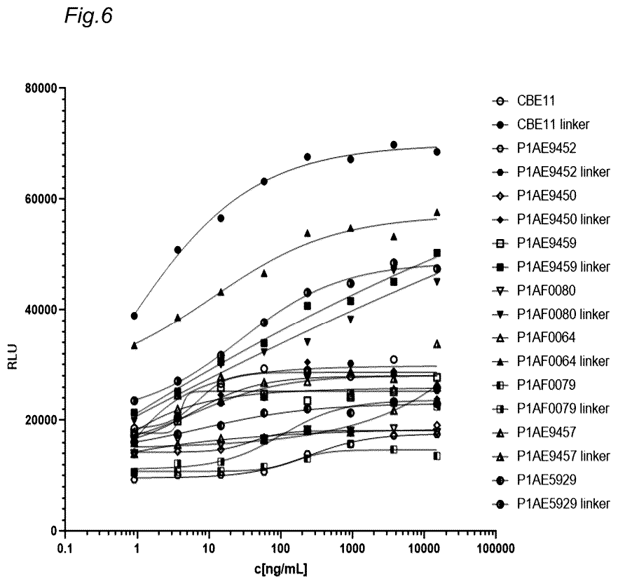

In detail, 25,000 cells were seeded per well in a 96-well plate, flat bottom, tissue culture treated, in complete HeLa medium on day 1. On day 2, the medium was removed and the anti-LTBR IgG antibodies (+/−-cross-linking antibody) were added in 100 ul complete media and incubated for 6 h. The plates were removed from the incubator and equilibrated at RT, as was done for the ONE-glow reagent. 100 μl/well ONE-glow reagent were added and test solutions were transferred to a white plate before reading luminescence using a Spectra Max plate reader (Molecular Devices). The results have been plotted in FIG. 6 and the data are summarized in table in Table 6.

TABLE 6
HeLa NFκB luc reporter assay data for anti-LTBR IgGs with and without cross-linking

| C [ng/ml] | 15000 | 3750 | 937.5 | 234.4 | 58.59 | 14.64 | 3.66 | 0.91 |
|---|---|---|---|---|---|---|---|---|
| CBE11 w/o × linker | 25901 | 30977 | 27885 | 29034 | 29317 | 26626 | 20642 | 18570 |
| CBE11 with × | 68553 | 69859 | 67201 | 67656 | 63207 | 56572 | 50895 | 38962 |
| linker | | | | | | | | |
| P1AE9452 | 17504 | 17252 | 15683 | 13938 | 10694 | 10179 | 10083 | 9332 |
| w/o × linker | | | | | | | | |
| P1AE9452 | 25358 | 28296 | 30272 | 28120 | 24179 | 23085 | 20849 | 16928 |
| with × linker | | | | | | | | |
| P1AE9450 | 19101 | 17297 | 17911 | 18126 | 16888 | 14741 | 14400 | 14135 |
| w/o × linker | | | | | | | | |
| P1AE9450 | 23835 | 28860 | 30148 | 30492 | 24133 | 24593 | 20089 | 16643 |
| with × linker | | | | | | | | |
| P1AE9459 | 27720 | 25156 | 24148 | 23522 | 24689 | 25919 | 19887 | 17664 |
| w/o × linker | | | | | | | | |
| P1AE9459 | 50329 | 45112 | 41638 | 40741 | 33930 | 30540 | 24921 | 21385 |
| with × linker | | | | | | | | |
| P1AF0080 | 17813 | 18480 | 18060 | 18288 | 16663 | 15484 | 16580 | 14018 |
| w/o × linker | | | | | | | | |
| P1AF0080 | 45046 | 47133 | 38220 | 34097 | 32263 | 30047 | 25184 | 19864 |
| with × linker | | | | | | | | |
| P1AF0064 | 25975 | 25543 | 24517 | 26942 | 24355 | 23562 | 22036 | 17825 |
| w/o × linker | | | | | | | | |
| P1AF0064 | 57689 | 53292 | 54818 | 53932 | 46693 | 43298 | 38697 | 33528 |
| with × linker | | | | | | | | |
| P1AF0079 | 13574 | 14751 | 15766 | 13106 | 11598 | 10825 | 10722 | 10671 |
| w/o × linker | | | | | | | | |
| P1AF0079 | 22486 | 23125 | 24863 | 18169 | 16378 | 12515 | 12210 | 10613 |
| with × linker | | | | | | | | |
| P1AE9457 | 15640 | 15286 | 14923 | 14796 | 14054 | 13207 | 12283 | 13195 |
| w/o × linker | | | | | | | | |
| P1AE9457 | 21506 | 23039 | 25444 | 21857 | 20872 | 17446 | 15264 | 13710 |
| with × linker | | | | | | | | |
| P1AE5929 | 23006 | 23358 | 21339 | 22069 | 21342 | 19073 | 17525 | 16105 |
| w/o × linker | | | | | | | | |
| P1AE5929 | 47441 | 48500 | 44811 | 43164 | 37697 | 31712 | 27081 | 23537 |
| with × linker | | | | | | | | |

### 1.8 Cellular Binding (FACS on Recombinant Human LTBR CHO Cells)

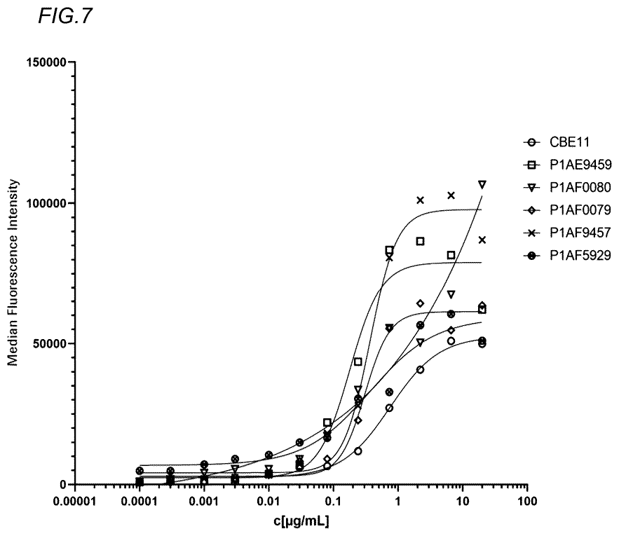

Binding to cell surface human LTBR was determined by FACS analysis on recombinant human LTBR CHO cells at 4° C. Cells were detached from cell culture flasks using accutase treatment and centrifuged at 500 g for 5 min followed by resuspension in cell culture media. Cells were then transferred to U-bottom PP plates and incubated with anti-LTBR IgG antibodies for 1 h at 4° C. in 100 BSA/PBS. Following additional washes, cell-bound anti-LTBR antibodies were detected using anti human IgG polyclonal antibodies coupled to Alexa Fluor 488 (F(ab′)2-Goat anti-Human IgG Fc secondary antibody, Alexa Fluor 488, Cat No. 10120, Life Technologies). Cell bound fluorescence was measured in a BD FACSCanto™ 11 Cell Analyzer using the appropriate filter settings. The results are plotted in FIG. 7 as median fluorescence values and the data are summarized in Table 7. Anti-LTBR IgGs were used in a titration with 3-fold dilution steps starting at 20 μg/ml.

TABLE 7
Assessment of cell surface LTBR binding for anti-
LTBR IgGs (plotting median fluorescence intensity)

| C [μg/ml] | CBE11 | P1AE9459 | P1AF0080 | P1AF0079 | P1AE9457 | P1AE5929 |
|---|---|---|---|---|---|---|
| 20 | 49925 | 62004 | 106578 | 63573 | 87016 | 51024 |
| 6.6 | 50905 | 81630 | 67392 | 54718 | 102798 | 60490 |
| 2.2 | 40762 | 86534 | 50343 | 64284 | 101098 | 56505 |
| 0.73 | 27172 | 83465 | 55483 | 55329 | 80728 | 32800 |
| 0.24 | 11809 | 43572 | 33559 | 22809 | 27937 | 30450 |
| 0.08 | 6517 | 22000 | 17181 | 9044 | 18163 | 16535 |
| 0.03 | 6233 | 6756 | 8969 | 8275 | 7162 | 14926 |
| 0.01 | 3964 | 3749 | 5425 | 3383 | 3383 | 10483 |
| 0.003 | 2345 | 1319 | 5440 | 1503 | 2444 | 9034 |
| 0.001 | 1025 | 1323 | 4064 | 853 | 2075 | 7095 |
| 0.0003 | 1323 | 1334 | 1816 | 689 | 1145 | 4811 |
| 0.0001 | 1086 | 841 | 1013 | 710 | 1262 | 4821 |
| EC50 | 0.682 | 0.147 | 0.672 | 0.321 | 0.336 | 0.206 |
| [μg/ml] | | | | | | |

### 1.9 Epitope Binning of Anti-LTBR IgG Antibodies

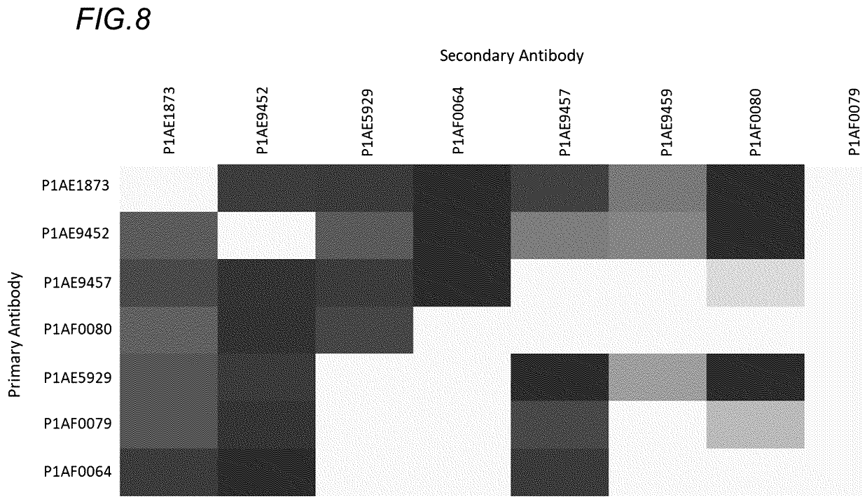

Epitope bins of the anti-LTBR IgG antibodies were characterized by competitive binding analysis using biolayer interferometry (BLI) on an Octet system (Molecular Devices LLC, USA). Briefly, monomeric human LTBR ECD (S28-M227) Fc-fusion avi biotinylated (P1AE2835) was captured on streptavidin sensors at a concentration of 1 μg/ml in HBSP buffer. In a first step, direct binding of the antibodies to the captured antigen was measured and the association signal for each antibody determined (“association direct binding”). Only stable binders with low dissociation rates were selected as primary antibodies. In a second step, competitive binding of the anti-LTBR IgG antibodies against the same set of ‘primary’ anti-LTBR IgG antibodies was analyzed. In brief, biotinylated human LTBR ECD at a concentration of 1 μg/ml in HBSP buffer was captured on streptavidin sensors followed by short dip washing. The primary antibody was bound to the antigen at a concentration of 10 μg/ml in HBSP buffer in order to saturate and block its binding epitope (“association antibody 1”). In a subsequent step, binding of the second antibody (10 μg/ml, in HBSP buffer) to the antigen/primary antibody complex was analyzed (“association antibody 2”). A relative binding value for binding of the second antibody was obtained by dividing the binding signal (report point at 120 sec from “association antibody 2” step) of the second antibody by the binding signal (report point at 120 sec from “association direct binding” step) from the direct binding. The evaluated antibodies were grouped into various epitope bins using the relative binding values and an in-house clustering tool that utilizes various clustering algorithms (e.g. UPGMA, WPGMA or Ward's method). The resulting epitope binning heat map is depicted in FIG. 8 with the respective binding values in percentage shown in Table 8. The data for 8 selected anti-LTBR IgGs subjected to epitope binning resulted in the assignment to four different epitope bins 1, 3, 4 and 5 (see Table 9). P1AE9452 falls into the same epitope bin 1 as CBE11 (P1AE1873).

TABLE 8
Binding of secondary antibodies in percentage as depicted in
the epitope binning matrix of the anti-LTBR IgG antibodies

| primary | secondary antibody | | | | | | | |
|---|---|---|---|---|---|---|---|---|
| antibody | P1AE1873 | P1AE9452 | P1AE5929 | P1AF0064 | P1AE9457 | P1AE9459 | P1AF0080 | P1AF0079 |
| P1AE1873 | 2.13 | 93.02 | 94.15 | 100.00 | 91.19 | 71.29 | 100.00 | 6.47 |
| P1AE9452 | 81.52 | 4.28 | 82.05 | 99.93 | 69.73 | 67.30 | 100.00 | 6.41 |
| P1AE9457 | 88.15 | 96.74 | 93.33 | 100.00 | 5.80 | 5.86 | 35.63 | 5.57 |
| P1AF0080 | 79.63 | 97.07 | 89.55 | 2.25 | 1.87 | 1.83 | 14.66 | 1.22 |
| P1AE5929 | 83.96 | 93.77 | 2.70 | 3.13 | 100.00 | 58.06 | 100.00 | 3.72 |
| P1AF0079 | 83.78 | 96.42 | 7.72 | 8.15 | 88.26 | 8.76 | 47.53 | 3.64 |
| P1AF0064 | 93.72 | 100.00 | 3.88 | 2.44 | 94.12 | 2.83 | 15.91 | 3.00 |

TABLE 9
Epitope bins of anti-LTBR IgG antibodies

| anti-LTBR IgG | epitope bin |
|---|---|
| P1AE1873 | 1 |
| P1AE5929 | 4 |
| P1AE9452 | 1 |
| P1AE9457 | 5 |
| P1AE9459 | 5 |
| P1AF0064 | 4 |
| P1AF0079 | 3 |
| P1AF0080 | 5 |

### 1.10 SPR Characterization of Anti-LTBR IgG Antibodies

Binding kinetics to human, cynomolgus and murine LTBR: Binding of anti-LTbR IgGs to human, cynomolgus and murine LTBR was investigated by surface plasmon resonance using a Biacore T200 or Biacore 8K instrument (Cytiva). All experiments were performed at 25° C. using HBS-P (Cytiva #BR-1006-71) as running and dilution buffer. An anti-human IgG PG LALA specific antibody was immobilized on a Series S CM5 Sensor Chip (GE Healthcare #29104988) using standard amine coupling chemistry. Anti-LTBR IgGs were captured on the surface leading to capture levels between 10 and 100 RU. Human, cynomolgus or murine LTBR (P1AE7979, P1AE4411, or P1AE4410, respectively) were injected for 120 s with concentrations from 3.7 up to 300 nM (1:3 dilution series) onto the surface (association phase) at a flow rate of 30 μl/min. The dissociation phase was monitored for 600 s by washing with running buffer. The surface was regenerated by injecting 10 mM NaOH for 60 s at a flow rate of 5 μl/min. Bulk refractive index differences were corrected by subtracting the response obtained from a reference surface. Blank injections were subtracted (double referencing). The recorded sensorgrams were fitted to a 1:1 Langmuir binding model using the BIAevaluation software. The association and dissociation rate constants as well as the respective affinities (equilibrium dissociation constant KD) have been summarized in Table 10 below.

TABLE 10
Binding kinetics of anti-LTBR IgG antibodies to human,
cynomolgus and murine LTBR as determined by SPR

| anti-LTBR | | | | KD | T1/2 |
|---|---|---|---|---|---|
| IgG | antigen | Ka (1/ms) | Kd (1/s) | (nM) | (min) |
| P1AE9452 | hu LTBR | 1.73E+05 | 5.26E−05 | 0.3 | 220 |
| | cyno LTBR | 1.60E+05 | 5.92E−05 | 0.4 | 195 |
| | mu LTBR | no/very weak binding | | | |
| P1AE9450 | hu LTBR | low activity/no 1:1 binding | | | |
| | cyno LTBR | low activity/no 1:1 binding | | | |
| | mu LTBR | no binding | | | |
| P1AE9459 | hu LTBR | 7.95E+05 | 2.17E−04 | 0.3 | 53 |
| | cyno LTBR | 6.41E+05 | 1.91E−04 | 0.3 | 61 |
| | mu LTBR | 7.48E+05 | 7.95E−03 | 10.6 | 1 |
| P1AF0080 | hu LTBR | 6.46E+05 | 1.94E−03 | 3.0 | 6 |
| | cyno LTBR | 5.31E+05 | 1.88E−03 | 3.5 | 6 |
| | mu LTBR | 6.33E+05 | 1.88E−02 | 29.7 | 1 |
| P1AF0064 | hu LTBR | 1.45E+06 | 6.75E−05 | 0.05 | 171 |
| | cyno LTBR | 1.19E+06 | 5.19E−05 | 0.04 | 223 |
| | mu LTBR | 8.84E+05 | 3.67E−02 | 41.6 | 0.3 |
| P1AF0079 | hu LTBR | 7.94E+04 | 2.49E−03 | 31 | 5 |
| | cyno LTBR | 6.76E+04 | 2.47E−03 | 37 | 5 |
| | mu LTBR | no binding | | | |
| P1AE9457 | hu LTBR | 6.85E+04 | 6.36E−04 | 9 | 18 |
| | cyno LTBR | 6.79E+04 | 6.51E−04 | 10 | 18 |
| | mu LTBR | no binding | | | |
| P1AE5929 | hu LTBR | 2.55E+05 | 5.22E−04 | 2 | 22 |
| | cyno LTBR | 1.87E+05 | 5.97E−04 | 3 | 19 |
| | mu LTBR | 3.82E+04 | 2.96E−03 | 77 | 19 |
| P1AF7213 | hu LTBR | 2.99E+05 | 1.76E−03 | 5.9 | 7 |
| | cyno LTBR | 2.42E+05 | 2.03E−03 | 8.4 | 6 |
| | mu LTBR | 4.98E+04 | 7.39E−03 | 148.3 | 2 |
| P1AE1873 | hu LTBR | 4.89E+06 | 5.43E−03 | 1 | 2 |

| | | |
|---|---|---|
| (CBE11) | cyno LTBR | no binding |
| | mu LTBR | no binding |

### 1.11 Reducing the Hydrophobicity of Anti-LTBR Antibody P1AE5929

As the anti-LTBR IgG P1AE5929 exhibited some degree of hydrophobicity, an attempt was made to generate a more hydrophilic variant thereof. Thus, a 3D model of the V-domains in P1AE5929 was generated and the surface was analyzed for hydrophobic patches. One such patch was clearly identified at the tip of CDR2 of the VH-domain centered around an isoleucine depicted in bold: IPIFG (SEQ ID NO:266). As some hydrophobic residues in this loop might be essential for the antigen binding, a conservative replacement was considered essential and a phenylalanine (F) to histidine (H) mutation has been introduced in order to conserve the aromatic moiety but being more polar with a histidine compared to a phenylalanine. This mutation resolved the hydrophobic patch by introduction of an exposed histidine in the neighborhood of two isoleucines and resulted in the hydrophobicity-repaired anti-LTBR antibody P1AF7213. Apparent hydrophobicity of P1AE5929 as assessed by hydrophobic interaction chromatography (HIC) could be reduced from 0.733 to 0.321 (relative retention time compared to hydrophobicity standards) and the actual retention time decreased from 41.1 min to 29.7 min in a 55 min HIC gradient for P1AF7213 whilst retaining comparable affinities to human, cynomolgus and murine LTBR.

### Example 2

### Generation of Anti-FAP/Anti-LTBR Bispecific Antibodies

### 2.1 Generation and Production of Anti-FAP/Anti-LTBR Bispecific Antibodies

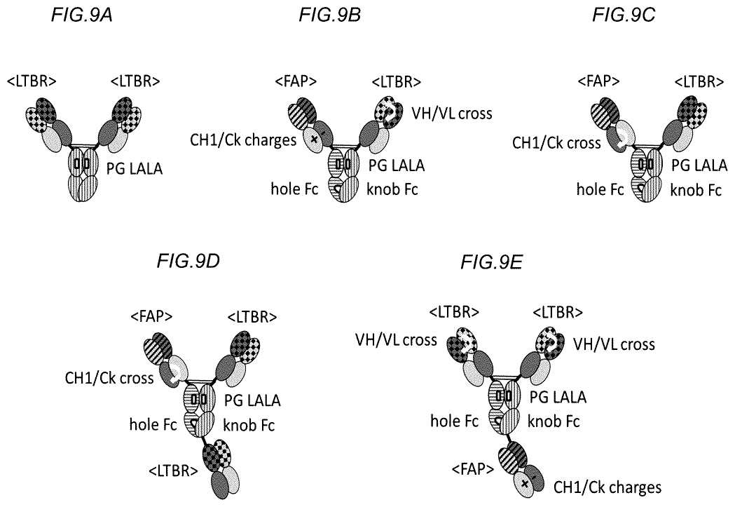

After the generation and characterization of the monospecific anti-LTBR IgGs (FIG. 9A), several preferred agonistic anti-LTBR IgGs have been converted into 1+1 (monovalent for LTBR) and 2+1 (bivalent for LTBR) tumor stroma-targeted (i.e. FAP-targeted) bispecific antibodies. The generation and preparation of anti-FAP clone 4B9 is disclosed in WO 2012/020006 A2, which is incorporated herein by reference.

Schematic illustrations of the different bispecific antibody structures made are shown in FIGS. 9B to 9E as well as in FIGS. 37B to 37E. Table 11 summarizes the identifiers of the anti-LTBR IgGs as well as 1+1 and 2+1 anti-FAP/anti-LTBR bispecific antibodies that have been generated on basis of the V-domain sequences of the respective IgGs and their resulting bispecific derivatives.

TABLE 11
Identifiers of the anti-LTBR antibodies and their
resulting bispecific derivatives

| | identifier 1 + 1 | identifier 2 + 1 |
|---|---|---|
| identifier IgG | bispecific antibody | bispecific antibody |
| P1AE1873 (CBE11) | P1AG1326 | P1AE1079 |
| P1AH0119 (BHA10) | P1AH5884 | P1AH5885 |
| P1AF5929 | P1AF0532 | — |
| P1AF7213 | P1AF9728 (P1AG7504) | P1AG5684, P1AG7576, |
| | | P1AG7509 |
| P1AE9450 | P1AF0535 (P1AG7506) | P1AF0544, P1AG7511 |
| P1AE9452 | P1AF0534 (P1AG7505) | P1AF9729 (P1AG7510) |
| P1AE9457 | P1AF5257 | — |
| P1AE9459 | P1AF0537 (P1AG7507), | P1AF0546, P1AG7512, |
| | P1AG0694 | P1AH5886 |
| P1AF0064 | P1AF9727 | — |
| P1AF0079 | P1AF5260 | |
| P1AF0080 | P1AF5261 (P1AG7508) | P1AG7513, P1AG5683, |
| | | P1AG7575 |

The bispecific anti-FAP/anti-LTBR antibodies were constructed as Fc knobs-into-holes IgGs with CrossMab technology. This means that for the generation of unsymmetric bispecific antibodies, Fc domain subunits contained either the “knob” or “hole” mutations to avoid mispairing of the heavy chains. In order to avoid mispairing of light chains in bispecific antibodies, exchange of VH/VL or CH1/Ckappa domains was introduced in one binding moiety (CrossFab technology). For P1AF9727, P1AF9728, P1AF9729 and P1AG0694, the CH1 and Ckappa domains of the Fab arm with FAP-specificity were crossed. For P1AG1326, P1AF0532, P1AF0534, P1AF5257, P1AF 5260, P1AF0535, P1AF0537, P1AF5261, P1AH5884, P1AH5885, and P1AH5886, the VH and VL domains of the Fab arm with LTBR-specificity were crossed and the CH1 and Ckappa domains of the Fab arm with FAP-specificity were equipped with complementary charges, two negatively charged glutamates in CH1 and a positively charged arginine and lysine in Ckappa. Pro329Gly, Leu234Ala and Leu235Ala mutations (PG-LALA) were introduced in the constant region of the human IgG1 heavy chains to abrogate binding to Fc gamma receptors.

The bispecific anti-FAP/anti-LTBR antibodies were either expressed in Expi293 (HEK) cells at Roche (P1AG1326, P1AF9728, P1AF0534, P1AF0535, P1AF0537, P1AF9727 and P1AE107) or in CHO K1 cells at evitria (P1AF9729 and P1AF5261). All bispecific anti-FAP/anti-LTBR antibodies were purified at Roche using a combined protein A affinity chromatography (Protein A (MabSelect™ SuRe™, Cytiva) and cation exchange chromatography (POROS™ XS) method followed by a preparative size-exclusion chromatography (HiLoad® 16/600 Superdex® S200, Cytiva). Purity of the purified bispecific antibodies was determined by analytical size-exclusion-chromatography (e.g. TSK G3000 SWXL) and CE-SDS (e.g. Caliper LabChip GXII). The identities of the bispecific antibodies were confirmed by LC-MS detecting the theoretical masses of the reduced and non-reduced antibodies. Endotoxin levels have been determined and were all below 0.33 EU/mg. The bispecific anti-FAP/anti-LTBR antibodies P1AH5884, P1AH5885, and P1AH5886 were produced at WuXi Biologics in CHO and purified by MabSelectSuRe LX protein A affinity chromatography, HiTrap SP HP cation exchange chromatography, and Superdex200 size-exclusion chromatography.

### 2.2 Binding of Anti-FAP/Anti-LTBR Antibodies to Human, Cynomolgus and Murine LTBR as Measured by ELISA

Nunc streptavidin coated plates (MicroCoat #11974998001) were coated with 25 μl/well biotinylated human, murine or cynomolgus LTBR extracellular domain (P1AE2401, P1AE2655, and P1AE2656, respectively) fused to a human IgG1 Fc-fragment at a concentration of 125 ng/ml and incubated overnight at 4° C. After washing 3×90 μl/well with PBST-buffer (10×PBS, Roche #11666789001+0.1% Tween 20), 25 μl anti-FAP/anti-LTBR bispecific antibodies were added in 1:3 dilutions starting at a concentration of 15 μg/ml and incubated 1 h at RT. After washing (3×90 μl/well with PBST-buffer), 25 μl/well anti hu kappa POD (Millipore, AP502P, 1:2000) was added and incubated at RT for 1 h. After washing (6×90 μl/well with PBST-buffer) 25 μl/well TMB substrate (Roche, 11835033001) were added. Measurements were performed at 370/492 nm.

Binding of anti-FAP/anti-LTBR-bispecific antibodies (monovalent binding for FAP and LTBR, except for P1AF9729 which is monovalent for FAP and bivalent for LTBR) to the extracellular domain of human, cynomolgus and murine LTBR was tested by direct ELISA (Table 12). All anti-FAP/anti-LTBR-bispecific antibodies show strong monovalent binding to human LTBR with EC₅₀ values in the range of 100 pM-1.5 nM. P1AF9729, which is bivalent for LTBR, is binding with an EC₅₀ of ˜50 pM. Binding to human, cynomolgus and murine LTBR of these bispecific antibodies is comparable to the binding of the monospecific IgGs (see Table 4) that contain the same LTBR binder (indicated by the ¹⁻⁴ in Table 12). As their IgG predecessors, all bispecific antibodies are cross-reactive to cynomolgus LTBR and a subset also cross-reacts with murine LTBR. Bivalent LTBR IgGs (see Table 4) do not show a shift in general to lower EC₅₀ values when compared to their monovalent LTBR counterparts in Table 12. Thus, for the bivalent IgGs, no binding via avidity is evident in this assay. In contrast, P1AF9729 comprises the same anti-LTBR agonist as P1AF0534 but with an additional LTBR-binding Fab-fragment, so the shift to a lower EC₅₀ is likely due to avidity.

TABLE 12
ELISA binding of bispecific anti-FAP/anti-LTBR
antibodies to human, cynomolgus and murine LTBR

| | human LTBR ECD | | cynomolgus LTBR ECD | | murine LTBR ECD | |
|---|---|---|---|---|---|---|
| | | max | | max | | max |
| | rel. EC₅₀ | binding | rel. EC₅₀ | binding | rel. EC₅₀ | binding |
| sample ID | (nM) | (OD) | (nM) | (OD) | (nM) | (OD) |
| P1AF9728 | 0.1340 | 3.4275 | 0.2011 | 3.435 | 4.1998 | 2.7609 |
| P1AF0534¹ | 0.1031 | 3.297 | 0.1011 | 3.5524 | | 2.1379 |
| P1AF0535² | 1.4189 | 3.0459 | 1.3617 | 3.1665 | | 0.3013 |
| P1AF0537³ | 0.0940 | 3.2212 | 0.1154 | 3.6737 | 0.3589 | 3.7791 |
| P1AF5261⁴ | 0.1826 | 3.1277 | 0.1836 | 3.4246 | 0.3158 | 3.097 |
| P1AF9727 | 0.1329 | 3.7425 | 0.0788 | 3.7563 | | 3.207 |
| P1AF9729¹ | 0.0457 | 3.6227 | 0.0233 | 3.7498 | 3.3701 | 3.1997 |

¹⁻⁴refer to LTBR IgGs in Table 4.

2.3 ELISA Measuring Ligand Competition for Bispecific Anti-FAP/Anti-LTBR Antibodies with Human Lymphotoxin α1β2 and Human LIGHT

Nunc streptavidin coated plates (MicroCoat #11974998001) were coated with 25 μl/well biotinylated human LTBR extracellular domain (P1AE2401) fused to a human IgG1 Fc-Fragment at a concentration of 125 ng/ml (500 ng/ml for LIGHT interaction) overnight at 4° C. After washing 3×90 μl/well with PBST-buffer (10×PBS, Roche #11666789001+0,1% Tween 20), 25 μl anti-FAP/anti-LTBR antibodies were added in 1:3 dilutions starting at a concentration of 15 μg/ml and incubated 1 h at RT. After washing with 3×90 μl/well PBST-buffer (10×PBS, Roche #11666789001+0,1% Tween 20), 25 μl ligand (either human lymphotoxin α1β2 (P1AE1235) or His6-tagged human LIGHT (R&D systems, 664-Li) were added at a concentration of 250 ng/ml for lymphotoxin m2 and 1000 ng/ml for LIGHT and incubated 1 h at RT. After washing (3×90 μl/well with PBST-buffer), 25 μl/well anti His6-POD (Bethyl #A190-114P, 1:10000) was added and incubated at RT for 1 h. After washing (6×90 μl/well with PBST-buffer), 25 μl/well TMB substrate (Roche, 11835033001) were added. Measurements were performed at 370/492 nm and summarized in Table 13. Listed are the relative IC₅₀ values (nM) as well as top and bottom ODs.

TABLE 13
Inhibition of LTBR-lymphotoxin α1β2 and LTBR-LIGHT interactions
by anti-FAP/anti-LTBR bispecific antibodies

| | human lymphotoxin α1β2 interaction | | | human LIGHT interaction | | |
|---|---|---|---|---|---|---|
| | rel. | rel. fit | rel. fit | rel. | rel. fit | rel. fit |
| | IC₅₀ | top | bottom | IC₅₀ | top | bottom |
| sample ID | (nM) | (OD) | (OD) | (nM) | (OD) | (OD) |
| P1AF9728 | 0.2455 | 1.7 | 0.9 | 3.4803 | 1.5 | 0.9 |
| P1AF0534¹ | | 1.7 | 1.3 | | | 1.4 |
| P1AF0535² | | | | | | |
| P1AF0537³ | 0.5332 | 1.6 | 0.4 | | 1.4 | 1.2 |
| P1AF5261⁴ | 0.8733 | 2.56 | 1.7 | | | 1.4 |
| P1AF9727 | 0.1375 | 1.7 | 0.1 | | 1.5 | 0.2 |
| P1AF9729¹ | 0.1404 | 1.6 | 0.6 | | 1.4 | 1.4 |

¹⁻⁴refer to LTBR IgGs in Table 5.

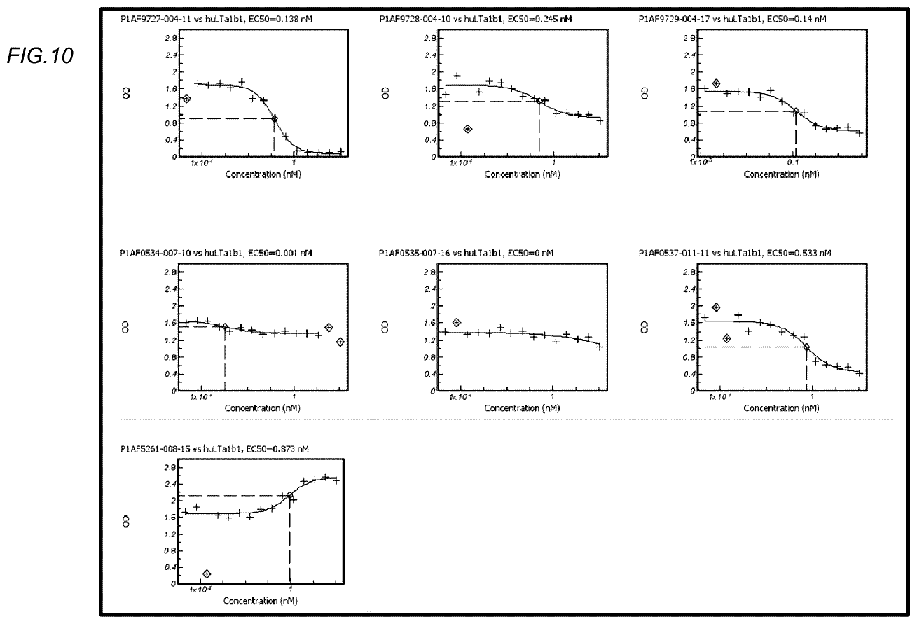

Inhibition of the protein-protein interaction between human LTBR and the human ligands lymphotoxin α1β2 and LIGHT was tested in a multistep ELISA assay. The following anti-FAP/anti-LTBR bispecific antibodies show inhibition of the LTBR-lymphotoxin α1β2 interaction with IC₅₀ values between ˜140 pM and ˜0.8 nM: P1AF9728, P1AF0537, P1AF9727, and P1AF9729. P1AF0534 and P1AF0535 show weaker to no inhibition of the interaction (FIG. 10). P1AF5261 (which comprises the anti-LTBR antibody of P1AF0080) shows an increase of the assay signal with increasing concentrations again, as already detected for this anti-LTBR antibody in the bivalent IgG format.

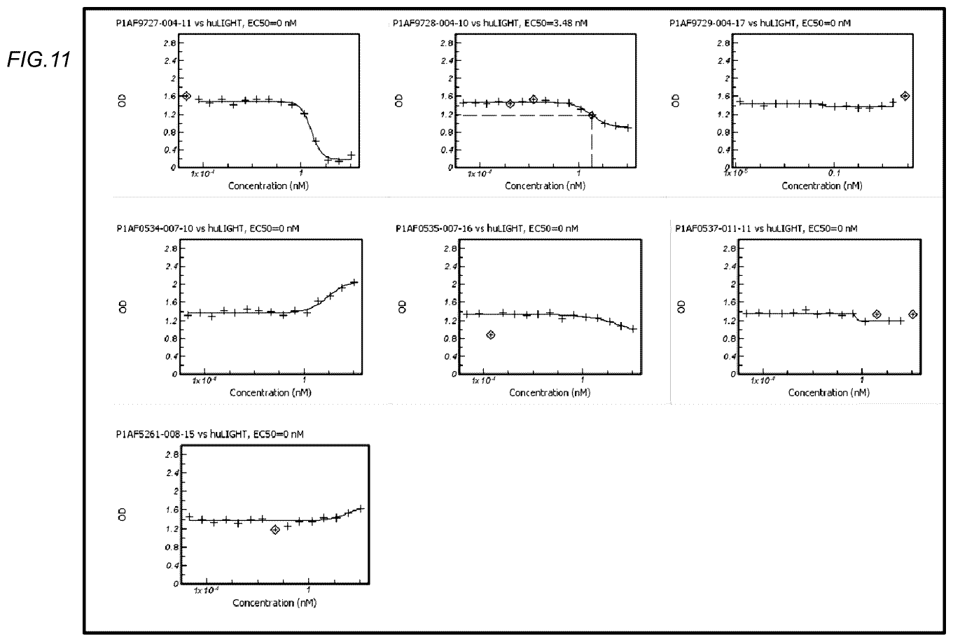

The LTBR-LIGHT interaction can also be inhibited by the anti-FAP/anti-LTBR bispecific antibodies in this biochemical assay, albeit to a lower extent than the LTBR-lymphotoxin α1β2 interaction. While P1AF9727 and P1AF9728 show significant inhibition, only weak inhibition at higher antibody concentrations was detected for P1AF0535 and no inhibition was detected for P1AF9729 and for P1AF0537. P1AF5261 shows a weak increase of the signal at higher antibody concentrations in contrast to the behavior of the bivalent IgG P1AF0080. P1AF0534 also shows an increase of the signal at higher concentrations (FIG. 11).

### 2.4 SPR Characterization of Anti-FAP/Anti-LTBR Bispecific Antibodies

Binding kinetics to human, cynomolgus and murine LTBR: Binding of anti-FAP/anti-LTBR bispecific antibodies to human, cynomolgus and murine LTBR was investigated by surface plasmon resonance using a Biacore T200 or Biacore 8K instrument (Cytiva). All experiments were performed at 25° C. using HBS-P (Cytiva #BR-1006-71) as running and dilution buffer. An anti-human IgG PG LALA specific antibody was immobilized on a Series S CM5 Sensor Chip (GE Healthcare #29104988) using standard amine coupling chemistry. Bispecific antibodies were captured on the surface leading to capture levels between 10 and 100 RU. Human, cynomolgus and murine LTBR (P1AE7979, P1AE4411, or P1AE4410, respectively) were injected for 120 s with concentrations from 3.7 up to 300 nM (1:3 dilution series) onto the surface (association phase) at a flow rate of 30 μl/min. The dissociation phase was monitored for 600 s by washing with running buffer. The surface was regenerated by injecting 10 mM NaOH for 60 sec at a flow rate of 5 μl/min. Bulk refractive index differences were corrected by subtracting the response obtained from a reference surface. Blank injections were subtracted (double referencing). The derived curves were fitted to a 1:1 Langmuir binding model using the BIAevaluation software. Association and dissociation rate constants as well as the respective affinities (equilibrium dissociation constant KD) are summarized in Table 14.

TABLE 14
Binding kinetics of the anti-FAP/anti-LTBR bispecific antibodies
to human, cynomolgus and murine LTBR as determined by SPR

| FAP LTBR | | | | KD | T1/2 |
|---|---|---|---|---|---|
| bispec antibodies | antigen | Ka (1/ms) | Kd (1/s) | (nM) | (min) |
| P1AF0534 | hu LTBR | 2.21E+05 | 5.18E−05 | 0.2 | 223 |
| | cyno LTBR | 1.95E+05 | 9.57E−05 | 0.5 | 121 |
| | mu LTBR | no/ very weak binding | | | |
| P1AF0535 | hu LTBR | low activity/no 1:1 binding | | | |
| | cyno LTBR | low activity/no 1:1 binding | | | |
| | mu LTBR | no binding | | | |
| P1AF0537 | hu LTBR | 7.84E+05 | 2.63E−04 | 0.3 | 44 |
| | cyno LTBR | 6.67E+05 | 2.05E−04 | 0.3 | 56 |
| | mu LTBR | 6.46E+05 | 7.52E−03 | 12.0 | 2 |
| P1AF0546 | Hu LTBR | 9.99E+05 | 2.25E−04 | 0.2 | 51 |
| P1AF5261 | hu LTBR | 9.22E+05 | 1.36E−03 | 1.5 | 8 |
| | cyno LTBR | 8.59E+05 | 1.27E−03 | 1.5 | 9 |
| | mu LTBR | 1.13E+06 | 1.62E−02 | 14.0 | 1 |
| P1AF9727 | hu LTBR | 1.19E+06 | 1.63E−04 | 0.1 | 71 |
| | cyno LTBR | 1.11E+06 | 1.46E−04 | 0.1 | 79 |
| | mu LTBR | 9.25E+05 | 9.40E−02 | 102.0 | 0.1 |
| P1AF9728 | hu LTBR | 2.51E+05 | 1.79E−03 | 7.1 | 6 |
| | cyno LTBR | 2.10E105 | 2.09E−03 | 9.9 | 6 |
| | mu LTBR | 6.86E+04 | 8.91E−03 | 130.0 | 1 |
| P1AE9729 | hu LTBR | 2.16E+05 | 8.60E−05 | 0.4 | 134 |
| | cyno LTBR | 1.86E+05 | 9.89E−05 | 0.5 | 117 |

| | |
|---|---|
| mu LTBR | No/very weak binding |

For selected 1+1 and 2+1 bispecific antibodies (P1AF0534, P1AF0537, P1AF5261, and P1AF0546, P1AG5683, respectively), monovalent binding (affinity) and bivalent binding (avidity) to human LTBR was further characterized by SPR. For measuring affinity, the bispecific antibodies were captured by an anti-human IgG PG LALA specific antibody that had been immobilized on a Series S CM3 Sensor Chip (GE Healthcare) using standard amine coupling chemistry and human LTBR (P1AE7979) was used as analyte. In contrast, for measuring avidity of the 2+1 bispecific constructs that are bivalent for LTBR, biotinylated human LTBR (P1AE7979) was captured on an SA streptavidin chip and the bispecific antibodies were used as analytes. The results have been summarized in Table 15A and Table 15B. Shown are the association and dissociation rates, affinities or apparent affinities (of the bispecific antibodies bivalent for LTBR) and half-life of the antibody:receptor complexes.

As expected, for the bispecific antibodies that are bivalent for LTBR (P1AF0546 and P1AG5683, respectively), an avidity effect can be observed: For P1AF0546, the dissociation rate constant kd of 2.25E-04 (1/s) and affinity of 0.2 nM, respectively (Table 15A), decreases to values out of the specifications of the instrument (Table 15B), whereas for P1AG5683, the dissociation rate constant of 1.23E-03 kd (1/s) and affinity of 1.1 nM, respectively (Table 15A), decreases to kd of 4.33E-05 (1/s) and an apparent affinity (avidity) of 0.01 nM (Table 15B).

TABLE 15A
Binding kinetics of the anti-FAP/
anti-LTBR bispecific antibodies to human LTBR
as determined by SPR in an ‘affinity’ assay set-up

| FAP-LTBR | | | K_{D} | t_{1/2} |
|---|---|---|---|---|
| bispec antibodies | kₐ (1/ms) | k_{d} (1/s) | (nM) | (min) |
| P1AF0534 | 2.41E+05 | 8.08E−05 | 0.3 | 143 |
| P1AF0537 | 8.85E+05 | 2.20E−04 | 0.2 | 53 |
| P1AF0546 | 9.99E+05 | 2.25E−04 | 0.2 | 51 |
| P1AF5261 | 9.86E+05 | 1.28E−03 | 1.3 | 9 |
| P1AG5683 | 1.14E+06 | 1.23E−03 | 1.1 | 9 |

TABLE 15B
Binding kinetics of the anti-FAP/anti-LTBR bispecific
antibodies to human LTBR as determined
by SPR in an ‘avidity’ assay set-up

| FAP-LTBR | | | K_{D} | t_{1/2} |
|---|---|---|---|---|
| bispec antibodies | kₐ (1/ms) | k_{d} (1/s) | (nM) | (min) |
| P1AF0534 | 3.05E+05 | 4.12E−05 | 0.1 | 280 |
| P1AF0537 | 9.20E+05 | 1.15E−04 | 0.1 | 100 |
| P1AF0546 | 2.03E+06 | oos* | oos* | oos* |
| P1AF5261 | 2.45E+06 | 1.35E−03 | 0.6 | 9 |
| P1AG5683 | 3.92E+06 | 4.33E−05 | 0.01 | 267 |

*out of specification

### 2.5 Binding Kinetics to Human FAP

Binding of the anti-FAP binding domain of the anti-FAP/anti-LTBR bispecific antibodies was investigated by surface plasmon resonance using a Biacore T200 (Cytiva). An anti-His IgG (Cytiva, #28995056 specific antibody was immobilized on a Series S CM5 Sensor Chip (GE Healthcare #29104988) using standard amine coupling chemistry. Antibodies were captured on the surface leading to a capture levels between 20 and 30 RU. Human FAP (P1AA5347) was injected for 180 s with concentrations from 0.37 up to 30 nM (1:3 dilution series) onto the surface (association phase) at a flow rate of 30 μl/min. The dissociation phase was monitored for 600 s by washing with running buffer. The surface was regenerated by injecting 10 mM Glycine pH 1.5 for 60 sec at a flow rate of 5 μl/min. Bulk refractive index differences were corrected by subtracting the response obtained from a reference surface. Blank injections were subtracted (double referencing). The derived curves were fitted to a 1:1 Langmuir binding model using the BIAevaluation software. Association and dissociation rate constants as well as the respective affinities (equilibrium dissociation constant K_{D}) have been summarized in Table 16. Shown are the association and dissociation rates, affinities and half-life of the antibody:FAP complexes.

TABLE 16
Binding kinetics of the anti-FAP/anti-LTBR bispecific
antibodies to human FAP as determined by SPR

| FAP-LTBR | | | K_{D} | t_{1/2} |
|---|---|---|---|---|
| bispec antibodies | kₐ (1/ms) | k_{d} (1/s) | (nM) | (min) |
| P1AF0534 | 2.25E+06 | 4.70E−04 | 209 | 25 |
| P1AF0535 | 1.78E+06 | 4.66E−04 | 262 | 25 |
| P1AF0537 | 2.09E+06 | 4.46E−04 | 213 | 26 |
| P1AF5261 | 2.08E+06 | 4.73E−04 | 228 | 24 |
| P1AF9727 | 2.35E+06 | 5.72E−04 | 243 | 20 |
| P1AF9728 | 2.54E+06 | 5.21E−04 | 205 | 22 |
| P1AF9729 | 2.05E+06 | 5.30E−04 | 259 | 22 |
| P1AG0694 | 2.77E+06 | 4.81E−04 | 174 | 24 |

### Example 3

### Generation of Bispecific Murine Surrogate Antibodies

### 3.1 Generation of Anti-FAP/Anti-LTBR Bispecific Murine Surrogate Antibodies

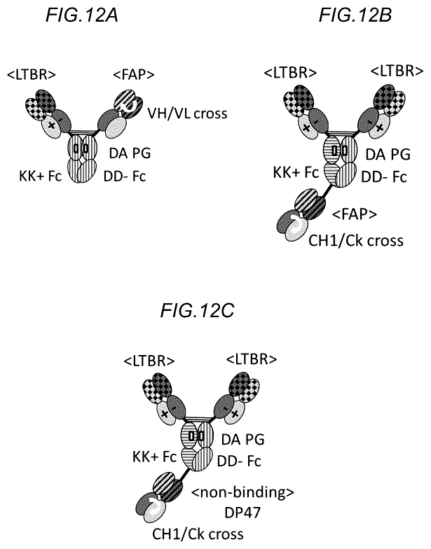

The human, cynomolgus and murine triple cross-reactive anti-LTBR agonist P1AF0080 was used to generate a bispecific 1+1 and a bispecific 2+1 (bivalent for LTBR) murine surrogate antibody for in vivo studies in mice. These anti-FAP/anti-LTBR bispecific murine surrogate antibodies, P1AF4664 and P1AF4674, respectively, are depicted in FIGS. 12A and 12B. Whereas their V-domains were kept human, all constant antibody domains are murine. The murine IgG1 Fc was heterodimerized by complementary charges in CH3 (KK+ and DD− chains) and silencing of the Fc's effector functions was achieved by the introduction of DA PG mutations in CH2.

P1AF4664 and P1AF4674 were produced by evitria using their proprietary vector system with conventional (non-PCR based) cloning techniques and using suspension-adapted CHO K1 cells (originally received from ATCC and adapted to serum-free growth in suspension culture at evitria). For the production, evitria used their proprietary, animal-component free and serum-free media (eviGrow and eviMake2) and their proprietary transfection reagent (eviFect). Supernatants were harvested by centrifugation and subsequent filtration (0.2 μm filter) and proteins were purified from the harvested supernatant by standard methods.

In brief, they were purified by protein A affinity chromatography (Protein A MabSelectSure) and preparative size-exclusion-chromatography (SEC) (HiLoad 50/600 S200) according to standard procedures and the manufacturers' instructions. Purity of the purified proteins was determined by analytical size-exclusion-chromatography (e.g. TSK G3000 SWXL) and CE-SDS (e.g. Caliper LabChip GXII). Endotoxin levels were determined and were ≤0.11 EU/mg and 0.07 EU/mg, respectively.

The human, cynomolgus and murine triple cross-reactive anti-LTBR agonist P1AE9459 was used to generate bispecific 2+1 (bivalent for LTBR) FAP-targeted or non-targeted (DP47) murine surrogate antibodies for in vivo studies in mice. These anti-FAP/anti-LTBR or non-targeted/anti-LTBR bispecific murine surrogate antibodies, P1AG5459 and P1AG5461, respectively, are as depicted in FIGS. 12B and 12C. Whereas their V-domains were kept human, all constant antibody domains are murine. The murine IgG1 Fc was heterodimerized by complementary charges in CH3 (KK+ and DD− chains) and silencing of the Fc's effector functions was achieved by the introduction of D265A P329G mutations in CH2.

P1AG5459 and P1AG5461 were produced and purified by WuXi Biologics. In brief, they were produced in CHO and purified by MabSelectSuRe LX protein A affinity chromatography and Superdex200 size-exclusion chromatography (P1AG5459) or MabSelectSuRe LX protein A affinity chromatography, HiTrap Butyl HP hydrophobic interaction chromatography, and Superdex200 size-exclusion chromatography (P1AG5461).

### 3.2 SPR Characterization of Anti-FAP/Anti-LTBR Bispecific Murine Surrogate Antibodies

### Binding Affinity and Avidity to Murine LTbR and Murine FAP:

As P1AF4664 is monovalent for LTBR and P1AF4674 is bivalent for LTBR, binding affinity and avidity were determined on a Biacore T200 instrument (Cytiva). All experiments were performed at 25° C. using HBS-P (Cytiva #BR-1006-71) as running and dilution buffer. To determine the affinity of these bispecific murine surrogate antibodies, a goat anti-mouse Fcγ specific antibody (JacksonImmunoResearch #115-005-071) was immobilized on a Series S CM3 Sensor Chip using standard amine coupling chemistry. The bispecific murine surrogate antibodies were captured on the surface and murine LTbR (P1AE4410) was injected in a single-cycle kinetics run with an association time of 120 s with concentrations from 3.7 up to 300 nM (1:3 dilution series) at a flow rate of 30 μl/min. The affinity to murine FAP was determined using the same sensor chip by injecting the murine FAP (P1AD9907) with a single concentration of 100 nM onto the surface with association time and flow rate as described above. The final dissociation phase was monitored for 600 s by washing with running buffer. Subsequently, the surface was regenerated by injecting 10 mM glycine pH 2.0 followed by 10 mM NaOH for 60 sec each at a flow rate of 5 μl/min. Bulk refractive index differences were corrected by subtracting the response obtained from a reference surface. Blank injections were subtracted (double referencing). The derived curves were fitted to a 1:1 Langmuir binding model using the BIAevaluation software.

To determine the avidity of the 2+1 bispecific murine surrogate antibody P1AF4674, approximately 100 RU of biotinylated murine LTbR (P1AE4410) were immobilized on a Series S Sensor Chip SA. The bispecific murine surrogate antibodies were injected as analytes and tested in a single-cycle kinetics run with an association time of 120 s with concentrations from 11.1 to 100 nM (1:3 dilution series) at a flow rate of 30 μl/min. The final dissociation phase was monitored for 1800 s by washing with running buffer. Subsequently, the surface was regenerated by injecting 10 mM glycine pH 2.0 for 60 sec each at a flow rate of 5 μl/min. Bulk refractive index differences were corrected by subtracting the response obtained from a reference surface. Blank injections were subtracted (double referencing). The derived curves were fitted to a 1:1 Langmuir binding model using the BIAevaluation software. In this assay set-up, P1AF4674 showed clear avidity (app. KD 0.2 nM) in contrast to the 1+1 bispecific murine surrogate antibody P1AF4664 (app. KD 5 nM). The equilibrium dissociation constants (KD) as well as the half-lives of the complexes (t½) for the monovalent (1+1) and bivalent (2+1) anti-FAP/anti-LTBR bispecific murine surrogate antibodies binding to murine LTBR or murine FAP are reported in Table 17.

TABLE 17
Equilibrium dissociation constants (KD) and half-lives
of the complexes (t1/2) for the anti-FAP/
anti-LTBR bispecific murine surrogate antibodies

| | murine LTbR | | murine LTbR | | murine FAP |
|---|---|---|---|---|---|
| mu FAP | affinity set-up | | avidity set-up | | affinity set-up |
| LTBR bispec | K_{D} | t1/2 | app. K_{D} | t1/2 | K_{D} |
| antibodies | (nM) | (s) | (nM) | (s) | (nM) |
| P1AF4664 (1 + 1) | 13 | 75 | 5 | 45 | <0.1 |
| P1AF4674 (2 + 1) | 13 | 71 | 0.2 | 413 | <0.1 |

### Example 4

### Functional Characterization of LTBR Antibodies

Activation of LTBR leads to the upregulation of inflammatory and developmental genes such as the adhesion molecule ICAM, by stromal cells. We characterized the biological activity of the novel LTBR agonists by measuring their ability to upregulate ICAM in human endothelial cells and cancer associated fibroblasts (CAFs) in vitro.

### 4.1 Effect of Human Anti-LTBR Antibodies on Endothelial Cells

Human umbilical vein endothelial cells (HUVEC) were treated with anti-LTBR antibodies in the presence or absence of an anti-Fc crosslinker and the levels of the adhesion molecule ICAM was measured by FACS analysis.

Briefly, HUVEC (15000 cells/well, cat. C2517AS, Lonza) were seeded in a gelatin coated 96 well plate in complete EGM-2 media (cat. CC3162, Lonza) and allowed to form a monolayer overnight. HUVEC monolayers were treated overnight with anti-LTBR antibodies preincubated for 30 minutes with or without anti human-Fc F(ab)2 crosslinker (cat. 109-006-008, Jackson ImmunoReasearch, antibody to crosslinker molar ratio 1:2) in assay media (EBM-2 media supplemented with 2% FBS, ascorbic acid, heparin, GA-1000 and heparin, cat. CC3156 and CC4176, Lonza). Treated HUVEC were detached with accutase (Gibco) and stained with a live/dead dye (Zombie Aqua fixable viability kit, cat. 423102 Biolegend) and with an anti-human ICAM-BV421 labelled antibody (BD cat. 564077, diluted 1:100). Samples were acquired on a flow cytometer (BD Fortessa). Median fluorescent intensity of ICAM expression was analysed in FlowJo and data were normalized to CBE11 using the following formula: normalized MFI=(MFI−MFImin)/(MFImax−MFImin), where MFImax was calculated as the median of the MFI values measured at the top concentrations of CBE11 (PAE1873) with crosslinker, and MFImin was calculated as the median of the MFI values measured at the lowest concentration of CBE11 (P1AE1873) without cross-linker.

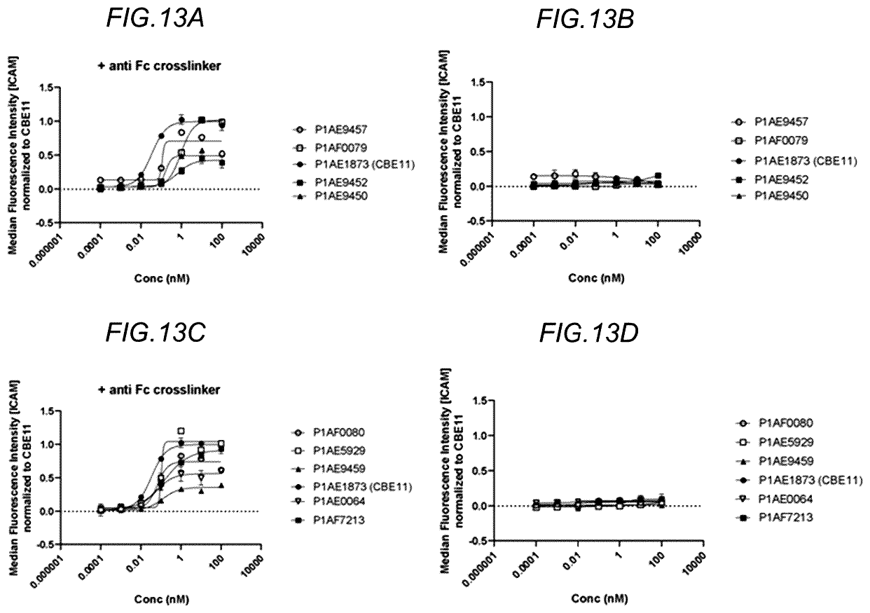

The novel anti-human LTBR antibodies induce ICAM upregulation on HUVEC in a dose dependent, and crosslinking dependent manner (FIGS. 13A to 13D).

### 4.2 Effect of Human Anti-LTBR Antibodies on Cancer Associated Fibroblasts

We further characterized the ability of the novel anti-LTBR antibodies to induce upregulation of adhesion molecules in a second stromal cell type in vitro, namely cancer associated fibroblasts. To this aim, CAFs cultures were treated with antibodies in the presence or absence of an anti-Fc crosslinker, and the levels of the adhesion molecule ICAM was measured by FACS analysis.

Briefly, immortalized cancer associated fibroblasts from prostate (12000 cells/well, hTERT PF179T CAF, ATCC CRL-3290) were seeded in 96 well plate in complete media (EMEM supplemented with 10% FBS, 0.075% sodium bicarbonate, 1 μg/mL puromycin). CAFs were treated over night with anti-LTBR antibodies preincubated for 30 minutes with or without anti human-Fc F(ab)₂ crosslinker (cat. 109-006-008, Jackson ImmunoReasearch, antibody to crosslinker molar ratio 1:2) in complete media. Treated CAFs were detached with accutase (Gibco) and stained with a NIR live/dead dye (LIVE/DEAD Fixable Near-IR Dead Cell Stain Kit, Thermo Fischer Scientific cat. L34975) and with an anti-human ICAM-BV421 labelled antibody (BD cat. 564077, diluted 1:100). Samples were acquired on a flow cytometer (BD Fortessa). Median fluorescent intensity of ICAM expression was analysed in FlowJo and data were normalized to CBE11 using the following formula: normalized MFI=(MFI−MFImin)/(MFImax−MFImin), where MFImax was calculated as the median of the MFI values measured at the top concentrations of CBE11 (PAE1873) with crosslinker, and MFImin was calculated as the median of the MFI values measured at the lowest concentration of CBE11 (P1AE1873) without cross-linker.

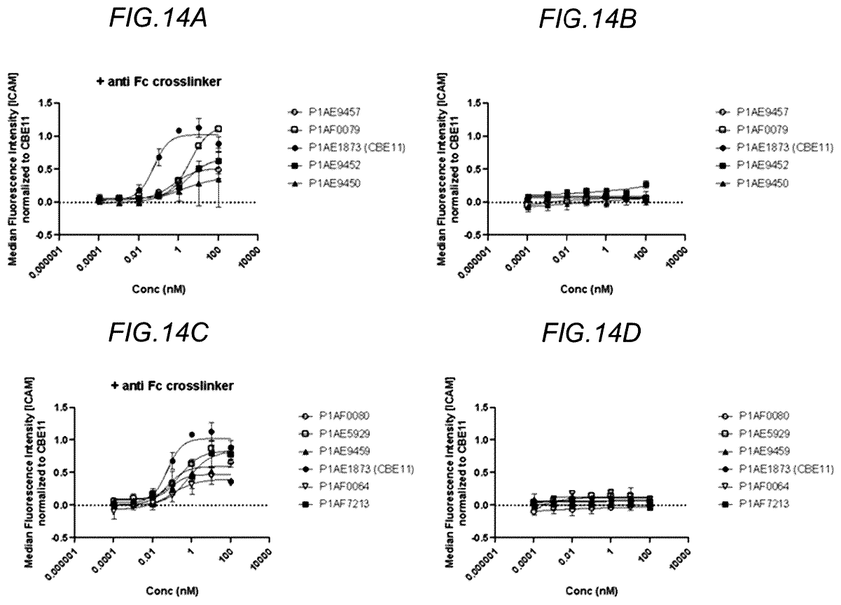

Anti-human LTBR antibodies induce ICAM upregulation on CAFs in a dose dependent, and crosslinking dependent manner (FIGS. 14A to 14D).

Taken together these examples show that novel anti-human LTBR agonistic antibodies are able to upregulate the adhesion molecule ICAM in a variety of stromal cells and their activity is strictly dependent on crosslinking. We next generated bispecific molecules comprising preferred novel LTBR agonists and a FAP antigen binding domain to obtain anti-FAP/anti-LTBR bispecific antibodies.

### Example 5

### Functional Characterization of Anti-FAP/Anti-LTBR Bispecific Antibodies

### 5.1 Anti-FAP/Anti-LTBR Bispecific Antibodies Bind Human, Cynomolgus and Murine LTBR on Cells

To test the ability of anti-FAP/anti-LTBR bispecific antibodies to bind human, cynomolgus monkey (cyno) and murine LTBR on cells, anti-FAP/anti-LTBR bispecific antibodies were incubated with cells engineered to express either human, cyno or mouse LTBR and their binding was measured by flow cytometry.

Briefly, CHO-K1 cells engineered to overexpress human, cyno or murine LTBR were detached with accutase (Gibco) and 100,000 cells/well were seeded in a 96 well plate and incubated with anti-FAP/anti-LTBR bispecific antibodies in culture medium (DMEM/F12, 10% FBS) for 1 h at 4° C. After washing, cell-bound molecules were detected using a PE labelled anti human-Fc antibody (AffiniPure F(ab′)₂ Fragment Goat Anti-Human IgG, Fcγ, Jackson Immunoresearch, cat. 109-116-098). Cell bound fluorescence was measured in an iQue flow cytometer (Sartorius). Median fluorescent intensity of PE was analysed in FlowJo and data were normalized to untreated controls (MFI/baseline MFI).

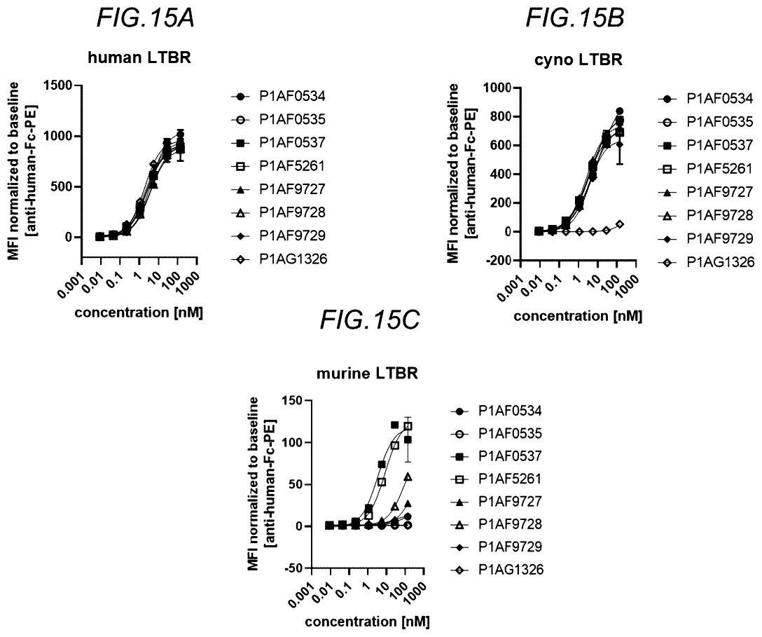

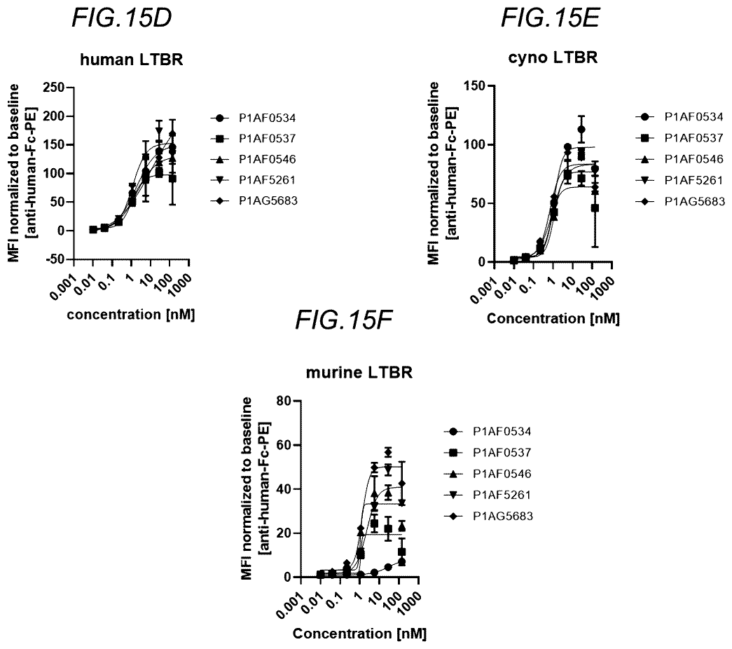

All bispecific molecules bind human and cyno LTBR with the exception of P1AG1326 which binds human LTBR but does not bind cyno LTBR (FIG. 15A and FIG. 15B as well as FIGS. 15D and 15E). These data are in line with SPR data described in Example 2.4.

Crossreactivity to cyno is an important advantage of the novel bispecific molecules, as it will allow the investigation of the toxicity profile in a relevant species. As LTBR is a widely expressed target, de-risking of potential toxicologic effects in vivo is of great importance.

P1AF0537, P1AF5261, P1AF0546, P1AG5683 and, to a lesser extent P1AF9728 and P1AF9727 additionally bind murine LTBR on cells (FIGS. 15C and 15F) allowing testing the efficacy and pharmacodynamic in murine syngeneic models.

### 5.2 Characterization of the FAP-Dependent Activity of Anti-FAP/Anti-LTBR Bispecific Antibodies In Vitro

Anti-FAP/anti-LTBR bispecific antibodies are designed to restrict LTBR activation to the tumor stroma by FAP-targeting. Activation of LTBR leads to the upregulation of inflammatory and developmental genes such as adhesion molecules (ICAM and VCAM) and chemoattractants (CXCL9, CXCL10 and CXCL11) by stromal cells. We characterized the biological activity of the anti-FAP/anti-LTBR bispecific antibodies by measuring their ability to upregulate adhesion molecules and chemoattractants in a FAP-dependent manner in human endothelial cells and cancer associated fibroblasts in vitro.

### 5.2.1 Effect of Anti-FAP/Anti-LTBR Bispecific Antibodies on Cancer Associated Fibroblasts

To test the ability of anti-FAP/anti-LTBR bispecific antibodies to activate cancer associated fibroblasts in a FAP-dependent manner in vitro, cultures of CAFs expressing endogenous FAP and CAFs deleted of FAP expression were treated with bispecific molecules and the levels of the adhesion molecule ICAM was measured by FACS analysis.

Briefly, FAP expression by immortalized cancer associated fibroblasts from prostate (hTERT PF179T CAF, ATCC CRL-3290) was knocked-out by CRISPR technology. Parental hTERT CAFs and FAP knock-out CAFs (hTERT CAFs_delFAP) were seeded in 96 well plate in complete media (EMEM, 10% FBS, 0.075% sodium bicarbonate, 1 μg/mL puromycin) at a density of 12000 cells per well. Cells were treated overnight with anti-FAP/anti-LTBR bispecific antibodies in complete media. Treated cells were detached with accutase (Gibco) and stained with a NIR live/dead dye (LIVE/DEAD Fixable Near-IR Dead Cell Stain Kit, Thermo Fischer Scientific cat. L34975) and with an anti-human ICAM-BV421 labelled antibody (BD cat. 564077, diluted 1:100). Samples were acquired on a flow cytometer (BD Fortessa). Median fluorescent intensity of ICAM expression was analysed in FlowJo and data were normalized to baseline untreated controls and expressed as fold change over baseline.

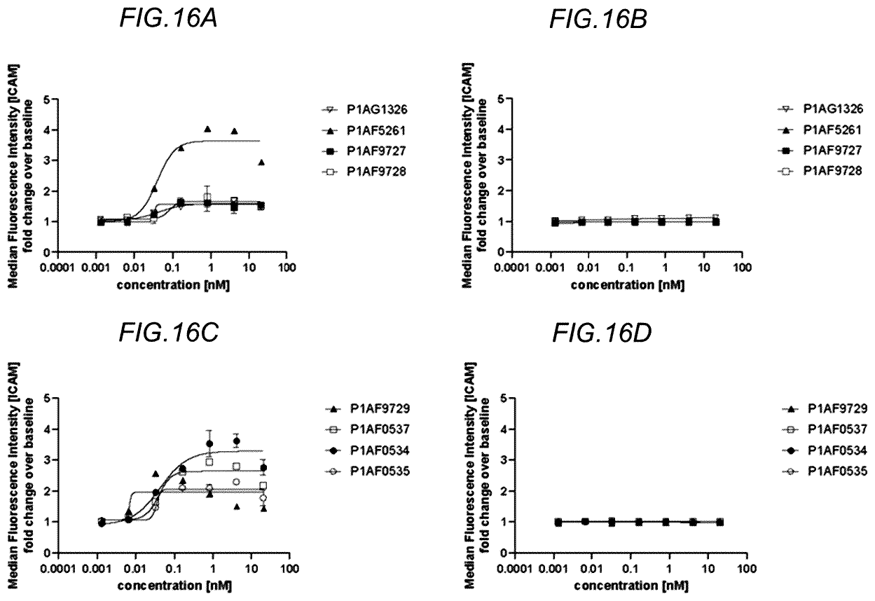

FAP-LTBR bispecific molecules induced ICAM upregulation on hTERT CAFs in a dose dependent, and FAP-dependent manner (FIGS. 16A to 16D).

### 5.2.2 Effect of Anti-FAP/Anti-LTBR Bispecific Antibodies on Endothelial Cells

To test the ability of anti-FAP/anti-LTBR bispecific antibodies to activate human endothelial cells in a FAP-dependent manner in vitro, co-cultures of human umbilical vein endothelial cells (HUVEC) and NIH-3T3 cells overexpressing human FAP were treated with bispecific molecules. The levels of the adhesion molecule ICAM on HUVEC was measured by FACS analysis and the amounts of the chemoattractants CXCL9, CXCL10 and CXCL11 secreted in the supernatant was measured by Bio-plex analysis.

Briefly, HUVEC (13000 cells/well, cat. C2517AS, Lonza) were seeded with NIH-3T3 or NIH-3T3 overexpressing FAP (2000 cells/well) in a gelatin coated 96 well plate in complete EGM-2 media (cat. CC3162, Lonza) and allowed to form a monolayer over night. Co-cultures were treated with anti-FAP/anti-LTBR bispecific antibodies in assay media (EBM-2 media supplemented with 2% FBS, ascorbic acid, heparin, GA-1000 and heparin, cat. CC3156 and CC4176, Lonza). After 24 hours, co-cultures were detached with accutase (Gibco) and stained with a NIR live/dead dye in PBS (LIVE/DEAD Fixable Near-IR Dead Cell Stain Kit, Thermo Fischer Scientific cat. L34975) and with an anti-human ICAM-BV421 (BD cat. 564077, diluted 1:100) and an anti-human-CD31-APC/Cy7 labelled antibody (Biolegend cat. 303120, diluted 1:300). Samples were acquired on a flow cytometer (BD Fortessa). The median fluorescent intensity of ICAM expression in the CD31+ population was analysed in FlowJo and data were normalized to baseline untreated controls and expressed as fold change over baseline. Conditioned media was collected after 48 hours and the concentration of CXCL9, CXCL10 and CXCL11 was measured using a custom Bio-plex multiplex chemokine assay kit (Biorad) following manufacturer instructions.

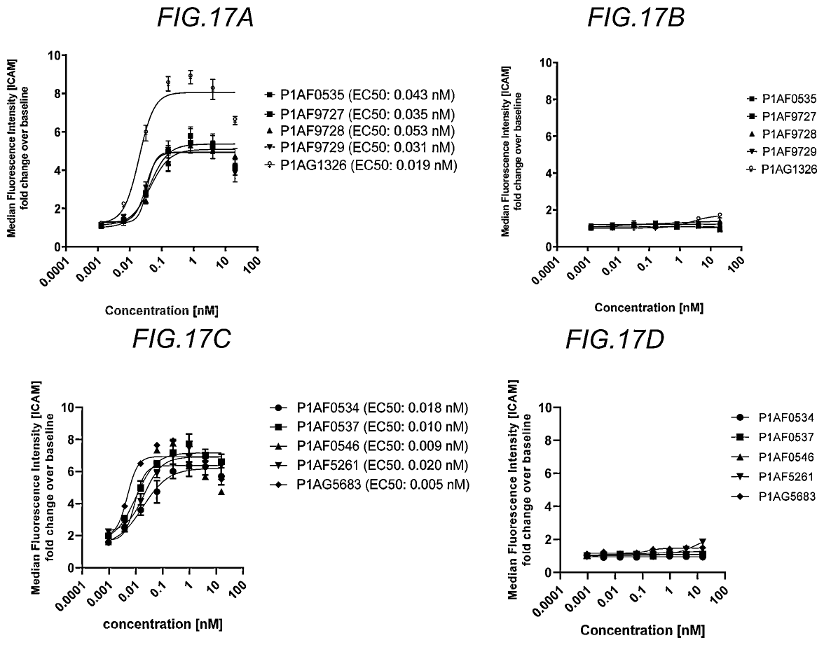

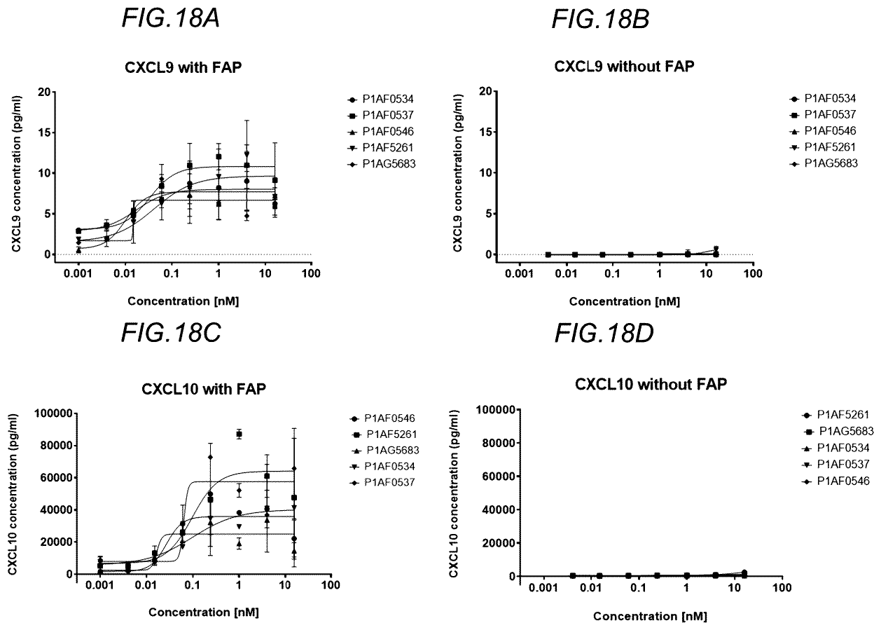

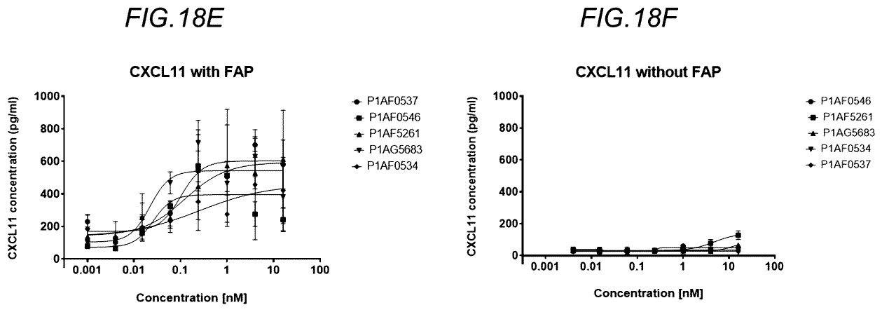

Anti-FAP/anti-LTBR bispecific antibodies induce ICAM upregulation on human endothelial cells in a dose dependent and FAP-dependent manner (FIGS. 17A to 17D). The bivalent bispecific molecules P1AF0546 and P1AG5683 have the lowest EC₅₀ values suggesting a higher potency of this 2+1 format. Anti-FAP/anti-LTBR bispecific antibodies also show a dose dependent and FAP dependent induction of CXCL9, CXCL10 and CXCL11 in the supernatant (FIGS. 18A to 18F). Taken together these data suggest that anti-FAP/anti-LTBR bispecific antibodies can modulate the endothelium to increase adhesion molecules and chemoattractants important to increase immune infiltration in the tumor. Importantly, the effect is dependent on the presence of FAP, proving that LTBR agonism can be restricted to the tumor microenvironment via FAP-targeting.

### 5.2.3 Effect of Anti-FAP/Anti-LTBR Bispecific Antibodies on T Cell Adhesion

The previous example showed that anti-FAP/anti-LTBR bispecific antibodies modulate human endothelium to upregulate adhesion molecules and chemoattractants, which are critical to the immune infiltration cascade. To test if these effects result in an increased T cell infiltration, we measured the first step of the immune infiltration cascade, namely the adhesion of immune cells to endothelium stimulated with anti-FAP/anti-LTBR bispecific antibodies.

Briefly, HUVEC were seeded with NIH-3T3 or NIH-3T3 overexpressing FAP and were stimulated with FAP-LTBR bispecific molecules (2 nM) as described in the previous example, or with TNFα (0.5 ng/mL, Peprotech). PBMCs were isolated from healthy donor buffy coats by density gradient centrifugation with Histopaque-1077 and frozen until use. Pan-T cells were purified from frozen PBMCs aliquots using a pan-T cell isolation kit (Miltenyi Biotec, cat. 130-096-535) and following manufacturer instructions. Purified T cells were rested overnight in T cell media (T cell optimizer media, Gibco cat. A1048501). Rested T cells were collected, washed in DPBS⁺⁺ (Gibco, cat. 14040182) supplemented with 0.2% BSA and labelled with Calcein AM (Invitrogen, cat. C3100MP) for 20 min at 37° C. After extensive washing, labelled T cells were resuspended in DPBS⁺⁺ 0.2% BSA and 100,000 cells/well were added to the treated cocultures that were previously washed with assay media. T cells were let adhere for 1 hour at 37° C. and non adherent cells were removed by inversion washes with DPBS⁺⁺ 0.2% BSA. Phase and green fluorescence images (5 per well) were acquired using an Incucyte S3 live cell analysis system (Sartorius) and adherent T cells were quantified as the area covered by green fluorescent T cells using the Incucyte image analysis software.

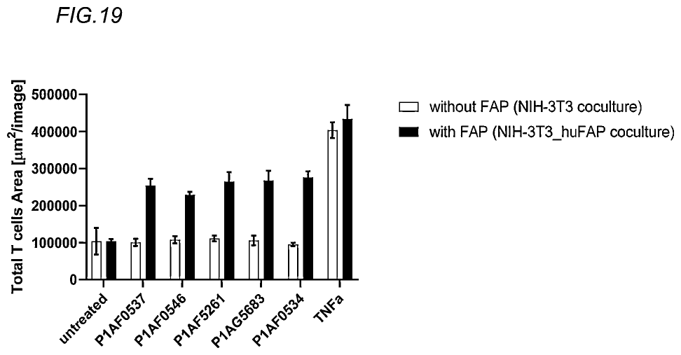

TNFα stimulation induces T cell adhesion independently of FAP and serves as a positive control. T cell adhesion was increased on endothelium treated with anti-FAP/anti-LTBR bispecific antibodies and the effect is dependent on the presence of FAP in the coculture system (FIG. 19).

Taken together these data show that LTBR agonism can be induced in a FAP-targeted manner and upregulates an inflammatory program by human endothelium that results in the induction of T cell adhesion, the first step in the immune infiltration cascade. Anti-FAP/anti-LTBR bispecific antibodies can therefore act as tumor microenvironment modulators to increase the recruitment of immune cells into the tumor.

### 5.3 Characterization of Anti-FAP/Anti-LTBR Bispecific Antibody Surrogates In Vitro and In Vivo

A subset of the novel FAP-LTBR bispecific molecules is crossreactive to murine LTBR. We generated murine surrogate molecules on a murine IgG backbone as described in previous examples and tested their in vitro and in vivo activity.

### 5.3.1 In Vitro Activity of Anti-FAP/Anti-LTBR Bispecific Antibody Surrogates on Murine Fibroblasts

To test the ability of anti-FAP/anti-LTBR surrogate molecules to activate murine stromal cells in vitro in a FAP-dependent manner, FAP-LTBR bispecific molecules were incubated with NIH-3T3 or NIH-3T3 FAP overexpressing cells and the upregulation of VCAM was measure by FACS analysis.

Briefly, NIH-3T3 or NIH-3T3 FAP overexpressing cells were seeded in a 96 well plate in complete media. Anti-FAP/anti-LTBR surrogate molecules or antibody 5G11 (anti mouse LTBR agonistic antibody, Hycult Biotech cat. hm1079) were diluted in low serum media (DMEM, 1% FBS) and cells were stimulated over night. Treated cells were detached with accutase (Gibco) and stained with a NIR live/dead dye (LIVE/DEAD Fixable Near-IR Dead Cell Stain Kit, Thermo Fischer Scientific cat. L34975) and with an anti-mouse-VCAM-Pacific Blue labelled antibody (Biolegend, cat. 105722). Samples were acquired on a flow cytometer (BD Fortessa). Median fluorescent intensity of VCAM expression was analysed in FlowJo and data were normalized to baseline untreated controls and expressed as fold change over baseline.

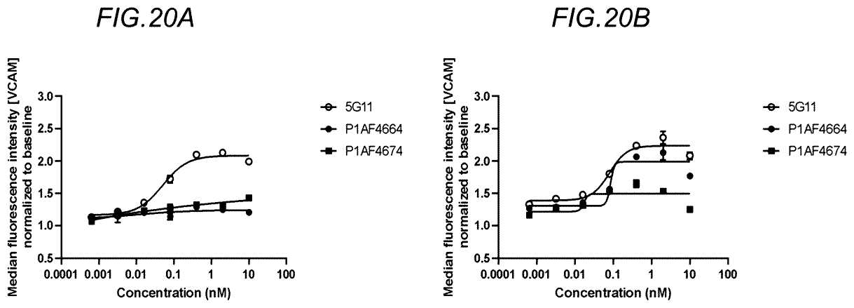

The anti-murine LTBR agonistic antibody 5G11 induces VCAM upregulation on both cell types, whereas P1AF4664 and to a lesser extent P1AF4674, are inactive on NIH-3 T3 cells and only induce VCAM in the presence of FAP expression (FIGS. 20A and 20B).

### 5.3.2 In Vitro Activity of Anti-FAP/Anti-LTBR Bispecific Antibody Surrogates on Endothelial Cells

To test the ability of anti-FAP/anti-LTBR surrogate molecules to activate endothelial cells in a FAP-dependent manner in vitro, co-cultures of human umbilical vein endothelial cells (HUVEC) and NIH-3T3 cells overexpressing human FAP were treated with bispecific molecules and the levels of the adhesion molecule ICAM on HUVEC was measured by FACS analysis.

Briefly, HUVEC (15000 cells/well, Lonza cat. C2517AS) were seeded together with parental NIH-3T3 or NIH-3T3 FAP overexpressing cells (2000 cells/well) in a gelatin coated 96-well plate in complete EGM-2 media (cat. CC3162, Lonza). Co-cultures were treated over night with FAP-LTBR bispecific molecules in assay media (EBM-2 media supplemented with 2% FBS, ascorbic acid, heparin, GA-1000 and heparin, cat. CC3156 and CC4176, Lonza). Treated co-cultures were detached with accutase (Gibco) and stained with a NIR live/dead dye in PBS (LIVE/DEAD Fixable Near-IR Dead Cell Stain Kit, Thermo Fischer Scientific cat. L34975) and with an anti-human ICAM-BV421 (BD cat. 564077, diluted 1:100) and an anti-human-CD31-APC/Cy7 labelled antibody (Biolegend cat. 303120, diluted 1:300). Samples were acquired on a flow cytometer (BD Fortessa). The median fluorescent intensity of ICAM expression in the CD31+ population was analysed in FlowJo.

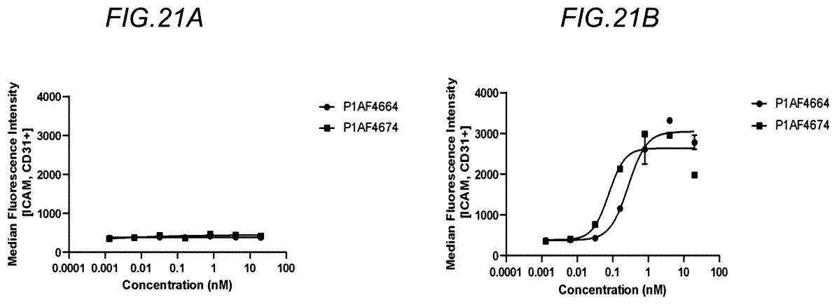

Anti-FAP/anti-LTBR surrogate molecules induce ICAM upregulation on human endothelial cells in a dose dependent and FAP-dependent manner (FIGS. 21A and 21B).

Taken together, these data show that the surrogate molecules are active in vitro and induce FAP-dependent LTBR agonism on stromal cells, similarly to the effect of the human backbone molecules. We next tested the activity of the surrogate molecules in vivo in syngeneic mouse tumor models.

5.3.3 In Vivo Efficacy and Pharmacodynamics Study with FAP-LTBR Surrogate Molecules in Colorectal Cancer and Pancreatic Cancer, Subcutaneous Tumor Models

The efficacy and pharmacodynamics effect on endothelial activation of FAP-LTBR surrogate molecules was assessed in mice bearing subcutaneous colorectal tumors (MC38-huCEA) expressing human carcinoembryonic antigen (CEA) and pancreatic tumors expressing human CEA (KPC4662-huCEA). The latter model is characterized by abundant FAP⁺ expressing stroma and scarce immune infiltrate at baseline, while the MC38-huCEA tumor model is characterized by moderate FAP expression in the stroma and a more abundant immune infiltrate at baseline.

KPC4662 cells were obtained from the University of Pennsylvania and engineered in-house in order to express human CEA. Cells were cultured in DMEM+FBS 10%+Hygromycin 500 μg/ml. MC38 cells expressing human CEA were obtained from the Beckman Research Institute of the City of Hope. Cells were cultured in DMEM+FBS 10%+Geneticin 500 μg/ml. Before injecting, cells were counted and 500,000 cells (MC38-huCEA) or 300,000 cells (KPC4662-huCEA) were injected in total volume of 100 μl, in a 1:1 mix with RPMI and Matrigel, subcutaneously in the flank of human CEA expressing mice. Tumor growth was measured at least twice weekly using a caliper and tumor volume was calculated as follows: Tumor volume=(W²/2)×L (W: Width, L: Length).

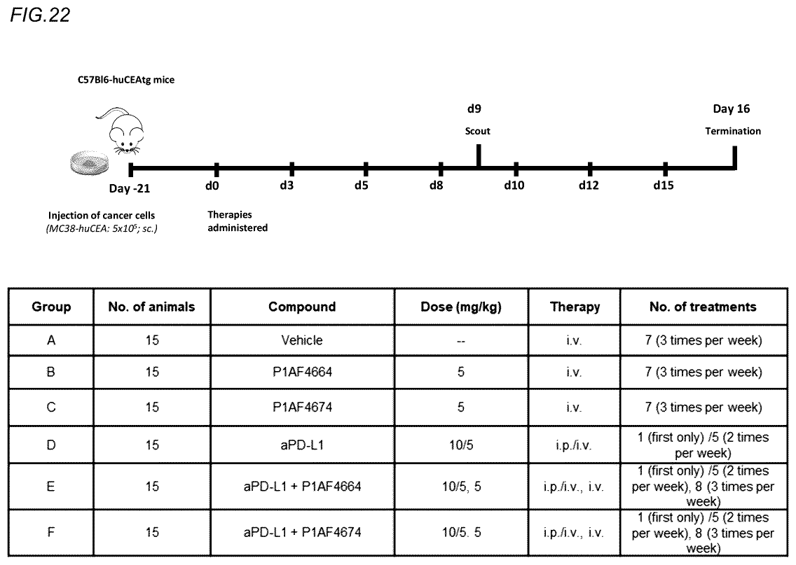

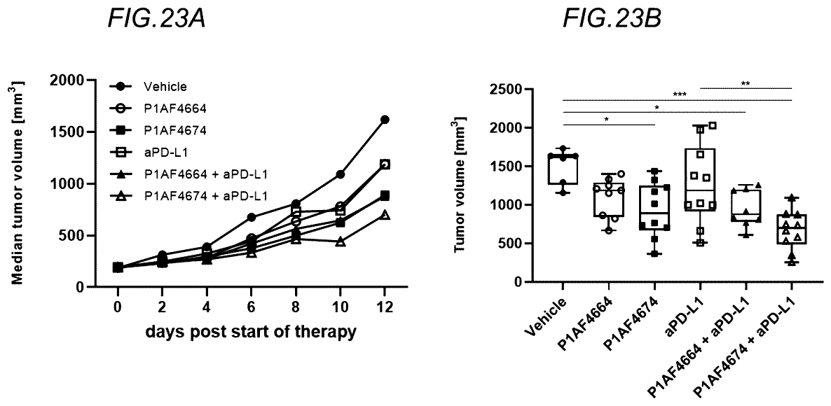

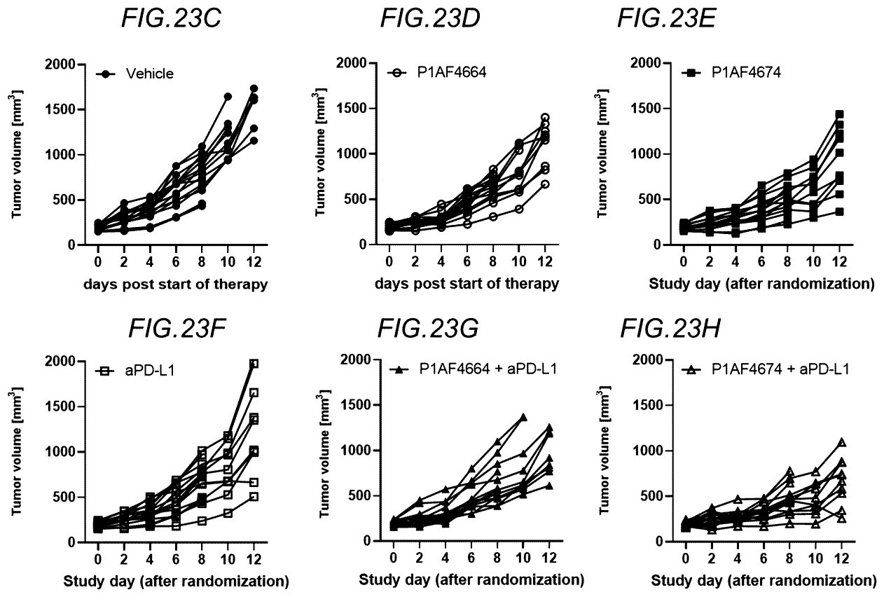

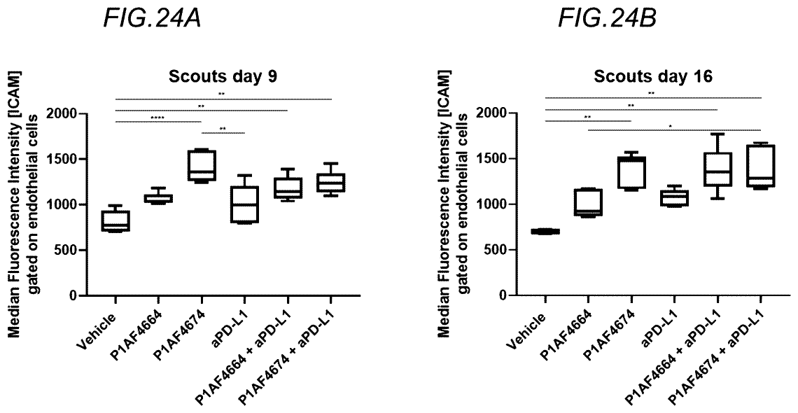

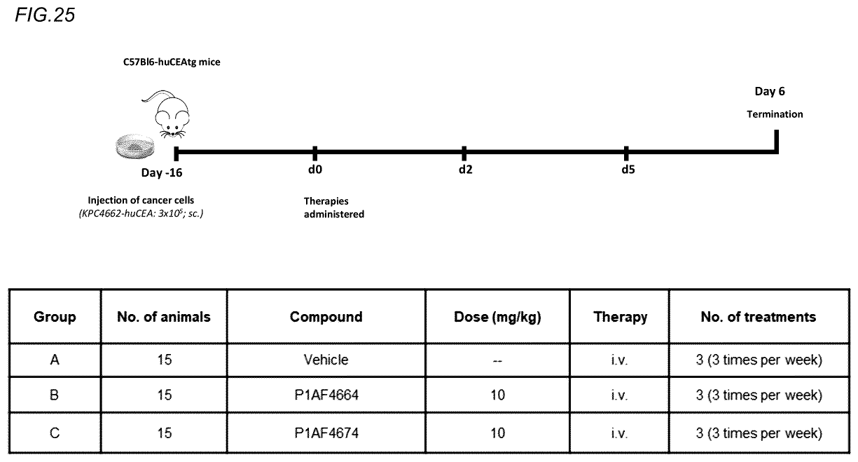

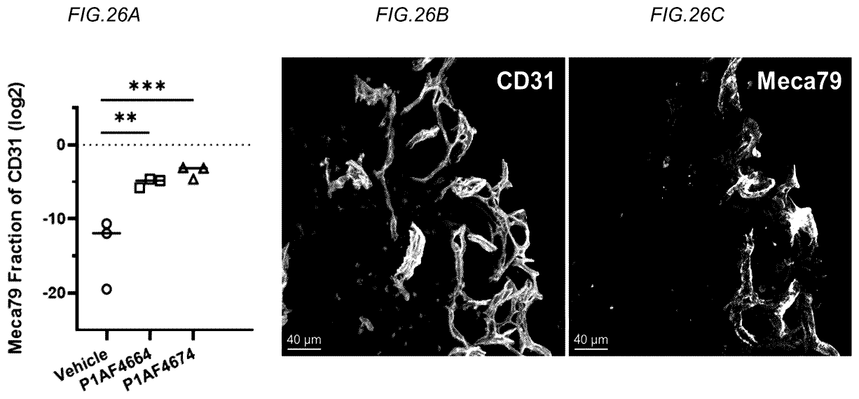

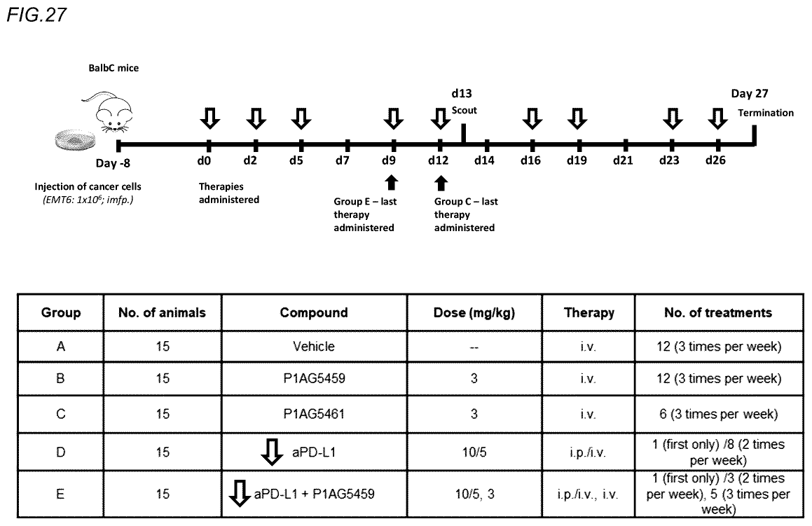

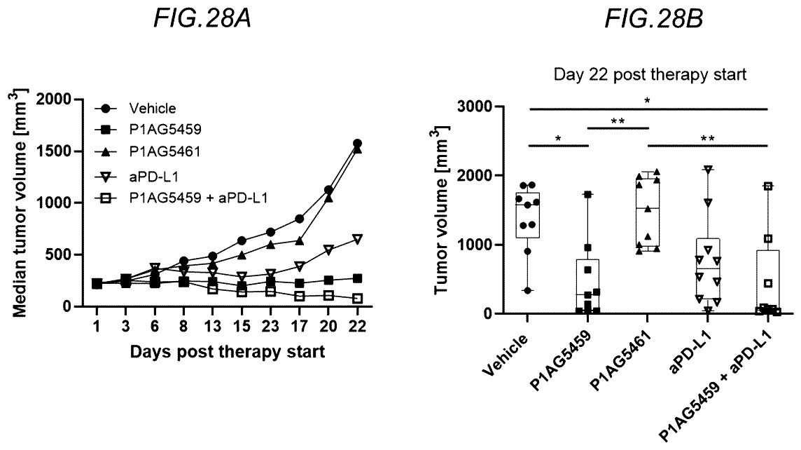

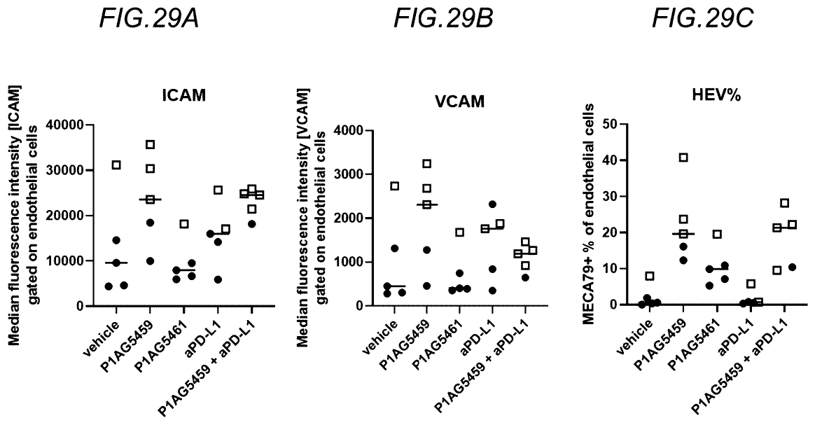

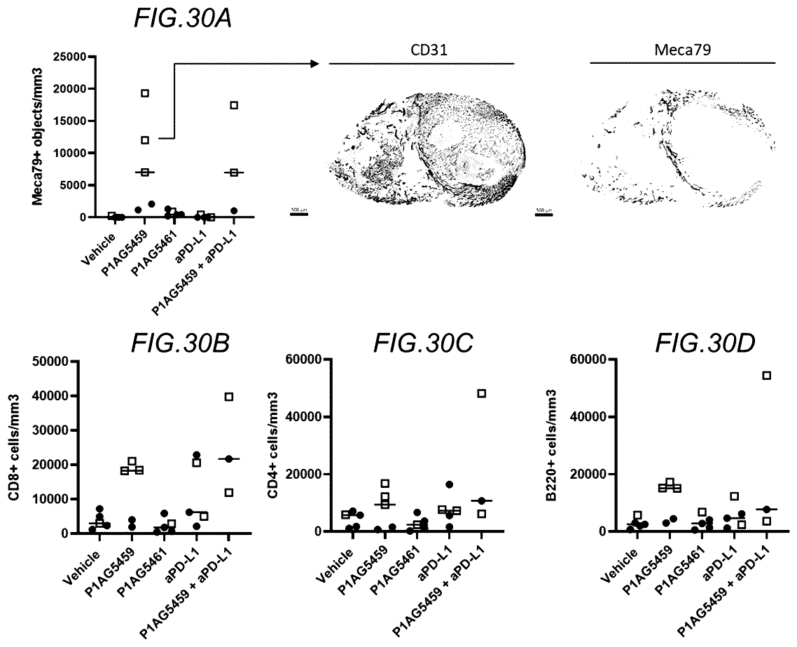

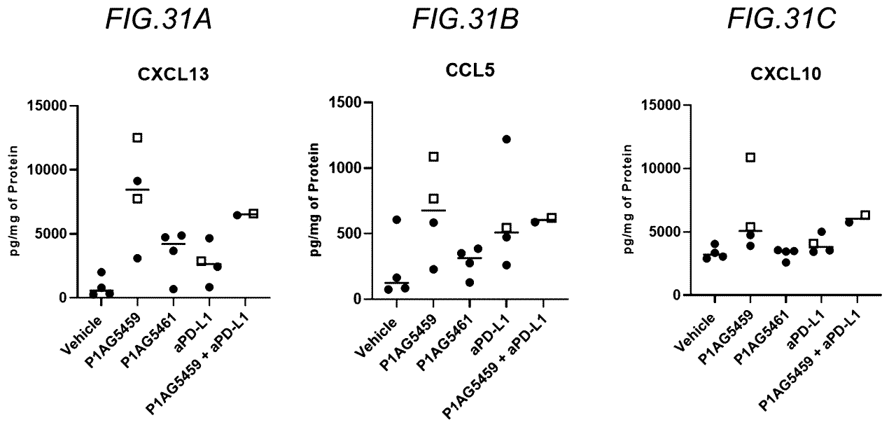

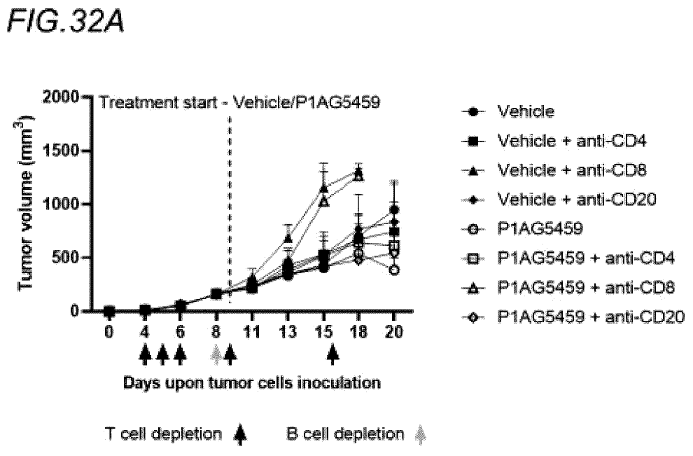

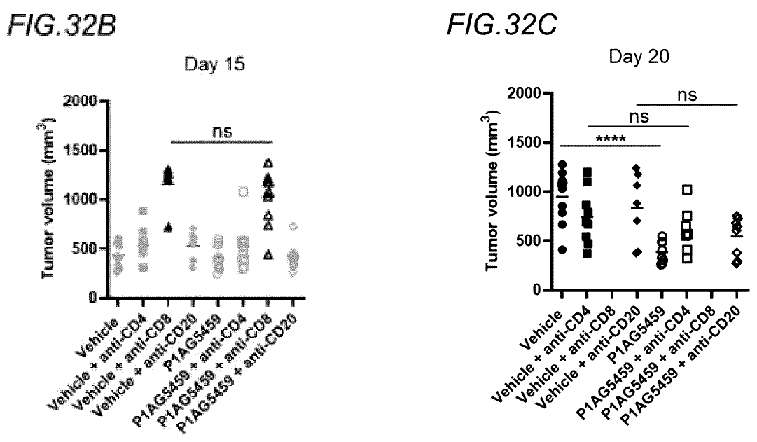

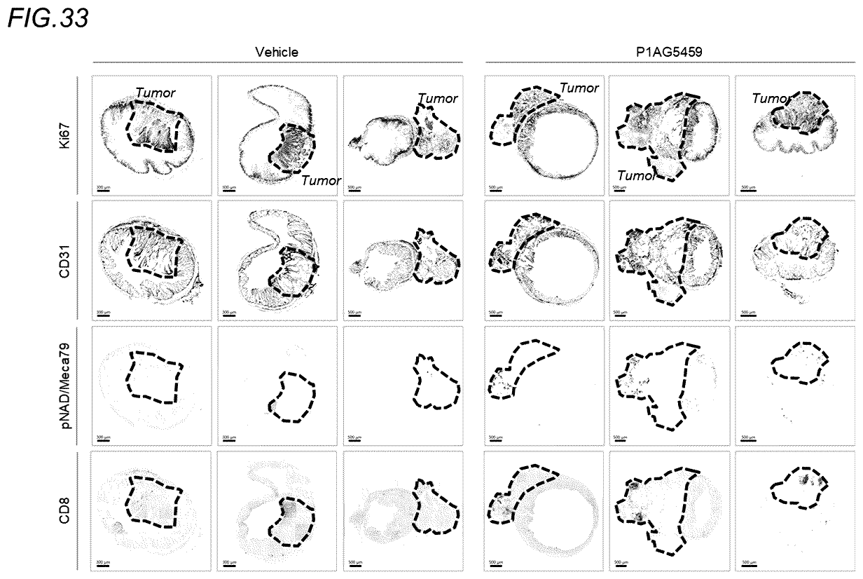

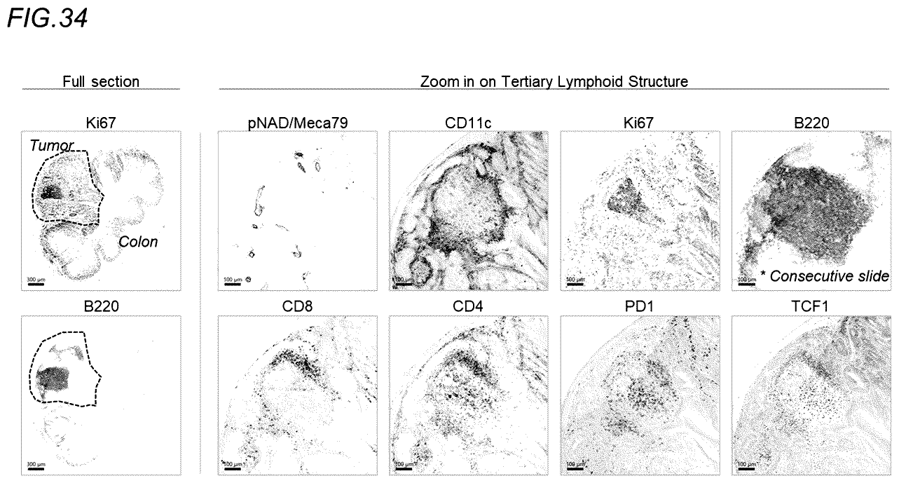

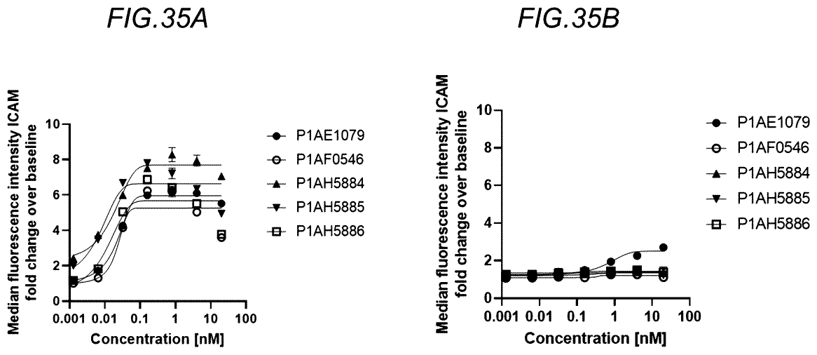

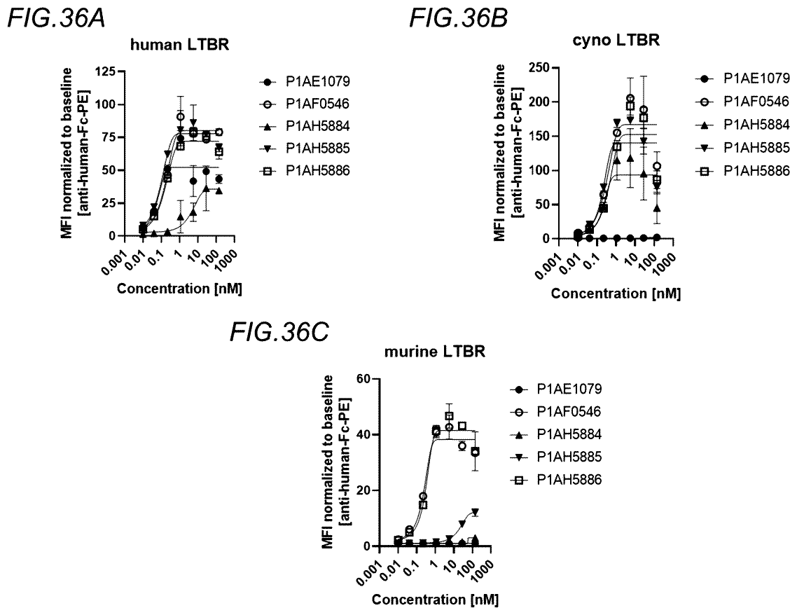

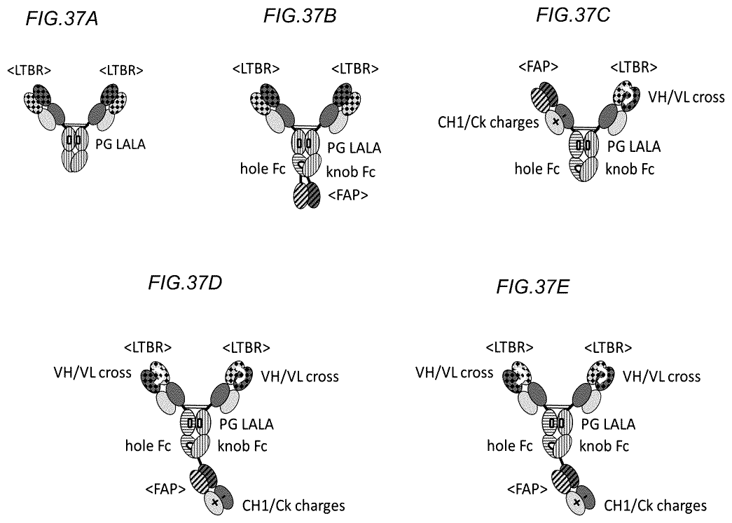

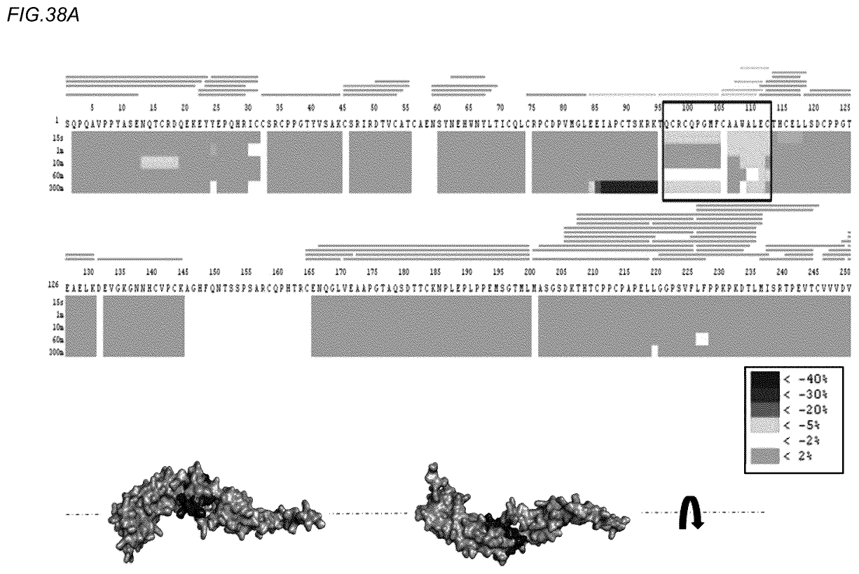

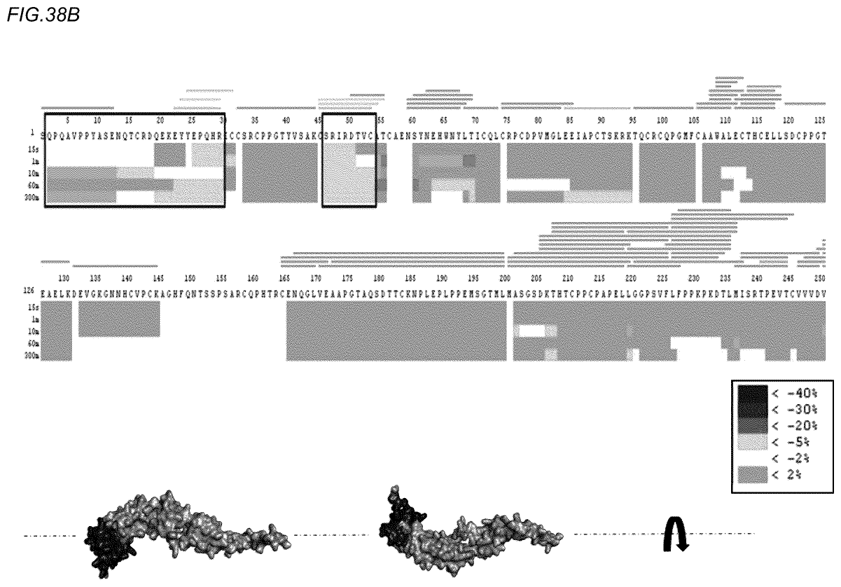

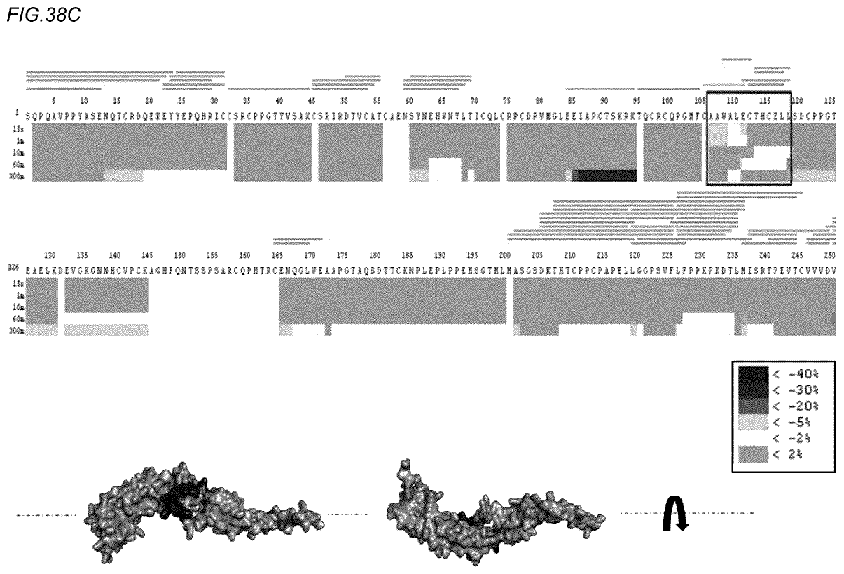

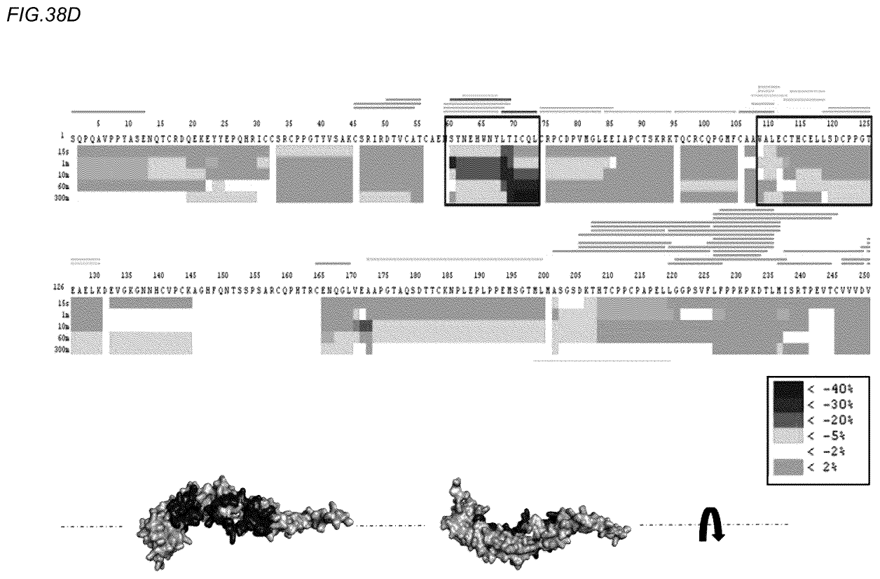

In the MC38-huCEA study (FIG. 22), mice were randomized 21 days at after cell injection, when tumor sizes reached on average approximately 195 mm³. All mice were injected i.v. with 200 μl of the appropriate solution of Vehicle, P1AF4664, P1AF4674, aPD-L1 (clone 6E11), aPD-L1+P1AF4664 and aPD-L1+P1AF4674 (P1AF4664, P1AF4674 3 times per week, 5 mg/kg and aPD-L1 twice per week 5 mg/kg, for aPD-L1 the first injection was given i.p. at 10 mg/kg). To obtain the proper amount of compounds per 200 μl, the stock solutions were diluted with Histidine buffer (20 mM Histidine, 140 mM NaCl, pH 6.0). At days 9 and 16 post treatment start, five mice, or the amount of surviving mice, were sacrificed. Tumors of these mice were processed for downstream analysis.

Tumor single cell suspensions for flow cytometry analysis were obtained by digesting tumor samples with Liberase and DNAse. Single cell suspensions were then stained for, among others, murine CD45, human CEA PerCP-Cy5.5, murine Podoplanin PE-Cy7, murine CD31 APC/Cy7 and ICAM APC. Samples were acquired on a BD Fortessa Flow Cytometer and data analyzed in FlowJo.

In the KPC4662-huCEA study (FIG. 25), after 16 days of initial tumor growth mice, when tumor sizes reached approximately 140 mm³, mice were randomized and treated three times per week with either Histidine buffer (vehicle) or P1AF4664 or P1AF4674 (10 mg/kg). All mice were injected i.v. with 200 μl of the appropriate solution. To obtain the proper amount of compounds per 200 μl, the stock solutions were diluted with Histidine buffer (20 mM Histidine, 140 mM NaCl pH 6.0). At day 6 post treatment start five mice of each group were sacrificed. Tumors of these mice were processed for downstream histological analysis.

For histological analysis by immunofluorescence (three-dimensional image processing, 3DIP) tumors were fixed in BD Cytofix solution, diluted 1:4 in PBS, for approximately 20 hours. After washing and transferring to PBS, tumors were embedded in 4% low-gelling temperature agarose. Tumor sections (70 μm thick) were cut from these blocks using a Leica VT1200s Vibratome equipped with common razor blades. Subsequently sections were permeabilized (TBS+0.3% Triton-X) and blocked using BSA and Mouse Serum (each 1%) for two hours before stained overnight (ca. 15 hours) at room temperature using among others the following antibodies: PNAd/Meca79 (AF647) and CD31 (AF594). Image acquisition was done on a Leica SP8 inverted confocal microscope. Image quantification was carried out in Imaris 9.6 (Bitplane).

For the tumor growth data, flow cytometry, as well as 3DIP analysis, plotting was done in GraphPad PRISM 8 and One-Way Anova tests, doing multiple comparisons between groups, were run with a Holm-Sidak correction for multiple testing.

In the MC38-huCEA (FIG. 22) study we observed that treatment with FAP-LTBR surrogate molecules P1AF4664 and P1AF4674 inhibits growth of MC38-huCEA tumors in monotherapy and improves the tumor growth inhibition of aPD-L1 (FIGS. 23A to 23C). The tumor growth kinetics show tumor growth inhibition of treated animals starting to be visible after around one week of treatment (FIGS. 23A and 23C). At 12 days post treatment start, there is a statistically significant difference of tumor volume between the Vehicle and P1AF4674, as well as between the Vehicle and both aPD-L1 combinations with P1AF4664 and P1AF4674. Furthermore, the combination of P1AF4674 and aPD-L1 shows statistically significant smaller tumor volume than aPD-L1 monotherapy (FIG. 23B). This indicates that the 2+1 format (P1AF4674) shows superior tumor growth inhibition compared to the 1+1 format (P1AF4664).

Flow cytometry analysis of tumor single cell suspensions at day 9 and day 16 post treatment start revealed upregulation of the adhesion molecule ICAM on tumor endothelium upon treatment with FAP-LTBR surrogate molecules (FIGS. 24A and 24B). P1AF4674, which is a bivalent LTBR agonist, is superior to the monovalent molecule P1AF4664.

In the KPC4662-huCEA study (FIG. 25), 6 days post treatment start, we observed through histological analysis a statistical significant increase in the presence of High Endothelial Venules (HEVs) in the tumor tissue upon treatment with FAP-LTBR surrogate molecules P1AF4664 and P1AF4674 (FIGS. 26A, 26B and 26C).

5.3.4 In Vivo Efficacy and Pharmacodynamics Study with FAP-LTBR Surrogate Molecule in Combination with Anti-PD-L1 in an Orthotopic Breast Cancer Model

The efficacy and pharmacodynamics effect on endothelial activation, cytokine secretion and immune infiltration of anti-FAP/anti-LTBR bispecific antibody P1AG5459, in monotherapy or combination with the checkpoint inhibitor aPD-L1, was assessed in mice bearing orthotopic breast tumors (EMT6). The model is characterized by abundant FAP⁺ expressing stroma and scarce immune infiltrate, mainly localized at the tumor edge at baseline.

EMT6 cells were obtained from ATCC (CRL-2755). Cells were cultured in DMEM+15% FCS. Before injecting, cells were counted and 1,000,000 cells were injected in total volume of 50 μl, in a 1:1 mix with RPMI and Matrigel, into the mammary fat pad of BALB/c mice. Tumor growth was measured at least twice weekly using a caliper and tumor volume was calculated as follows: Tumor volume=(W²/2)×L (W: Width, L: Length). In this study responders and non-responders were defined as having a tumor volume, at the analyzed timepoint, below or above the tumor volume at start of the therapy respectively.

Tumor single cell suspensions for flow cytometry analysis and histological analysis was performed as described in 5.3.3 with the addition of antibodies to CD8 (BV421), CD4 (BV570) and B220 (BV711) to the histology panel.

Chemokine levels in tumor lysates were assessed using a BCA kit (Thermo Scientific, Pierce BCA Protein Assay Kit, #23225, US) according to manufacturer's instructions and the plate was read on the Spectramax i3 ELISA reader (Molecular Devices, US). Then the Bio-Plex Pro™ Mouse Chemokine Panel 33-Plex (Ser. No. 12/002,231, BioRad, US) was used for cytokine and chemokine level measurement in the protein lysate. The samples were prepared according to the manufacturer's instructions in a 96-well mag-plates (Bio-Plex Pro™ Flat Bottom Plates, #171025001, BioRad, US). The assay was read on the Flexmap 3D® machine (Luminex, US) following the instructions of the xPonent® software, version 4.2, provided with the machine.

In the EMT6 study (FIG. 27) we observed that treatment with the FAP-LTBR surrogate molecule P1AG5459 effectively controls EMT6 orthotopic tumors in monotherapy and further improves the partial tumor growth inhibition of anti PD-L1 treatment, inducing tumor regression (FIG. 28A-B). At the end of the study, 3 mice in the monotherapy arm and 5 mice in the combination arm were tumor free. Importantly, the tumor growth inhibition effect is not observed when mice are treated with an untargeted (DP47)-LTBR antibody (P1AG5461, FIG. 28A-B), demonstrating the FAP-dependent effect of the bispecific anti-FAP/anti-LTBR bispecific antibody in vivo. Flow cytometry analysis of tumor single cell suspension at day 13 after start of therapy revealed FAP-dependent upregulation of the adhesion molecules ICAM (FIG. 29A) and VCAM (FIG. 29B) on tumor endothelium in all groups treated with P1AG5459, but not with P1AG5461. Upregulation of adhesion molecules on tumor endothelium could facilitate transendothelial migration of immune cells from blood into the tissue. Moreover, an abundant Meca79+ HEV population can be detected in tumors treated with P1AG5459 (FIG. 29C). HEVs are specialized vessels for the extravasation of naïve and memory T cells in lymphoid organs and have been recently linked to increased immune infiltration in tumors (Asrir et al, Cancer Cell. 2022 Mar. 14; 40(3):318-334.e9. doi: 10.1016/j.ccell.2022.01.002). The observed PD readouts were heterogeneous, but a higher effect on the levels of adhesion molecules on endothelial cells, and a higher percentage of HEVs are observed in tumors characterized as responders (open squares, Q) as compared to non responders (closed circles, ●, FIG. 29A-C). Immunofluorescent histological analysis confirmed the significant increase in the presence of Meca79+ differentiated high endothelial venules (FIG. 30A) in tumors treated with P1AG5459 alone or in combination with aPD-L1 antibody. HEVs are much less detected in tumors treated with P1AG5461 confirming the FAP dependent activity of FAP LTBR in vivo. Histological analysis further revealed that treatment with P1AG5459 increases the infiltration of B cells (FIG. 30D), CD8 (FIG. 30B) and CD4 T cells (FIG. 30C) into the tumor. Increased immune infiltrate was also heterogenous, but a higher level of immune infiltration is observed in responder tumors (open squares, Q) as compared to non responders (closed circles, ●). The increased immune infiltrate is FAP-dependent, as it is not observed upon treatment with the untargeted molecule P1AG5461. Analysis of tumor lysates revealed an increase in the amounts of the chemokines CXCL13 (FIG. 31A), CCL5 (FIG. 31B) and CXCL10 (FIG. 31C) in response to P1AG5459 treatment. Chemokine upregulation at the tumor site in response to FAP-LTBR treatment can mediate the attraction of more immune cells at the tumor site.

5.3.5 In Vivo Efficacy Study with FAP-LTBR Surrogate Molecule and T and B Cell Depletion Groups in an Orthotopic Breast Cancer Model.

The dependency of anti-tumor efficacy of P1AG5459, in monotherapy, was assessed in mice depleted of CD4 T cells, CD8 T cells or CD20⁺ expressing B cells and bearing orthotopic breast tumors (EMT6). The model is characterized by abundant FAP⁺ expressing stroma and scarce immune infiltrate, mainly localized at the tumor edge at baseline.

EMT6 cells were obtained from ATCC (CRL-2755). Cells were cultured in DMEM+15% FCS. Before injecting, cells were counted and 100,000 cells were injected in total volume of 50 μl, in a 1:1 mix with RPMI and Matrigel, into the mammary fat pad of BALB/c mice. Tumor growth was measured at least twice weekly using a caliper and tumor volume was calculated as follows: Tumor volume=(W²/2)×L (W: Width, L: Length). CD4/CD8 T cell depletion was carried out by injecting 150 microgram anti-CD4 (GK1.5) or anti-CD8 (2.43) on days 4, 5, 6, 9 and 16 intra peritoneal. B cell depletion was carried out by injecting 250 microgram anti-CD20 (SA271G2) on day 8 intra venous.

For the tumor growth data plotting was done in GraphPad PRISM 8 and One-Way Anova tests, doing multiple comparisons between indicated groups, were run with a Holm-Sidak correction for multiple testing.

In this study we confirmed that treatment with the FAP-LTBR surrogate molecule P1AG5459 effectively controls EMT6 orthotopic tumors in monotherapy (see FIGS. 32A to 32C). We also show that in the absence of CD8 T cells, Vehicle and P1AG5459 treated tumors grow similarly fast. At Day 15, when there are still more than 3 animals left per group, a statistical analysis indicated no statistical significant difference in tumor volume between CD8 depleted Vehicle and P1AG5459 groups, showing that CD8 T cells are necessary to mediate P1AG5459 efficacy in the EMT6 tumor model (FIG. 32B). Similarly, there was no statistical significant difference in tumor volume between CD4 depleted Vehicle and P1AG5459 groups and between CD20⁺ expressing B cells depleted vehicle and P1AG5459 groups, which were analysed at Day 20 (FIG. 32C), suggesting that CD4 and B cells also play a role in mediating the efficacy of P1AG5459.

5.3.6 In Vivo Pharmacodynamics Study with a FAP-LTBR Surrogate Molecule in an Orthotopic Model of CRC Harboring Mutations in Apc/KrasG12D/p53 shRNA/Smad4

The pharmacodynamics effects of P1AG5459, in monotherapy, was assessed in mice bearing orthotopic CRC tumors (AKPS organoid model). The model is characterized by abundant FAP⁺ expressing stroma and very scarce immune infiltrate, if at all, localized at the tumor edge at baseline.

AKPS Colorectal Cancer organoids (mouse intestinal organoids engineered via CRISPR-Cas9 to harbor mutations in Apc, KrasG12D (Knock in), p53 (Knock out) and Smad4) were obtained from Genentech. Organoids were cultured in medium containing B-27™ Supplement (Gibco™), N₂, Advanced DMEM/F-12 (Gibco™) plus glutamine and HEPES. Before injecting, organoids were counted and 200 organoids were injected in total volume of 50 μl of the same medium described above, into the submucosa of the Colon (via colonoscopy) of C57BL/6-N mice. Tumor growth was monitored weekly via colonoscopy.

Tumor harvest was carried out when mice needed to be sacrificed according to local guidelines or at last at day 50 post implantation. For histological analysis by immunofluorescence (three-dimensional image processing, 3DIP) tumors were fixed in BD Cytofix™ fixation buffer solution, diluted 1:4 in PBS, for approximately 20 hours. After washing and transferring to PBS, tumors were embedded in 4% low-gelling temperature agarose. Tumor sections (70 μm thick) were cut from these blocks using a Leica VT1200s Vibratome equipped with common razor blades. Subsequently sections were permeabilized (TBS+0.3% Triton-X) and blocked using BSA and Mouse Serum (each 1%) for two hours before stained overnight (ca. 15 hours) at room temperature using among others the following antibodies: Ki67 (AF532), B220 (AF647), CD8 (BV421), CD4 (BV570), CD11c (BV480), PD1 (PE), TCF1 (AF488), PNAd/Meca79 (AF647) and CD31 (AF594). Image acquisition was done on a Leica SP8 inverted confocal microscope. Image preparation was carried out in Imaris 9.9 (Bitplane). All but one staining was carried out on the same section (indicated in FIG. 34).

As observed in other mouse models, we confirmed that treatment with P1AG5459 induces the differentiation of Meca79+ HEVs (FIG. 33). Importantly, HEVs develop within the tumor, but not in the adjacent normal colon tissue (FIG. 33). Moreover, treatment with P1AG5459 dramatically increased the number of CD8 T cells in the tumor (FIG. 33). Further, in one sample of a mouse receiving P1AG5459 treatment that was sacrificed at day 50, we found that the tumor immune infiltrate organized itself into a Tertiary Lymphoid Structure (TLS), characterized by the presence of CD11c myeloid cells, distinct B and T cell zones, a proliferating B cell center and containing activated T cells (PD1+) and T cells expressing the stemness marker TCF1 (FIG. 34).

This study showed not only that P1AG5459 treatment can increase the content of immune cells and HEVs within the tumor, but it also enables the formation of Tertiary Lymphoid Structures that are known to correlate with improved response to Checkpoint Inhibitor treatment in various cancers (Schumacher and Thommen, DOI: 10.1126/science.abf9419).

Together, the data described in Example 5 show that the novel anti-FAP/anti-LTBR bispecific antibodies are efficacious in vivo and are able to control tumor growth in a model of colorectal cancer and immune excluded breast cancer. Anti-FAP/anti-LTBR bispecific antibodies synergized with the checkpoint inhibitor anti PD-L1, suggesting that anti-FAP/anti-LTBR bispecific antibodies can be utilized to improve response to checkpoint inhibitors in the clinic. The data also show that anti-FAP/anti-LTBR bispecific antibodies modulate the tumor microenvironment, especially the tumor vasculature, by upregulating adhesion molecules and differentiating vessels towards a HEV phenotype, and by inducing the secretion of chemoattractants. Collectively these effects improve immune infiltration into the tumor and the increase in immune cell content is mediating the anti tumor efficacy of the novel anti-FAP/anti-LTBR bispecific antibodies, which we demonstrated in the breast cancer orthotopic model. Furthermore, the novel anti-FAP/anti-LTBR bispecific antibodies can drive the formation of Tertiary Lymphoid Structures, as observed in an orthotopic colorectal cancer model.

### Example 6

### Comparison and Superiority of Anti-FAP/Anti-LTBR Bispecific Antibodies to Known LTBR Antibodies

### 6.1 Effect of Anti-FAP/Anti-LTBR Bispecific Antibodies on Endothelial Cells In Vitro

To functionally compare anti-FAP/anti-LTBR bispecific antibodies containing anti-LTBR antibody P1AE9459 or known anti-LTBR antibodies CBE11 (as described in U.S. Pat. No. 7,429,644 B2) and BHA10 (as described in U.S. Pat. No. 7,429,645 B2), co-cultures of human umbilical vein endothelial cells (HUVEC) and NIH-3T3 cells overexpressing human FAP were treated with bispecific molecules and the levels of the adhesion molecule ICAM on HUVEC was measured by FACS analysis as described in 5.2.2.

All bispecific molecules tested upregulate ICAM on endothelial cells in the presence of FAP with similar potency (FIG. 35A). Most molecules are inactive in the absence of FAP (FIG. 35B), however a bispecific antibody containing CBE11 as an LTBR agonist antigen binding domain (P1AE1079) shows some activity in the absence of FAP at high doses suggesting superiority of bispecific formats containing the novel LTBR agonistic antibody or BHA10 over antibody clone CBE11.

### 6.2 Anti-FAP/Anti-LTBR Bispecific Antibodies Bind Human, Cynomolgus and Murine LTBR on Cells

To compare the ability of bispecific antibodies containing the anti-LTBR antibody P1AE9459 and known anti-LTBR antibodies CBE11 and BHA10 to bind human, cynomolgus monkey (cyno) and murine LTBR on cells, anti-FAP/anti-LTBR bispecific antibodies were incubated with cells engineered to express either human, cyno or mouse LTBR and their binding was measured by flow cytometry as described in 5.1.

All anti-FAP/anti-LTBR bispecific antibodies bind to human LTBR on cells similarly, however P1AE1079 binds to human LTBR less efficiently than bispecific antibodies containing BHA10 or anti-LTBR antibody P1AE9459. Moreover, bispecific antibodies with monovalent binding to LTBR (1+1 format) containing BHA10 bind less well to human LTBR than molecules with bivalent binding to LTBR (for BHA10 as well as for novel anti-LTBR antibody P1AE9459) (FIG. 36A).

Bispecific antibodies containing novel anti-LTBR antibody P1AE9459 or BHA10 bind similarly to cyno LTBR, however a bispecific molecule containing CBE11 does not bind to cyno LTBR (FIG. 36B) in agreement to Example 5.1. Crossreactivity to cyno is an important advantage of the novel bispecific antibody, as it will allow the investigation of the toxicity profile in a relevant species. As LTBR is a widely expressed target, de-risking of potential toxicologic effects in vivo is of great importance.

Interestingly, only bispecific molecules containing novel anti-LTBR antibody P1AE9459 bind to murine LTBR on cells, whereas bispecific molecules containing known antibodies CBE11 or BHA10 do not crossreact to murine LTBR (FIG. 36C). These data suggest that novel anti-LTBR antibody P1AE9459 binds a distinct epitope that allows crossreactivity across three species despite low identity between human and mouse LTBR (69% alignment of the human and mouse ECD). Binding to murine LTBR is an important advantage of the novel bispecific antibodies, as it allows for direct investigation of in vivo efficacy and pharmacodynamics effects of the same binder in relevant syngeneic models, requiring only the murinization of the backbone. Syngeneic models reflecting different human tumor biologies can guide the exploration of relevant combination strategies and patient selection strategies to inform clinical development.

### 6.3 Comparison of the Binding to LTBR by Surface Plasmon Resonance (SPR)

The binding behaviour of new LTBR agonistic antibody P1AE9459 was compared with that of known LTBR antibodies CBE11 and BHA10. Binding of anti-LTbR IgG antibodies to human, cynomolgus and murine LTBR was investigated by surface plasmon resonance using a Biacore T200 or Biacore 8K instrument (Cytiva). All experiments were performed at 25° C. using HBS-P (Cytiva #BR-1006-71) as running and dilution buffer. An anti-human IgG PG LALA specific antibody was immobilized on a Series S CM5 or CM3 Sensor Chip using standard amine coupling chemistry. Anti-LTBR IgGs were captured on the surface leading to capture levels between 10 and 100 RU. Human, cynomolgus or murine LTBR (P1AE7979, P1AE4411, or P1AE4410, respectively) were injected for 120 s with concentrations from 3.7 up to 300 nM (1:3 dilution series) onto the surface (association phase) at a flow rate of 30 μl/min. The dissociation phase was monitored for 600 s by washing with running buffer. The surface was regenerated by injecting 10 mM NaOH for 60 s at a flow rate of 5 μl/min. Bulk refractive index differences were corrected by subtracting the response obtained from a reference surface. Blank injections were subtracted (double referencing). The recorded sensorgrams were fitted to a 1:1 Langmuir binding model using the BIAevaluation software. The association and dissociation rate constants as well as the respective affinities (equilibrium dissociation constant KD) have been summarized in Table 18 below. The novel LTBR agonistic antibody P1AE9459 is cross-reactive to human, cynomolgus, and murine LTBR whereas CBE11 binds to human LTBR only and BHA10 is cross-reactive to human and cynomolgus LTBR but does not bind to murine LTBR. The affinities of P1AE9459 and BHA10 to human LTBR are comparable (0.3 nM vs. 0.5 nM, respectively), whereas the affinity of CBE11 to human LTBR is ˜3-fold lower than that of P1AE9459. In comparison to the known antibodies CBE11 and BHA10, P1AE9459 is the most affine antibody to human and cynomolgus LTBR and the only one that is cross-reactive to the murine receptor.

TABLE 18
Comparison of binding kinetics of anti-LTBR IgG antibodies to
human, cynomolgus and murine LTBR as determined by SPR

| anti-LTBR | | | | KD | T1/2 |
|---|---|---|---|---|---|
| IgG | antigen | Ka (1/ms) | Kd (1/s) | (nM) | (min) |
| P1AE9459 | hu LTBR | 7.95E+05 | 2.17E−04 | 0.3 | 53 |
| (9459) | cyno LTBR | 6.41E+05 | 1.91E−04 | 0.3 | 61 |
| | mu LTBR | 7.48E+05 | 7.95E−03 | 10.6 | 1 |
| P1AE1873 | hu LTBR | 4.89E+06 | 5.43E−03 | 1 | 2 |
| (CBE11) | cyno LTBR | no binding | | | |
| | mu LTBR | no binding | | | |
| P1AH0119 | hu LTBR | 1.72E+06 | 8.53E−04 | 0.5 | 14 |
| (BHA10) | cyno LTBR | 1.31E+06 | 1.54E−03 | 1.18 | 8 |

| | |
|---|---|
| mu LTBR | no binding |

### 6.4 Epitope Determination by Hydrogen/Deuterium Exchange (HDX) Mass Spectrometry

The epitopes of LTBR agonistic antibody P1AE9459 as well as CBE11 (P1AE1873) and BHA10 (P1AH0119) were determined by hydrogen/deuterium exchange mass spectrometry. For comparison, the binding interface of the single-chain construct of the natural ligand human lymphotoxin α1β2 (P1AE1235) on human LTBR (P1AH2680) that was cloned from serine 28 to methionine 227 was also determined.

Human LTBR was labeled with deuterium by incubation in buffered, deuterated water (D₂O) either in the absence of an antibody or ligand (protein state 1) or in the presence of the lead antibody P1AE9459 (protein state 2) or in the presence of antibody CBE11 (protein state 3) or in the presence of BHA10 (protein state 4) or in the presence of the single-chain construct of the natural ligand human lymphotoxin α1β2 (protein state 5) for various durations. The following buffers and instrumentation were used: H₂O-buffer (20 mM Histidine, 140 mM NaCl, pH 6.0 in H₂O), D₂O-buffer (20 mM Histidine, 140 mM NaCl, pH 6.0 in D₂O), and quench-buffer (4 M Urea, 0.17 M KH₂PO₄, 0.3 M tris-(2-carboxyethyl)phosphine hydrochloride (TCEP), pH 2.8). For pipetting and labelling, a PAL HTC autosampler from Leap Technologies was used. All labelling samples (0 min, 15 seconds, 1, 10, 60 and 300 minutes labeling) were performed in triplicates.

After various labeling times, the exchange reaction was quenched by addition of quenching buffer. Subsequently, all samples were protease-digested by Nepenthesin-2. The resulting peptides were separated by reversed-phase UPLC and identified by MS/MS detection using a Thermo Orbitrap Fusion™ Lumos™ Tribid™ mass spectrometer (Thermo Fisher Scientific). For data evaluation, in order to identify peptides with different deuteration levels of LTBR in protein states 1, 2, 3, 4, and 5, respectively, the commercially available software tools Peaks™ Studio from Bioinformatics Solutions, Inc. (BSI) and HDExaminer from Sierra Analytics were used.

In more detail, analyses of deuterium labeled samples were performed as follows: labelled and quenched samples were loaded onto a cooled (0° C.) Waters® nanoACQUITY UPLC® system (Waters Corp., Milford, MA). Digestion with aspartic proteinase nepenthesin-2 (Affipro, #AP-PC-004) was performed online at 20° C. The obtained peptides were trapped and desalted at 0° C. for 3 min (0.23% formic acid in water, 200 μl/min, Waters™ VanGuard C18, 1.7 μm, 2.1×5 mm). Subsequently a reversed phase separation (Waters™ BEH C18 column, 1.7 μm, 1×100 mm) using a gradient from 8-35% with 0.23% formic acid in acetonitrile (pH 2.5) in 7 min for all samples, at a flow rate of 40 μl/min, was performed.

Mass analysis was performed by the Thermo Orbitrap Fusion™ Lumos™ Tribid™ mass spectrometer using positive ion-electrospray ionization. For peptide identification using the 0 min-labeling-time sample, higher-energy collisional dissociation (HCD) was used for the first replicate, electron transfer dissociation (ETD) for the second replicate and electron-transfer/higher-energy collision dissociation (EThcD) for the third replicate, all in data dependent acquisition (DDA) mode. For all labelling measurements, a full scan mode was sufficient.

In terms of data evaluation, Peaks™ Studio, Bioinformatics Solutions Inc. (BSI) was used for peptide identification according to the instructions of the provider. HDExaminer, Sierra Analytics was used to determine deuterium incorporation in peptides from the different labeling times in comparison with the undeuterated control (0 min labelling time) according to instructions of the provider. The deuteration difference map of HDExaminer, comparing deuteration levels in the bound proteins states 2, 3, 4 and 5 respectively with the unbound protein state 1, was used for determination of the epitopes.

The deuteration difference maps as determined for the different LTBR antibodies as well as for the natural ligand human lymphotoxin α1β2 (P9AE1235) are shown in FIGS. 38A to 38D. A summary of the corresponding epitope regions is shown in Table 19 below. The epitope of P1AE9459 is overlapping but distinct from the epitope of antibody BHA1. Antibody CBE11 binds to a completely different epitope.

TABLE 19
Summary of the epitope regions as determined by HDX mass spectrometry

| LTBR antibody | | AA (from-to) on P1AH2680 |
|---|---|---|
| or ligand | Epitope Region | (UniProt P36941-1) |
| P1AE9459 | -QCRCQPGMFCAAWALEC- | 96-112 (123-139) |
| | (SEQ ID NO: 351) | |
| P1AH0119 | -AWALECTHCELL- | 107-118 (134-145) |
| (BHA10) | (SEQ ID NO: 352) | |
| P1AE1873 | -QTCRDQEKEYYEPQHR- | 14-29 (41-56) |
| (CBE11) | (SEQ ID NO: 353) | |
| | -SRIRDTVC- | 46-53 (73-80) |
| | (SEQ ID NO: 354) | |
| P1AE1235 (hu | -SYNEHWNYLTICQL- | 60-73 (87-100) |
| lymphotoxin α1β2) | (SEQ ID NO: 355) | |
| | -WALECTHCELLSDCPPGT- | 108-125 (135-152) |
| | (SEQ ID NO: 356) | |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-38. (canceled)

39. A bispecific and agonistic lymphotoxin beta receptor (LTBR) antibody, wherein the bispecific and agonistic LTBR antibody comprises:
(a) a first antigen binding domain that specifically binds to Fibroblast Activation Protein (FAP),
(b) a second antigen binding domain that specifically binds to human LTBR, and
(c) a Fc domain of human IgG1 subclass comprising one or more amino acid substitution that reduces the binding affinity of the antigen binding molecule to an Fc receptor and/or effector function,
wherein the second antigen binding domain that specifically binds to LTBR comprises:
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32; or
(ii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; or
(iii) a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80; or
(iv) a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88; or
(v) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(vi) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(vii) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(viii) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

40. The bispecific and agonistic LTBR antibody of claim 39, wherein the bispecific and agonistic LTBR antibody activates LTBR upon binding to FAP.

41. The bispecific and agonistic LTBR antibody of claim 39, wherein the bispecific and agonistic LTBR antibody comprises a third antigen binding domain that binds to human LTBR.

42. The bispecific and agonistic LTBR antibody of claim 41, wherein the third antigen binding domain that binds to human LTBR is identical to the second antigen binding domain.

43. The bispecific and agonistic LTBR antibody of claim 39, wherein the first antigen binding domain that specifically binds to FAP comprises
(i) a heavy chain variable region (V_{H}FAP) comprising a heavy chain complementary determining region (CDR-H1) comprising the amino acid sequence of SEQ ID NO:3, a CDR-H2 comprising the amino acid sequence of SEQ ID NO:4, and a CDR-H3 comprising the amino acid sequence of SEQ ID NO:5, and a light chain variable region (V_{L}FAP) comprising a light chain complementarity determining region (iv) CDR-L1 comprising the amino acid sequence of SEQ ID NO:6, a CDR-L2 comprising the amino acid sequence of SEQ ID NO:7, and a CDR-L3 comprising the amino acid sequence of SEQ ID NO:8, or
(ii) a heavy chain variable region (V_{H}FAP) comprising a CDR-H1 comprising the amino acid sequence of SEQ ID NO:19, a CDR-H2 comprising the amino acid sequence of SEQ ID NO:20, and a CDR-H3 comprising the amino acid sequence of SEQ ID NO:21, and a light chain variable region (V_{L}FAP) comprising a CDR-L1 comprising the amino acid sequence of SEQ ID NO:22, a CDR-L2 comprising the amino acid sequence of SEQ ID NO:23, and a CDR-L3 comprising the amino acid sequence of SEQ ID NO:24.

44. The bispecific and agonistic LTBR antibody of claim 39, wherein the first antigen binding domain that specifically binds to FAP comprises a heavy chain variable region (V_{H}FAP) comprising an amino acid sequence that is at least about 95% identical to the amino acid sequence of SEQ ID NO:9, and a light chain variable region (V_{L}FAP) comprising an amino acid sequence that is at least about 95% identical to the amino acid sequence of SEQ ID NO:10, or it comprises a heavy chain variable region (V_{H}FAP) comprising an amino acid sequence that is at least about 95% identical to the amino acid sequence of SEQ ID NO:25, and a light chain variable region (V_{L}FAP) comprising an amino acid sequence that is at least about 95% identical to the amino acid sequence of SEQ ID NO:26.

45. The bispecific and agonistic LTBR antibody of claim 39, wherein the first antigen binding domain that specifically binds to FAP comprises a heavy chain variable region (V_{H}FAP) comprising the amino acid sequence of SEQ ID NO:9 and a light chain variable region (V_{L}FAP) comprising the amino acid sequence of SEQ ID NO:10 or it comprises a heavy chain variable region (V_{H}FAP) comprising the amino acid sequence of SEQ ID NO:25 and a light chain variable region (V_{L}FAP) comprising the amino acid sequence of SEQ ID NO:26.

46. The bispecific and agonistic LTBR antibody of claim 39, wherein the bispecific and agonistic LTBR antibody comprises:
(a) a first antigen binding domain that specifically binds to FAP, comprising a heavy chain variable region (V_{H}FAP) comprising the amino acid sequence of SEQ ID NO:9 and a light chain variable region (V_{L}FAP) comprising the amino acid sequence of SEQ ID NO:10, and
(b) a second antigen binding domain that specifically binds to human LTBR, comprising a heavy chain variable region (V_{H} LTBR) comprising an amino acid sequence of SEQ ID NO:33 and a light chain variable region (V_{L} LTBR) comprising an amino acid sequence of SEQ ID NO:34.

47. The bispecific and agonistic LTBR antibody of claim 39, wherein the bispecific and agonistic LTBR antibody comprises:
(a) a first Fab fragment that binds to FAP,
(b) a second Fab fragment that binds to human LTBR, and
(c) a Fc domain of human IgG1 subclass comprising one or more amino acid substitution that reduces the binding affinity of the antigen binding molecule to an Fc receptor and/or effector function.

48. The bispecific and agonistic LTBR antibody of claim 47, wherein the second Fab fragment that specifically binds to human LTBR is a crossFab fragment.

49. The bispecific and agonistic LTBR antibody of claim 39, wherein the bispecific and agonistic LTBR antibody comprises:
(a) a first Fab fragment that binds to FAP;
(b) a second and a third Fab fragment that bind to human LTBR; and
(c) a Fc domain of human IgG1 subclass comprising one or more amino acid substitution that reduces the binding affinity of the antigen binding molecule to an Fc receptor and/or effector function,
wherein the first Fab fragment that binds to FAP is fused at its N-terminus to the C-terminus of one of the Fc domain subunits and the second and a third Fab fragment that specifically bind to LTBR are each fused at its C-terminus to the N-terminus of one of the Fc domain subunits.

50. The bispecific and agonistic LTBR antibody of claim 39, comprising:
(a) a first heavy chain comprising the amino acid sequence of SEQ ID NO:199;
(b) a second heavy chain comprising the amino acid sequence of SEQ ID NO:201;
(c) two light chains, each comprising the amino acid sequence of SEQ ID NO:200; and
(d) one light chain comprising the amino acid sequence of SEQ ID NO:202.

51. An agonistic lymphotoxin beta receptor (LTBR) antibody that binds to human LTBR and to cynomolgus LTBR, wherein said antibody comprises
(i) a heavy chain variable region (V_{H} LTBR) comprising a heavy chain complementary determining region (CDR-H1) comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32; or
(ii) a heavy chain variable region (V_{H} LTBR) comprising a CDR-H1 comprising the amino acid sequence of SEQ ID NO:43, a CDR-H2 comprising the amino acid sequence of SEQ ID NO:44, and a CDR-H3 comprising the amino acid sequence of SEQ ID NO:45, and a light chain variable region (V_{L} LTBR) comprising a CDR-L1 comprising the amino acid sequence of SEQ ID NO:46, a CDR-L2 comprising the amino acid sequence of SEQ ID NO:47, and a CDR-L3 comprising the amino acid sequence of SEQ ID NO:48; or
(iii) a heavy chain variable region (V_{H} LTBR) comprising a CDR-H1 comprising the amino acid sequence of SEQ ID NO:75, a CDR-H2 comprising the amino acid sequence of SEQ ID NO:76, and a CDR-H3 comprising the amino acid sequence of SEQ ID NO:77, and a light chain variable region (V_{L} LTBR) comprising a CDR-L1 comprising the amino acid sequence of SEQ ID NO:78, a CDR-L2 comprising the amino acid sequence of SEQ ID NO:79, and a CDR-L3 comprising the amino acid sequence of SEQ ID NO:80; or
(iv) a heavy chain variable region (V_{H} LTBR) comprising a CDR-H1 comprising the amino acid sequence of SEQ ID NO:83, a CDR-H2 comprising the amino acid sequence of SEQ ID NO:84 or SEQ ID NO:92, and a CDR-H3 comprising the amino acid sequence of SEQ ID NO:85, and a light chain variable region (V_{L} LTBR) comprising a CDR-L1 comprising the amino acid sequence of SEQ ID NO:86, a CDR-L2 comprising the amino acid sequence of SEQ ID NO:87, and a CDR-L3 comprising the amino acid sequence of SEQ ID NO:88; or
(v) a heavy chain variable region (V_{H} LTBR) comprising a CDR-H1 comprising the amino acid sequence of SEQ ID NO:35, a CDR-H2 comprising the amino acid sequence of SEQ ID NO:36, and a CDR-H3 comprising the amino acid sequence of SEQ ID NO:37, and a light chain variable region (V_{L} LTBR) comprising a CDR-L1 comprising the amino acid sequence of SEQ ID NO:38, a CDR-L2 comprising the amino acid sequence of SEQ ID NO:39, and a CDR-L3 comprising the amino acid sequence of SEQ ID NO:40, or
(vi) a heavy chain variable region (V_{H} LTBR) comprising a CDR-H1 comprising the amino acid sequence of SEQ ID NO:51, a CDR-H2 comprising the amino acid sequence of SEQ ID NO:52, and a CDR-H3 comprising the amino acid sequence of SEQ ID NO:53, and a light chain variable region (V_{L} LTBR) comprising a CDR-L1 comprising the amino acid sequence of SEQ ID NO:54, a CDR-L2 comprising the amino acid sequence of SEQ ID NO:55, and a CDR-L3 comprising the amino acid sequence of SEQ ID NO:56, or
(vii) a heavy chain variable region (V_{H} LTBR) comprising a CDR-H1 comprising the amino acid sequence of SEQ ID NO:59, a CDR-H2 comprising the amino acid sequence of SEQ ID NO:60, and a CDR-H3 comprising the amino acid sequence of SEQ ID NO:61, and a light chain variable region (V_{L} LTBR) comprising a CDR-L1 comprising the amino acid sequence of SEQ ID NO:62, a CDR-L2 comprising the amino acid sequence of SEQ ID NO:63, and a CDR-L3 comprising the amino acid sequence of SEQ ID NO:64, or
(viii) a heavy chain variable region (V_{H} LTBR) comprising a CDR-H1 comprising the amino acid sequence of SEQ ID NO:67, a CDR-H2 comprising the amino acid sequence of SEQ ID NO:68, and a CDR-H3 comprising the amino acid sequence of SEQ ID NO:69, and a light chain variable region (V_{L} LTBR) comprising a CDR-L1 comprising the amino acid sequence of SEQ ID NO:70, a CDR-L2 comprising the amino acid sequence of SEQ ID NO:71, and a CDR-L3 comprising the amino acid sequence of SEQ ID NO:72.

52. The agonistic LTBR antibody of claim 51, wherein the agonistic antibody has at least one of the following properties:
(a) binds to human LTBR and to cynomolgus LTBR with less than a 2-fold difference in affinity; or
(b) binds to human LTBR extracellular domain with an EC₅₀ of less than 4 nM as measured by ELISA and binds to the cynomolgus LTBR extracellular domain with an EC₅₀ of less than 5 nM as measured by ELISA; or
(c) requires cross-linking for its agonistic activity to activate human LTBR; or
(d) requires cross-linking for its agonistic activity to induce ICAM upregulation in human umbilical vein endothelial cells or cancer associated fibroblasts; or
(e) inhibits the interaction between human LTBR and its human ligands lymphotoxin α1β2 and LIGHT.

53. The agonistic LTBR antibody of claim 51, wherein the agonistic antibody comprises
(i) a heavy chain variable region (V_{H} LTBR) comprising the amino acid sequence of SEQ ID NO:33 and a light chain variable region (V_{L} LTBR) comprising the amino acid sequence of SEQ ID NO:34, or
(ii) a heavy chain variable region (V_{H} LTBR) comprising the amino acid sequence of SEQ ID NO:49 and a light chain variable region (V_{L} LTBR) comprising the amino acid sequence of SEQ ID NO:50, or
(iii) a heavy chain variable region (V_{H} LTBR) comprising the amino acid sequence of SEQ ID NO:81 and a light chain variable region (V_{L} LTBR) comprising the amino acid sequence of SEQ ID NO:82, or
(iv) a heavy chain variable region (V_{H} LTBR) comprising the amino acid sequence of SEQ ID NO:89 and a light chain variable region (V_{L} LTBR) comprising the amino acid sequence of SEQ ID NO:90, or
(v) a heavy chain variable region (V_{H} LTBR) comprising the amino acid sequence of SEQ ID NO:97 and a light chain variable region (V_{L} LTBR) comprising the amino acid sequence of SEQ ID NO:98, or
(vi) a heavy chain variable region (V_{H} LTBR) comprising the amino acid sequence of SEQ ID NO:41 and a light chain variable region (V_{L} LTBR) comprising the amino acid sequence of SEQ ID NO:42, or
(vii) a heavy chain variable region (V_{H} LTBR) comprising the amino acid sequence of SEQ ID NO:57 and a light chain variable region (V_{L} LTBR) comprising the amino acid sequence of SEQ ID NO:58, or
(viii) a heavy chain variable region (V_{H} LTBR) comprising the amino acid sequence of SEQ ID NO:65 and a light chain variable region (V_{L} LTBR) comprising the amino acid sequence of SEQ ID NO:66,
(ix) a heavy chain variable region (V_{H} LTBR) comprising the amino acid sequence of SEQ ID NO:73 and a light chain variable region (V_{L} LTBR) comprising the amino acid sequence of SEQ ID NO:74.

54. The agonistic LTBR antibody of any one of claim 51, wherein the agonistic antibody further binds to murine LTBR, optionally wherein the agonistic antibody binds to the murine LTBR extracellular domain with an EC₅₀ of less than 1 nM as measured by ELISA.

55. The agonistic LTBR antibody of claim 51, wherein said antibody binds to the epitope region of SEQ ID NO:351 on human LTBR.

56. The agonistic LTBR antibody of claim 51, wherein said antibody comprises a heavy chain variable region (V_{H} LTBR) comprising a CDR-H1 comprising the amino acid sequence of SEQ ID NO:27, a CDR-H2 comprising the amino acid sequence of SEQ ID NO:28, and a CDR-H3 comprising the amino acid sequence of SEQ ID NO:29, and a light chain variable region (V_{L} LTBR) comprising a light chain complementarity determining region (iv) CDR-L1 comprising the amino acid sequence of SEQ ID NO:30, a CDR-L2 comprising the amino acid sequence of SEQ ID NO:31, and a CDR-L3 comprising the amino acid sequence of SEQ ID NO:32.

57. The agonistic LTBR antibody of claim 51, wherein said antibody comprises a heavy chain variable region (V_{H} LTBR) comprising the amino acid sequence of SEQ ID NO:33 and a light chain variable region (V_{L} LTBR) comprising the amino acid sequence of SEQ ID NO:34.

58. The agonistic LTBR antibody of claim 51, wherein the agonistic antibody comprises a Fc domain of human origin.

59. The agonistic LTBR antibody of claim 51, wherein the agonistic antibody comprises a Fc domain of human IgG1 subclass comprising one or more amino acid substitution that reduces the binding affinity of the antigen binding molecule to an Fc receptor and/or effector function.

60. The agonistic LTBR antibody of claim 51, wherein the agonistic antibody comprises a Fc domain of human IgG1 subclass with the amino acid mutations L234A, L235A and P329G (numbering according to Kabat EU index).

61. One or more isolated polynucleotides encoding the bispecific and agonistic LTBR antibody of claim 39.

62. An expression vector comprising the one or more isolated polynucleotides of claim 61.

63. A prokaryotic or eukaryotic host cell comprising the one or more isolated polynucleotide of claim 61 or an expression vector comprising the one or more isolated polynucleotides of claim 61.

64. A method of producing a bispecific and agonistic LTBR antibody, comprising the steps of a) culturing the prokaryotic or eukaryotic host cell of claim 63 under conditions suitable for the expression of the agonistic LTBR antibody and b) optionally recovering the bispecific and agonistic LTBR antibody.

65. A pharmaceutical composition comprising the bispecific and agonistic LTBR antibody of claim 39 and a pharmaceutically acceptable excipient.

66. A method of treating an individual having cancer comprising administering to the individual an effective amount of the bispecific and agonistic LTBR antibody of claim 39.

67. One or more isolated polynucleotides encoding the agonistic LTBR antibody of claim 51.

68. An expression vector comprising the one or more isolated polynucleotides of claim 67.

69. A prokaryotic or eukaryotic host cell comprising the one or more isolated polynucleotides of claim 67 or an expression vector comprising the one or more isolated polynucleotides of claim 67.

70. A method of producing an antigen binding molecule, comprising the steps of a) culturing the prokaryotic or eukaryotic host cell of claim 69 under conditions suitable for the expression of the agonistic LTBR antibody and b) optionally recovering the agonistic LTBR antibody.

71. A pharmaceutical composition comprising the agonistic LTBR antibody of claim 51.

72. A method of treating an individual having cancer comprising administering to the individual an effective amount of the agonistic LTBR antibody of claim 51.


SEQUENCE LISTING (361 sequences)

SEQ ID NO: 1 - protein, 435 aa, Homo sapiens
MLLPWATSAPGLAWGPLVLGLFGLLAASQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMLAVLLPLAFFLLLATVFSCIWKSHPSLCRKLGSLLKRRPQGEGPNPVAGSWEPPKAHPYFPDLVQPLLPISGDVSPVSTGLPAAPVLEAGVPQQQSPLDLTREPQLEPGEQSQVAHGTNGIHVTGGSMTITGNIYIYNGPVLGGPPGPGDLPATPEPPYPIPEEGDPGPPGLSTPHQEDGKAWHLAETEHCGATPSNRGPRNQFITHD

SEQ ID NO: 2 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 3 - protein, 5 aa, synthetic construct
  REGION 1..5: note = FAP(4B9) CDR-H1
SYAMS

SEQ ID NO: 4 - protein, 17 aa, synthetic construct
  REGION 1..17: note = FAP(4B9) CDR-H2
AIIGSGASTYYADSVKG

SEQ ID NO: 5 - protein, 8 aa, synthetic construct
  REGION 1..8: note = FAP(4B9) CDR-H3
GWFGGFNY

SEQ ID NO: 6 - protein, 12 aa, synthetic construct
  REGION 1..12: note = FAP(4B9) CDR-L1
RASQSVTSSYLA

SEQ ID NO: 7 - protein, 7 aa, synthetic construct
  REGION 1..7: note = FAP(4B9) CDR-L2
VGSRRAT

SEQ ID NO: 8 - protein, 9 aa, synthetic construct
  REGION 1..9: note = FAP(4B9) CDR-L3
QQGIMLPPT

SEQ ID NO: 9 - protein, 117 aa, synthetic construct
  REGION 1..117: note = FAP(4B9) VH
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 10 - protein, 108 aa, synthetic construct
  REGION 1..108: note = FAP(4B9) VL
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 11 - protein, 5 aa, synthetic construct
  REGION 1..5: note = FAP (28H1) CDR-H1
SHAMS

SEQ ID NO: 12 - protein, 16 aa, synthetic construct
  REGION 1..16: note = FAP (28H1) CDR-H2
AIWASGEQYYADSVKG

SEQ ID NO: 13 - protein, 8 aa, synthetic construct
  REGION 1..8: note = FAP (28H1) CDR-H3
GWLGNFDY

SEQ ID NO: 14 - protein, 12 aa, synthetic construct
  REGION 1..12: note = FAP (28H1) CDR-L1
RASQSVSRSYLA

SEQ ID NO: 15 - protein, 7 aa, synthetic construct
  REGION 1..7: note = FAP (28H1) CDR-L2
GASTRAT

SEQ ID NO: 16 - protein, 9 aa, synthetic construct
  REGION 1..9: note = FAP (28H1) CDR-L3
QQGQVIPPT

SEQ ID NO: 17 - protein, 116 aa, synthetic construct
  REGION 1..116: note = FAP (28H1) VH
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSS

SEQ ID NO: 18 - protein, 108 aa, synthetic construct
  REGION 1..108: note = FAP (28H1) VL
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 19 - protein, 5 aa, synthetic construct
  REGION 1..5: note = FAP (1G1a) CDR-H1
DYNMD

SEQ ID NO: 20 - protein, 17 aa, synthetic construct
  REGION 1..17: note = FAP (1G1a) CDR-H2
DIYPNTGGTIYNQKFKG

SEQ ID NO: 21 - protein, 10 aa, synthetic construct
  REGION 1..10: note = FAP (1G1a) CDR-H3
FRGIHYAMDY

SEQ ID NO: 22 - protein, 15 aa, synthetic construct
  REGION 1..15: note = FAP (1G1a) CDR-L1
RASESVDNYGLSFIN

SEQ ID NO: 23 - protein, 7 aa, synthetic construct
  REGION 1..7: note = FAP (1G1a) CDR-L2
GTSNRGS

SEQ ID NO: 24 - protein, 9 aa, synthetic construct
  REGION 1..9: note = FAP (1G1a) CDR-L3
QQSNEVPYT

SEQ ID NO: 25 - protein, 119 aa, synthetic construct
  REGION 1..119: note = FAP (1G1a) VH
QVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSS

SEQ ID NO: 26 - protein, 111 aa, synthetic construct
  REGION 1..111: note = FAP (1G1a) VL
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIK

SEQ ID NO: 27 - protein, 5 aa, synthetic construct
  REGION 1..5: note = LTBR (9459) CDR-H1
SYTMN

SEQ ID NO: 28 - protein, 17 aa, synthetic construct
  REGION 1..17: note = LTBR (9459) CDR-H2
GVAPSGDRTYYTDSVKG

SEQ ID NO: 29 - protein, 6 aa, synthetic construct
  REGION 1..6: note = LTBR (9459) CDR-H3
GFAFTN

SEQ ID NO: 30 - protein, 11 aa, synthetic construct
  REGION 1..11: note = LTBR (9459) CDR-L1
RASQGISSALA

SEQ ID NO: 31 - protein, 7 aa, synthetic construct
  REGION 1..7: note = LTBR (9459) CDR-L2
DASSLDS

SEQ ID NO: 32 - protein, 9 aa, synthetic construct
  REGION 1..9: note = LTBR (9459) CDR-L3
QQYNNYPLT

SEQ ID NO: 33 - protein, 115 aa, synthetic construct
  REGION 1..115: note = LTBR (9459) VH
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSS

SEQ ID NO: 34 - protein, 107 aa, synthetic construct
  REGION 1..107: note = LTBR (9459) VL
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMK

SEQ ID NO: 35 - protein, 5 aa, synthetic construct
  REGION 1..5: note = LTBR (9452) CDR-H1
SYPMT

SEQ ID NO: 36 - protein, 17 aa, synthetic construct
  REGION 1..17: note = LTBR (9452) CDR-H2
LISPSGATTYYADSVKG

SEQ ID NO: 37 - protein, 19 aa, synthetic construct
  REGION 1..19: note = LTBR (9452) CDR-H3
NHYPDDVIIKNYYFYGVDV

SEQ ID NO: 38 - protein, 11 aa, synthetic construct
  REGION 1..11: note = LTBR (9452) CDR-L1
RASQDISNYLN

SEQ ID NO: 39 - protein, 7 aa, synthetic construct
  REGION 1..7: note = LTBR (9452) CDR-L2
AASSLQS

SEQ ID NO: 40 - protein, 9 aa, synthetic construct
  REGION 1..9: note = LTBR (9452) CDR-L3
QQTHTIPYS

SEQ ID NO: 41 - protein, 128 aa, synthetic construct
  REGION 1..128: note = LTBR (9452) VH
EVQLLESGGGLVQPGGSLRLSCAAAGFTFSSYPMTWVRQAPGKGLEWVSLISPSGATTYYADSVKGRFTISRDTSKNTLYLQMNSLRAEDTAVYYCAKNHYPDDVIIKNYYFYGVDVWGQGTTVTVSS

SEQ ID NO: 42 - protein, 107 aa, synthetic construct
  REGION 1..107: note = LTBR (9452) VL
DIQMTQSPSSLSASVGDRVTITCRASQDISNYLNWYQQKPGKAPKSLIYAASSLQSGVPSRFSGSGSGTDFTLTISSLQPEDFANYYCQQTHTIPYSFGQGTNLEIK

SEQ ID NO: 43 - protein, 5 aa, synthetic construct
  REGION 1..5: note = LTBR (0080) CDR-H1
YYAMT

SEQ ID NO: 44 - protein, 16 aa, synthetic construct
  REGION 1..16: note = LTBR (0080) CDR-H2
VINSGGGTYYTDSVKG

SEQ ID NO: 45 - protein, 6 aa, synthetic construct
  REGION 1..6: note = LTBR (0080) CDR-H3
ASTADV

SEQ ID NO: 46 - protein, 11 aa, synthetic construct
  REGION 1..11: note = LTBR (0080) CDR-L1
QASQGISNYLS

SEQ ID NO: 47 - protein, 7 aa, synthetic construct
  REGION 1..7: note = LTBR (0080) CDR-L2
AASSLES

SEQ ID NO: 48 - protein, 9 aa, synthetic construct
  REGION 1..9: note = LTBR (0080) CDR-L3
QQYNSYSLT

SEQ ID NO: 49 - protein, 114 aa, synthetic construct
  REGION 1..114: note = LTBR (0080) VH
EVQLLESGGGLVQPGGSLRLSCAASGFTFSYYAMTWVRQAPGKGLEWIGVINSGGGTYYTDSVKGRFTMSRDNSKNTLYLQVNSLRAEDTAVYYCAKASTADVWGQGTTVTVSS

SEQ ID NO: 50 - protein, 107 aa, synthetic construct
  REGION 1..107: note = LTBR (0080) VL
DIQMTQSPSSLSASVGDRVTITCQASQGISNYLSWYQQKPGKAPKRLIFAASSLESGVPSRFSGSGSGTDFTLTISSLQPEDFATYYCQQYNSYSLTFGGGTKLEIK

SEQ ID NO: 51 - protein, 5 aa, synthetic construct
  REGION 1..5: note = LTBR (9450) CDR-H1
SYVMK

SEQ ID NO: 52 - protein, 17 aa, synthetic construct
  REGION 1..17: note = LTBR (9450) CDR-H2
SISGSGGRPFYADSVKG

SEQ ID NO: 53 - protein, 4 aa, synthetic construct
  REGION 1..4: note = LTBR (9450) CDR-H3
DMDV

SEQ ID NO: 54 - protein, 11 aa, synthetic construct
  REGION 1..11: note = LTBR (9450) CDR-L1
RASQDISSFLK

SEQ ID NO: 55 - protein, 7 aa, synthetic construct
  REGION 1..7: note = LTBR (9450) CDR-L2
AASALQS

SEQ ID NO: 56 - protein, 9 aa, synthetic construct
  REGION 1..9: note = LTBR (9450) CDR-L3
QQYNTYPLT

SEQ ID NO: 57 - protein, 113 aa, synthetic construct
  REGION 1..113: note = LTBR (9450) VH
EVQLLESGGGLVQPGGSLRLSCAASGFTVSSYVMKWFRQAPGKGLEWVSSISGSGGRPFYADSVKGRFTISRDNSKNALYLQMNSLRAEDTAVYYCANDMDVWGQGTTVTVSS

SEQ ID NO: 58 - protein, 107 aa, synthetic construct
  REGION 1..107: note = LTBR (9450) VL
DIQMTQSPSSLSASVGDRVTITCRASQDISSFLKWYQQKPGKAPTLLIYAASALQSGVPSRFSGSGSGTDFTLTISSLQPEDFATYYCQQYNTYPLTFGGGTKVDIK

SEQ ID NO: 59 - protein, 5 aa, synthetic construct
  REGION 1..5: note = LTBR (0079) CDR-H1
SYAMS

SEQ ID NO: 60 - protein, 17 aa, synthetic construct
  REGION 1..17: note = LTBR (0079) CDR-H2
VITGSSSSIDYADSVKG

SEQ ID NO: 61 - protein, 14 aa, synthetic construct
  REGION 1..14: note = LTBR (0079) CDR-H3
GGSGVWSISTDFDY

SEQ ID NO: 62 - protein, 11 aa, synthetic construct
  REGION 1..11: note = LTBR (0079) CDR-L1
QASQDISNYLN

SEQ ID NO: 63 - protein, 7 aa, synthetic construct
  REGION 1..7: note = LTBR (0079) CDR-L2
AASSLHS

SEQ ID NO: 64 - protein, 9 aa, synthetic construct
  REGION 1..9: note = LTBR (0079) CDR-L3
QQSHSTPPT

SEQ ID NO: 65 - protein, 123 aa, synthetic construct
  REGION 1..123: note = LTBR (0079) VH
EVQLLESGGALVQPGGSLRLSCAASGFTFSSYAMSWVRQAPGKGLEWVSVITGSSSSIDYADSVKGRFTISRDNSKNTLYLQMNSLRAEDTAVYYCAKGGSGVWSISTDFDYWGQGTLVTVSS

SEQ ID NO: 66 - protein, 107 aa, synthetic construct
  REGION 1..107: note = LTBR (0079) VL
DIQMTQSPSFLSASVGDRVTITCQASQDISNYLNWYQQKPGKAPKHLIYAASSLHSGVPSRFSGSGSGAYFTLTITSLQPEDFATYYCQQSHSTPPTFGPGTKVDIK

SEQ ID NO: 67 - protein, 5 aa, synthetic construct
  REGION 1..5: note = LTBR (9457) CDR-H1
NYAMT

SEQ ID NO: 68 - protein, 17 aa, synthetic construct
  REGION 1..17: note = LTBR (9457) CDR-H2
EISGSGGSTYYADSVKG

SEQ ID NO: 69 - protein, 11 aa, synthetic construct
  REGION 1..11: note = LTBR (9457) CDR-H3
DDDGAYGTMDV

SEQ ID NO: 70 - protein, 11 aa, synthetic construct
  REGION 1..11: note = LTBR (9457) CDR-L1
RASQDISNYLN

SEQ ID NO: 71 - protein, 7 aa, synthetic construct
  REGION 1..7: note = LTBR (9457) CDR-L2
AASALQS

SEQ ID NO: 72 - protein, 9 aa, synthetic construct
  REGION 1..9: note = LTBR (9457) CDR-L3
QHYKSYPYT

SEQ ID NO: 73 - protein, 121 aa, synthetic construct
  REGION 1..121: note = LTBR (9457) VH
EVQLLESGGGLVQPGGSLRLSCAASGFTFSNYAMTWVRQAPGKGLEWVSEISGSGGSTYYADSVKGRFTISRDNSKNTLYLQMNSLRAEDTAVYYCAKDDDGAYGDTMDVWGQGTTVTVSS

SEQ ID NO: 74 - protein, 107 aa, synthetic construct
  REGION 1..107: note = LTBR (9457) VL
DIQMTQSPSSLSASIGDRVTITCRASQDISNYLNWYQQKPGKAPKLLIYAASALQSGVPSRFSGSGSGTDFTLTISSLQPEDFATYYCQHYKSYPYTFGQGTKVEIR

SEQ ID NO: 75 - protein, 5 aa, synthetic construct
  REGION 1..5: note = LTBR (0064) CDR-H1
NTYIH

SEQ ID NO: 76 - protein, 17 aa, synthetic construct
  REGION 1..17: note = LTBR (0064) CDR-H2
RIDPTNGNTICTEKFKN

SEQ ID NO: 77 - protein, 11 aa, synthetic construct
  REGION 1..11: note = LTBR (0064) CDR-H3
TTLTTPDSFAY

SEQ ID NO: 78 - protein, 11 aa, synthetic construct
  REGION 1..11: note = LTBR (0064) CDR-L1
KASQNIYKYLN

SEQ ID NO: 79 - protein, 7 aa, synthetic construct
  REGION 1..7: note = LTBR (0064) CDR-L2
STNILQT

SEQ ID NO: 80 - protein, 8 aa, synthetic construct
  REGION 1..8: note = LTBR (0064) CDR-L3
QKYYSGFT

SEQ ID NO: 81 - protein, 120 aa, synthetic construct
  REGION 1..120: note = LTBR (0064) VH
EVQLQQYGAELGKPGTSVKLSCKVSGYNIGNTYIHWVIQRPGKGLEWIGRIDPTNGNTICTEKFKNKATLTADLSSNTAYMQLSQLKSDDTAIYFCAPTTLTTPDSFAYWGQGTLVTVSS

SEQ ID NO: 82 - protein, 106 aa, synthetic construct
  REGION 1..106: note = LTBR (0064) VL
DIQMTQSPSQLSASVGDRVTLSCKASQNIYKYLNWYQQKLGEAPRLLIYSTNILQTGIPSRFSGSGSGTDYTLTISSLQPEDAATYFCQKYYSGFTFGSGTKLEMK

SEQ ID NO: 83 - protein, 5 aa, synthetic construct
  REGION 1..5: note = LTBR (5929) CDR-H1
SYAIS

SEQ ID NO: 84 - protein, 17 aa, synthetic construct
  REGION 1..17: note = LTBR (5929) CDR-H2
GIIPIFGTANYAQKFQG

SEQ ID NO: 85 - protein, 11 aa, synthetic construct
  REGION 1..11: note = LTBR (5929) CDR-H3
PSYYLDGWLDY

SEQ ID NO: 86 - protein, 17 aa, synthetic construct
  REGION 1..17: note = LTBR (5929) CDR-L1
KSSQSVLYSSNNKNYLA

SEQ ID NO: 87 - protein, 7 aa, synthetic construct
  REGION 1..7: note = LTBR (5929) CDR-L2
WASTRES

SEQ ID NO: 88 - protein, 9 aa, synthetic construct
  REGION 1..9: note = LTBR (5929) CDR-L3
SQYYTTHYT

SEQ ID NO: 89 - protein, 120 aa, synthetic construct
  REGION 1..120: note = LTBR (5929) VH
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARPSYYLDGWLDYWGQGTTVTVSS

SEQ ID NO: 90 - protein, 113 aa, synthetic construct
  REGION 1..113: note = LTBR (5929) VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCSQYYTTHYTFGQGTKVEIK

SEQ ID NO: 91 - protein, 5 aa, synthetic construct
  REGION 1..5: note = LTBR (7213) CDR-H1
SYAIS

SEQ ID NO: 92 - protein, 17 aa, synthetic construct
  REGION 1..17: note = LTBR (7213) CDR-H2
GIIPIHGTANYAQKFQG

SEQ ID NO: 93 - protein, 11 aa, synthetic construct
  REGION 1..11: note = LTBR (7213) CDR-H3
PSYYLDGWLDY

SEQ ID NO: 94 - protein, 17 aa, synthetic construct
  REGION 1..17: note = LTBR (7213) CDR-L1
KSSQSVLYSSNNKNYLA

SEQ ID NO: 95 - protein, 7 aa, synthetic construct
  REGION 1..7: note = LTBR (7213) CDR-L2
WASTRES

SEQ ID NO: 96 - protein, 9 aa, synthetic construct
  REGION 1..9: note = LTBR (7213) CDR-L3
SQYYTTHYT

SEQ ID NO: 97 - protein, 120 aa, synthetic construct
  REGION 1..120: note = LTBR (7213) VH
QVQLVQSGAEVKKPGSSVKVSCKASGGTFSSYAISWVRQAPGQGLEWMGGIIPIHGTANYAQKFQGRVTITADKSTSTAYMELSSLRSEDTAVYYCARPSYYLDGWLDYWGQGTTVTVSS

SEQ ID NO: 98 - protein, 113 aa, synthetic construct; identical to SEQ ID NO: 90
  REGION 1..113: note = LTBR (7213) VL

SEQ ID NO: 99 - protein, 120 aa, synthetic construct
  REGION 1..120: note = LTBR (CBE11) VH
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSS

SEQ ID NO: 100 - protein, 107 aa, synthetic construct
  REGION 1..107: note = LTBR (CBE11) VL
DIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIK

SEQ ID NO: 101 - protein, 5 aa, synthetic construct
  REGION 1..5: note = LTBR (CBE11) CDR-H1
DYYMY

SEQ ID NO: 102 - protein, 17 aa, synthetic construct
  REGION 1..17: note = LTBR (CBE11) CDR-H2
TISDGGSYTYYPDSVKG

SEQ ID NO: 103 - protein, 11 aa, synthetic construct
  REGION 1..11: note = LTBR (CBE11) CDR-H3
EENGNFYYFDY

SEQ ID NO: 104 - protein, 11 aa, synthetic construct
  REGION 1..11: note = LTBR (CBE11) CDR-L1
KAGQDIKSYLS

SEQ ID NO: 105 - protein, 7 aa, synthetic construct
  REGION 1..7: note = LTBR (CBE11) CDR-L2
YATRLAD

SEQ ID NO: 106 - protein, 9 aa, synthetic construct
  REGION 1..9: note = LTBR (CBE11) CDR-L3
LQHGESPWT

SEQ ID NO: 107 - protein, 443 aa, synthetic construct
  REGION 1..443: note = P1AF0532 HC knob LTBR crossed
DIVMTQSPDSLAVSLGERATINCKSSQSVLYSSNNKNYLAWYQQKPGQPPKLLIYWASTRESGVPDRFSGSGSGTDFTLTISSLQAEDVAVYYCSQYYTTHYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 108 - protein, 227 aa, synthetic construct
  REGION 1..227: note = P1AF0532 LC LTBR crossed
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARPSYYLDGWLDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 445 aa, synthetic construct
  REGION 1..445: note = P1AF0532 HC hole FAP with charges
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 110 - protein, 215 aa, synthetic construct
  REGION 1..215: note = P1AF0532 LC FAP with charges
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 448 aa, synthetic construct
  REGION 1..448: note = P1AF9728 HC knob LTBR
QVQLVQSGAEVKKPGSSVKVSCKASGGTFSSYAISWVRQAPGQGLEWMGGIIPIHGTANYAQKFQGRVTITADKSTSTAYMELSSLRSEDTAVYYCARPSYYLDGWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 112 - protein, 220 aa, synthetic construct
  REGION 1..220: note = P1AF9728 LC LTBR
DIVMTQSPDSLAVSLGERATINCKSSQSVLYSSNNKNYLAWYQQKPGQPPKLLIYWASTRESGVPDRFSGSGSGTDFTLTISSLQAEDVAVYYCSQYYTTHYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 449 aa, synthetic construct
  REGION 1..449: note = P1AF9728 HC hole FAP crossed
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 114 - protein, 213 aa, synthetic construct
  REGION 1..213: note = P1AF9728 LC FAP crossed
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 115 - protein, 449 aa, synthetic construct
  REGION 1..449: note = P1AG7504 HC knob LTBR
QVQLVQSGAEVKKPGSSVKVSCKASGGTFSSYAISWVRQAPGQGLEWMGGIIPIHGTANYAQKFQGRVTITADKSTSTAYMELSSLRSEDTAVYYCARPSYYLDGWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 220 aa, synthetic construct; identical to SEQ ID NO: 112
  REGION 1..220: note = P1AG7504 LC LTBR

SEQ ID NO: 117 - protein, 450 aa, synthetic construct
  REGION 1..450: note = P1AG7504 HC hole FAP crossed
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 118 - protein, 214 aa, synthetic construct
  REGION 1..214: note = P1AG7504 LC FAP crossed
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 119 - protein, 437 aa, synthetic construct
  REGION 1..437: note = P1AF0535 HC knob LTBR crossed
DIQMTQSPSSLSASVGDRVTITCRASQDISSFLKWYQQKPGKAPTLLIYAASALQSGVPSRFSGSGSGTDFTLTISSLQPEDFATYYCQQYNTYPLTFGGGTKVDIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 120 - protein, 220 aa, synthetic construct
  REGION 1..220: note = P1AF0535 LC LTBR crossed
EVQLLESGGGLVQPGGSLRLSCAASGFTVSSYVMKWFRQAPGKGLEWVSSISGSGGRPFYADSVKGRFTISRDNSKNALYLQMNSLRAEDTAVYYCANDMDVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 445 aa, synthetic construct; identical to SEQ ID NO: 109
  REGION 1..445: note = P1AF0535 HC hole FAP with charges

SEQ ID NO: 122 - protein, 215 aa, synthetic construct; identical to SEQ ID NO: 110
  REGION 1..215: note = P1AF0535 LC FAP with charges

SEQ ID NO: 123 - protein, 438 aa, synthetic construct
  REGION 1..438: note = P1AG7506 HC knob LTBR crossed
DIQMTQSPSSLSASVGDRVTITCRASQDISSFLKWYQQKPGKAPTLLIYAASALQSGVPSRFSGSGSGTDFTLTISSLQPEDFATYYCQQYNTYPLTFGGGTKVDIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 220 aa, synthetic construct; identical to SEQ ID NO: 120
  REGION 1..220: note = P1AG7506 LC LTBR crossed

SEQ ID NO: 125 - protein, 446 aa, synthetic construct
  REGION 1..446: note = P1AG7506 HC hole FAP charges
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 126 - protein, 215 aa, synthetic construct; identical to SEQ ID NO: 110
  REGION 1..215: note = P1AG7506 LC FAP charges

SEQ ID NO: 127 - protein, 437 aa, synthetic construct
  REGION 1..437: note = P1AF0534 HC knob LTBR crossed
DIQMTQSPSSLSASVGDRVTITCRASQDISNYLNWYQQKPGKAPKSLIYAASSLQSGVPSRFSGSGSGTDFTLTISSLQPEDFANYYCQQTHTIPYSFGQGTNLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 128 - protein, 235 aa, synthetic construct
  REGION 1..235: note = P1AF0534 LC LTBR crossed
EVQLLESGGGLVQPGGSLRLSCAAAGFTFSSYPMTWVRQAPGKGLEWVSLISPSGATTYYADSVKGRFTISRDTSKNTLYLQMNSLRAEDTAVYYCAKNHYPDDVIIKNYYFYGVDVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 445 aa, synthetic construct; identical to SEQ ID NO: 109
  REGION 1..445: note = P1AF0534 HC hole FAP with charges

SEQ ID NO: 130 - protein, 215 aa, synthetic construct; identical to SEQ ID NO: 110
  REGION 1..215: note = P1AF0534 LC FAP with charges

SEQ ID NO: 131 - protein, 438 aa, synthetic construct
  REGION 1..438: note = P1AG7505 HC knob LTBR crossed
DIQMTQSPSSLSASVGDRVTITCRASQDISNYLNWYQQKPGKAPKSLIYAASSLQSGVPSRFSGSGSGTDFTLTISSLQPEDFANYYCQQTHTIPYSFGQGTNLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 235 aa, synthetic construct; identical to SEQ ID NO: 128
  REGION 1..235: note = P1AG7505 LC LTBR crossed

SEQ ID NO: 133 - protein, 446 aa, synthetic construct; identical to SEQ ID NO: 125
  REGION 1..446: note = P1AG7505 HC hole FAP charges

SEQ ID NO: 134 - protein, 215 aa, synthetic construct; identical to SEQ ID NO: 110
  REGION 1..215: note = P1AG7505 LC FAP charges

SEQ ID NO: 135 - protein, 437 aa, synthetic construct
  REGION 1..437: note = P1AF5257 HC knob LTBR crossed
DIQMTQSPSSLSASIGDRVTITCRASQDISNYLNWYQQKPGKAPKLLIYAASALQSGVPSRFSGSGSGTDFTLTISSLQPEDFATYYCQHYKSYPYTFGQGTKVEIRSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 136 - protein, 228 aa, synthetic construct
  REGION 1..228: note = P1AF5257 LC LTBR crossed
EVQLLESGGGLVQPGGSLRLSCAASGFTFSNYAMTWVRQAPGKGLEWVSEISGSGGSTYYADSVKGRFTISRDNSKNTLYLQMNSLRAEDTAVYYCAKDDDGAYGDTMDVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 445 aa, synthetic construct; identical to SEQ ID NO: 109
  REGION 1..445: note = P1AF5257 HC hole FAP charges

SEQ ID NO: 138 - protein, 215 aa, synthetic construct; identical to SEQ ID NO: 110
  REGION 1..215: note = P1AF5257 LC FAP charges

SEQ ID NO: 139 - protein, 437 aa, synthetic construct
  REGION 1..437: note = P1AF0537 HC knob LTBR crossed
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 140 - protein, 222 aa, synthetic construct
  REGION 1..222: note = P1AF0537 LC LTBR crossed
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 445 aa, synthetic construct; identical to SEQ ID NO: 109
  REGION 1..445: note = P1AF0537 HC hole FAP with charges

SEQ ID NO: 142 - protein, 215 aa, synthetic construct; identical to SEQ ID NO: 110
  REGION 1..215: note = P1AF0537 LC FAP with charges

SEQ ID NO: 143 - protein, 438 aa, synthetic construct
  REGION 1..438: note = P1AG7507 HC knob LTBR crossed
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 222 aa, synthetic construct; identical to SEQ ID NO: 140
  REGION 1..222: note = P1AG7507 LC LTBR crossed

SEQ ID NO: 145 - protein, 446 aa, synthetic construct; identical to SEQ ID NO: 125
  REGION 1..446: note = P1AG7507 HC hole FAP charges

SEQ ID NO: 146 - protein, 215 aa, synthetic construct; identical to SEQ ID NO: 110
  REGION 1..215: note = P1AG7507 LC FAP charges

SEQ ID NO: 147 - protein, 448 aa, synthetic construct
  REGION 1..448: note = P1AG0694 HC knob LTBR
QVQLVQSGAEVKKPGASVKVSCKASGYNIGNTYIHWVRQAPGKGLEWMGRIDPTNGNTIYAQKFQGRVTSTRDTSISTAYMELSRLRSDDTVVYYCAPTTLTTPDSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 148 - protein, 213 aa, synthetic construct
  REGION 1..213: note = P1AG0694 LC LTBR
DIQMTQSPSSLSASVGDRVTITCRASQNIYKYLNWYQQKPGKAPKLLIYSTNILQSGVPSRFSGSGSGTDFTLTISSLQPEDFATYFCQKYYSGFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 149 - protein, 449 aa, synthetic construct; identical to SEQ ID NO: 113
  REGION 1..449: note = P1AG0694 HC hole FAP crossed

SEQ ID NO: 150 - protein, 213 aa, synthetic construct; identical to SEQ ID NO: 114
  REGION 1..213: note = P1AG0694 LC FAP crossed

SEQ ID NO: 151 - protein, 448 aa, synthetic construct
  REGION 1..448: note = P1AF9727 HC knob LTBR
EVQLQQYGAELGKPGTSVKLSCKVSGYNIGNTYIHWVIQRPGKGLEWIGRIDPTNGNTISTEKFKNKATLTADLSSNTAYMQLSQLKSDDTAIYFCAPTTLTTPDSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 152 - protein, 213 aa, synthetic construct
  REGION 1..213: note = P1AF9727 LC LTBR
DIQMTQSPSQLSASVGDRVTLSCKASQNIYKYLNWYQQKLGEAPRLLIYSTNILQTGIPSRFSGSGSGTDYTLTISSLQPEDAATYFCQKYYSGFTFGSGTKLEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 449 aa, synthetic construct; identical to SEQ ID NO: 113
  REGION 1..449: note = P1AF9727 HC hole FAP crossed

SEQ ID NO: 154 - protein, 213 aa, synthetic construct; identical to SEQ ID NO: 114
  REGION 1..213: note = P1AF9727 LC FAP crossed

SEQ ID NO: 155 - protein, 437 aa, synthetic construct
  REGION 1..437: note = P1AF5260 HC knob LTBR crossed
DIQMTQSPSFLSASVGDRVTITCQASQDISNYLNWYQQKPGKAPKHLIYAASSLHSGVPSRFSGSGSGAYFTLTITSLQPEDFATYYCQQSHSTPPTFGPGTKVDIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 156 - protein, 230 aa, synthetic construct
  REGION 1..230: note = P1AF5260 LC LTBR crossed
EVQLLESGGALVQPGGSLRLSCAASGFTFSSYAMSWVRQAPGKGLEWVSVITGSSSSIDYADSVKGRFTISRDNSKNTLYLQMNSLRAEDTAVYYCAKGGSGVWSISTDFDYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 445 aa, synthetic construct; identical to SEQ ID NO: 109
  REGION 1..445: note = P1AF5260 HC hole FAP charges

SEQ ID NO: 158 - protein, 215 aa, synthetic construct; identical to SEQ ID NO: 110
  REGION 1..215: note = P1AF5260 LC FAP charges

SEQ ID NO: 159 - protein, 437 aa, synthetic construct
  REGION 1..437: note = P1AF5261 HC knob LTBR crossed
DIQMTQSPSSLSASVGDRVTITCQASQGISNYLSWYQQKPGKAPKRLIFAASSLESGVPSRFSGSGSGTDFTLTISSLQPEDFATYYCQQYNSYSLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 160 - protein, 221 aa, synthetic construct
  REGION 1..221: note = P1AF5261 LC LTBR crossed
EVQLLESGGGLVQPGGSLRLSCAASGFTFSYYAMTWVRQAPGKGLEWIGVINSGGGTYYTDSVKGRFTMSRDNSKNTLYLQVNSLRAEDTAVYYCAKASTADVWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 445 aa, synthetic construct; identical to SEQ ID NO: 109
  REGION 1..445: note = P1AF5261 HC hole FAP charges

SEQ ID NO: 162 - protein, 215 aa, synthetic construct; identical to SEQ ID NO: 110
  REGION 1..215: note = P1AF5261 LC FAP charges

SEQ ID NO: 163 - protein, 438 aa, synthetic construct
  REGION 1..438: note = P1AG7508 HC knob LTBR crossed
DIQMTQSPSSLSASVGDRVTITCQASQGISNYLSWYQQKPGKAPKRLIFAASSLESGVPSRFSGSGSGTDFTLTISSLQPEDFATYYCQQYNSYSLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 221 aa, synthetic construct; identical to SEQ ID NO: 160
  REGION 1..221: note = P1AG7508 LC LTBR crossed

SEQ ID NO: 165 - protein, 446 aa, synthetic construct; identical to SEQ ID NO: 125
  REGION 1..446: note = P1AG7508 HC hole FAP charges

SEQ ID NO: 166 - protein, 215 aa, synthetic construct; identical to SEQ ID NO: 110
  REGION 1..215: note = P1AG7508 LC FAP charges

SEQ ID NO: 167 - protein, 685 aa, synthetic construct
  REGION 1..685: note = P1AG5684(2+1) HC knob LTBR - FAP
DIVMTQSPDSLAVSLGERATINCKSSQSVLYSSNNKNYLAWYQQKPGQPPKLLIYWASTRESGVPDRFSGSGSGTDFTLTISSLQAEDVAVYYCSQYYTTHYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 168 - protein, 227 aa, synthetic construct
  REGION 1..227: note = P1AG5684(2+1) LC LTBR (2x)crossed
QVQLVQSGAEVKKPGSSVKVSCKASGGTFSSYAISWVRQAPGQGLEWMGGIIPIHGTANYAQKFQGRVTITADKSTSTAYMELSSLRSEDTAVYYCARPSYYLDGWLDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 444 aa, synthetic construct
  REGION 1..444: note = P1AG5684(2+1) HC hole LTBR crossed
DIVMTQSPDSLAVSLGERATINCKSSQSVLYSSNNKNYLAWYQQKPGQPPKLLIYWASTRESGVPDRFSGSGSGTDFTLTISSLQAEDVAVYYCSQYYTTHYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 170 - protein, 215 aa, synthetic construct; identical to SEQ ID NO: 110
  REGION 1..215: note = P1AG5684(2+1) LC FAP charges

SEQ ID NO: 171 - protein, 685 aa, synthetic construct
  REGION 1..685: note = P1AG7576(2+1) HC knob LTBR - FAP
DIVMTQSPDSLAVSLGERATINCKSSQSVLYSSNNKNYLAWYQQKPGQPPKLLIYWASTRESGVPDRFSGSGSGTDFTLTISSLQAEDVAVYYCSQYYTTHYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 172 - protein, 227 aa, synthetic construct; identical to SEQ ID NO: 168
  REGION 1..227: note = P1AG7576(2+1) LC LTBR (2x)crossed

SEQ ID NO: 173 - protein, 444 aa, synthetic construct; identical to SEQ ID NO: 169
  REGION 1..444: note = P1AG7576(2+1) HC hole LTBR crossed

SEQ ID NO: 174 - protein, 215 aa, synthetic construct; identical to SEQ ID NO: 110
  REGION 1..215: note = P1AG7576(2+1) LC FAP charges

SEQ ID NO: 175 - protein, 684 aa, synthetic construct
  REGION 1..684: note = P1AG7509(2+1) HC knob LTBR - FAP
DIVMTQSPDSLAVSLGERATINCKSSQSVLYSSNNKNYLAWYQQKPGQPPKLLIYWASTRESGVPDRFSGSGSGTDFTLTISSLQAEDVAVYYCSQYYTTHYTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 176 - protein, 227 aa, synthetic construct; identical to SEQ ID NO: 168
  REGION 1..227: note = P1AG7509(2+1) LC LTBR (2x)crossed

SEQ ID NO: 177 - protein, 444 aa, synthetic construct; identical to SEQ ID NO: 169
  REGION 1..444: note = P1AG7509(2+1) HC hole LTBR crossed

SEQ ID NO: 178 - protein, 215 aa, synthetic construct; identical to SEQ ID NO: 110
  REGION 1..215: note = P1AG7509(2+1) LC FAP charges

SEQ ID NO: 179 - protein, 679 aa, synthetic construct
  REGION 1..679: note = P1AF0544(2+1) HC knob LTBR - FAP
DIQMTQSPSSLSASVGDRVTITCRASQDISSFLKWYQQKPGKAPTLLIYAASALQSGVPSRFSGSGSGTDFTLTISSLQPEDFATYYCQQYNTYPLTFGGGTKVDIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 180 - protein, 220 aa, synthetic construct; identical to SEQ ID NO: 120
  REGION 1..220: note = P1AF0544(2+1) LC LTBR (2x)crossed

SEQ ID NO: 181 - protein, 439 aa, synthetic construct
  REGION 1..439: note = P1AF0544(2+1) HC hole LTBR crossed
DIQMTQSPSSLSASVGDRVTITCRASQDISSFLKWYQQKPGKAPTLLIYAASALQSGVPSRFSGSGSGTDFTLTISSLQPEDFATYYCQQYNTYPLTFGGGTKVDIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 182 - protein, 215 aa, synthetic construct; identical to SEQ ID NO: 110
  REGION 1..215: note = P1AF0544(2+1) LC FAP charges

SEQ ID NO: 183 - protein, 678 aa, synthetic construct
  REGION 1..678: note = P1AG7511(2+1) HC knob LTBR - FAP
DIQMTQSPSSLSASVGDRVTITCRASQDISSFLKWYQQKPGKAPTLLIYAASALQSGVPSRFSGSGSGTDFTLTISSLQPEDFATYYCQQYNTYPLTFGGGTKVDIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 184 - protein, 220 aa, synthetic construct; identical to SEQ ID NO: 120
  REGION 1..220: note = P1AG7511(2+1) LC LTBR (2x)crossed

SEQ ID NO: 185 - protein, 438 aa, synthetic construct
  REGION 1..438: note = P1AG7511(2+1) HC hole LTBR crossed
DIQMTQSPSSLSASVGDRVTITCRASQDISSFLKWYQQKPGKAPTLLIYAASALQSGVPSRFSGSGSGTDFTLTISSLQPEDFATYYCQQYNTYPLTFGGGTKVDIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 186 - protein, 215 aa, synthetic construct; identical to SEQ ID NO: 110
  REGION 1..215: note = P1AG7511(2+1) LC FAP charges

SEQ ID NO: 187 - protein, 699 aa, synthetic construct
  REGION 1..699: note = P1AF9729(2+1) HC knob LTBR (2x)
EVQLLESGGGLVQPGGSLRLSCAAAGFTFSSYPMTWVRQAPGKGLEWVSLISPSGATTYYADSVKGRFTISRDTSKNTLYLQMNSLRAEDTAVYYCAKNHYPDDVIIKNYYFYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVQPGGSLRLSCAAAGFTFSSYPMTWVRQAPGKGLEWVSLISPSGATTYYADSVKGRFTISRDTSKNTLYLQMNSLRAEDTAVYYCAKNHYPDDVIIKNYYFYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 188 - protein, 214 aa, synthetic construct
  REGION 1..214: note = P1AF9729(2+1) LC LTBR (2x)
DIQMTQSPSSLSASVGDRVTITCRASQDISNYLNWYQQKPGKAPKSLIYAASSLQSGVPSRFSGSGSGTDFTLTISSLQPEDFANYYCQQTHTIPYSFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 189 - protein, 449 aa, synthetic construct; identical to SEQ ID NO: 113
  REGION 1..449: note = P1AF9729(2+1) HC hole FAP crossed

SEQ ID NO: 190 - protein, 213 aa, synthetic construct; identical to SEQ ID NO: 114
  REGION 1..213: note = P1AF9729(2+1) LC FAP crossed

SEQ ID NO: 191 - protein, 708 aa, synthetic construct
  REGION 1..708: note = P1AG7510(2+1) HC knob LTBR (2x)
EVQLLESGGGLVQPGGSLRLSCAAAGFTFSSYPMTWVRQAPGKGLEWVSLISPSGATTYYADSVKGRFTISRDTSKNTLYLQMNSLRAEDTAVYYCAKNHYPDDVIIKNYYFYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGEVQLLESGGGLVQPGGSLRLSCAAAGFTFSSYPMTWVRQAPGKGLEWVSLISPSGATTYYADSVKGRFTISRDTSKNTLYLQMNSLRAEDTAVYYCAKNHYPDDVIIKNYYFYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCD

SEQ ID NO: 192 - protein, 214 aa, synthetic construct; identical to SEQ ID NO: 188
  REGION 1..214: note = P1AG7510(2+1) LC LTBR (2x)

SEQ ID NO: 193 - protein, 450 aa, synthetic construct; identical to SEQ ID NO: 117
  REGION 1..450: note = P1AG7510(2+1) HC hole FAP crossed

SEQ ID NO: 194 - protein, 214 aa, synthetic construct; identical to SEQ ID NO: 118
  REGION 1..214: note = P1AG7510(2+1) LC FAP crossed

SEQ ID NO: 195 - protein, 679 aa, synthetic construct
  REGION 1..679: note = P1AF0546(2+1) HC knob LTBR - FAP
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 196 - protein, 222 aa, synthetic construct; identical to SEQ ID NO: 140
  REGION 1..222: note = P1AF0546(2+1) LC LTBR (2x)crossed

SEQ ID NO: 197 - protein, 437 aa, synthetic construct
  REGION 1..437: note = P1AF0546(2+1) HC hole LTBR crossed
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 198 - protein, 215 aa, synthetic construct; identical to SEQ ID NO: 110
  REGION 1..215: note = P1AF0546(2+1) LC FAP charges

SEQ ID NO: 199 - protein, 678 aa, synthetic construct
  REGION 1..678: note = P1AG7512(2+1) HC knob LTBR - FAP
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 200 - protein, 222 aa, synthetic construct; identical to SEQ ID NO: 140
  REGION 1..222: note = P1AG7512(2+1) LC LTBR (2x)crossed

SEQ ID NO: 201 - protein, 438 aa, synthetic construct
  REGION 1..438: note = P1AG7512(2+1) HC hole LTBR crossed
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 202 - protein, 215 aa, synthetic construct; identical to SEQ ID NO: 110
  REGION 1..215: note = P1AG7512(2+1) LC FAP charges

SEQ ID NO: 203 - protein, 678 aa, synthetic construct
  REGION 1..678: note = P1AG7513(2+1) HC knob LTBR - FAP
DIQMTQSPSSLSASVGDRVTITCQASQGISNYLSWYQQKPGKAPKRLIFAASSLESGVPSRFSGSGSGTDFTLTISSLQPEDFATYYCQQYNSYSLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 204 - protein, 221 aa, synthetic construct; identical to SEQ ID NO: 160
  REGION 1..221: note = P1AG7513(2+1) LC LTBR (2x)crossed

SEQ ID NO: 205 - protein, 438 aa, synthetic construct
  REGION 1..438: note = P1AG7513(2+1) HC hole LTBR crossed
DIQMTQSPSSLSASVGDRVTITCQASQGISNYLSWYQQKPGKAPKRLIFAASSLESGVPSRFSGSGSGTDFTLTISSLQPEDFATYYCQQYNSYSLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 206 - protein, 215 aa, synthetic construct; identical to SEQ ID NO: 110
  REGION 1..215: note = P1AG7513(2+1) LC FAP charges

SEQ ID NO: 207 - protein, 679 aa, synthetic construct
  REGION 1..679: note = P1AG5683(2+1) HC knob LTBR - FAP
DIQMTQSPSSLSASVGDRVTITCQASQGISNYLSWYQQKPGKAPKRLIFAASSLESGVPSRFSGSGSGTDFTLTISSLQPEDFATYYCQQYNSYSLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 208 - protein, 221 aa, synthetic construct; identical to SEQ ID NO: 160
  REGION 1..221: note = P1AG5683(2+1) LC LTBR (2x)crossed

SEQ ID NO: 209 - protein, 438 aa, synthetic construct; identical to SEQ ID NO: 205
  REGION 1..438: note = P1AG5683(2+1) HC hole LTBR crossed

SEQ ID NO: 210 - protein, 215 aa, synthetic construct; identical to SEQ ID NO: 110
  REGION 1..215: note = P1AG5683(2+1) LC FAP charges

SEQ ID NO: 211 - protein, 679 aa, synthetic construct
  REGION 1..679: note = P1AG7575(2+1) HC knob LTBR - FAP
DIQMTQSPSSLSASVGDRVTITCQASQGISNYLSWYQQKPGKAPKRLIFAASSLESGVPSRFSGSGSGTDFTLTISSLQPEDFATYYCQQYNSYSLTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 212 - protein, 221 aa, synthetic construct; identical to SEQ ID NO: 160
  REGION 1..221: note = P1AG7575(2+1) LC LTBR (2x)crossed

SEQ ID NO: 213 - protein, 438 aa, synthetic construct; identical to SEQ ID NO: 205
  REGION 1..438: note = P1AG7575(2+1) HC hole LTBR crossed

SEQ ID NO: 214 - protein, 215 aa, synthetic construct; identical to SEQ ID NO: 110
  REGION 1..215: note = P1AG7575(2+1) LC FAP charges

SEQ ID NO: 215 - protein, 437 aa, synthetic construct
  REGION 1..437: note = P1AG1326 HC knob LTBR crossed
DIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 216 - protein, 227 aa, synthetic construct
  REGION 1..227: note = P1AG1326 LC LTBR crossed
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 217 - protein, 445 aa, synthetic construct; identical to SEQ ID NO: 109
  REGION 1..445: note = P1AG1326 HC hole FAP charges

SEQ ID NO: 218 - protein, 215 aa, synthetic construct; identical to SEQ ID NO: 110
  REGION 1..215: note = P1AG1326 LC FAP charges

SEQ ID NO: 219 - protein, 437 aa, synthetic construct
  REGION 1..437: note = P1AF4664 HC KK+ LTBR charges (murine 1‘surrogate)
EVQLLESGGGLVQPGGSLRLSCAASGFTFSYYAMTWVRQAPGKGLEWIGVINSGGGTYYTDSVKGRFTMSRDNSKNTLYLQVNSLRAEDTAVYYCAKASTADVWGQGTTVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPG

SEQ ID NO: 220 - protein, 214 aa, synthetic construct
  REGION 1..214: note = P1AF4664 LC LTBR charges (murine 1‘surrogate)
DIQMTQSPSSLSASVGDRVTITCQASQGISNYLSWYQQKPGKAPKRLIFAASSLESGVPSRFSGSGSGTDFTLTISSLQPEDFATYYCQQYNSYSLTFGGGTKLEIKRADAAPTVSIFPPSSRKLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 221 - protein, 433 aa, synthetic construct
  REGION 1..433: note = P1AF4664 HC DD- FAP crossed (murine 1‘surrogate)
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 222 - protein, 223 aa, synthetic construct
  REGION 1..223: note = P1AF4664 LC FAP crossed (murine 1‘surrogate)
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 223 - protein, 680 aa, synthetic construct
  REGION 1..680: note = P1AF4674 HC KK+ LTBR charges - FAP crossed (murine 2‘surrogate)
EVQLLESGGGLVQPGGSLRLSCAASGFTFSYYAMTWVRQAPGKGLEWIGVINSGGGTYYTDSVKGRFTMSRDNSKNTLYLQVNSLRAEDTAVYYCAKASTADVWGQGTTVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 224 - protein, 214 aa, synthetic construct; identical to SEQ ID NO: 220
  REGION 1..214: note = P1AF4674 LC LTBR charges (murine 2‘surrogate)

SEQ ID NO: 225 - protein, 437 aa, synthetic construct
  REGION 1..437: note = P1AF4674 HC DD- LTBR charges (murine 2‘surrogate)
EVQLLESGGGLVQPGGSLRLSCAASGFTFSYYAMTWVRQAPGKGLEWIGVINSGGGTYYTDSVKGRFTMSRDNSKNTLYLQVNSLRAEDTAVYYCAKASTADVWGQGTTVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 226 - protein, 212 aa, synthetic construct
  REGION 1..212: note = P1AF4674 LC FAP crossed (murine 2‘surrogate)
EIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 227 - protein, 448 aa, synthetic construct
  REGION 1..448: note = P1AE5929 HC (hu IgG1 PG LALA)
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARPSYYLDGWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 228 - protein, 220 aa, synthetic construct; identical to SEQ ID NO: 112
  REGION 1..220: note = P1AE5929 LC (hu IgG1 PG LALA)

SEQ ID NO: 229 - protein, 448 aa, synthetic construct
  REGION 1..448: note = P1AF7213 HC (hu IgG1 PG LALA)
QVQLVQSGAEVKKPGSSVKVSCKASGGTFSSYAISWVRQAPGQGLEWMGGIIPIHGTANYAQKFQGRVTITADKSTSTAYMELSSLRSEDTAVYYCARPSYYLDGWLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 230 - protein, 220 aa, synthetic construct; identical to SEQ ID NO: 112
  REGION 1..220: note = P1AF7213 LC (hu IgG1 PG LALA)

SEQ ID NO: 231 - protein, 456 aa, synthetic construct
  REGION 1..456: note = P1AE9452 HC (hu IgG1 PG LALA)
EVQLLESGGGLVQPGGSLRLSCAAAGFTFSSYPMTWVRQAPGKGLEWVSLISPSGATTYYADSVKGRFTISRDTSKNTLYLQMNSLRAEDTAVYYCAKNHYPDDVIIKNYYFYGVDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 232 - protein, 214 aa, synthetic construct; identical to SEQ ID NO: 188
  REGION 1..214: note = P1AE9452 LC (hu IgG1 PG LALA)

SEQ ID NO: 233 - protein, 441 aa, synthetic construct
  REGION 1..441: note = P1AE9450 HC (hu IgG1 PG LALA)
EVQLLESGGGLVQPGGSLRLSCAASGFTVSSYVMKWFRQAPGKGLEWVSSISGSGGRPFYADSVKGRFTISRDNSKNALYLQMNSLRAEDTAVYYCANDMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 234 - protein, 214 aa, synthetic construct
  REGION 1..214: note = P1AE9450 LC (hu IgG1 PG LALA)
DIQMTQSPSSLSASVGDRVTITCRASQDISSFLKWYQQKPGKAPTLLIYAASALQSGVPSRFSGSGSGTDFTLTISSLQPEDFATYYCQQYNTYPLTFGGGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 443 aa, synthetic construct
  REGION 1..443: note = P1AE9459 HC (hu IgG1 PG LALA)
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 236 - protein, 214 aa, synthetic construct
  REGION 1..214: note = P1AE9459 LC (hu IgG1 PG LALA)
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 442 aa, synthetic construct
  REGION 1..442: note = P1AF0080 HC (hu IgG1 PG LALA)
EVQLLESGGGLVQPGGSLRLSCAASGFTFSYYAMTWVRQAPGKGLEWIGVINSGGGTYYTDSVKGRFTMSRDNSKNTLYLQVNSLRAEDTAVYYCAKASTADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 238 - protein, 214 aa, synthetic construct
  REGION 1..214: note = P1AF0080 LC (hu IgG1 PG LALA)
DIQMTQSPSSLSASVGDRVTITCQASQGISNYLSWYQQKPGKAPKRLIFAASSLESGVPSRFSGSGSGTDFTLTISSLQPEDFATYYCQQYNSYSLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 239 - protein, 448 aa, synthetic construct
  REGION 1..448: note = P1AF0064 HC (hu IgG1 PG LALA)
EVQLQQYGAELGKPGTSVKLSCKVSGYNIGNTYIHWVIQRPGKGLEWIGRIDPTNGNTICTEKFKNKATLTADLSSNTAYMQLSQLKSDDTAIYFCAPTTLTTPDSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 240 - protein, 213 aa, synthetic construct; identical to SEQ ID NO: 152
  REGION 1..213: note = P1AF0064 LC (hu IgG1 PG LALA)

SEQ ID NO: 241 - protein, 451 aa, synthetic construct
  REGION 1..451: note = P1AF0079 HC (hu IgG1 PG LALA)
EVQLLESGGALVQPGGSLRLSCAASGFTFSSYAMSWVRQAPGKGLEWVSVITGSSSSIDYADSVKGRFTISRDNSKNTLYLQMNSLRAEDTAVYYCAKGGSGVWSISTDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 242 - protein, 214 aa, synthetic construct
  REGION 1..214: note = P1AF0079 LC (hu IgG1 PG LALA)
DIQMTQSPSFLSASVGDRVTITCQASQDISNYLNWYQQKPGKAPKHLIYAASSLHSGVPSRFSGSGSGAYFTLTITSLQPEDFATYYCQQSHSTPPTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 449 aa, synthetic construct
  REGION 1..449: note = P1AE9457 HC (hu IgG1 PG LALA)
EVQLLESGGGLVQPGGSLRLSCAASGFTFSNYAMTWVRQAPGKGLEWVSEISGSGGSTYYADSVKGRFTISRDNSKNTLYLQMNSLRAEDTAVYYCAKDDDGAYGDTMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 244 - protein, 214 aa, synthetic construct
  REGION 1..214: note = P1AE9457 LC (hu IgG1 PG LALA)
DIQMTQSPSSLSASIGDRVTITCRASQDISNYLNWYQQKPGKAPKLLIYAASALQSGVPSRFSGSGSGTDFTLTISSLQPEDFATYYCQHYKSYPYTFGQGTKVEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 448 aa, synthetic construct
  REGION 1..448: note = P1AE1873 (CBE11) HC (hu IgG1 PG LALA)
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 246 - protein, 214 aa, synthetic construct
  REGION 1..214: note = P1AE1873 (CBE11) LC (hu IgG1 PG LALA)
DIQMTQSPSSLSASVGDRVTITCKAGQDIKSYLSWYQQKPGKAPKLLIYYATRLADGVPSRFSGSGSGTDYTLTISSLQPEDFATYYCLQHGESPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - protein, 242 aa, synthetic construct
  REGION 1..242: note = Fc-depleter kh NC avi B (P1AA0981) (knob chain)
GLNDIFEAQKIEWHEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 244 aa, synthetic construct
  REGION 1..244: note = Fc-depleter kh NC avi B P1AA0981) (hole chain)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKSGGLNDIFEAQKIEWHE

SEQ ID NO: 249 - protein, 492 aa, synthetic construct
  REGION 1..492: note = human lymphotoxin alpha/beta single-chain avi his (P1AE1235)
KPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFALGGSGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGGSGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVGGSGLNDIFEAQKIEWHEGGHHHHHH

SEQ ID NO: 250 - protein, 484 aa, synthetic construct
  REGION 1..484: note = murine lymphotoxin alpha/beta single-chain avi his (P1AE1236)
KPAAHLVGYPSKQNSLLWRASTDRAFLRHGFSLSNNSLLIPTSGLYFVYSQVVFSGESCSPRAIPTPIYLAHEVQLFSSQYPFHVPLLSAQKSVYPGLQGPWVRSMYQGAVFLLSKGDQLSTHTDGISHLHFSPSSVFFGAFALGGSGLPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVGGSGLPAAHLIGAWMSGQGLSWEASQEEAFLRSGAQFSPTHGLALPQDGVYYLYCHVGYRGRTPPAGRSRARSLTLRSALYRAGGAYGRGSPELLLEGAETVTPVVDPIGYGSLWYTSVGFGGLAQLRSGERVYVNISHPDMVDYRRGKTFFGAVMVGGSGLNDIFEAQKIEWHEGGHHHHHH

SEQ ID NO: 251 - protein, 448 aa, synthetic construct
  REGION 1..448: note = monomeric huLTBR ECD (S28-M227) Fc-fusion avi biotinylated (P1AE2835) (knob chain)
SQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMASGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 252 - protein, 227 aa, synthetic construct
  REGION 1..227: note = monomeric huLTBR ECD (S28-M227) Fc-fusion avi biotinylated (P1AE2835) (hole chain)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 253 - protein, 444 aa, synthetic construct
  REGION 1..444: note = monomeric muLTBR ECD (S28-L223) Fc-fusion avi biotinylated (P1AE4410) (knob chain)
SQPQLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLLASGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 254 - protein, 227 aa, synthetic construct; identical to SEQ ID NO: 252
  REGION 1..227: note = monomeric muLTBR ECD (S28-L223) Fc-fusion avi biotinylated (P1AE4410)(hole chain)

SEQ ID NO: 255 - protein, 448 aa, synthetic construct
  REGION 1..448: note = monomeric cynoLTBR ectodomain (S28-M227) Fc fusion avi biotinylated (P1AE4411) (knob chain)
SQPQVVPPYGSENQTCRDQEKEYYEPRHRICCSRCPPGTYVSAKCSRSRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCEDQGLVEAAPGTAQSDTTCRNPSESLPPEMSGTMLMASGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 256 - protein, 227 aa, synthetic construct; identical to SEQ ID NO: 252
  REGION 1..227: note = monomeric cynoLTBR ectodomain (S28-M227) Fc fusion avi biotinylated (P1AE4411) (hole chain)

SEQ ID NO: 257 - protein, 419 aa, synthetic construct
  REGION 1..419: note = monomeric N-terminal human LTbR (ECD full length) hu IgG1 Fc-fusion kih HRYF avi (P1AE1217)(knob chain)
QTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKASGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 258 - protein, 227 aa, synthetic construct; identical to SEQ ID NO: 252
  REGION 1..227: note = monomeric N-terminal human LTbR (ECD full length) hu IgG1 Fc-fusion kih HRYF avi (P1AE1217)(hole chain)

SEQ ID NO: 259 - protein, 15 aa, synthetic construct
  REGION 1..15: note = Avi tag
GLNDIFEAQKIEWHE

SEQ ID NO: 260 - protein, 456 aa, synthetic construct
  REGION 1..456: note = human LTBR ECD (S28-M227) N- and C-terminally extended - Fc-fusion wt kih HRYF - Avi - His biotinylated 261(P1AE7979)(knob chain)
SQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLMASGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHEGGHHHHHH

SEQ ID NO: 261 - protein, 227 aa, synthetic construct; identical to SEQ ID NO: 252
  REGION 1..227: note = human LTBR ECD (S28-M227) N- and C-terminally extended - Fc-fusion wt kih HRYF - Avi - His biotinylated (P1AE7979)(hole chain)

SEQ ID NO: 262 - protein, 419 aa, synthetic construct
  REGION 1..419: note = monomeric N-terminal cyno LTbR (ECD full length) hu IgG1 Fc-fusion kih HRYF avi biotinylated (P1AE2656)(knob chain)
QTCRDQEKEYYEPRHRICCSRCPPGTYVSAKCSRSRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCEDQGLVEAAPGTAQSDTTCRASGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 263 - protein, 227 aa, synthetic construct; identical to SEQ ID NO: 252
  REGION 1..227: note = monomeric N-terminal cyno LTbR (ECD full length) hu IgG1 Fc-fusion kih HRYF avi biotinylated (P1AE2656)(hole chain)

SEQ ID NO: 264 - protein, 768 aa, synthetic construct
  REGION 1..768: note = human FAP - Avi - His biotinylated (P1AA5347)
RPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSVDEQLYFQGGSGLNDIFEAQKIEWHEARAHHHHHH

SEQ ID NO: 265 - protein, 770 aa, synthetic construct
  REGION 1..770: note = murine FAP - Avi - His biotinylated (P1AD9907)
RPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGILSGRSQNHLYTHMTHFLKQCFSLSDVDEQLYFQGGSGLNDIFEAQKIEWHEARAHHHHHH

SEQ ID NO: 266 - protein, 5 aa, synthetic construct
  REGION 1..5: note = LTBR (5929) CDR-H2 patch
IPIFG

SEQ ID NO: 267 - DNA, 40 bp, synthetic construct
  misc_feature 1..40: note = HUJH5.HFc-DO3
  source 1..40: mol_type = other DNA
cttgggcccttggtacttgctgaggagacggtgaccaggg

SEQ ID NO: 268 - DNA, 40 bp, synthetic construct
  misc_feature 1..40: note = HUJH6.HFc-Do3
  source 1..40: mol_type = other DNA
cttgggcccttggtacttgctgaggagacggtgaccgtgg

SEQ ID NO: 269 - DNA, 42 bp, synthetic construct
  misc_feature 1..42: note = BcPCR_FHLC_leader.fw (TG)
  source 1..42: mol_type = other DNA
acagaggaagcttgccgccaccatggacatgagggtccccgc

SEQ ID NO: 270 - DNA, 24 bp, synthetic construct
  misc_feature 1..24: note = HuCK.Do.2AA
  source 1..24: mol_type = other DNA
tgaagacagatggtgcagccacag

SEQ ID NO: 271 - DNA, 26 bp, synthetic construct
  misc_feature 1..26: note = WTRat.VH.FW11
  source 1..26: mol_type = other DNA
sargtvmagctgvwgsagtcwggrss

SEQ ID NO: 272 - DNA, 26 bp, synthetic construct
  misc_feature 1..26: note = WTRat.VH.FW12
  source 1..26: mol_type = other DNA
saggtgmagctsswgsarwcwggrss

SEQ ID NO: 273 - DNA, 26 bp, synthetic construct
  misc_feature 1..26: note = WTRat.VH.FW13
  source 1..26: mol_type = other DNA
sargtkmarctgswgsagtcwggrss

SEQ ID NO: 274 - DNA, 26 bp, synthetic construct
  misc_feature 1..26: note = WTRat.VH.FW14
  source 1..26: mol_type = other DNA
smggtgmagctkswksagtcwggwss

SEQ ID NO: 275 - DNA, 26 bp, synthetic construct
  misc_feature 1..26: note = WTRat.VH.FW15
  source 1..26: mol_type = other DNA
aggtkmmkctgawrsartcwggmsst

SEQ ID NO: 276 - DNA, 26 bp, synthetic construct
  misc_feature 1..26: note = WTRat.VH.FW16
  source 1..26: mol_type = other DNA
saggtrmagctgvwrsagtcwggrsv

SEQ ID NO: 277 - DNA, 26 bp, synthetic construct
  misc_feature 1..26: note = WTRat.VH.FW17
  source 1..26: mol_type = other DNA
aggtgcagctdvwrsagwcwggrssw

SEQ ID NO: 278 - DNA, 19 bp, synthetic construct
  misc_feature 1..19: note = 682.rev do
  source 1..19: mol_type = other DNA
gggaagatgaagacagatg

SEQ ID NO: 279 - DNA, 25 bp, synthetic construct
  misc_feature 1..25: note = WTRat.VK.FW1
  source 1..25: mol_type = other DNA
gryrtyswgwtgacmcardctccdb

SEQ ID NO: 280 - DNA, 25 bp, synthetic construct
  misc_feature 1..25: note = WTRat.VK.FW2
  misc_feature 15: note = n is a, c, g, or t
  source 1..25: mol_type = other DNA
gmtrttgtkmtsamncardcwccab

SEQ ID NO: 281 - DNA, 29 bp, synthetic construct
  misc_feature 1..29: note = WTRat.CKappa.Do2
  source 1..29: mol_type = other DNA
gacgggtgaggatgatgtcttatgaacaa

SEQ ID NO: 282 - protein, 761 aa, Mus musculus
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 283 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 284 - protein, 5 aa, Homo sapiens
EPKSC

SEQ ID NO: 285 - protein, 6 aa, synthetic construct
  REGION 1..6: note = CH1 connector C-terminal end D-variant
EPKSCD

SEQ ID NO: 286 - protein, 6 aa, synthetic construct
  REGION 1..6: note = CH1 connector C-terminal end S-variant
EPKSCS

SEQ ID NO: 287 - protein, 10 aa, synthetic construct
  REGION 1..10: note = MISC_FEATURE - X being S or P
  SITE 8: note = misc_feature - X can be any naturally occurring amino acid
DKTHTCPXCP

SEQ ID NO: 288 - protein, 7 aa, synthetic construct
  REGION 1..7: note = MISC_FEATURE - X being S or P
  SITE 5: note = misc_feature - X can be any naturally occurring amino acid
HTCPXCP

SEQ ID NO: 289 - protein, 5 aa, synthetic construct
  REGION 1..5: note = MISC_FEATURE - X being S or P
  SITE 3: note = misc_feature - X can be any naturally occurring amino acid
CPXCP

SEQ ID NO: 290 - protein, 107 aa, Homo sapiens
APELLGGPSVFLFPPKPKDTLMISRTPEVTCVWDVSHEDPEVKFNWYVDGVEVHNAKTKPREEQESTYRWSVLTVLHQDWLNGKEYKCKVSNKALPAPIEKTISKAK

SEQ ID NO: 291 - protein, 105 aa, Homo sapiens
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 292 - protein, 330 aa, Homo sapiens [sequence omitted]

SEQ ID NO: 293 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 294 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 295 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 296 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 297 - protein, 415 aa, Mus musculus
MRLPRASSPCGLAWGPLLLGLSGLLVASQPQLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLLLAILLSLVLFLLFTTVLACAWMRHPSLCRKLGTLLKRHPEGEESPPCPAPRADPHFPDLAEPLLPMSGDLSPSPAGPPTAPSLEEVVLQQQSPLVQARELEAEPGEHGQVAHGANGIHVTGGSVTVTGNIYIYNGPVLGGTRGPGDPPAPPEPPYPTPEEGAPGPSELSTPYQEDGKAWHLAETETLGCQDL

SEQ ID NO: 298 - protein, 5 aa, synthetic construct
  REGION 1..5: note = Peptide linker (G4S)
GGGGS

SEQ ID NO: 299 - protein, 10 aa, synthetic construct
  REGION 1..10: note = Peptide linker (G4S)2
GGGGSGGGGS

SEQ ID NO: 300 - protein, 10 aa, synthetic construct
  REGION 1..10: note = Peptide linker (SG4)2
SGGGGSGGGG

SEQ ID NO: 301 - protein, 14 aa, synthetic construct
  REGION 1..14: note = Peptide linker G4(SG4)2
GGGGSGGGGSGGGG

SEQ ID NO: 302 - protein, 10 aa, synthetic construct
  REGION 1..10: note = peptide linker
GSPGSSSSGS

SEQ ID NO: 303 - protein, 15 aa, synthetic construct
  REGION 1..15: note = (G4S)3 peptide linker
GGGGSGGGGSGGGGS

SEQ ID NO: 304 - protein, 20 aa, synthetic construct
  REGION 1..20: note = (G4S)4 peptide linker
GGGGSGGGGSGGGGSGGGGS

SEQ ID NO: 305 - protein, 8 aa, synthetic construct
  REGION 1..8: note = peptide linker
GSGSGSGS

SEQ ID NO: 306 - protein, 8 aa, synthetic construct
  REGION 1..8: note = peptide linker
GSGSGNGS

SEQ ID NO: 307 - protein, 8 aa, synthetic construct
  REGION 1..8: note = peptide linker
GGSGSGSG

SEQ ID NO: 308 - protein, 6 aa, synthetic construct
  REGION 1..6: note = peptide linker
GGSGSG

SEQ ID NO: 309 - protein, 4 aa, synthetic construct
  REGION 1..4: note = peptide linker
GGSG

SEQ ID NO: 310 - protein, 8 aa, synthetic construct
  REGION 1..8: note = peptide linker
GGSGNGSG

SEQ ID NO: 311 - protein, 8 aa, synthetic construct
  REGION 1..8: note = peptide linker
GGNGSGSG

SEQ ID NO: 312 - protein, 6 aa, synthetic construct
  REGION 1..6: note = peptide linker
GGNGSG

SEQ ID NO: 313 - protein, 118 aa, synthetic construct [sequence omitted]

SEQ ID NO: 314 - protein, 107 aa, synthetic construct [sequence omitted]

SEQ ID NO: 315 - protein, 121 aa, synthetic construct
  REGION 1..121: note = VH (PD-L1)
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 316 - protein, 108 aa, synthetic construct
  REGION 1..108: note = VL (PD-L1)
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 317 - DNA, 37 bp, synthetic construct
  misc_feature 1..37: note = rbHC.up
  source 1..37: mol_type = other DNA
aagcttgccaccatggagactgggctgcgctggcttc

SEQ ID NO: 318 - protein, 681 aa, synthetic construct
  REGION 1..681: note = P1AG5459 HC KK LTBR with charges FAP crossed (murine 2‘surrogate)
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 319 - protein, 214 aa, synthetic construct
  REGION 1..214: note = P1AG5459 LC LTBR with charges (murine 2‘surrogate)
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKRADAAPTVSIFPPSSRKLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 320 - protein, 438 aa, synthetic construct
  REGION 1..438: note = P1AG5459 HC DD- LTBR with charges (murine 2‘surrogate)
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKEQMAKDKVSLTCMITNFFPEDITVEWQWNGQPAENYDNTQPIMDTDGSYFVYSDLNVQKSNWEAGNTFTCSVLHEGLHNHHTEKSLSHSPG

SEQ ID NO: 321 - protein, 212 aa, synthetic construct; identical to SEQ ID NO: 226
  REGION 1..212: note = P1AG5459 LC FAP crossed (murine 2‘surrogate)

SEQ ID NO: 322 - protein, 680 aa, synthetic construct
  REGION 1..680: note = P1AG5461 HC KK LTBR with charges DP47 crossed (non targeted murine 2‘surrogate)
EVQLVESGGGLVQPGGSLRLSCAASGFTFSSYTMNWVRQAPGKGLEWVSGVAPSGDRTYYTDSVKGRFTISRDNAKNTLDLQMTSLRAEDAAIYICAQGFAFTNWGQGTMVTVSSAKTTPPSVYPLAPGSAAQTNSMVTLGCLVEGYFPEPVTVTWNSGSLSSGVHTFPAVLQSDLYTLSSSVTVPSSTWPSQTVTCNVAHPASSTKVDEKIVPRDCGCKPCICTVPEVSSVFIFPPKPKDVLTITLTPKVTCVVVAISKDDPEVQFSWFVDDVEVHTAQTKPREEQINSTFRSVSELPIMHQDWLNGKEFKCRVNSAAFGAPIEKTISKTKGRPKAPQVYTIPPPKKQMAKDKVSLTCMITNFFPEDITVEWQWNGQPAENYKNTQPIMKTDGSYFVYSKLNVQKSNWEAGNTFTCSVLHEGLHNHHTEKSLSHSPGGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 323 - protein, 214 aa, synthetic construct; identical to SEQ ID NO: 319
  REGION 1..214: note = P1AG5461 LC LTBR with charges (non-targeted murine 2‘surrogate)

SEQ ID NO: 324 - protein, 438 aa, synthetic construct; identical to SEQ ID NO: 320
  REGION 1..438: note = P1AG5461 HC DD- LTBR with charges (non-targeted murine 2‘surrogate)

SEQ ID NO: 325 - protein, 212 aa, synthetic construct
  REGION 1..212: note = P1AG5461 LC DP47 crossed (non-targeted murine 2‘surrogate)
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDC

SEQ ID NO: 326 - protein, 586 aa, synthetic construct
  REGION 1..586: note = P1AE1079(2+1) HC knob LTBR (CBE11) FAP VH
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSS

SEQ ID NO: 327 - protein, 214 aa, synthetic construct; identical to SEQ ID NO: 246
  REGION 1..214: note = P1AG1079(2+1) LC LTBR (CBE11)

SEQ ID NO: 328 - protein, 577 aa, synthetic construct
  REGION 1..577: note = P1AG1079(2+1) HC hole LTBR (CBE11) FAP VL
EVQLVESGGGLVKPGGSLRLSCAASGFTFSDYYMYWFRQAPGKGLEWVATISDGGSYTYYPDSVKGRFTISRDNAKNSLYLQMSSLRAEDTAVYYCAREENGNFYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSGGGGSGGGGSEIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIK

SEQ ID NO: 329 - protein, 438 aa, synthetic construct
  REGION 1..438: note = P1AH5884 (1+1) HC knob LTBR (BHA10) crossed
DIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 330 - protein, 446 aa, synthetic construct; identical to SEQ ID NO: 125
  REGION 1..446: note = P1AH5884 (1+1) HC hole FAP with charges

SEQ ID NO: 331 - protein, 223 aa, synthetic construct
  REGION 1..223: note = P1AH5884 (1+1) LC LTBR (BHA10) crossed
QVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 332 - protein, 215 aa, synthetic construct; identical to SEQ ID NO: 110
  REGION 1..215: note = P1AH5884 (1+1) LC FAP with charges

SEQ ID NO: 333 - protein, 678 aa, synthetic construct
  REGION 1..678: note = P1AH5885 (2+1) HC knob LTBR (BHA10) crossed, FAP with charges
DIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGEVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 334 - protein, 438 aa, synthetic construct
  REGION 1..438: note = P1AH5885 (2+1) HC hole LTBR (BHA10) crossed
DIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 335 - protein, 223 aa, synthetic construct; identical to SEQ ID NO: 331
  REGION 1..223: note = P1AH5885 (2+1) LC LTBR (BHA10) crossed

SEQ ID NO: 336 - protein, 215 aa, synthetic construct; identical to SEQ ID NO: 110
  REGION 1..215: note = P1AH5885 (2+1) LC FAP with charges

SEQ ID NO: 337 - protein, 680 aa, synthetic construct
  REGION 1..680: note = P1AH5886 (2+1) HC knob LTBR (9459) crossed, FAP (212) with charges
DIQMTQSPSSLSASVGDRVTITCRASQGISSALAWFQQKPGKAPKRLIFDASSLDSGVPSRFSGSGSGTDFTLTISSLQPEDFATYYCQQYNNYPLTFGGGTKVEMKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGQVQLVQSGAEVKKPGASVKVSCKASGYTLTDYNMDWVRQAPGQGLEWIGDIYPNTGGTIYNQKFKGRVTMTIDTSTSTVYMELSSLRSEDTAVYYCTRFRGIHYAMDYWGQGTTVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCD

SEQ ID NO: 338 - protein, 438 aa, synthetic construct; identical to SEQ ID NO: 201
  REGION 1..438: note = P1AH5886 (2+1) HC hole LTBR (9459) crossed

SEQ ID NO: 339 - protein, 222 aa, synthetic construct; identical to SEQ ID NO: 140
  REGION 1..222: note = P1AH5886 (2+1) LC LTBR (9459) crossed

SEQ ID NO: 340 - protein, 218 aa, synthetic construct
  REGION 1..218: note = P1AH5886 (2+1) LC FAP (212) with charges
EIVLTQSPATLSLSPGERATLSCRASESVDNYGLSFINWFQQKPGQAPRLLIYGTSNRGSGIPARFSGSGSGTDFTLTISSLEPEDFAVYFCQQSNEVPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 341 - protein, 445 aa, synthetic construct
  REGION 1..445: note = P1AH0119 HC (LTBR BHA10 hu IgG1 PG LALA)
QVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 342 - protein, 214 aa, synthetic construct
  REGION 1..214: note = P1AH0119 LC (LTBR BHA10 hu IgG1 PG LALA)
DIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 5 aa, synthetic construct
  REGION 1..5: note = LTBR (BHA10) CDR-H1
TYYLH

SEQ ID NO: 344 - protein, 17 aa, synthetic construct
  REGION 1..17: note = LTBR (BHA10) CDR-H2
WIYPGNVHAQYNEKFKG

SEQ ID NO: 345 - protein, 7 aa, synthetic construct
  REGION 1..7: note = LTBR (BHA10) CDR-H3
SWEGFPY

SEQ ID NO: 346 - protein, 11 aa, synthetic construct
  REGION 1..11: note = LTBR (BHA10) CDR-L1
KASQNVGINVA

SEQ ID NO: 347 - protein, 7 aa, synthetic construct
  REGION 1..7: note = LTBR (BHA10) CDR-L2
SASYRYS

SEQ ID NO: 348 - protein, 9 aa, synthetic construct
  REGION 1..9: note = LTBR (BHA10) CDR-L3
QQYDTYPFT

SEQ ID NO: 349 - protein, 116 aa, synthetic construct
  REGION 1..116: note = LTBR (BHA10) VH
QVQLVQSGAEVKKPGSSVKVSCKASGYTFTTYYLHWVRQAPGQGLEWMGWIYPGNVHAQYNEKFKGRVTITADKSTSTAYMELSSLRSEDTAVYYCARSWEGFPYWGQGTTVTVSS

SEQ ID NO: 350 - protein, 107 aa, synthetic construct
  REGION 1..107: note = LTBR (BHA10) VL
DIQMTQSPSSLSASVGDRVTITCKASQNVGINVAWYQQKPGKAPKSLISSASYRYSGVPSRFSGSGSGTDFTLTISSLQPEDFATYFCQQYDTYPFTFGQGTKVEIK

SEQ ID NO: 351 - protein, 17 aa, Homo sapiens
QCRCQPGMFCAAWALEC

SEQ ID NO: 352 - protein, 12 aa, Homo sapiens
AWALECTHCELL

SEQ ID NO: 353 - protein, 16 aa, Homo sapiens
QTCRDQEKEYYEPQHR

SEQ ID NO: 354 - protein, 8 aa, Homo sapiens
SRIRDTVC

SEQ ID NO: 355 - protein, 14 aa, Homo sapiens
SYNEHWNYLTICQL

SEQ ID NO: 356 - protein, 18 aa, Homo sapiens
WALECTHCELLSDCPPGT

SEQ ID NO: 357 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 358 - protein, 167 aa, Homo sapiens
DGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 359 - protein, 200 aa, Homo sapiens
SQPQAVPPYASENQTCRDQEKEYYEPQHRICCSRCPPGTYVSAKCSRIRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCENQGLVEAAPGTAQSDTTCKNPLEPLPPEMSGTMLM

SEQ ID NO: 360 - protein, 196 aa, Mus musculus
SQPQLVPPYRIENQTCWDQDKEYYEPMHDVCCSRCPPGEFVFAVCSRSQDTVCKTCPHNSYNEHWNHLSTCQLCRPCDIVLGFEEVAPCTSDRKAECRCQPGMSCVYLDNECVHCEEERLVLCQPGTEAEVTDEIMDTDVNCVPCKPGHFQNTSSPRARCQPHTRCEIQGLVEAAPGTSYSDTICKNPPEPGAMLL

SEQ ID NO: 361 - protein, 200 aa, Macaca fascicularis
SQPQVVPPYGSENQTCRDQEKEYYEPRHRICCSRCPPGTYVSAKCSRSRDTVCATCAENSYNEHWNYLTICQLCRPCDPVMGLEEIAPCTSKRKTQCRCQPGMFCAAWALECTHCELLSDCPPGTEAELKDEVGKGNNHCVPCKAGHFQNTSSPSARCQPHTRCEDQGLVEAAPGTAQSDTTCRNPSESLPPEMSGTMLM